(19) United States
(12) Patent Application Publication
(10) Pub. No.: US 2026/0139264 A1
(43) Pub. Date: May 21, 2026

(54) Compositions And Methods For Production Of Salidroside, Icariside D2, And Precursors Of Salidroside And Icariside D2

(71) Applicant: Whitehead Institute for Biomedical Research, Cambridge, MA (US)

(72) Inventors: Michael Torrens-Spence, Cambridge, MA (US); Jing-Ke Weng, Belmont, MA (US)

(21) Appl. No.: 19/258,235

(22) Filed: Jul. 2, 2025

Related U.S. Application Data
(63) Continuation of application No. 17/847,313, filed on Jun. 23, 2022, now Pat. No. 12,385,055.
Application No. 17/847,313 is a continuation of application No. 16/224,257, filed on Dec. 18, 2018, now Pat. No. 11,408,009.
(60) Provisional application No. 62/607,271, filed on Dec. 18, 2017.

(51) Int. Cl. C12N 15/82; C12N 15/52; C12N 15/70; C12N 15/74; C12N 15/81; C12P 17/06

(52) U.S. Cl. CPC C12N 15/8257; C12N 15/52; C12N 15/70; C12N 15/743; C12N 15/81; C12N 15/8243; C12N 15/8245; C12P 17/06; C12Y 114/13068; C12Y 204/0117; C12Y 401/01

(57) ABSTRACT

Transgenic host cells, vectors useful for making transgenic host cells, and kits useful for making transgenic host cells are described. Also described are transgenic plants. In some embodiments, transgenic host cells express a 4-hydroxyphenylacetaldehyde synthase (4HPAAS). In some embodiments, transgenic host cells express a tyrosol:UDP-glucose 8-O-glucosyltransferase (T8GT). The transgenic host cells are useful for biosynthesis of one or more of salidroside, icariside D2, tyrosol, and 4-hydroxypenylacetaldehyde.

### CROSS-REFERENCE TO RELATED APPLICATIONS

This application is a continuation of U.S. application Ser. No. 17/847,313, filed on Jun. 23, 2022, which is a continuation of U.S. application Ser. No. 16/224,257, filed on Dec. 18, 2018, now U.S. Pat. No. 11,408,009, issued on Aug. 9, 2022, which claims the benefit of U.S. Provisional Application No. 62/607,271, filed on Dec. 18, 2017. The entire teachings of the above applications are incorporated herein by reference.

### INCORPORATION BY REFERENCE OF MATERIAL IN XML

This application incorporates by reference the Sequence Listing contained in the following eXtensible Markup Language (XML) file being submitted concurrently herewith:
a) File name: 0399.2060-003_SL.xml; created: Jan. 28, 2026; 387,981 Bytes in size.

### BACKGROUND

Salidroside, also known as tyrosol 8-O-glucoside, is naturally produced by plants within the Rhodiola genus. Salidroside is of particular interest and value because of its unique reported biological activities (Cifani et al., 2010; Guan et al., 2012; Panossian et al., 2014). However, commercially available salidroside in its pure form is currently obtained through a lengthy purification process from its native plant host, which poses a significant bottleneck hindering further clinical development of salidroside as a potential therapeutic agent. Accordingly, improved methods of making salidroside are needed.

### SUMMARY

Salidroside is a bioactive tyrosine-derived phenolic natural product found in medicinal plants under the Rhodiola genus. In addition to their anti-fatigue and anti-anoxia roles in traditional medicine, Rhodiola total extract and salidroside have also displayed medicinal properties as anti-cardiovascular disease, and anti-cancer, agents. The resulting surge in global demand of Rhodiola plants and salidroside has driven some species close to extinction.

Described herein is a Rhodiola salidroside biosynthetic pathway that was elucidated utilizing comprehensive transcriptomics and metabolomics datasets for Rhodiola rosea. This pathway includes a pyridoxal phosphate (PLP)-dependent 4-hydroxyphenylacetaldehyde synthase (4HPAAS) that directly converts tyrosine to 4-HPAA. Genes encoding the subsequent 4-HPAA reductase (4HPAR) and tyrosol:UDP-glucose 8-O-glucosyltransferase (T8GT), respectively, were further identified to complete salidroside biosynthesis in Rhodiola. As described herein, heterologous production of salidroside can be achieved in yeast Saccharomyces cerevisiae as well as in plant Nicotiana benthamiana through transgenic expression of Rhodiola salidroside biosynthetic genes. Accordingly, the methods and compositions described herein provide useful tools for engineering sustainable production of salidroside in heterologous hosts.

Described herein are vectors and kits that include vectors. Those vectors include a nucleic acid encoding one or more of a 4-hydroxyphenylacetaldehyde synthase (4HPAAS), a 4-hydroxyphenylacetaldehyde reductase (4HPAR), a tyrosol:UDP-glucose 8-O-glucosyltransferase (T8GT), and a tyrosol:UDP-glucose 4-O-glucosyltransferase (T4GT). Described herein are methods of using the vectors and kits to make a transgenic host cell having a transgene encoding one or more of a 4H1PAAS, a 4HPAR, a T8GT, and a T4GT. Described herein are methods of making one or more of 4-hydroxyphenylacetaldehyde (4-HPAA), tyrosol, tyrosol 8-O-glucoside (salidroside), and icariside D2 in a transgenic host cell. The tyrosol, salidroside, and/or icariside D2 can subsequently be obtained, e.g., by separation and purification processes. A variety of transgenic host cells can be used, such as yeast cells, plant cells, and bacterial cells. In some embodiments, the tyrosol, tyrosol 8-O-glucoside (salidroside), or icariside D2 can be obtained in greater quantities than by purification from the native plant host. In some embodiments, the tyrosol, tyrosol 8-O-glucoside (salidroside), or icariside D2 can be obtained more cost-effectively than by purification from the native plant host.

Certain embodiments provide a vector that includes a nucleic acid encoding a 4-hydroxyphenylacetaldehyde synthase (4HPAAS), wherein the 4H1PAAS has at least 70% sequence identity to either SEQ ID NO: 2 (Rhodiola rosea 4HPAAS), or a biologically active fragment thereof. The 4H1PAAS includes: a) an amino acid residue selected from the group consisting of F, L, I, M and V at a position corresponding to the F residue at position 343 in SEQ ID NO: 2; b) an amino acid residue selected from the group consisting of N and D at a position corresponding to the H residue at position 198 in SEQ ID NO: 2; or c) a combination thereof.

Certain embodiments provide a vector that includes a nucleic acid encoding a 4-hydroxyphenylacetaldehyde reductase (4HPAR), wherein the 4HPAR includes at least 70% amino acid sequence identity to SEQ ID NO: 4, or a biologically active fragment thereof.

Certain embodiments provide a vector that includes a nucleic acid encoding a tyrosol:UDP-glucose 8-O-glucosyltransferase (T8GT). In some embodiments, the T8GT comprises a plant secondary product glycosyltransferase (PSPG) motif. In some embodiments, the T8GT comprises at least 70% amino acid sequence identity to one or more of SEQ ID NO: 14, SEQ ID NO: 16, SEQ ID NO: 18, and SEQ ID NO: 20, or a biologically active fragment thereof.

Certain embodiments provide a vector that includes a nucleic acid encoding a tyrosol:UDP-glucose 4-O-glucosyltransferase (T4GT). In some embodiments, the T4GT comprises a plant secondary product glycosyltransferase (PSPG) motif. In some embodiments, the T4GT comprises at least 70% amino acid sequence identity to one or more of SEQ ID NO: 6, SEQ ID NO: 8, SEQ ID NO: 10, SEQ ID NO: 12, and SEQ ID NO: 14, or a biologically active fragment thereof.

Certain embodiments provide a kit that includes: a) a vector that includes a nucleic acid encoding a 4-hydroxyphenylacetaldehyde synthase (4HPAAS); b) a vector that includes a nucleic acid encoding a 4-hydroxyphenylacetaldehyde reductase (4HPAR); and c) one or more of i) a vector that includes a nucleic acid encoding a tyrosol:UDP-glucose 8-O-glucosyltransferase (T8GT) and ii) a vector that includes a nucleic acid encoding a tyrosol:UDP-glucose 4-O-glucosyltransferase (T4GT). In some embodiments, the kit includes both a T8GT and a T4GT.

Some embodiments provide a host cell that includes a transgene encoding a tyrosol:UDP-glucose 8-O-glucosyltransferase (T8GT). In some embodiments, the host cell further includes a transgene encoding 4-hydroxyphenylacetaldehyde reductase (4HPAR). In some embodiments, the host cell further includes a transgene encoding a 4-hydroxyphenylacetaldehyde synthase (4HPAAS). In some embodiments, the host cell further includes a transgene encoding both a 4HPAR and a 4H1PAAS. In some embodiments, a single transgene encodes multiple genes, such as one or more of the T8GT, the 4HPAR, and the 4HPAAS. In some embodiments, separate transgenes encode one or more of T8GT, 4HPAR, and 4HPAAS.

Some embodiments provide a host cell that includes a transgene encoding a 4-hydroxyphenylacetaldehyde synthase (4HPAAS). In some embodiments, the host cell further includes a transgene encoding 4-hydroxyphenylacetaldehyde reductase (4HPAR). In some embodiments, the host cell further includes a transgene encoding tyrosol:UDP-glucose 8-O-glucosyltransferase (T8GT). In some embodiments, the host cell further includes a transgene encoding tyrosol:UDP-glucose 4-O-glucosyltransferase (T4GT). In some embodiments, the host cell further includes a transgene encoding both a 4HPAR and a T8GT. In some embodiments, the host cell further includes a transgene encoding both a 4HPAR and a T4GT. In some embodiments, a single transgene encodes multiple genes, such as one or more of the 4H1PAAS, the 4HPAR, the T8GT, and the T4GT. In some embodiments, separate transgenes encode one or more of the 4H1PAAS, the 4HPAR, the T8GT, and the T4GT.

Some embodiments provide a method of making a transgenic host cell. The method can include introducing a vector into the host cell, wherein the vector includes a nucleic acid encoding a tyrosol:UDP-glucose 8-O-glucosyltransferase (T8GT). The method can further include introducing into the host cell a vector that includes a nucleic acid encoding a 4-hydroxyphenylacetaldehyde reductase (4HPAR). The method can further include introducing into the host cell a vector that includes a nucleic acid encoding a 4-hydroxyphenylacetaldehyde synthase (4HPAAS).

Some embodiments provide a method of making a transgenic host cell. The method can include introducing a vector into the host cell, wherein the vector includes a nucleic acid encoding a 4-hydroxyphenylacetaldehyde synthase (4HPAAS). The method can further include introducing into the host cell a vector that includes a nucleic acid encoding a 4-hydroxyphenylacetaldehyde reductase (4HPAR). The method can further include introducing into the host cell a vector that includes a nucleic acid encoding tyrosol:UDP-glucose 8-O-glucosyltransferase (T8GT) or a tyrosol:UDP-glucose 4-O-glucosyltransferase (T4GT). In some embodiments, the method can further include introducing into the host cell a vector that includes a nucleic acid encoding a T8GT and a nucleic acid encoding a T4GT.

Certain embodiments provide a method of making tyrosol 8-O-glucoside (salidroside). In some embodiments, the salidroside is made in a host cell. In certain embodiments, the salidroside is made in a cell-free system or cell lysate. The method can include expressing in a host cell a transgene that encodes a tyrosol:UDP-glucose 8-O-glucosyltransferase (T8GT). In some embodiments, the host cell includes tyrosol, either produced endogenously or provided to the cell exogenously.

In some embodiments, the host cell further expresses a transgene that encodes a 4-hydroxyphenylacetaldehyde reductase (4HPAR). In some embodiments, the host cell further expresses a transgene that encodes a 4-hydroxyphenylacetaldehyde synthase (4HPAAS). In some embodiments, tyrosol is secreted by the host cell into the cell culture media, from which it can be obtained.

Certain embodiments provide a method of making 4-hydroxyphenylacetaldehyde (4-HPAA). In some embodiments, the 4-HPAA is made in a host cell. In some embodiments, the 4-H1PAA is made in a cell-free system or lysate. The method can include expressing in the host cell a transgene that encodes a 4-hydroxyphenylacetaldehyde synthase (4HPAAS). In some embodiments, the host cell includes L-tyrosine, produced endogenously or provided to the cell exogenously. In some embodiments, the method further includes making tyrosol in the host cell, and the host cell further expresses a transgene encoding a 4-hydroxyphenylacetaldehyde reductase (4H1PAR). In some embodiments, tyrosol is secreted by the host cell into the cell culture media, from which it can be obtained.

In some embodiments, the host cell is a yeast cell, such as Saccharomyces cerevisiae. In some embodiments, the host cell is a plant cell, such as a cell a from a Nicotiana benthamiana plant. In some embodiments, the host cell is a bacterial cell, such as Escherichia coli or Agrobacterium tumefaciens.

In some embodiments, nucleic acids encoding two or more of 4HPAAS, 4HPAR, T8GT, and T4GT are included in a single vector. In some embodiments, the transgene encoding an enzyme (e.g., 4H1PAAS, 4HPAR, T8GT, and T4GT) can be integrated into the genome of the host transgenic cell.

Certain embodiments provide a transgenic plant, such as a Nicotiana benthamiana plant, that includes a transgene encoding a tyrosol:UDP-glucose 8-O-glucosyltransferase (T8GT).

Some embodiments provide a transgenic plant, such as a Nicotiana benthamiana plant, that includes a transgene encoding a tyrosol:UDP-glucose 4-O-glucosyltransferase (T4GT).

Some embodiments provide a transgenic plant, such as a Nicotiana benthamiana plant, that includes a transgene encoding a 4-hydroxyphenylacetaldehyde synthase (4HPAAS).

Certain embodiments provide a transgenic plant, such as a Nicotiana benthamiana plant, that includes a transgene encoding a 4-hydroxyphenylacetaldehyde reductase (4HPAR).

Certain embodiments provide an isolated deoxyribonucleic acid (DNA) coding sequence encoding a tyrosol:UDP-glucose 8-O-glucosyltransferase (T8GT). In some embodiments, the nucleic acid includes SEQ ID NO: 13. In some embodiments, the nucleic acid includes SEQ ID NO: 15. In some embodiments, the nucleic acid includes SEQ ID NO: 17. In some embodiments, the nucleic acid includes SEQ ID NO: 19.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-C show salidroside biosynthesis in R. rosea. FIG. 1A is a photograph of greenhouse-grown R. rosea. FIG. 1B is a chromatogram showing metabolic profiling of R. rosea root and crown tissues by LC-HRAM-MS. Enrichment of tyrosol and salidroside is observed in the root. Extracted ion chromatogram (XIC) is shown with mass windows set to display the [M−H]⁻ ion for tyrosol and the [M+NH₄]⁺ ion for salidroside. The identity of the metabolites was verified in comparison to authentic standards. FIG. 1C is a schematic showing alternative salidroside biosynthetic pathways in Rhodiola.

FIGS. 2A-C show identification and characterization of the Rr4HPAAS. FIG. 2A is a simplified maximum likelihood (ML) phylogenetic tree of land plant AAADs. A fully annotated version of this tree is shown in FIG. 11. The three major groups of the tree have been annotated as the basal (green), TyDC (blue) and TDC (red) clades based on taxonomic distribution, cladding, and conservation of the substrate-specifying active site residue. Representative characterized enzymes are labeled at the tree branches, while the R. rosea TDC, AAS and 4H1PAAS are displayed in bold. The scale measures evolutionary distances in substitutions per amino acid. FIG. 2B is LC-UV chromatograms of the reaction product of L-tyrosine and Rr4HPAAS enzyme (with and without NaBH₄ reduction) in comparison to enzyme assay conducted using PsTyDC as a control. The identity of the products was verified by comparison with authentic standards. FIG. 2C is a graph showing kinetic characterization of Rr4HPAAS against various aromatic amino acid substrates.

FIGS. 3A-C show identification and characterization of two R. rosea 4HPARs. FIG. 3A is a simplified ML phylogenetic tree of angiosperm ADHs. A fully annotated version of this tree is shown in FIG. 14. Major clades are annotated based on representative characterized enzymes when possible. The two R. rosea 4HPARs and the previously characterized SIPARs are labeled at the tree branches. The scale measures evolutionary distances in substitutions per amino acid. FIG. 3B is LC-MS chromatograms of the reaction product of 4-HPAA and 0.2 g recombinant Rr4HPAR1 after incubation for various time points. FIG. 3C is LC-MS chromatograms of the reaction product of 4-HPAA and 15 g recombinant Rr4HPAR2 after incubation for various time points. The identity of the tyrosol product was verified by comparison with an authentic standard.

FIGS. 4A-C show identification and characterization of R. rosea tyrosol-modifying UGTs. FIG. 4A is a maximum likelihood (ML) phylogenetic tree of 34 R. rosea UGTs together with 88 full-length UGTs encoded by the A. thaliana genome. UGTs that show T4GT and T8GT activities are denoted by black circles and stars, respectively. Bootstrap values (based on 500 replicates) are indicated at the major nodes. The scale measures evolutionary distances in substitutions per amino acid. FIG. 4B a chart showing relative in vivo T4GT and T8GT activities of R. rosea UGTs as examined in engineered yeast. FIG. 4C is a graph showing Michaelis-Menten kinetic characterization of four R. rosea tyrosol-modifying UGTs.

FIGS. 5A-C show heterologous production of tyrosine-derived metabolites in transgenic N. benthamiana as detected by LC-HRAM-MS. FIG. 5A is a chromatogram showing that N. benthamiana transiently expressing Rr4HPAAS or Pc4HPAAS produces both salidroside and icariside D2. FIG. 5B is a chromatogram showing that N. benthamiana transiently expressing PsTyDC produces tyramine. FIG. 5C is a chromatogram showing that N. benthamiana leaves transiently co-expressing Rr4HPAAS and RrT4GT or RrT8GT produce predominantly icariside D2 or salidroside, respectively. XICs are shown with mass windows set to display the [M+NH₄]⁺ ion for salidroside and icariside D2, and the [M+H]⁺ ion for tyramine. The identity of the metabolites was verified by comparison with authentic standards.

FIG. 6 shows the chemical structures of a number of Rhodiola glycosylated natural products.

FIG. 7 is a multiple sequence alignment highlighting the sequence regions that can influence enzyme substrate selectivity in select plant AAAD family members. Sequences represent various enzymes from the TyDC and TDC clades. The residue framed in black (identified as Gly 370 for C. roseus) can impact substrate selectivity (indolic vs. phenolic). Columns framed in blue indicate greater than 70% conservation of residue physico-chemical properties. Identical amino acids are in white font boxed in red, while similar residues are displayed in red font. FIG. 7 includes SEQ ID Nos. 207 through 219, in order from top-to-bottom.

FIGS. 8A-B show total ion count of the root and crown R. rosea extractions. FIG. 8A is a chromatogram of positive ion mode metabolites. FIG. 8B is a chromatogram of negative ion mode metabolites.

FIG. 9 is a chromatogram showing relative abundance of rosavin between R. rosea tissue types. The differential location of the natural product rosavin [M+NH₄]⁺ ion between R. rosea root and crown. The identity of rosavin was verified by comparison to an authentic standard

FIG. 10 is a multiple sequence alignment highlighting the residue that dictate decarboxylation and aldehyde synthase chemistry in plant AAADs family members. Sequences represent plant AAADs performing either decarboxylation chemistry or aldehyde synthase chemistry (highlighted in green). The three identified R. rosea AAAD sequences are also displayed. Investigation of the activity influencing residue (boxed in black) suggests that the R. rosea sequence from the TDC clade likely catalyzes decarboxylation chemistry while the basal and TyDC clade R. rosea AAS catalyze aldehyde synthase chemistry. Columns framed in blue indicate greater than 70% conservation of residue physico-chemical properties. Identical amino acids are in white font boxed in red, while similar residues are displayed in red font. FIG. 10 includes SEQ ID Nos. 220 through 240, in order from top-to-bottom.

FIG. 11 is a phylogenetic tree of embryophyte AAADs. This tree is populated with sequences from all Phytozome V12 embryophyte species, the three AAAD like sequences from the R. rosea transcriptomes (shown in bold) and all attainable characterized NCBI AAAD sequences (also shown in bold). Green, red and blue branches correspond to the basal, TDC and TyDC clades, respectively. These clades were determined through the application of the indolic substrate selective active site glycine (red clade), the phenolic substrate selective serine (blue clade), their taxonomic distribution (green clade exists in all sampled species and is most closely related to chlorophytes species) and representative characterized sequences.

FIG. 12 is a graph showing relative hydrogen peroxide production for the Rr4HPAAS, the RrAAS and the PsTyDC. 100 μL reaction mixtures containing 50 μg of recombinant enzyme, 2 mM L-tyrosine, 50 mM Tris pH 8.0 and 200 μM PLP were incubated at 30° C. for various time points prior to quenching with 100 μL of 0.8 μM formic acid. Hydrogen peroxide levels of quenched reaction mixtures were subsequently analyzed using Pierce Quantitative Peroxide Assay Kit against a standard curve of hydrogen peroxide.

FIG. 13 is a chart showing relative TPM for the Rr4HPAAS transcript between the root and crown transcriptomes.

FIG. 14 is a phylogenetic tree of angiosperm ADHs. This tree is populated with sequences from various Phytozome V12 angiosperm species, ADLs like sequences from the R. rosea transcriptomes and two characterized S/PAR sequences. Characterized A. thaliana enzymes, the two S/PARs and the two Rr4HPARs are show in bold. The different colors have been applied to distinguish between various clades.

FIG. 15 is a chromatogram showing the enzymatic reduction of phenylacetaldehyde to phenylethyl alcohol by Rr4HPAR1 and Rr4HPAR2. Chromatogram of positive ion mode metabolites between 50 and 300 m/z show the depletion of phenylacetaldehyde and production of phenylethyl alcohol when exposed to NADPH and Rr4HPAR1 or RrPAR2. Reactions were carried out in 200 μL 50 mM Tris, pH 8.0 in the presence of 2 mM phenylacetaldehyde, 5 mM NADPH and 50 μg of recombinant enzyme. The reactions were incubated at 30° C. for 25 minutes prior to quenching with 200 μL of 0.8 M formic acid, extracted with 100 μL of ethyl acetate and analyzed by gas chromatography-mass spectrometry. Phenylethyl alcohol was verified by comparison to an authentic standard.

FIG. 16 is a chromatogram showing the enzymatic reduction of 4-HPAA to tyrosol by Rr4HPAR1 and Rr4HPAR2. LC-UV chromatograms of products generated from coupled enzyme assays conducted using Rr4HPAAS in combination with Rr4HPAR1 or Rr4HPAR2, respectively. Enzyme assay conducted using Rr4HPAAS alone is included as a control. 100 μl reaction mixtures containing 50 mM Tris pH 8.0, 4 mM tyrosine, 2 μg cataylase and 100 g of Rr4HPAAS were incubated at 30 degrees C. for 1 hour. 10 mM NADPH and 10 g of Rr4HPAR1 or Rr4HPAR2 was then added and incubated for an additional 15 minutes at which point the reactions were quenched with an equal volume of 0.8 M formic acid and analyzed by LC-UV. The identity of the product was verified by comparing the elution profile and UV spectrum to that of an authentic tyrosol standard.

FIG. 17 is a phylogenetic tree of the 113 curated full-length non redundant R. rosea transcriptome UGTs. Sequences profiled for tyrosol glycosylation activity in yeast have been annotated RrUGT1-34.

FIG. 18 is a chromatogram of the tyrosol [M−H]⁻ ion generated in transgenic yeast expressing the Rr4HPAAS, the Rr4HPAAS+Rr4HPAR1 or Rr4HPAAS+Rr4HPAR2. The identity was verified by comparison to commercially purchased tyrosol.

FIGS. 19A-B show R. rosea tyrosol UGTs as compared to previously characterized Rhodiola tyrosol UGTs. FIG. 19A is a chromatogram of the tyrosol glycoside [M+NH₄]⁺ production using the newly described RrUGT3, RrUGT33 and the previously described R. sachalinensis UGTs (GenBank: AAS55083 and EU567325). FIG. 19B is a graph showing relative icariside D2 and salidroside production from RrT8HGT, RrT4GHT RsAAS55083 and RsEU567325. The identity of the ions was confirmed by comparison to NMR verified standards.

FIG. 20 is the structure of salidroside.

FIG. 21 is the structure of icariside D2.

FIG. 22 is a ¹H NMR spectrum (400 MHz, CDCl₃) of salidroside isolated from N. benthamiana leaves overexpressing R. rosea salidroside biosynthetic genes. δ: 9.16 (1H, s, OH), 7.03 (2H, d, J=8.4, 4-H, 8-H), 6.65 (2H, d, J=8.4, 5-H, 7-H), 4.92 (3H, m, Glu-OH), 4.47 (1H, s, Glu-OH), 4.16 (1H, d, J=7.6, 1′-H), 3.87 (1H, m, 1-H), 3.65 (1H, m, 6′-H), 3.56 (1H, m, 1-H), 3.42 (1H, m, 6′-H), 3.12 (1H, m, 3′-H), 3.07 (1H, m, 5′-H), 3.04 (1H, m, 4′-H), 2.95 (1H, m, 2′-H), 2.73 (2H, m, 2-H).

FIG. 23 is a ¹³C NMR spectrum (100 MHz, CDCl₃) of salidroside isolated from N. benthamiana leaves overexpressing R. rosea salidroside biosynthetic genes. δ: 155.6 (6-C), 129.7 (4, 8-C), 128.6 (3-C), 115.0 (5, 7-C), 102.8 (1′-C), 76.9 (3′-C), 76.8 (5′-C), 73.4 (2′-C), 70.1 (1-C), 69.9 (4′-C), 61.1 (6′-C), 34.8 (2-C).

FIG. 24 is a heteronuclear multiple bond correlation (HMBC) spectrum of salidroside isolated from N. benthamiana leaves overexpressing R. rosea salidroside biosynthetic genes.

FIG. 25 is a ¹H NMR spectrum (400 MHz, CDCl₃) of icariside D2 isolated from N. benthamiana leaves overexpressing R. rosea salidroside biosynthetic genes. δ: 7.11 (2H, d, J=8.8, 4-H, 8-H), 6.92 (2H, d, J=8.8, 5-H, 7-H), 5.28 (1H, s, Glu-OH), 5.09 (1H, s, Glu-OH), 5.02 (1H, s, Glu-OH), 4.78 (1H, d, J=7.2, 1′-H), 4.61 (1H, m, 1-H), 4.56 (1H, m, 1-H), 3.68 (1H, s, 6′-H), 3.55 (1H, m, OH), 3.45 (1H, s, 6′-H), 3.14-3.32 (4H, m, 2′, 3′, 4′, 5′-H), 2.66 (2H, m, 2-H).

FIG. 26 is a ¹³C NMR spectrum (100 MHz, CDCl₃) of icariside D2 isolated from N. benthamiana leaves overexpressing R. rosea salidroside biosynthetic genes. δ: 155.8 (6-C), 132.7 (3-C), 129.7 (4, 8-C), 116.1 (5, 7-C), 100.6 (1′-C), 77.0 (3′-C), 76.6 (5′-C), 73.3 (2′-C), 69.7 (4′-C), 62.4 (1-C), 60.7 (6′-C), 38.2 (2-C).

FIG. 27 is a chromatogram of the tyrosol [M−H]⁻ ion generated in transgenic N. benthamiana expressing the Rr4HPAA. The addition of either the RrT4HGT or the RrT8HGT depletes the tyrosol substrate in the production of icariside D2 or salidroside. The identity of the ions was verified against authentic standards.

FIG. 28 is a chart showing titer of salidroside producing S. cerevisiae strains with and without substrate feeding. The first bar of the bar graph represents salidroside titer in wild type (WT) BY4743 yeast while the second bar demonstrates salidroside titer from the yeast strains expressing the native Rr4HPAAS and RrT8GT genes in separate pTEF 2 plasmids. The third bar of the graph illustrates the salidroside titer from the S. cerevisiae strain transformed with a pTDH3 promoter 2 multi gene plasmid containing coRr4HPAAS and coRrT8GT genes. The fourth and fifth bars show salidroside production from the aforementioned codon optimized multi gene plasmid with the addition of either 4 mM L-tyrosine or 4 mM tyrosol. The final bar shows the salidroside production from a strain containing the multi gene coRr4HPAAS and coRrT8GT plasmid additionally transformed with a second 2 pTDH3 ARO4 K229L and ARO7 G141S multi gene plasmid.

FIG. 29A-B are chromatograms of the salidroside [M+NH₄]⁺ ion generated in transgenic S. cerevisiae. FIG. 29A is a chromatogram of salidroside production in wild type (WT), native Rr4HPAAS and RrT8GT expressing, coRr4HPAAS and coRrT8GT expressing or ARO4 K229L, ARO7 G141S, coRr4HPAAS and coRrT8GT expressing S. cerevisiae strains. FIG. 29B is a chromatogram of salidroside production in S. cerevisiae expressing coRr4HPAAS and coRrT8GT with and without the addition of L-tyrosine and tyrosol.

FIG. 30 is a graph showing total ion count of salidroside producing transgenic S. cerevisiae. Salidroside, labeled in the chromatogram, appears as one of the principle metabolites.

FIG. 31 is a multiple sequence alignment of key residues within biochemically characterized plant AAADs. The multiple sequence alignment of FIG. 31 shows portions of the full alignment of FIG. 32. FIG. 31 includes SEQ ID Nos. 241 through 257 corresponding to sequences beginning at position 190, in order from top-to-bottom; and SEQ ID Nos. 275-291 corresponding to sequences beginning at position 332, in order from top-to-bottom.

FIG. 32 is a multiple sequence alignment of key residues within biochemically characterized plant AAADs. FIG. 32 includes SEQ ID Nos. 258 through 274, in order from top-to-bottom.

FIG. 33 is a chart showing sequence conservation for plant AAAD activity dictating residues. Multiple sequence alignments of the queried AAAD sequences evaluated for active site conservation using WebLogo. Polar amino acids are green, basic amino acids are blue, acidic amino acids are red and hydrophobic amino acids are black. The y-axis units (bits) display the maximum entropy for the given residue. The representative residues from the Rr4HPAAS MF674522 sequence are listed below with residue numbers.

FIGS. 34A-C show product formation of PsTyDC and mutants. FIG. 34A is a chromatogram showing the reduced enzyme product of tyrosine incubated with wildtype PsTyDC. FIG. 34B is a chromatogram showing the reduced enzyme product of tyrosine incubated with PsTyDC Y350F. FIG. 34C is a chromatogram showing the reduced enzyme product of tyrosine incubated with PsTyDC H204N.

FIG. 35 is a depiction of active site conformations of Catharanthus roseus tryptophan decarboxylase. In this homodimer, the A chain is shown in green while the B chain is shown in blue. The active site ligand (tryptophan) is shown in yellow. The active site lysine bound pyridoxal phosphate (LLP) cofactor is visible in the B chain.

### DETAILED DESCRIPTION

A description of example embodiments follows.

### Rhodiola and Salidroside Biosynthesis

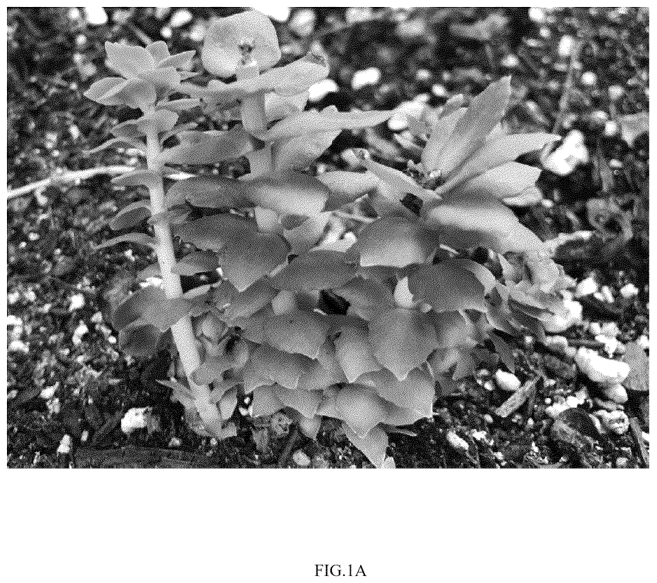

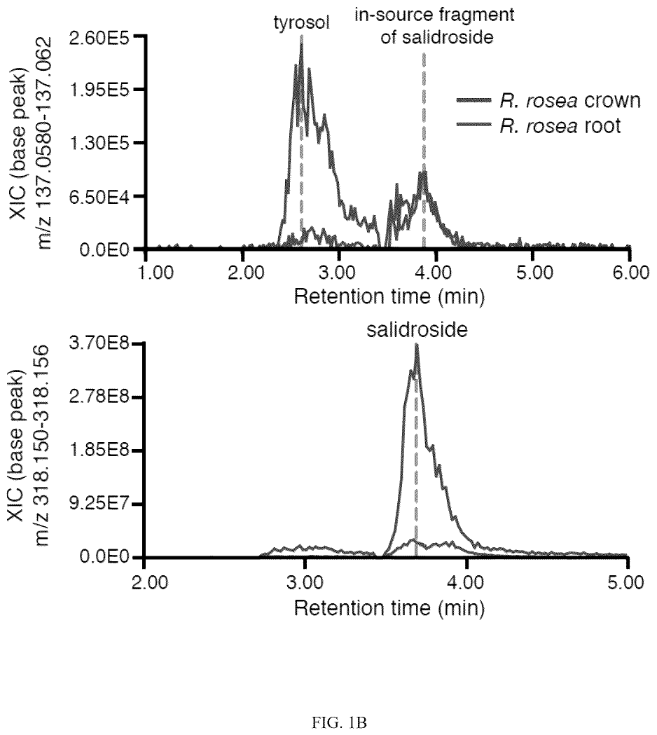

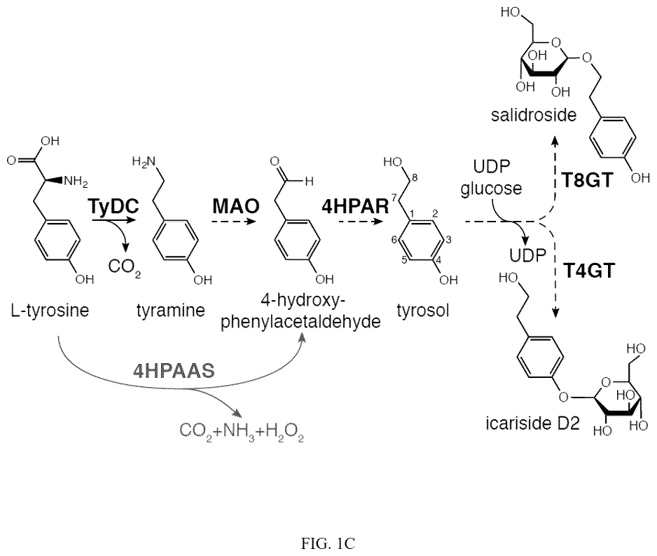

The Rhodiola genus consists of approximately 90 species of high-altitude and cold tolerant perennial plants of the Crassulaceae family native to the arctic regions of Eurasia and North America (FIG. 1A). Select species from this genus have a long history in traditional medicine with purported roles in bolstering immunity, memory and learning, while ameliorating depression, altitude sickness and fatigue (Fu, 2009; Lei et al., 2006). Recent studies of Rhodiola extract have also demonstrated antioxidant and anti-inflammatory properties with potential applications in the prevention of cardiovascular diseases and cancer (Gauger et al., 2010; Khanum et al., 2005; Skopinska-Rozewska et al., 2008; Tu et al., 2008; Zhang et al., 2007). Extensive phytochemical analysis of Rhodiola has identified a number of specialized glycosides, including rosiridin, rhodionin, rosarin, rosin, rosavin and salidroside (FIG. 6) (Du and Xie, 1995; Rohloff, 2002; Yang et al., 2012; Yousef et al., 2006). Salidroside, or tyrosol 8-O-glucoside, is of particular interest and value because of its unique reported biological activities (Cifani et al., 2010; Guan et al., 2012; Panossian et al., 2014). However, commercially available salidroside in its pure form is currently obtained through a lengthy purification process from its native plant host, which poses a significant bottleneck hindering further clinical development of salidroside as a potential therapeutic agent. Moreover, surging global demand of wild Rhodiola plants as a herbal supplement has led to overharvesting of these ecologically vulnerable plants from their native habitats with some species now threatened by extinction (Booker et al., 2016; Dorji, 2016).

Metabolic engineering is a promising approach to gain access to high-value plant natural products as an alternative to direct compound isolation from plant hosts (O'Connor, 2015). Previous attempts to engineer salidroside biosynthesis in heterologous hosts have utilized a selection of plant and yeast enzymes to assemble artificial salidroside biosynthetic pathways (Bai et al., 2014; Chung et al., 2017). Although these studies demonstrated the feasibility of engineering salidroside production in bacterial hosts (Bai et al., 2014), an unresolved native salidroside biosynthetic pathway inplanta hinders further development and improvement of salidroside biosynthetic strategies in bacteria and other alternative chassis organisms. In postulated salidroside biosynthetic pathway, the salidroside aglycone tyrosol is generated from tyrosine through sequential decarboxylation, oxidative deamination, and aldehyde reduction reactions, catalyzed by three discrete enzymes, tyrosine decarboxylase (TyDC), monoamine oxidase (MAO) and 4H1PAR, respectively (FIG. 1C) (Lan et al., 2013). Tyrosol is then glycosylated at its 8-OH group by a regio-specific uridine 5′-diphospho-glucosyltransferase (UGT) to yield salidroside. Although the proposed salidroside pathway seems plausible, to date, only one enzyme of this proposed pathway, Rhodiola crenulata TyDC (RcTyDC) (GenBank AFN89854.1), has been previously recombinantly expressed and experimentally examined (Lan et al., 2013). Overexpression of this TyDC-like gene in R. crenulata hairy roots culture led to increased accumulation of salidroside (Lan et al., 2013).

TyDCs, together with tryptophan decarboxylases (TDCs) and aromatic acetaldehyde synthases (AASs), encompass a large family of PLP-dependent enzymes broadly referred to as the plant AAAD family (Facchini et al., 2000; Kaminaga et al., 2006). As their respective names imply, TyDCs, TDCs and AAS catalyze discrete decarboxylation or decarboxylation-deamination reactions using specific aromatic amino acids as substrates.

To resolve Rhodiola salidroside biosynthesis, tissue-specific transcriptomics and metabolomics datasets were generated for R. rosea. Using a combination of differential expression analysis, phylogenetic analysis, biochemical characterization, and heterologous expression, a set of Rhodiola genes encoding 4H1PAAS, 4H1PAR, and T8GT to complete salidroside biosynthesis from tyrosine were identified. In addition, a number of regio-specific T4GTs capable of producing icariside D2 were identified. The newly acquired knowledge about phenolic glycoside biosynthesis in Rhodiola allowed reconstitution of salidroside or icariside D2 biosynthesis in yeast S. cerevisiae as well as in the plant N. benthamiana.

### Aromatic Amino Acid Decarboxylases (AAAD) Family of Enzymes

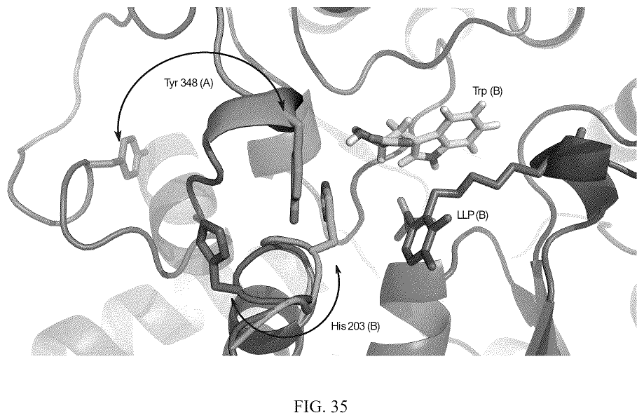

TyDCs, together with tryptophan decarboxylases (TDCs) and aromatic acetaldehyde synthases (AASs), encompass a large family of PLP-dependent enzymes broadly referred to as the plant AAAD family (Facchini et al., 2000; Kaminaga et al., 2006). Thus, the AAAD family encompasses enzymes with aromatic amino acid decarboxylase activity and enzymes with aromatic acetaldehyde synthase activity. (Torrens-Spence et al., 2012; Torrens-Spence et al., 2013). Without wishing to be bound by theory, the catalytic mechanism of the AAAD family of enzymes is contingent on the conformational change of two active site loops, which is illustrated in FIG. 35 with respect to a tryptophan decarboxylase from Catharanthus roseus. The large loop from the A chain (342-359) undergoes a dramatic conformational change from a solvent exposed active site “open” conformation to an active site obscured “closed” conformation. Concurrently, a small loop from chain B (201-205) undergoes a crank shaft conformational change to move from a solvent exposed “open” conformation to a pyridoxal phosphate (LLP) associated “closed” conformation. Key residues in these dynamic loops play important roles in the catalytic mechanism of AAAD enzymes. In the tryptophan decarboxylase from Catharanthus roseus, tyrosine 348 (Chain A) functions as a catalytic acid to donate a proton to the carbanion intermediate in the decarboxylation reaction mechanism while histidine 203 (Chain B) functions as a molecular chaperon responsible for coordinating and enabling proton donation of the acid tyrosine 348. Substitution of either residue abolishes the protonation and enables a peroxy-aldimine intermediate through the attack of molecular oxygen which spontaneously decomposes to yield the corresponding aromatic acetylaldehyde, peroxide and ammonia aldehyde synthase products. Consequently, substitutions at either location function as a primary sequence means for biochemical functional prediction. One of skill in the art will understand that the precise location within the sequence (here, tyrosine at 348 and histidine at 203) varies among related enzymes within the AAAD family.

### Nucleic Acids

As used herein, the term “nucleic acid” refers to a polymer comprising multiple nucleotide monomers (e.g., ribonucleotide monomers or deoxyribonucleotide monomers). “Nucleic acid” includes, for example, DNA (e.g., genomic DNA and cDNA), RNA, and DNA-RNA hybrid molecules. Nucleic acid molecules can be naturally occurring, recombinant, or synthetic. In addition, nucleic acid molecules can be single-stranded, double-stranded or triple-stranded. In certain embodiments, nucleic acid molecules can be modified. In the case of a double-stranded polymer, “nucleic acid” can refer to either or both strands of the molecule.

The terms “nucleotide” and “nucleotide monomer” refer to naturally occurring ribonucleotide or deoxyribonucleotide monomers, as well as non-naturally occurring derivatives and analogs thereof. Accordingly, nucleotides can include, for example, nucleotides comprising naturally occurring bases (e.g., adenosine, thymidine, guanosine, cytidine, uridine, inosine, deoxyadenosine, deoxythymidine, deoxyguanosine, or deoxycytidine) and nucleotides comprising modified bases known in the art.

As used herein, “wildtype” refers to the canonical amino acid sequence as found in nature. As those of skill in the art would appreciate, a nucleic acid sequence can be modified, e.g., for codon optimization in a host cell (e.g., bacteria, yeast, and plant host cells).

As used herein, the term “sequence identity,” refers to the extent to which two nucleotide sequences, or two amino acid sequences, have the same residues at the same positions when the sequences are aligned to achieve a maximal level of identity, expressed as a percentage. For sequence alignment and comparison, typically one sequence is designated as a reference sequence, to which a test sequences are compared. The sequence identity between reference and test sequences is expressed as the percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide or amino acid residue at 70% of the same positions over the entire length of the reference sequence.

Alignment of sequences for comparison to achieve maximal levels of identity can be readily performed by a person of ordinary skill in the art using an appropriate alignment method or algorithm. In some instances, the alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology).

When using a sequence comparison algorithm, test and reference sequences are input into a computer, subsequent coordinates are designated, if necessary, and sequence algorithm program parameters are designated. The sequence comparison algorithm then calculates the percent sequence identity for the test sequence(s) relative to the reference sequence, based on the designated program parameters. A commonly used tool for determining percent sequence identity is Protein Basic Local Alignment Search Tool (BLASTP) available through National Center for Biotechnology Information, National Library of Medicine, of the United States National Institutes of Health. (Altschul et al., 1990).

In various embodiments, two nucleotide sequences, or two amino acid sequences, can have at least, e.g., 70%, 75%, 80%, 85%, 86%, 87%, 88%, 89%, 90%, 91%, 92%, 93%, 94%, 95%, 96%, 97%, 98%, 99%, or more, sequence identity. When ascertaining percent sequence identity to one or more sequences described herein, the sequences described herein are the reference sequences.

Some embodiments of the invention relate to a nucleic acid coding sequence (e.g., dsDNA, cDNA) encoding one or more of the enzymes described herein, including those nucleic acid sequences provided in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, and SEQ ID NO: 19.

### Enzymes

As used herein, the term 4-hydroxyphenylacetaldehyde synthase (4HPAAS) refers to an enzyme that catalyzes conversion of L-tyrosine to 4-hydroxyphenylacetaldehyde. Methods and assays for determining whether an enzyme catalyzes conversion of L-tyrosine to 4-hydroxyphenylacetaldehyde are known in the art, and include enzyme activity assays and liquid chromatography to assess retention time of metabolites, as described herein. Chemical structure can also be assessed by nuclear magnetic resonance (NMR) or liquid chromatography-mass spectrometry. An example of a 4HPAAS is SEQ ID NO: 2, which is the amino acid sequence of a 4HPAAS identified in Rhodiola rosea (Rr4HPAAS). In some embodiments, a 4HPAAS has at least about 70%, 75%, 80%, 85%, 86%, 87%, 88%, 89%, 90%, 91%, 92%, 93%, 94%, 95%, 96%, 97%, 98%, 99%, or more sequence identity to SEQ ID NO: 2, or a biologically active fragment thereof. In some embodiments, a 4HPAAS has: a) an amino acid residue selected from the group consisting of F, L, I, M and V at a position corresponding to the F residue at position 343 in SEQ ID NO: 2; b) an amino acid residue selected from the group consisting of N and D at a position corresponding to the H residue at position 198 in SEQ ID NO: 2; or c) a combination thereof. Typically, a 4HPAAS has at least 70% sequence identity to SEQ ID NO: 2, or a biologically active fragment thereof, and also: a) an amino acid residue selected from the group consisting of F, L, I, M and V at a position corresponding to the F residue at position 343 in SEQ ID NO: 2; b) an amino acid residue selected from the group consisting of N and D at a position corresponding to the H residue at position 198 in SEQ ID NO: 2; or c) a combination thereof. An example of a nucleic acid coding sequence that encodes a 4HPAAS is SEQ ID NO: 1, which encodes an amino acid having SEQ ID NO: 2. Many different nucleic acids can encode the 4H1PAAS of SEQ ID NO: 2 due to the degeneracy of the genetic code. Nucleic acids can also differ from SEQ ID NO: 1, for example, as a result of one or more substitutions (e.g., silent substitutions).

In some embodiments, modified enzymes can be used in the methods and host cells described herein to provide 4H1PAAS activity in those host cells and methods. Typically, those modified enzymes have a) an amino acid residue selected from the group consisting of F, L, I, M and V at a position corresponding to the F residue at position 343 in SEQ ID NO: 2; b) an amino acid residue selected from the group consisting of N and D at a position corresponding to the H residue at position 198 in SEQ ID NO: 2; or c) a combination thereof. In certain embodiments, modified Papaver somniferum tyrosine decarboxylase (PsTyDC) enzymes comprising a substitution of the active site histidine (e.g., with N or D) at the position corresponding to the H residue at position 198 in SEQ ID NO: 2, and/or the active site tyrosine (e.g., with F, L, I, M or V) corresponding to the F residue at position 343 in SEQ ID NO: 2, can be used in the methods and host cells described herein to provide 4H1PAAS activity in those host cells and methods. In some embodiments, modified nucleic acids encoding the modified enzymes can be used in the vectors, kits, and methods described herein. In some embodiments, those nucleic acids may be codon optimized for expression in a host cell.

As used herein, the term 4-hydroxyphenylacetaldehyde reductase (4HPAR) refers to an enzyme that catalyzes conversion of 4-hydroxyphenylacetaldehyde to tyrosol. Methods and assays for determining whether an enzyme catalyzes conversion of 4-hydroxyphenylacetaldehyde to tyrosol are known in the art, and include enzyme activity assays and liquid chromatography to assess retention time of metabolites, as described herein. Chemical structure can also be assessed by nuclear magnetic resonance (NMR) or liquid chromatography-mass spectrometry. An example of a 4HPAR is SEQ ID NO: 4, which is the amino acid sequence of a 4HPAR identified in Rhodiola rosea (Rr4HPAR). In some embodiments, a 4H1PAR has at least about 70%, 75%, 80%, 85%, 86%, 87%, 88%, 89%, 90%, 91%, 92%, 93%, 94%, 95%, 96%, 97%, 98%, 99%, or more sequence identity to SEQ ID NO: 4, or a biologically active fragment thereof. An example of a nucleic acid that encodes a 4HPAR is SEQ ID NO: 3, which encodes an amino acid having SEQ ID NO: 4. Many different nucleic acids can encode the 4HPAR of SEQ ID NO: 4 due to the degeneracy of the genetic code. Nucleic acids can also differ from SEQ ID NO: 3, for example, as a result of one or more substitutions (e.g., conservative substitutions, non-conservative substitutions), deletions, or insertions, or a combination thereof, with respect to the wild-type Rr4HPAR sequence (SEQ ID NO: 3).

As used herein, the term tyrosol:UDP-glucose 8-O-glucosyltransferase (T8GT) refers to an enzyme that catalyzes conversion of tyrosol to tyrosol 8-O-glucoside (salidroside). Methods and assays for determining whether an enzyme catalyzes conversion of tyrosol to tyrosol 8-O-glucoside (salidroside) are known in the art, and include enzyme activity assays and liquid chromatography to assess retention time of metabolites, as described herein. Chemical structure can also be assessed by nuclear magnetic resonance (NMR) or liquid chromatography-mass spectrometry. Examples of T8GTs are SEQ ID NO: 14, SEQ ID NO: 16, SEQ ID NO: 18, and SEQ ID NO: 20, which are the amino acid sequences of T8GTs identified in Rhodiola rosea (RrT8GTs). In some embodiments, a T8GT has at least about 70%, 75%, 80%, 85%, 86%, 87%, 88%, 89%, 90%, 91%, 92%, 93%, 94%, 95%, 96%, 97%, 98%, 99%, or more sequence identity to one or more of SEQ ID NO: 14, SEQ ID NO: 16, SEQ ID NO: 18, and SEQ ID NO: 20, or biologically active fragments thereof. Examples of nucleic acids that encode T8GTs are SEQ ID NO: 13, SEQ ID NO: 15, SEQ ID NO: 17, and SEQ ID NO: 19, which encode amino acids having SEQ ID NO: 14, SEQ ID NO: 16, SEQ ID NO: 18, and SEQ ID NO: 20, respectively. Many different nucleic acids can encode the T8GTs due to the degeneracy of the genetic code. Nucleic acids can also differ, for example, as a result of one or more substitutions (e.g., silent substitutions), with respect to any of the wild-type RrT8GT nucleic acid sequences.

As used herein, the term tyrosol:UDP-glucose 4-O-glucosyltransferase (T4GT) refers to an enzyme that catalyzes conversion of tyrosol to tyrosol 4-O-glucoside (icariside D2). Methods and assays for determining whether an enzyme catalyzes conversion of tyrosol to tyrosol 4-O-glucoside (icariside D2) are known in the art, and include enzyme activity assays and liquid chromatography to assess retention time of metabolites, as described herein. Chemical structure can also be assessed by nuclear magnetic resonance (NMR) or liquid chromatography-mass spectrometry. Examples of T4GTs are SEQ ID NO: 6, SEQ ID NO: 8, SEQ ID NO: 10, SEQ ID NO: 12, and SEQ ID NO: 14, which are the amino acid sequences of T4GTs identified in Rhodiola rosea (RrT4GTs). In some embodiments, a T4GT has at least about 70%, 75%, 80%, 85%, 86%, 87%, 88%, 89%, 90%, 91%, 92%, 93%, 94%, 95%, 96%, 97%, 98%, 99%, or more sequence identity to one or more of SEQ ID NO: 6, SEQ ID NO: 8, SEQ ID NO: 10, SEQ ID NO: 12, and SEQ ID NO: 14, or biologically active fragments thereof. Examples of nucleic acids that encode T8GTs are SEQ ID NO: 5, SEQ ID NO: 7, SEQ ID NO: 9, SEQ ID NO: 11, and SEQ ID NO: 13, which encode amino acids having SEQ ID NO: 6, SEQ ID NO: 8, SEQ ID NO: 10, SEQ ID NO: 12, and SEQ ID NO: 14, respectively. Many different nucleic acids can encode the T4GTs due to the degeneracy of the genetic code. Nucleic acids can also differ, for example, as a result of one or more substitutions (e.g., silent substitutions) with respect to any of the wild-type RrT4GT nucleic acid sequences. Vectors

The terms “vector”, “vector construct” and “expression vector” mean the vehicle by which a DNA or RNA sequence (e.g. a foreign gene) can be introduced into a host cell, so as to transform the host and promote expression (e.g. transcription and translation) of the introduced sequence. Vectors typically comprise the DNA of a transmissible agent, into which foreign DNA encoding a protein is inserted by restriction enzyme technology. A common type of vector is a “plasmid”, which generally is a self-contained molecule of double-stranded DNA that can readily accept additional (foreign) DNA and which can readily introduced into a suitable host cell. A large number of vectors, including plasmid and fungal vectors, have been described for replication and/or expression in a variety of eukaryotic and prokaryotic hosts.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. A polynucleotide or polypeptide is expressed recombinantly, for example, when it is expressed or produced in a foreign host cell under the control of a foreign or native promoter, or in a native host cell under the control of a foreign promoter. Gene delivery vectors generally include a transgene (e.g., nucleic acid encoding an enzyme) operably linked to a promoter and other nucleic acid elements required for expression of the transgene in the host cells into which the vector is introduced. Suitable promoters for gene expression and delivery constructs are known in the art. For bacterial host cells, suitable promoters, include, but are not limited to promoters obtained from the E. coli lac operon, Streptomyces coelicolor agarase gene (dagA), Bacillus subtilis levansucrase gene (sacB), Bacillus lichenformis alpha-amylase gene (amyL), Bacillus stearothermophilus maltogenic amylase gene (amyM), Bacillus amyloliquefaciens alpha-amylase gene (amyQ), Bacillus lichenformis penicillinase gene (penP), Bacillus subtilis xy1A and xy1B genes, and prokaryotic beta-lactamase gene (See e.g., Villa-Kamaroff et al., Proc. Natl. Acad. Sci. USA 75: 3727-3731, 1978), as well as the tac promoter (See e.g., DeBoer et al., Proc. Natl. Acad. Sci. USA 80: 21-25, 1983). Examples of promoters for filamentous fungal host cells, include, but are not limited to promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Examples of yeast cell promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (See e.g., Romanos et al., Yeast 8:423-488, 1992). The selection of a suitable promoter is within the skill in the art. The recombinant plasmids can also comprise inducible, or regulatable, promoters for expression of an enzyme in cells.

Various gene delivery vehicles are known in the art and include both viral and non-viral (e.g., naked DNA, plasmid) vectors. Viral vectors suitable for gene delivery are known to those skilled in the art. Such viral vectors include, e.g., vector derived from the herpes virus, baculovirus vector, lentiviral vector, retroviral vector, adenoviral vector and adeno-associated viral vector (AAV). Vectors derived from plant viruses can also be used, such as the viral backbones of the RNA viruses Tobacco mosaic virus (TMV), Potato virus X (PVX) and Cowpea mosaic virus (CPMV), and the DNA geminivirus Bean yellow dwarf virus. The viral vector can be replicating or non-replicating.

Non-viral vectors include naked DNA and plasmids, among others. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and such vectors may be introduced into many appropriate host cells, using methods disclosed or cited herein or otherwise known to those skilled in the relevant art.

In certain embodiments, the vector comprises a transgene operably linked to a promoter. The transgene encodes a biologically active molecule, such as an enzyme described herein.

To facilitate the introduction of the gene delivery vector into host cells, the vector can be combined with different chemical means such as colloidal dispersion systems (macromolecular complex, nanocapsules, microspheres, beads) or lipid-based systems (oil-in-water emulsions, micelles, liposomes).

Some embodiments relate to a vector comprising a nucleic acid encoding any enzyme described herein. In certain embodiments, the vector is a plasmid, and includes any one or more plasmid sequences such as, e.g., a promoter sequence, a selection marker sequence, or a locus-targeting sequence. Suitable plasmid vectors include p423TEF 2μ, p425TEF 2μ, and p426TEF 2μ. Another suitable vector is pHis8-4 (Whitehead Institute, Cambridge, Massachusetts, United States of America), which is identified as SEQ ID NO: 94. Another suitable vector is pEAQ-HT, which is identified as SEQ ID NO: 95. Another suitable vector is pJKW 1410, which is identified as SEQ ID NO: 96. pJKW 1410 is a backbone vector used to construct the multi gene yeast expression vector used for salidroside production in the work described in the Examples.

Although the genetic code is degenerate in that most amino acids are represented by multiple codons (called “synonyms” or “synonymous” codons), it is understood in the art that codon usage by particular organisms is nonrandom and biased towards particular codon triplets. Accordingly, in some embodiments, the vector includes a nucleotide sequence that has been optimized for expression in a particular type of host cell (e.g., through codon optimization). Codon optimization refers to a process in which a polynucleotide encoding a protein of interest is modified to replace particular codons in that polynucleotide with codons that encode the same amino acid(s), but are more commonly used/recognized in the host cell in which the nucleic acid is being expressed. In some aspects, the polynucleotides described herein are codon optimized for expression in a bacterial cell, e.g., E. coli. In some aspects, the polynucleotides described herein are codon optimized for expression in a yeast cell, e.g., S. cerevisiae.

### Host Cells

A wide variety of host cells can be used, including fungal cells, bacterial cells, plant cells, insect cells, and mammalian cells.

In some embodiments, the host cell is a fungal cell, such as a yeast cell and an Aspergillus spp cell. A wide variety of yeast cells are suitable, such as cells of the genus Pichia, including Pichia pastoris and Pichia stipitis; cells of the genus Saccharomyces, including Saccharomyces cerevisiae; cells of the genus Schizosaccharomyces, including Schizosaccharomyces pombe; and cells of the genus Candida, including Candida albicans.

In some embodiments, the host cell is a bacterial cell. A wide variety of bacterial cells are suitable, such as cells of the genus Escherichia, including Escherichia coli; cells of the genus Bacillus, including Bacillus subtilis; cells of the genus Pseudomonas, including Pseudomonas aeruginosa; and cells of the genus Streptomyces, including Streptomyces griseus.

In some embodiments, the host cell is a plant cell. A wide variety of cells from a plant are suitable, including cells from a Nicotiana benthamiana plant. In other embodiments, the plant belongs to a genus selected from the group consisting of Arabidopsis, Beta, Glycine, Helianthus, Solanum, Triticum, Oryza, Brassica, Medicago, Prunus, Malus, Hordeum, Musa, Phaseolus, Citrus, Piper, Sorghum, Daucus, Manihot, Capsicum, and Zea.

In some embodiments, the host cell is an insect cell, such as a Spodoptera frugiperda cell, such as Spodoptera frugiperda Sf9 cell line and Spodoptera frugiperda Sf21

In some embodiments, the host cell is a mammalian cell.

In some embodiments, the host cell is an Escherichia coli cell, and the vector is pHis8-4. In some embodiments, the host cell is a Nicotiana benthamiana cell, and the vector is pEAQ-HT. In some embodiments, the cell is a Saccharomyces cerevisiae cell, and the vector is a p423TEF 2μ plasmid, a p425TEF 2μ plasmid, or a p426TEF 2μ plasmid.

As used herein, the term “host cell” encompasses cells in cell culture and also cells within an organism (e.g., a plant).

Some embodiments relate to a host cell comprising a vector as described herein. In certain embodiments, the host cell is an Escherichia coli cell, a Nicotiana benthamiana cell, or a Saccharomyces cerevisiae cell.

In some embodiments, the hosts cells are cultured in a cell culture medium, such as a standard cell culture medium known in the art to be suitable for the particular host cell. In some embodiments, the culture medium is supplemented with one or more of L-tyrosine, 4-hydroxyphenylacetaldehyde (4-HPAA), and tyrosol. In some embodiments, the culture medium is supplemented with tyrosine, for example, between 0.1 mM and 100 mM L-tyrosine. In some embodiments, the culture medium is supplemented with 4-HPAA, for example, between 0.1 mM and 100 mM of 4-HPAA. In some embodiments, the culture medium is supplemented with tyrosol, for example, between 0.1 mM and 100 mM of tyrosol.

### Methods of Making Transgenic Host Cells

Described herein are methods of making a transgenic host cell. The transgenic host cells can be made, for example, by introducing one or more of the vector embodiments described herein into the host cell.

In one embodiment, the method comprises introducing into a host cell a vector that includes a nucleic acid encoding a tyrosol:UDP-glucose 8-O-glucosyltransferase (T8GT). In another embodiment, the method can also include introducing into the host cell a vector that includes a nucleic acid encoding a 4-hydroxyphenylacetaldehyde reductase (4HPAR) in addition to introducing a nucleic acid encoding a T8GT. In another embodiment, the method can further include introducing into the host cell a vector that includes a nucleic acid encoding a 4-hydroxyphenylacetaldehyde synthase (4HPAAS) in addition to introducing one or more nucleic acids encoding one or more of T8GT and 4HPAR.

In another embodiment, the method comprises introducing a vector that includes a nucleic acid encoding a 4-hydroxyphenylacetaldehyde synthase (4HPAAS). In another embodiment, the method can further include introducing into the host cell a vector that includes a nucleic acid encoding a 4-hydroxyphenylacetaldehyde reductase (4HPAR) in addition to introducing a nucleic acid encoding a 4HPAAS. In another embodiment, the method can further include introducing into the host cell a vector that includes one or more of a nucleic acid encoding tyrosol:UDP-glucose 8-O-glucosyltransferase (T8GT) and a tyrosol:UDP-glucose 4-O-glucosyltransferase (T4GT), in addition to introducing a nucleic acid encoding one or more of a 4HPAAS and a 4HPAR. In some embodiments, the method can further include introducing into the host cell a vector that includes a nucleic acid encoding a T8GT and a nucleic acid encoding a T4GT, in addition to introducing a nucleic acid encoding one or more of a 4HPAAS and a 4HPAR.

In some embodiments, nucleic acids encoding two or more of 4HPAAS, 4HPAR, T8GT, and T4GT are included in a single vector, such that a single vector encoding one or more enzymes is introduced into a host cell.

In some embodiments, one or more of the nucleic acids are integrated into the genome of the host cell. In some embodiments, the nucleic acids to be integrated into a host genome can be introduced into the host cell using any of a variety of suitable methodologies known in the art, including, for example, CRISPR-based systems (e.g., CRISPR/Cas9; CRISPR/Cpf1), TALEN systems and Agrobacterium-mediated transformation. However, as those skilled in the art would recognize, transient transformation techniques can be used that do not require integration into the genome of the host cell. In some embodiments, nucleic acid (e.g., plasmids) can be introduced that are maintained as episomes, which need not be integrated into the host cell genome.

In certain embodiments, the nucleic acid is introduced into a tissue, cell, or seed of a plant cell. Various methods of introducing nucleic acid into the tissue, cell, or seed of plants are known to one of ordinary skill in the art, such as protoplast transformation. The particular method can be selected based on several considerations, such as, e.g., the type of plant used. For example, the floral dip method, as described herein, is a suitable method for introducing genetic material into a plant. In certain embodiments, the nucleic acid can be delivered into the plant by an Agrobacterium.

In some embodiments, a host cell is selected or engineered to have increased activity of the synthesis pathway for one or more of L-tyrosine, 4-hydroxyphenylacetaldehydr (4-HPAA) and tyrosol. In some embodiments, a host cell is selected or engineered to have increased activity of the synthesis pathway for L-tyrosine. In some embodiments a host cell may be selected or engineered to have reduced feedback inhibition of one or more enzymes in the L-tyrosine synthesis pathway. In some embodiments, the host cell is engineered to increase uptake of a precursor, such as L-tyrosine, 4-HPAA, or tyrosol, from the medium.

### Methods of Making Salidroside, Icariside D2, and Salidroside Precursors

Described herein are methods of making salidroside, icariside D2, and salidroside precursors. Salidroside, icariside D2, and salidroside precursors can be produced by expressing one or more of the enzymes described herein in a host cell.

Some embodiments provide a method of making tyrosol 8-O-glucoside (salidroside) in a host cell. The method can include expressing in a host cell a transgene that encodes a tyrosol:UDP-glucose 8-O-glucosyltransferase (T8GT). In some embodiments, the host cell synthesizes tyrosol and includes, either endogenously or transgenically, enzymes to synthesize tyrosol. In some embodiments, tyrosol is provided in the culture media. In some embodiments, the host cell further expresses a transgene that encodes a 4-hydroxyphenylacetaldehyde reductase (4HPAR). In some embodiments, the host cell further expresses a transgene that encodes a 4-hydroxyphenylacetaldehyde synthase (4HPAAS).

Some embodiments provide a method of making tyrosol 8-O-glucoside (salidroside) in a host cell. The method can include expressing in a host cell a transgene that encodes a 4-hydroxyphenylacetaldehyde synthase (4HPAAS) and a transgene that encodes a 4-hydroxyphenylacetaldehyde reductase (4HPAR). The host cell expresses, either endogenously or transgenically, one or more enzymes that catalyze conversion of tyrosol to tyrosol 8-O-glucoside (salidroside).

Certain embodiments provide a method of making 4-hydroxyphenylacetaldehyde (4-HPAA) in a host cell. The method can include expressing in the host cell a transgene that encodes a 4-hydroxyphenylacetaldehyde synthase (4HPAAS). In some embodiments, the host cell includes L-tyrosine, produced endogenously or provided to the cell exogenously. In some embodiments, L-tyrosine is provided in the cell culture medium. In some embodiments, the method further includes making tyrosol in the host cell, and the host cell further expresses a transgene encoding a 4-hydroxyphenylacetaldehyde reductase (4HPAR).

In some embodiments, particularly those optimized for producing salidroside, the host cell can have low or absent T4GT activity in order to reduce competition from T4GT for the substrate tyrosol. In some embodiments, the host cell is engineered to reduce or eliminate expression of T4GT.

In some embodiments, particularly those optimized for producing icariside D2, the host cell can have low or absent T8GT activity in order to reduce competition from T8GT for the substrate tyrosol. In some embodiments, the host cell is engineered to reduce or eliminate expression of T8GT.

In some embodiments, a host cell (e.g., a bacterial host cell) endogenously expresses enzymes that catalyze the production of salidroside or icariside D2 from tyrosol. For example, some bacteria express UGTs that exhibit T8GT and/or T4GT activity (Fan et al., 2017). In some embodiments, nucleic acids encoding the bacterial-derived T8GTs can be used in vectors and methods described herein. In some embodiments, host cells and methods can express a T8GT that is a bacterial-derived T8GT.

In some embodiments, one or more copies of one or more of the nucleic acids are integrated into the genome of the host cell. However, as those skilled in the art would recognize, transient transformation techniques can be used that do not require integration into the genome of the host cell.

Methods of obtaining, or extracting, salidroside, icariside D2, and precursors of salidroside and icariside D2 are described herein and are well known to one or ordinary skill in the art. For example, as described herein, salidroside, icariside D2, and/or precursors of salidroside and icariside D2 can be separated by liquid chromatography. Larger scale separation can be obtained by, e.g., simulated moving bed (SMB) chromatography and/or ion exchange chromatography. Any of the methods described herein can further include isolating salidroside, icariside D2, and/or a salidroside precursor from a host cell. Any of the methods described herein can include harvesting tissue (e.g., leaves, roots) of a transgenic plant described herein and processing the harvested tissue to obtain salidroside, icariside D2, and/or a precursor of salidroside and icariside D2 therefrom.

### Values and Ranges

Unless otherwise indicated or otherwise evident from the context and understanding of one of ordinary skill in the art, values that are expressed as ranges can assume any specific value or subrange within the stated ranges in various embodiments, unless the context clearly dictates otherwise. “About” in reference to a numerical value generally refers to a range of values that fall within ±8%, in some embodiments ±6%, in some embodiments ±4%, in some embodiments ±2%, in some embodiments ±1%, in some embodiments ±0.5% of the value unless otherwise stated or otherwise evident from the context.

### EXEMPLIFICATION

### Example #1: Results

Generating Metabolomics and Transcriptomics Resources for R. rosea

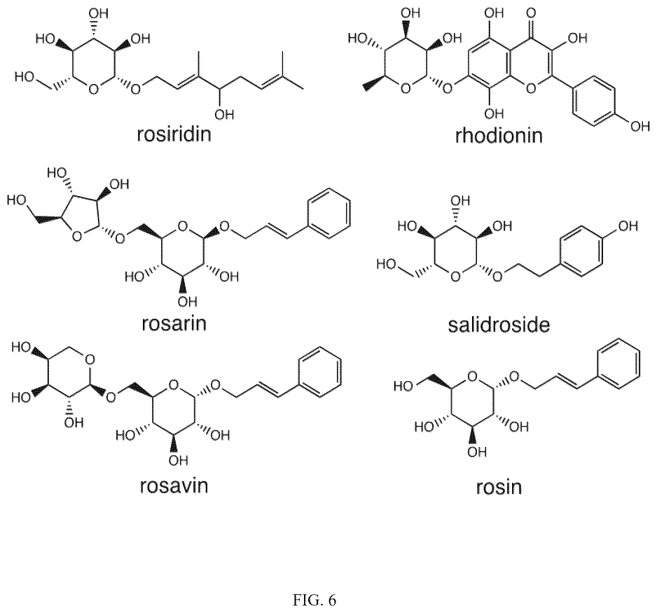

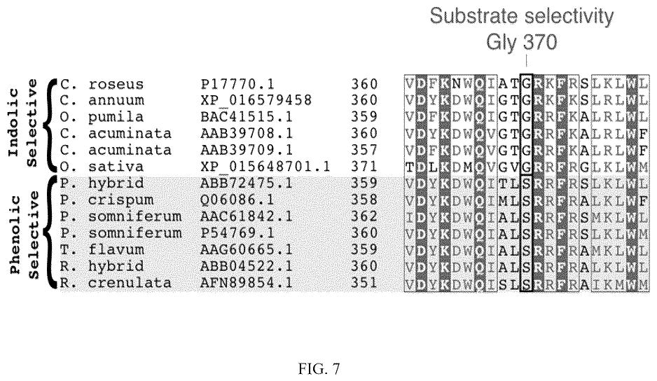

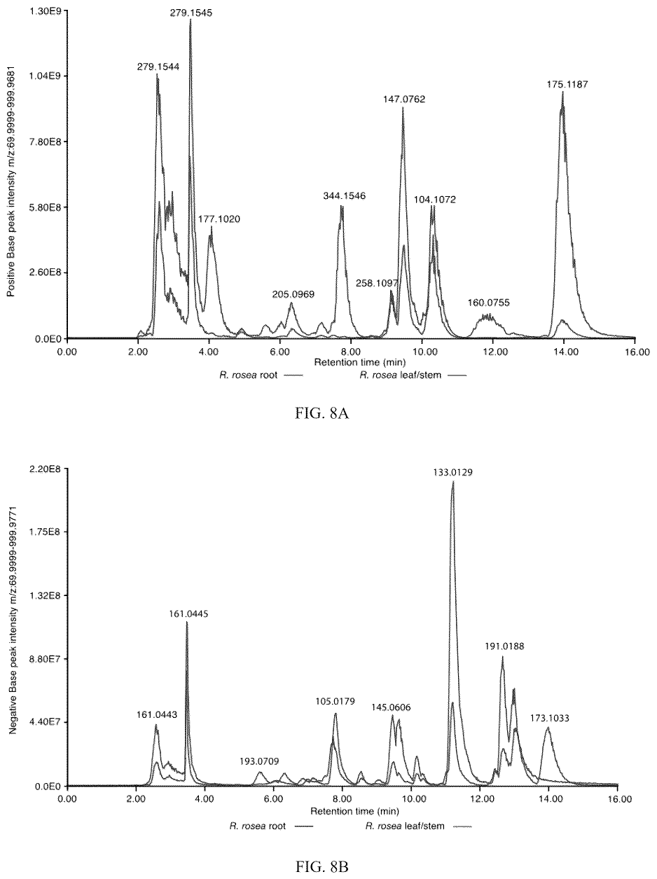

To survey the metabolic profile of Rhodiola cultivated under lab conditions, crown tissue (aerial tissue including leaves and stems) and root tissue were collected separately from a three-month old greenhouse-grown R. rosea plant (FIG. 1A). The fresh tissues were extracted by 50% methanol, and analyzed by untargeted liquid chromatography high-resolution accurate-mass mass spectrometry (LC-HRAM-MS). This analysis confirmed the presence of tyrosol, rosavin and salidroside in greenhouse-grown R. rosea, all of which accumulate at much higher levels in the root compared to the crown (FIGS. 1, 8, and 9).

The higher accumulation of these metabolites in the root suggests that the requisite biosynthetic genes may also obey a similar tissue-specific expression pattern. An RNA-Seq experiment was then performed using total RNAs prepared from the two tissues. This experiment yielded about 30 million paired-end sequencing reads (100×100 bp) per sample. While 84,645 and 105,132 unique transcripts were assembled de novo from the crown and root tissues separately, a total of 128,623 unique transcripts were assembled combining all raw sequencing reads from both tissues. The combined transcriptome was evaluated as 90.3% complete by the metric of Benchmarking Universal Single-Copy Orthologs (BUSCO) (Simao et al., 2015). The Transcripts Per Million (TPM) value of unique transcripts in each tissue type was calculated to infer the relative expression level of the corresponding genes (Li et al., 2010). The identification and prioritization of candidate salidroside biosynthetic genes from the R. rosea transcriptome were based upon our hypothetical salidroside biosynthetic model, subsequent large-scale phylogenetic analyses, and the relative expression level of plausible candidate genes in the two examined tissue types. The biochemical function of selected candidate genes was further investigated both in vitro and in vivo.

R. rosea Contains a Neofunctionalized 4HPAAS

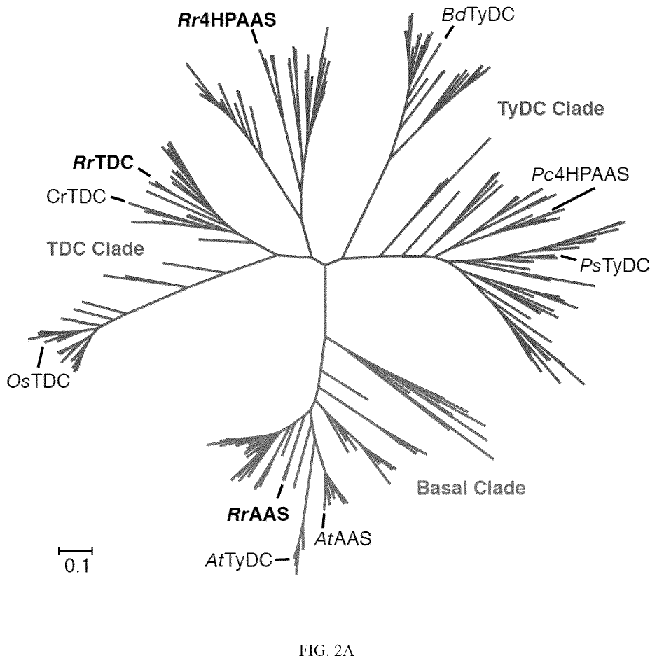

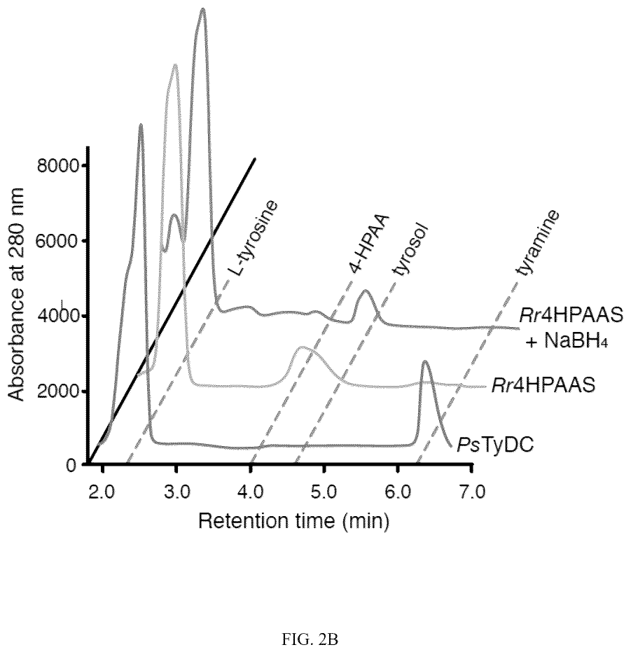

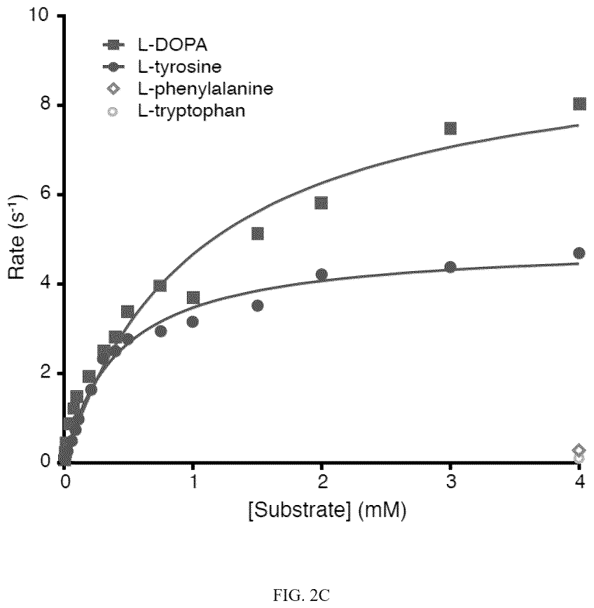

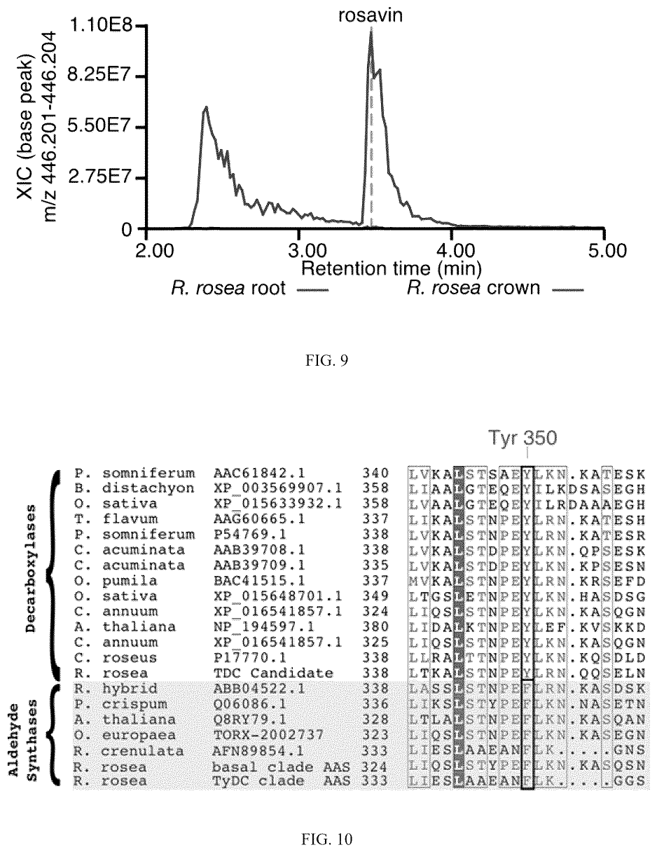

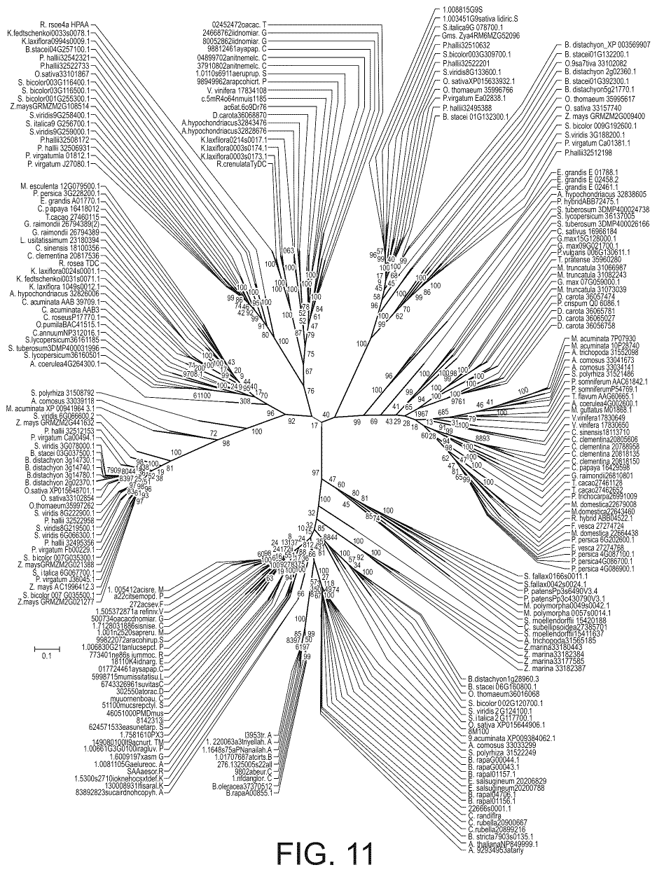

A BLAST search using PsTyDC as the query against the R. rosea transcriptome identified three AAAD homologs. Using the sequence motifs correlating to AAAD substrate specificity and catalytic mechanism (Torrens-Spence et al., 2014; Torrens-Spence et al., 2013), two of the three R. rosea AAAD homologs were predicted to possibly function as AASs, and the other is likely to catalyze decarboxylation chemistry (FIG. 10). A phylogenetic analysis including the three R. rosea AAAD homologs together with other AAAD sequences from taxonomically diverse plant species was conducted (FIGS. 2B and 11). Whereas the predicted R. rosea decarboxylase candidate clusters within the TDC clade (red) containing largely previously known TDCs, the two R. rosea AAS candidates fall into two distinct clades, designated as the basal clade (green) and the TyDC clade (blue), respectively (FIGS. 2B and 11). It is noted that the TyDC-type AAS candidate isolated in this study is likely orthologous to the RcTyDC previously reported by Bai et al. (Bai et al., 2014), sharing 96% sequence identity at the protein level.

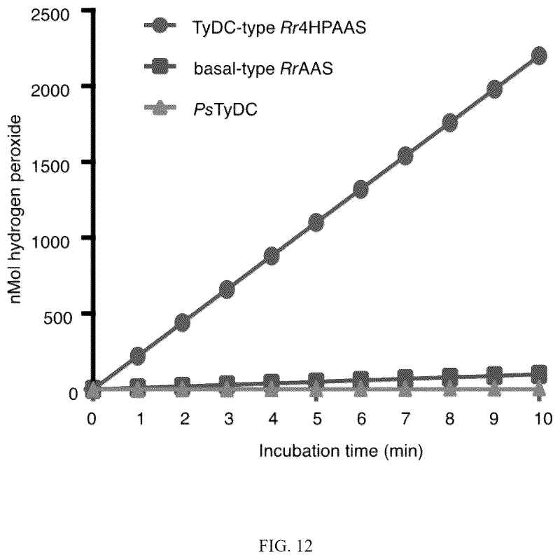

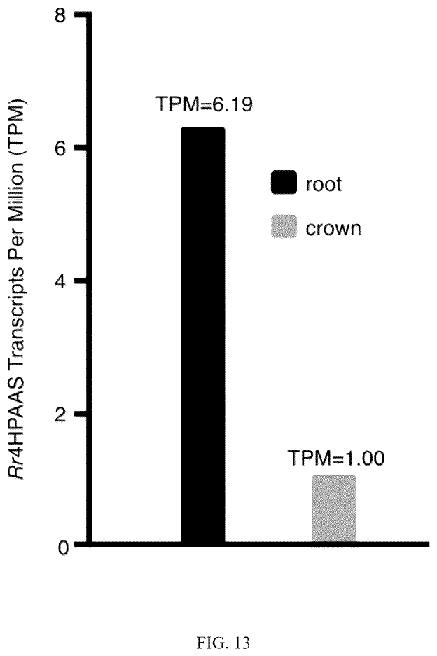

To experimentally assess the biochemical activities of the two R. rosea AAS candidates, full-length open reading frame corresponding to both the basal and TyDC-type AAS candidate genes from R. rosea cDNA were cloned. Their encoded proteins were recombinantly expressed in E. coli, purified to homogeneity, and tested for enzymatic activity using L-tyrosine as the substrate. Both enzymes readily yield hydrogen peroxide, a co-product of AAS as opposed to canonical TyDC (Kaminaga et al., 2006), while the TyDC-type AAS candidate exhibits much higher activity than the basal AAS candidate (FIG. 12). To confirm the chemical identity of the AAS reaction products, the enzyme assays were analyzed by LC coupled with a UV detector (FIG. 2B). Incubation of L-tyrosine with both AAS candidate enzymes led to the production of 4-HPAA, which is distinct from the tyramine product yielded by PsTyDC as a control (FIG. 2B). The identity of the 4-HPAA product was further confirmed by sodium borohydride reduction of 4-HPAA to yield tyrosol (FIG. 2B). Notably, the transcript corresponding to the TyDC-type AAS candidate is highly enriched in the root versus the crown (FIG. 13), whereas such pattern was not observed for the basal AAS candidate. In light of these results, the TyDC-type AAS candidate is likely the primary AAS involved in salidroside biosynthesis in R. rosea root. The TyDC-type AAS candidate is referred to as Rr4HPAAS hereafter.

The Michaelis-Menten kinetics of Rr4HPAAS was measured against four aromatic amino acids, namely L-tyrosine, L-3,4-dihydroxyphenylalanine (L-DOPA), L-phenylalanine, and L-tryptophan (FIG. 2C and Table 1). Rr4HPAAS demonstrates the highest catalytic efficiency toward L-tyrosine (k_{cat}/Kₘ=11.7 s⁻¹ mM⁻¹) followed by L-DOPA (k_{cat}/Kₘ=9.1 s⁻¹ mM⁻¹), whereas L-phenylalanine and L-tryptophan are much less preferred substrates (FIG. 2C and Table 1). L-DOPA and any potential phenolic compound derived from it were not detected in the R. rosea metabolomics datasets, and thus the kinetic characteristics of Rr4HPAAS is consistent with its role in salidroside biosynthesis. These results also suggest that the previously reported RcTyDC was likely functionally mischaracterized (Bai et al., 2014).

### Identification and Biochemical Characterization of Rhodiola Phenolic Aldehyde Reductases

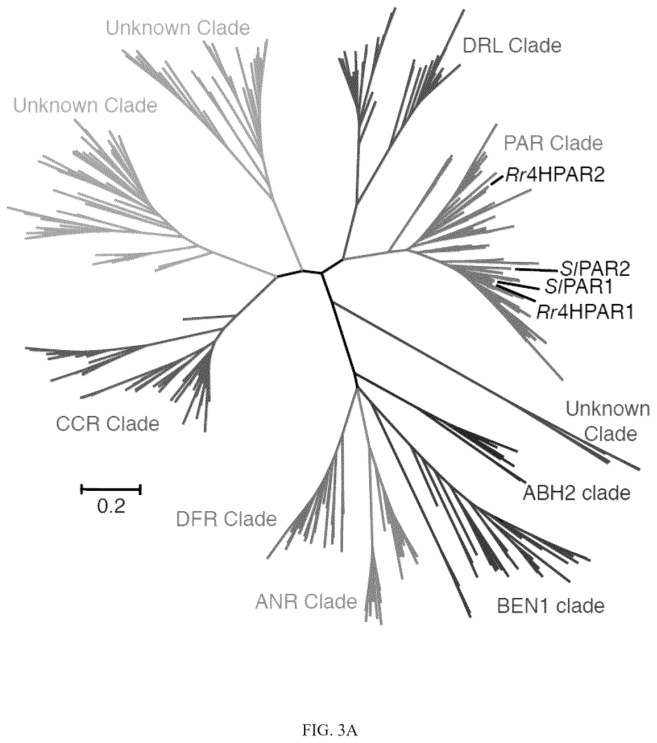

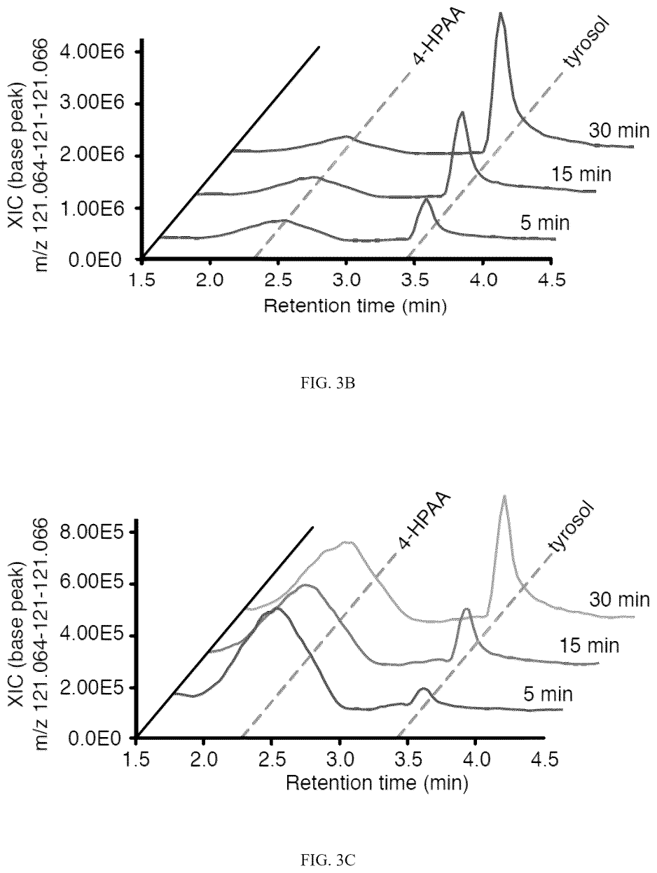

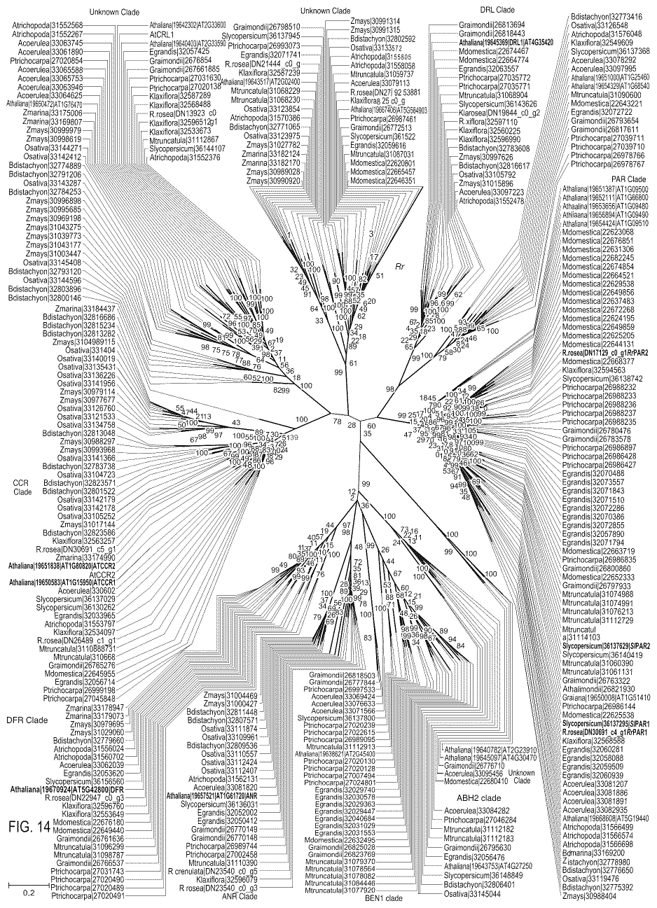

To identify R. rosea enzymes involved in the next step of salidroside biosynthesis, a BLAST search was conducted using the previously characterized Solanum lycopersicum PAR (SIPAR, GenBank: ABR15768.1) as a query (Tieman et al., 2007) against our R. rosea transcriptome. A phylogenetic analysis was performed using the returned R. rosea hits together with other homologous ADHs from select plant species (FIGS. 3A and 14). This analysis revealed two R. rosea ADH homologs, referred to as RrPAR-like1 and RrPAR-like2, that cluster phylogenetically with SIPAR (Tieman et al., 2007), and share 76% and 58% protein sequence identity to S/PAR, respectively. Both genes were cloned from R. rosea cDNA as candidate genes encoding 4HPAR.

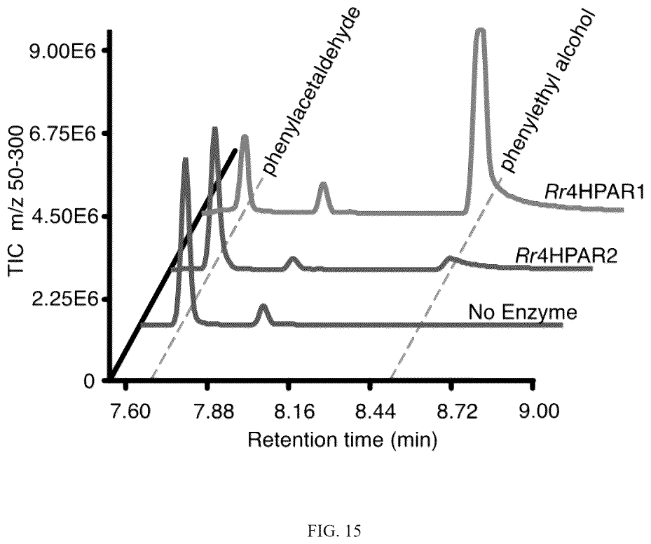

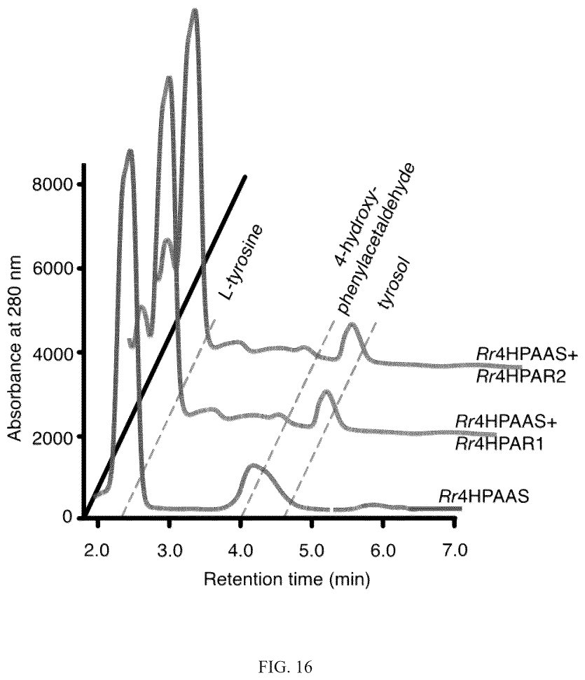

To examine the biochemical activity of the two 4HPAR candidates, recombinant enzymes were expressed in E. coli, purified to homogeneity, and assayed against 4-HPAA or phenylacetaldehyde in the presence of NADPH as the co-substrate. Both enzymes are capable of reducing phenylacetaldehyde to phenylethanol with RrPAR-like1 displaying higher activity (FIG. 15). Likewise, RrPAR-like1 exhibited orders of magnitude higher specific activity towards 4-HPAA (6.9 mol min⁻¹ mg⁻¹) than RrPAR-like2 (8.4 nmol min⁻¹ mg⁻¹) (FIGS. 3B, 3C, and 16). Nonetheless, RrPAR-like1 and RrPAR-like2 were renamed as Rr4HPAR1 and Rr4HPAR2, respectively, as both enzymes displayed 4-HPAA reductase activity.

Identification of Regio-Specific Tyrosol-Modifying UGTs from R. rosea

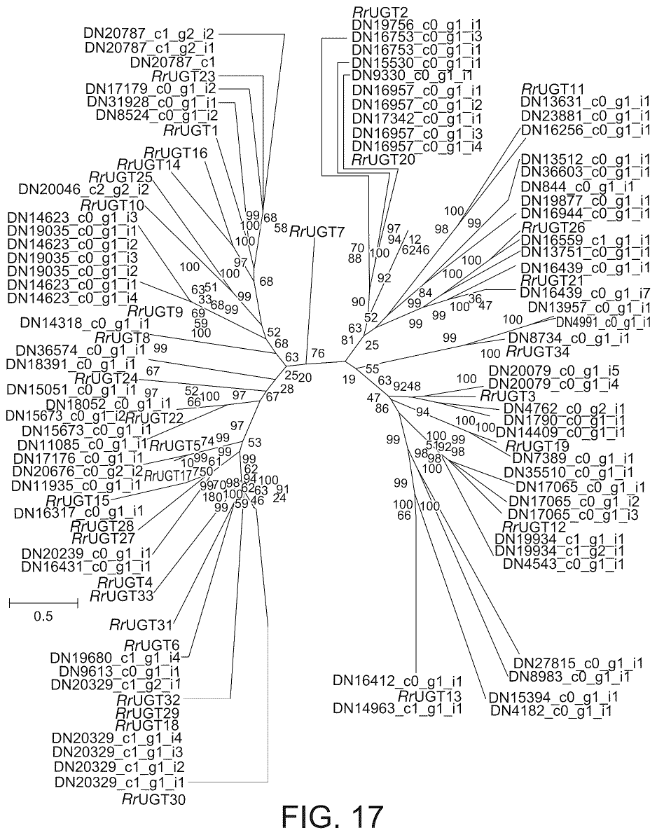

To complete the salidroside biosynthetic pathway, candidate UGT genes encoding T8GT were identified. The UGT superfamily is one of the largest enzyme families in the plant kingdom (Li et al., 2001). The plant secondary product glycosyltransferase (PSPG) motif is described in Gachon et al., 2005, particularly at FIG. 2A and associated text. The transcriptome was queried using a UGT superfamily signature motif (Li et al., 2001), and conducted an unbiased phylogenetic analysis using 113 curated non-redundant full-length UGT homologs retrieved from the R. rosea transcriptome (FIG. 17). Thirty-four candidate UGT genes were then prioritized for further functional analysis according to a combination of criteria including phylogenetic distribution pattern and transcript levels in the root and crown transcriptome datasets.

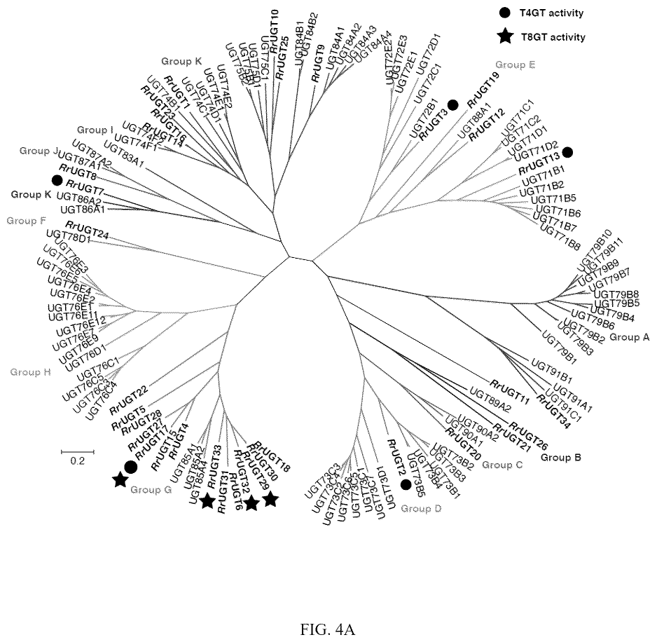

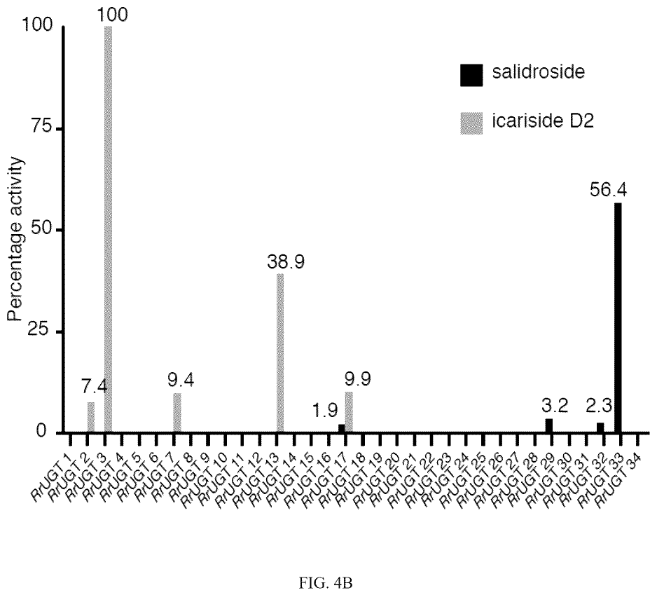

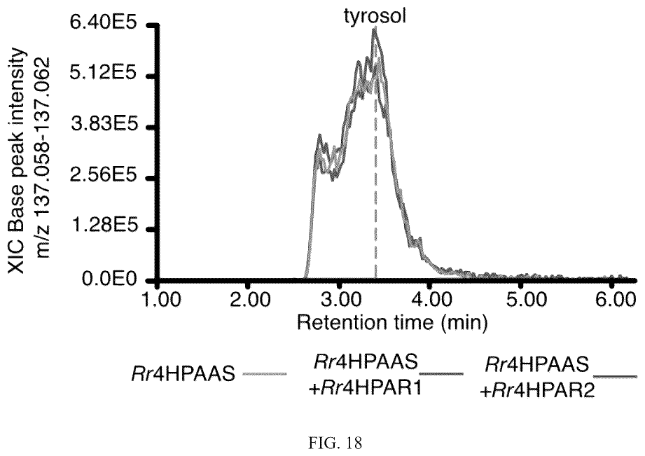

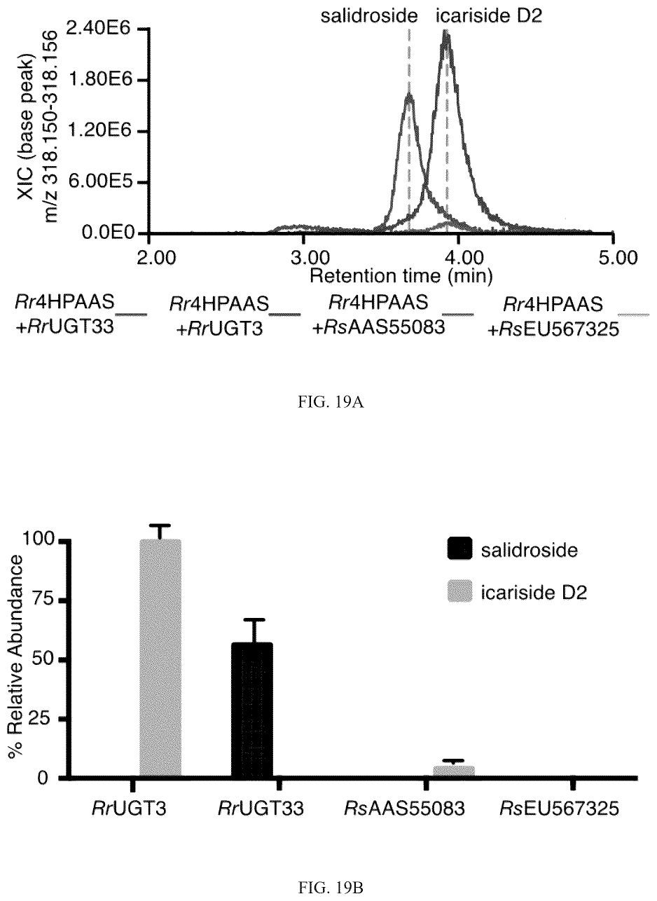

To facilitate functional assessment of a large number of UGT candidates, an in vivo tyrosol glycosylation assay in the yeast S. cerevisiae was devised. Initial iterations of the tyrosol-producing yeast strains were generated by transforming wild type S. cerevisiae BY4743 with separate 2-micron TEF-promoter expression plasmids containing Rr4HPAAS and Rr4HPAR1, respectively. It was later observed that yeast contains endogenous ADH activity sufficient to reduce 4-hydroxyphenylacetaladehyde produced by Rr4HPAAS to tyrosol. Therefore, the Rr4HPAR1-containing plasmid was omitted in the final tyrosol-producing strain (FIG. 18). Each of the 34 R. rosea UGT candidate genes, carried on the yeast 2-micron TEF-promoter expression plasmids, was transformed into the background strain expressing Rr4HPAAS. After auxotrophic selection, colonies were cultured, harvested and subjected to metabolic profiling by LC-HRAM-MS. From this screen, we identified three UGTs (RrUGT29, 32, and 33) with regio-specific T8GT activity, four UGTs (RrUGT2, 3, 7, and 13) with regio-specific T4GT activity, and RrUGT17 with both T8GT and T4GT activities (FIG. 4B). Further phylogenetic analysis of the 34 cloned R. rosea UGTs against the 88 unique and complete A. thaliana UGTs suggests a correlation between the cladding of the UGTs and their respective biochemical activities (FIG. 4A) (Li et al., 2001). The UGTs that contain T4GT activity appear to be phylogenetically diverse with representative enzymes falling into the D, G, E and K groups, while all of identified T8GTs cluster within the G group (FIG. 4A). These results also show that RrUGT3 and RrUGT33, the most active T4GT and T8GT, respectively, display significantly higher regio-specific tyrosol glycoside-producing activities than the two previously reported UGTs from R. sachalinensis (GenBank. AAS55083 and EU567325) (FIG. 19) (Ma et al., 2007; Yu et al., 2011).

Using recombinant enzymes produced and purified from E. coli, the kinetic parameters for the salidroside-producing RrUGT29 and RrUGT33 and the icariside D2-producing RrUGT2 and RrUGT3 were measured (FIG. 4C and Table 1). RrUGT33 exhibits the highest T8GT catalytic efficiency with a k_{cat}/Kₘ value of 420.6 s⁻¹ mM⁻¹ and was subsequently referred to as RrT8GT (Table 1). In contrast, RrUGT3 exhibits the greatest T4GT catalytic efficiency with a k_{cat}/Kₘ value of 117.2 s⁻¹ mM⁻¹ and was subsequently referred to as RrT4GT (Table 1).

Heterologous Production of Salidroside and Icariside D2 in N. benthamiana

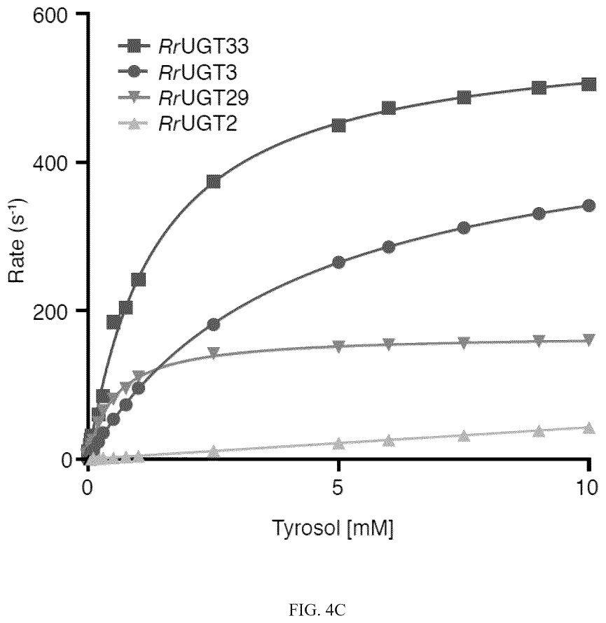

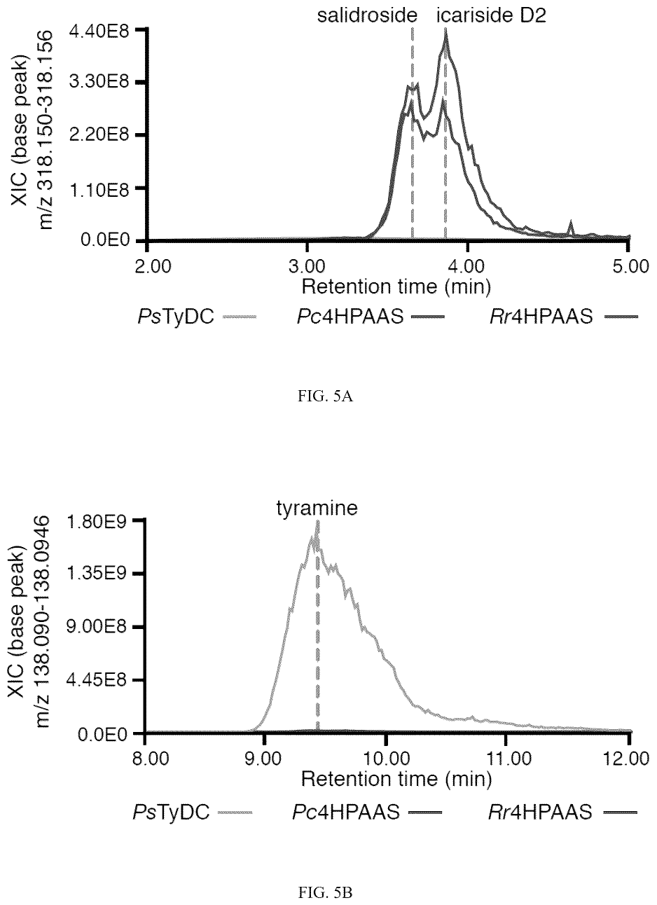

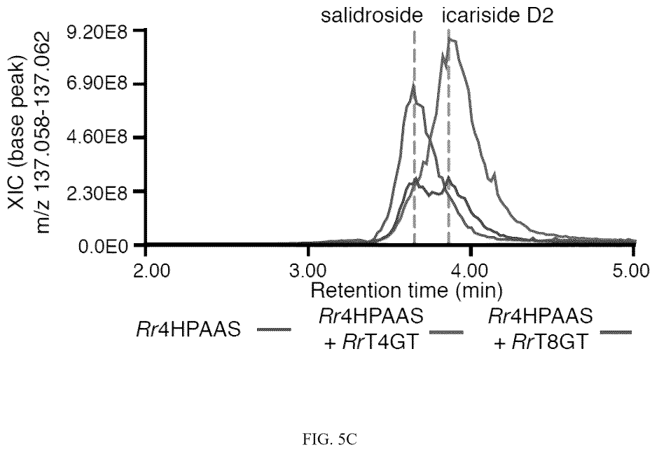

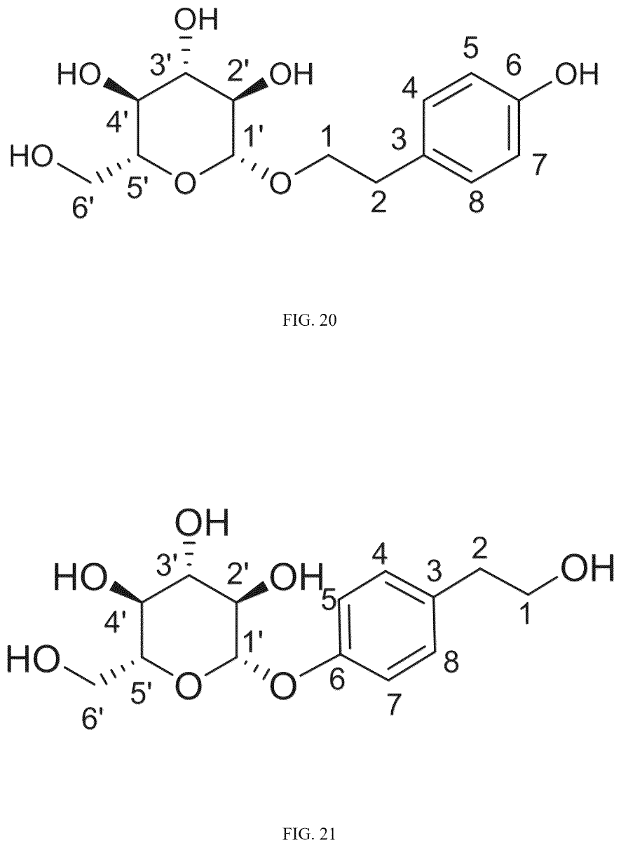

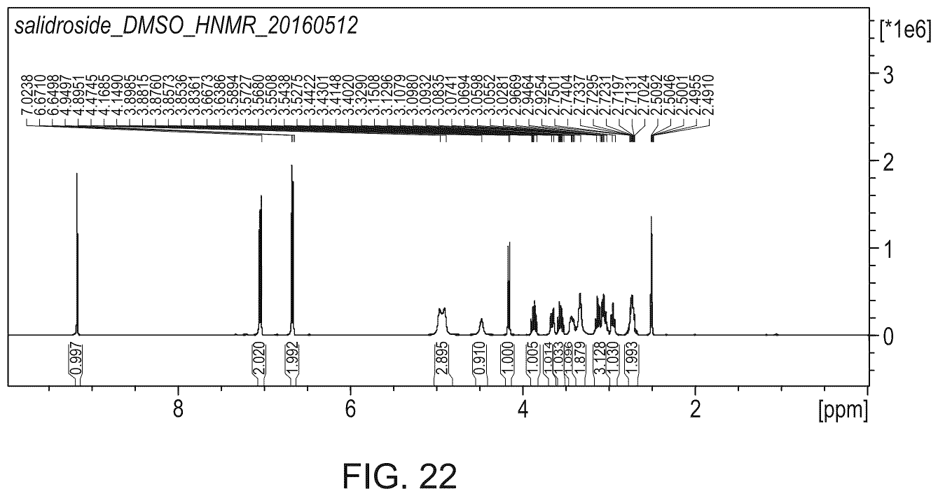

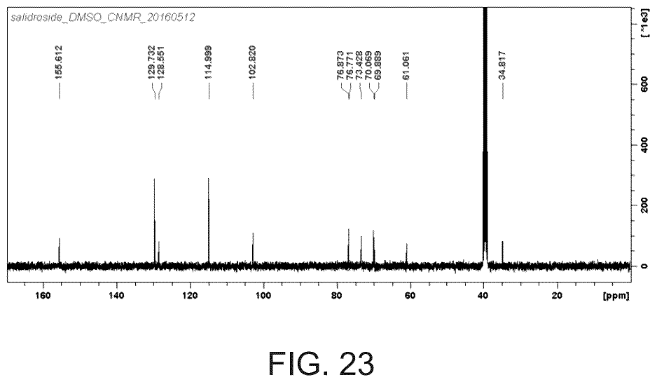

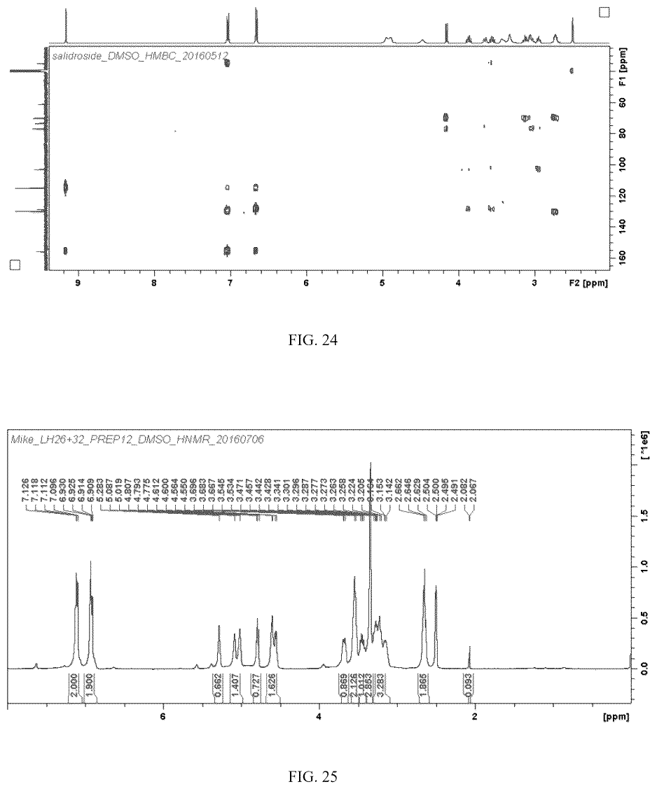

To further evaluate the biochemical function of R. rosea tyrosol glycoside biosynthetic genes inplanta, these genes were expressed in N. benthamiana leaves using the Agrobacterium tumefaciens-mediated transient protein production technique (Sainsbury et al., 2009) followed by LC-HRAM-MS-based metabolic profiling. To first demonstrate the biochemical function of Rr4HPAAS in planta, Rr4HPAAS alone was transiently expressed in N. benthamiana leaves. PsTyDC and the previously reported Petroselinum crispum 4HPAAS (Pc4HPAAS, GenBank. AAA33861) (Torrens-Spence et al., 2012) were also tested in parallel as controls. Interestingly, expression of Rr4HPAAS or Pc4HPAAS in N. benthamiana led to significant accumulation of both salidroside and icariside D2 in N. benthamiana leaves (FIG. 5A). The chemical identity of these compounds was confirmed by both LC-HRAM-MS and nuclear magnetic resonance (NMR) analyses (FIGS. 20-26). This result suggests that 4-HPAA produced by transgenic 4H1PAAS can be readily metabolized by endogenous N. benthamiana reductase and glycosyltransferase enzymes to yield both salidroside and icariside D2. In contrast, the expression of PsTyDC yielded tyramine in high abundance in N. benthamiana leaves with no measurable production of tyrosol glycosides (FIG. 5B). Next, the in planta regio-specificity of RrT8GT and RrT4GT in tyrosol glycosylation was evaluated. Co-expression of either RrT8GT or RrT4GT with Rr4HPAAS led to regio-specific glycosylation of tyrosol and accumulation of salidroside or icariside D2, respectively (FIG. 5C). Meanwhile, the accumulation of free tyrosol was reduced in these plants compared to those with Rr4HPAAS expression alone (FIG. 27). Notably, the paired expression of Rr4HPAAS and one of the two regio-specific R. rosea tyrosol glycosyltransferases yielded up to 2% dry weight for salidroside or icariside D2 production in N. benthamiana leaves. This set of in planta experiments demonstrate that Rr4HPAAS and regio-specific RrT8GT are specialized metabolic enzymes underpinning salidroside biosynthesis in Rhodiola. Although icariside D2 does not naturally accumulate in Rhodiola, the identification of the regio-specific RrT4GT adds to the tool box for metabolic engineering of valuable tyrosol-derived glycosides.

Optimization of Salidroside Production in S. cerevisiae

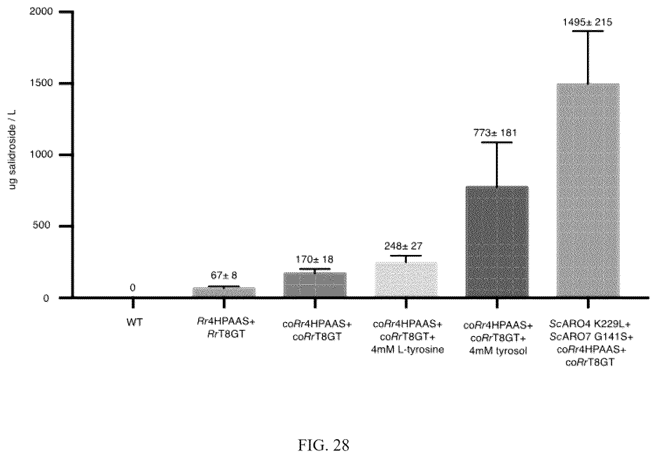

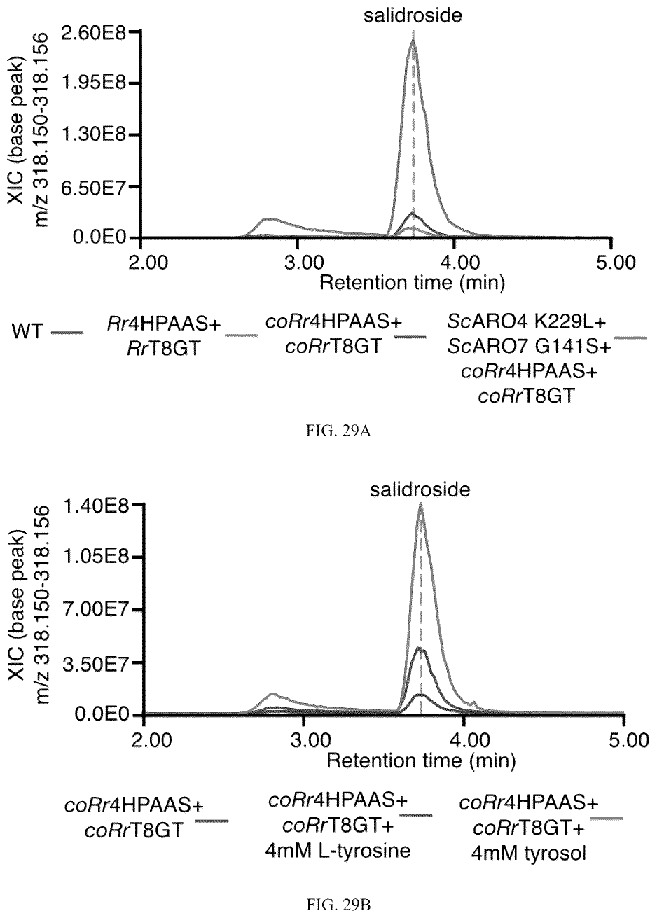

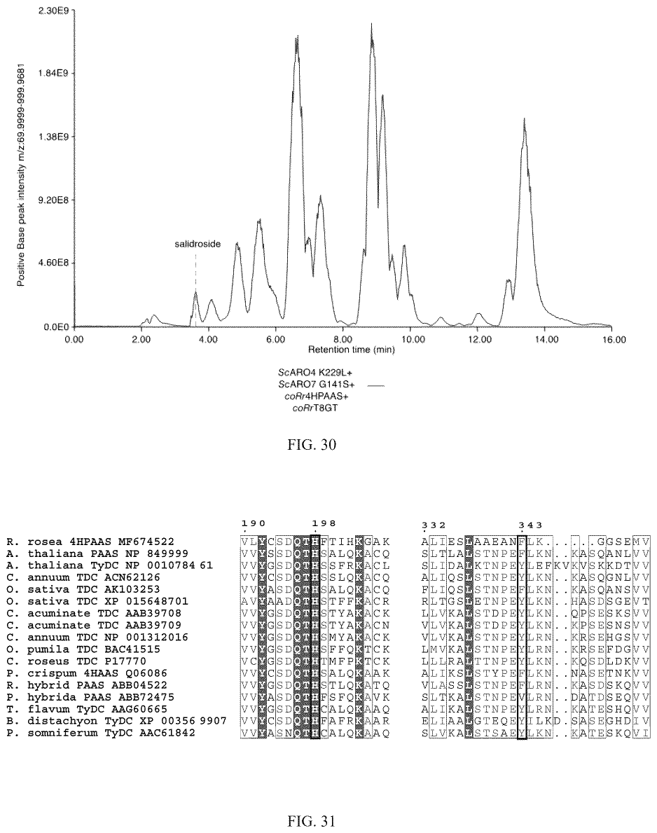

The complete elucidation of salidroside biosynthesis in Rhodiola provides new opportunities for bioengineering of sustainable salidroside production in heterologous hosts. Although N. benthamiana has been used for the commercial production of high value natural products and recombinant proteins, its scalability currently does not match to industrial yeast fermentation. To increase the salidroside titer in yeast, the Rr4HPAAS and RrT8GT genes were optimized according to S. cerevisiae codons, and assembled in a custom 2 plasmid for constitutive expression driven by pTDH3 promoter in yeast (FIGS. 28 and 29A). The increased promoter strength and codon optimization of Rr4HPAAS (coRr4HPAAS) and RrT8GT (coRrT8GT) resulted in a 2.5-fold increase in salidroside titer as compared to the initial strain. To probe the potential bottlenecks in salidroside biosynthesis in yeast, we next fed the culture containing the codon optimized construct with either L-tyrosine or tyrosol. Both feeding experiments demonstrated significant increase in salidroside titer, suggesting that improved tyrosine flux may further improve salidroside titer (FIGS. 28 and 29B). Thus, a yeast strain was engineered to include the previously described feedback-insensitive mutants of the yeast L-tyrosine pathway enzymes ARO4 and ARO7 (Gold et al., 2015). Incorporation of both ARO4 K229L and ARO7 G141S into the prior best engineered yeast strain produced salidroside as one of the most abundant metabolites with a titer of 1.5 mg L⁻¹, when grown for 48 h in 4% glucose 2×yeast nitrogen base in shake flasks (FIGS. 28, 29A, and 30). In summary, this preliminary metabolic engineering exercise in yeast yielded a prototype salidroside-producing strain, which can be improved through additional rounds of targeted and untargeted genetic modifications to further increase titer.

### Example #1: Discussion

As described herein, the R. rosea ortholog of the previously reported RcTyDC is a 4HPAAS, which catalyzes the direct conversion of tyrosine to 4-HPAA. This discovery therefore corrected a major long-standing misconception about the biosynthetic route towards tyrosol, an important precursor for many important phenolic natural products in plants (Chapple et al., 1986; Wyk, 2010).

UGTs play important roles in plant specialized metabolism as they alter the solubility, reactivity, bioactivity, intercellular and subcellular transport of a wide array of plant metabolites by glycosylation (Jones and Vogt, 2001). The resulting glycosides also have profound impact on human health with diverse pharmacological and nutraceutical indications (Jones and Vogt, 2001). Since natural product glycosides often contain distinct pharmacokinetic properties as compared to their aglycones, chemical derivatization via glycosylation has received considerable attention in pharmaceutical research (Gantt et al., 2011). Several R. rosea UGTs capable of producing salidroside and icariside D2 from the aglycone tyrosol in a regio-specific manner were identified. In this gene-mining process, a library was established containing phylogenetically diverse UGTs from R. rosea, which likely contain enzymes responsible for the biosynthesis of other phenolic glycosides from Rhodiola, such as rosiridin, rhodionin, rosarin, rosin, and rosavin (FIG. 6).

Unlike bacterial natural product biosynthetic pathways, which are encoded by operons ubiquitously present in bacterial genomes, enzyme-encoding genes of a given plant specialized metabolic pathway often scatter randomly across the plant genome, making metabolic pathway elucidation unattainable simply by genome mining. Plants, like many other multicellular eukaryotes, contain rich tissue types where specific natural products accumulate under developmental and environmental regulations. In recent years, this feature of plant specialized metabolism has been exploited for pathway and enzyme discovery in medicinal plants that lack classical genetic tools (Torrens-Spence et al., 2016). Through mining transcriptomics and metabolomics datasets generated separately from the root and crown tissues of R. rosea, candidate salidroside biosynthetic genes were prioritized based on correlation between transcript and metabolite abundances in these two tissues. Extended phylogenomics analyses of the involved enzyme families further provided additional information that facilitates salidroside biosynthetic gene discovery. The biochemical functions of the identified candidate enzymes were then examined in vitro using recombinantly expressed proteins, and in vivo through expression of the candidate enzymes in heterologous hosts, e.g. yeast and N. benthamiana in this case. Collectively, this work describes a rare de novo elucidation of the complete biosynthetic pathway of a given plant natural product. The workflow adopted in this study is generally applicable for future investigation of other largely unexplored specialized metabolic pathways in non-model plants, and will ultimately contribute to a capability of synthesizing structurally diverse plant natural products through the means of metabolic engineering.

### Materials and Methods

### Reagents

Salidroside, tyrosine, tyramine, tyrosol, phenylacetaldehyde, phenylethyl alcohol, sodium borohydride, NADPH, UDP-glucose, and PLP were purchased from Sigma-Aldrich. 4-HPAA was purchased from Santa Cruz Biotechnology, Inc.

### Plant Materials

R. rosea seeds were purchased from Horizon Herbs. Seeds were stratified at 4° C. for three days, and germinated in potting soil. R. rosea, P. crispum, and N. benthamiana plants were grown under a 16-h-light/8-h-dark photoperiod at 23° C. in a local greenhouse.

RNA Isolation, Library Preparation, Transcriptome Assembly, cDNA Production and Molecular Cloning

Tissue of seventy-day-old R. rosea plants were harvested for total RNA extraction using the Qiagen's RNeasy Mini Kit (Qiagen). RNA quality was assessed by Bioanalyzer (Agilent Technologies). For the RNAseq experiment, strand-specific mRNA libraries were prepared using total RNA prepared separately from the root and crown tissue using the TruSeq Stranded mRNA Library Prep Kit (Illumina), and sequenced on a HiSeq2000 sequencer (Illumina) in paired-end mode (PE100). Sequence FASTQ files were trimmed for sequencing adaptors using Trimmomatic (Bolger et al., 2014) and assembled into de novo transcriptomes using Trinity in strand-specific mode (Grabherr et al., 2011). Gene expression statistics (TPM values) were determined by RSEM (Li and Dewey, 2011). Completeness of the combined R. rosea root and crown transcriptome was evaluated using the BUSCO tool, with ‘embryophyta_odb9’ set as lineage and ‘arabidopsis’ set as model species (Simao et al., 2015). Putative coding regions were predicted using Transdecoder (Haas et al., 2013). Transcripts and predicted protein sequences were annotated with TPM values and closest BLAST hits using in-house scripts. Transcriptome mining was performed on a local BLAST server (Anurag Priyam, 2015). First-strand cDNAs were synthesized by RT-PCR using total RNA sample as template and the Invitrogen SuperScript™ III kit (Invitrogen) with the oligo(dT)20 primer. The coding sequences (CDS) of candidate genes were amplified from cDNAs by PCR using gene-specific primers (Table 2). Select R. rosea and R. sachalinensis genes were also synthesized as gBlocks (IDT) with yeast codon optimization. Gibson assembly was used to ligate PCR amplicons or gBlocks into several base vectors. These include pHis8-4, a bacterial expression vector containing an N-terminal 8×His tag followed by a tobacco etch virus (TEV) cleavage site for recombinant protein production in E. coli; pEAQ-HT, a binary vector designed for transient expression of heterologous proteins in N. benthamiana (Peyret and Lomonossoff, 2013); p423TEF, p425TEF and p426TEF 2 plasmids (Mumberg et al., 1995) with various auxotrophic growth markers for constitutive expression in S. cerevisiae; and a custom plasmid containing 2, pTDH3, tTDH1, HIS3 for constitutive multi gene expression in S. cerevisiae (Lee et al., 2015).

### Sequence Alignment and Phylogenetic Analysis

The protein multiple sequence alignments were generated using ClustalW2 with default settings (Thompson et al., 2002). ESPript 3.0 (Gouet et al., 2003) was used to display the multiple sequence alignments. The phylogeny was inferred using the Maximum Likelihood method based on the Poisson correction model (L, 1965). The bootstrap consensus unrooted trees were inferred from 500 replicates to represent the phylogeny of the analyzed enzyme families (Sanderson and Wojciechowski, 2000). The phylogenetic analysis of the AAAD family includes 242 sequences from the Phytozome V12 embryophyte species with fully sequenced genome (A. thaliana, G. raimondii, P. trichocarpa, M domestica, M. truncatula, E. grandis, K. laxiflora, S. lycopersicum, A. coerulea, Z. mays, B. distachyon, O. sativa, Z. marina, and A. trichopoda), the R. rosea transcriptome, and previously characterized AAAD proteins. The phylogenetic analysis of ADHs includes 346 PAR homologs from the Phytozome V12 embryophyte species and R. rosea transcriptome. The phylogenetic analysis of UGTs contains 113 non-redundant full-length UGT homologs from the R. rosea transcriptome. A second UGT tree was also generated using the 34 cloned R. rosea UGTs in addition to the 88 full length and unique UGTs from Arabidopsis thaliana (Li et al., 2001). Initial trees for the heuristic search were obtained automatically by applying Neighbor-Join and BioNJ algorithms to a matrix of pairwise distances estimated using a JTT model, and then selecting the topology with superior log likelihood value. All phylogenetic analyses were conducted in MEGA7 (Kumar et al., 2016).

Agrobacterium-Mediated Transient Expression of Heterologous Proteins in N. benthamiana

A. tumefaciens (LBA4404) containing the transgene construct was grown to optical density (OD) 600 of 1.6 in 50 mL of YM medium (0.4 g/L yeast extract, 10 g/L mannitol, 0.1 g/L NaCl, 0.2 g/L MgSO₄·7H₂O, 0.5 g/L K₂HPO₄·3H₂O), washed with washing buffer (10 mM MES (2-(N-morpholino)ethanesulfonic acid), pH 5.6), and resuspended in MMA buffer (10 mM MES, pH 5.6, 10 mM MgCl₂, 100 μM acetosyringone) to OD 600 of 0.8. For co-expressing multiple genes, individual A. tumefaciens cultures containing the unique transgene constructs were grown, pelleted, and washed separately. The cultures were then resuspended together at a higher optical density so that each individual culture was present at a concentration equivalent to OD 600 of 0.8. 1 mL of culture was used to infiltrate the underside of six-week-old N. benthamiana leaves.

### Metabolomic Profiling by LC-HRAM-MS

Crown tissue and root tissue of a three-month-old R. rosea plant was harvested and stored at −80° C. before subsequent metabolomic analysis. Various transgene-carrying S. cerevisiae BY4743 strains and transiently transformed N. benthamiana plants were generated to test the activity of candidate genes involved in the tyrosol glycoside biosynthesis. 3 mL of saturated S. cerevisiae culture was used to inoculate 50 mL of synthetic minimal media (SD) in a shake flask. After 24 hours of shaking at 30° C., the culture was pelleted by centrifugation, washed with water, and stored at −80° C. before further processing. N. benthamiana leaf tissue was harvested 5 days after Agrobacterium infiltration and was stored at −80° C. before further processing. Frozen yeast or plant tissue was disrupted with a TissueLyser (Qiagen) using acid-washed metal beads in 50% methanol (500 μL per 100 mg fresh weight). The extracts were then analyzed by LC-HRAM-MS. Metabolite profiling was conducted on a QExactive benchtop orbitrap mass spectrometer equipped with an Ion Max source and a HESI II probe, which was coupled to a Dionex UltiMate 3000 UPLC system (Thermo Fisher Scientific). 2 μL of each sample was injected onto a 150×2.1 mm ZIC-pHILIC column (5 μm particle size, EMD Millipore). Solvent A was 20 mM ammonium carbonate, 0.1% ammonium hydroxide; solvent B was acetonitrile. The column oven and autosampler tray were held at 25° C. and 4° C., respectively. The chromatographic gradient was run at a flow rate of 0.15 mL/min as follows: 0-20 min, linear gradient from 80% to 20% solvent B; 20-20.5 min, linear gradient from 20% to 80% solvent B; 20.5-28 min, hold at 80% solvent B. The mass spectrometer was operated in full-scan, polarity-switching mode with the spray voltage set to 3.0 kV, the heated capillary held at 275° C., and the HESI probe held at 350° C. The sheath gas flow was set to 40 units, the auxiliary gas flow was set to 15 units, and the sweep gas flow was set to 1 unit. The MS data acquisition was performed in a range of 70-1000 m/z, with the resolution set at 70,000, the AGC target at 10e6, and the maximum injection time at 20 msec. The raw data was converted to mzML format using MSConvert (Chambers et al., 2012), and analyzed using MetaboAnalyst (Xia and Wishart, 2016) and MZmine2 (Pluskal et al., 2010).

### Small Molecule Isolation and NMR

For large-scale compound isolation from Agrobacterium-transformed N. benthamiana leaves, 15 g (dry weight) of N. benthamiana leaves (harvested 5 days post infection) were extracted with 70% EtOH. The solvent was evaporated from the extracts under reduced pressure using a rotary evaporator (Buchi). The residue was suspended in 100 mL of water, and extracted successively with hexane, chloroform and butanol. The water-soluble portion was separated by Sephadex LH20 using a H₂O/MeOH gradient of 0-100% MeOH. Fractions 26-32 and 36-44 were combined separately for further purification by a preparative HPLC (Shimadzu) equipped with a SPD-20A UV-VIS detector and a 150×21.2 mm 100 Å Kinetex 5 C18 column (Phenomenex). 7 mg of salidroside and 13 mg of icariside D2 were purified using water (solvent A) and a 60-minute gradient of 5-80% acetonitrile (solvent B) at a flow rate of 10 mL/min. The samples were dried by lyophilization and subjected to NMR analysis in DMSO-d₆. The solution NMR spectra were recorded on a Bruker AVANCE-400 NMR spectrometer with a Spectro Spin superconducting magnet.

### Recombinant Protein Production and Purification

BL21(DE3) E. coli containing appropriate constructs were grown at 37° C. in terrific broth (TB) to OD 600 of 0.9, induced with 0.15 mM isopropyl-β-D-thiogalactoside (IPTG), and allowed to grow for an additional 20 h at 18° C. Cells were harvested by centrifugation, washed with phosphate buffered saline (PBS) (137 mM NaCl, 2.7 mM KCl, 10 mM Na₂HPO₄ and 1.8 mM KH₂PO₄), resuspended in 150 mL of lysis buffer (50 mM Tris pH 8.0, 0.5 M NaCl, 20 mM imidazole, and 0.5 mM dithiothreitol (DTT)), and lysed with five passes through a M-110L microfluidizer (Microfluidics). The resulting crude protein lysate was clarified by centrifugation prior to Qiagen Ni-NTA gravity flow chromatographic purification. After loading the clarified lysate, His-tagged recombinant protein-bound Ni-NTA resin was washed with 20 column volumes of lysis buffer, and eluted with 1 column volume of elution buffer (50 mM Tris pH 8.0, 0.5 M NaCl, 250 mM imidazole and 0.5 mM DTT). 1 mg of His-tagged TEV protease was added to the eluted protein, followed by dialysis at 4° C. for 16 h in dialysis buffer (50 mM Tris pH 8.0, 0.1 M NaCl, 20 mM imidazole and 2 mM DTT). After dialysis, protein solution was then passed through Ni-NTA resin to remove uncleaved protein and His-tagged TEV. The recombinant protein was further purified by gel filtration on a fast protein liquid chromatography (FPLC) system (GE Healthcare Life Sciences). The principle peaks were collected, verified for molecular weight by SDS-PAGE, and stored in storage buffer (20 mM Tris pH 8.0, 25 mM NaCl, and 0.5 mM DTT) at a protein concentration of 10 mg/mL. The purity of the recombinant protein was evaluated by ImageJ densitometric analysis using bovine serum albumin as the standard (Schneider et al., 2012). 200 μM PLP was added to all buffers during the purification of all AAAD family enzymes.

### Enzyme Assays

The AAS enzyme assays were performed in 100 μL of reaction buffer (50 mM Tris, pH 8.0) containing 50 μg of recombinant enzyme, 200 μM PLP. Kinetic reactions were incubated with a range of amino acid substrate concentrations (1 μM-4 mM) at 30° C. for 30 minutes prior to quenching with 100 μL of 0.8 M formic acid. The reaction mixture was centrifuged, and the supernatant was analyzed by Pierce Quantitative Peroxide Assay Kit (Pierce) against a standard curve of hydrogen peroxide to demonstrate AAS activity or determine AAS kinetic parameters. Rr4HPAAS reactions were also analyzed by LC-MS-UV. 50 μL of reaction mixture was analyzed by an Ultimate 3000 liquid chromatography system (Dionex), equipped with a 150 mm C18 Column (Kinetex 2.6 μm silica core shell C18 100 Å pore, Phenomenex) and coupled to an UltiMate 3000 diode-array detector (DAD) in-line UV-Vis spectrophotometer (Dionex) and a TSQ Quantum Access MAX triple-quadrupole mass spectrometer (Thermo-Scientific). To resolve chromatographically L-tyrosine, tyrosol and 4-HPAAA, compounds were separated through the use of an isocratic mobile phase containing 50 mM monopotassium phosphate pH 4.6, 15% (v/v) acetonitrile and 0.5 mM octyl sulfate. Rr4HPAAS product formation was quantified using the UV absorbance at 280 nm and compared to analytical standards using the diode array detector wavelength at a wavelength range of 200-500 nm and chromatographic retention time. The reduction of aldehyde products was achieved by addition of saturated sodium borohydride in ethanol or by addition of 10 mM NADPH and 10 g of Rr4HPAR1 or Rr4HPAR2.

The phenylacetaldehyde reductase activity assays using Rr4HPAR1 and RrPAR2 were carried out in 200 L reaction buffer (50 mM Tris, pH 8.0) at the presence of 2 mM phenylacetaldehyde, 5 mM NADPH and 5 g of recombinant enzyme. The reactions were incubated at 30° C. for various time points, quenched with an equal volume of 0.8 M formic acid, and extracted by 100 μL of ethyl acetate. The organic phase was then analyzed by gas chromatography-mass spectrometry (GC-MS) using an 5% Phenyl Methyl Silox column (30 m x 250 m×0.25 m, Agilent) with a temperature gradient as follows: 0-1 min 45° C., 4-13.33 min 45-185° C. The quadrupole MS was set to EI mode, electron energy at 70 eV, MS-source temperature at 230° C., MS-quad temperature at 150° C., scan mass range at 50-300 m/z and SIM for 120 m/z and 122 m/z. EI-MS spectra were compared against analytical standards. The 4-HPAA reductase activity assays were carried out in 100 μL of 50 mM Tris pH 8.0 with the addition of 5 mM 4-HPAA, 10 mM NADPH and 0.2 ag of Rr4HPAR1 or 15 jg of Rr4HPAR2. The reactions were incubated at 30° C. and then quenched at various time points with the addition of 100 μL methanol. The reaction mixture was then centrifuged and analyzed by LC-MS. Compounds were separated by reversed-phase chromatography with a ramp gradient of solvent A (0.1% formic acid in H₂O) and solvent B (0.1% formic acid in acetonitrile): 10% solvent B for 0.5 min, 5-40% solvent B over 8.5 min, 95% solvent B for 1.8 min followed by a final equilibration of 10% solvent B for 1 min with a flow rate at 0.7 mL/min. Product formation was measured using select ion monitoring in positive mode for a centroid center mass of 121.065 with a scan width of 0.002. The specific activity was determined at a five-minute reaction time point, and quantified against a standard curve of tyrosol.

Kinetic characterization of UGTs was conducted in 200 L reaction buffer (50 mM Tris, pH 8.0) containing 10 mM UDP-glucose and various concentrations of tyrosol (0.01-5.0 mM). Reactions were started with addition of recombinant enzyme, incubated at 30° C. for 10 minutes, and quenched by addition of 200 μL of methanol. The reaction mixed was then analyzed by LC-HRAR-MS as described above. Compounds were separated by reversed-phase chromatography with a ramp gradient of solvent A (0.1% formic acid in H20) and solvent B (0.1% formic acid in acetonitrile): 5% solvent B for 0.5 min, 5-55% solvent B over 6 min, 55-5% solvent B over 1.0 min and a final equilibration of 5% solvent B for 1 min with a flow rate at 0.6 mL/min. Product formation was measured using select ion monitoring in positive mode for a centroid center mass of 318.15. Product mass was calculated by comparison to a standard curve of the NMR verified plant purified salidroside and icariside D2 samples.

Kinetic constants such as Kₘ and Vmax were determined by fitting raw data to the Michaelis-Menten equation using the nonlinear regression function in Prism (version 7.0).

### Accession Codes

The sequences of R. rosea genes reported in this article are deposited into NCBI GenBank under the following accession numbers: Rr4HPAAS (MF674522), RrAAS (MF674523), Rr4HPAR1-2 (MF674524-MF674525) and RrUDP1-34 (MF674526-MF674558, MG385659). Raw RNA-Seq reads have been submitted to NCBI SRA (SRR5936536 and SRR5936537). The de novo transcriptomes assembled from the raw reads have been submitted to NCBI TSA (GFVD00000000 for merged transcriptome, GFVE00000000 for crown transcriptome, and GFVF00000000 for root transcriptome). Raw and mzTab format feature called metabolomic data from the R. rosea crown and root have been uploaded to the EBI MetaboLights database (MTBLS566).

TABLE 1
Kinetic parameters of characterized enzymes.

| | Enzyme | | | | |
|---|---|---|---|---|---|
| | RrT4GT | RrT8GT | RrUGT29 | Rr4HPAAS | Rr4HPAAS |
| | Substrate | | | | |
| | tyrosol | tyrosol | tyrosol | L-tyrosine | L-DOPA |
| k_{cat}(sec⁻¹) | 481.60 ± 3.91 | 576.20 ± 5.68 | 167.5 ± 0.82 | 4.92 ± 0.08 | 9.52 ± 0.37 |
| Kₘ(mM) | 4.11 ± 0.08 | 1.37 ± 0.05 | 0.53 ± 0.01 | 0.42 ± 0.02 | 1.04 ± 0.10 |
| k_{cat}/Kₘ(sec⁻¹ mM⁻¹) | 117.18 | 420.58 | 316.04 | 11.71 | 9.15 |

TABLE 2
Cloning primers.

| | Vector/ | SEQ ID | |
|---|---|---|---|
| Gene | direction | NO: | Sequence |
| Rr4HPAAS | pHis8-4 | 97 | GAAAACTTGTACTTCCAGGCCCATGGCATGGGC |
| | Forward | | AGCTTGCCTTCTCCTAATG |
| Rr4HPAAS | pHis8-4 | 98 | CTCGAATTCGGATCCGCCATGGCTAAGACACGA |
| | Reverse | | TGCTTTGAGCTGTTTCTTG |
| Rr4HPAAS | pEAQ-HT | 99 | GTATATTCTGCCCAAATTCGCGACCGGTATGGGC |
| | Forward | | AGCTTGCCTTCTCCTAATG |
| Rr4HPAAS | pEAQ-HT | 100 | GAAAATTTAATGAAACCAGAGTTAAAGGCCTCG |
| | Reverse | | AGCTAAGACACGATGCTTTGAGCTGTTTCTTG |
| Rr4HPAAS | p423TEF | 101 | GCATAGCAATCTAATCTAAGTTTTCTAGAACTAG |
| | Forward | | TATGGGCAGCTTGCCTTCTCC |
| Rr4HPAAS | p423TEF | 102 | CAGCCCGGGGGATCCACTAGTCTAAGACACGAT |
| | Reverse | | GCTTTGAGCTGTTTCTTG |
| RrAAS | pHis8-4 | 103 | GAAAACTTGTACTTCCAGGCCCATGGCATGGAG |
| | Forward | | GAGGAGTTGAAGCCG |
| RrAAS | pHis8-4 | 104 | CTCGAATTCGGATCCGCCATGGTCATGCATTTAT |
| | Reverse | | ATGCTTTTGTAGCAGTGAAGTG |
| RrPAR1 | pHis8-4 | 105 | GAAAACTTGTACTTCCAGGCCCATGGCATGAGTT |
| | Forward | | TAAGCGGAGCGGGG |
| RrPAR1 | pHis8-4 | 106 | CTCGAATTCGGATCCGCCATGGTCAGAGTTTGGC |
| | Reverse | | GAAACCCTTTTCC |
| RrPAR1 | p425TEF | 107 | GCATAGCAATCTAATCTAAGTTTTCTAGAACTAG |
| | Forward | | TATGAGTTTAAGCGGAGCGGGG |
| RrPAR1 | p425TEF | 108 | CAGCCCGGGGGATCCACTAGTTCAGAGTTTGGC |
| | Reverse | | GAAACCCTTTTCC |
| RrPAR2 | pHis8-4 | 109 | GAAAACTTGTACTTCCAGGCCCATGGCATGGGTT |
| | Forward | | TATCTGAAGAGAAGAAGTTAG |
| RrPAR2 | pHis8-4 | 110 | CTCGAATTCGGATCCGCCATGGTCATTTGTCTTT |
| | Reverse | | CAAACTTTCGACAGTGTCTC |
| RrUGT1 | p426TEF | 111 | CAATCTAATCTAAGTTTTCTAGAACTAGTATGGT |
| | Forward | | GACGAAAAAAACTCACATTCTTATCC |
| RrUGT1 | p426TEF | 112 | CAGCCCGGGGGATCCACTAGTTCAGGTAAGACC |
| | Reverse | | AGACACAAACTTGAC |
| RrUGT2 | p426TEF | 113 | CAATCTAATCTAAGTTTTCTAGAACTAGTATGGG |
| | Forward | | TTCTGATTCACGGCCTC |
| RrUGT2 | p426TEF | 114 | CAGCCCGGGGGATCCACTAGTCTAGGACAAAGT |
| | Reverse | | CTCTCTTCTCAACTTCAATTC |
| RrUGT2 | pHis8-4 | 115 | GAAAACTTGTACTTCCAGGCCCATGGCATGGGTT |
| | Forward | | CTGATTCACGGCCTC |
| RrUGT2 | pHis8-4 | 116 | CTCGAATTCGGATCCGCCATGGCTAGGACAAAG |
| | Reverse | | TCTCTCTTCTCAACTTCAATTC |
| RrUGT2 | pEAQ-HT | 117 | GTATATTCTGCCCAAATTCGCGACCGGTATGGGT |
| | Forward | | TCTGATTCACGGCCTC |
| RrUGT2 | pEAQ-HT | 118 | GAAAATTTAATGAAACCAGAGTTAAAGGCCTCG |
| | Reverse | | AG CTAGGACAAAGTCTCTCTTCTCAACTTC |
| RrUGT3 | p426TEF | 119 | CAATCTAATCTAAGTTTTCTAGAACTAGTATGTC |
| | Forward | | AGGCACACCACACATCG |
| RrUGT3 | p426TEF | 120 | CAGCCCGGGGGATCCACTAGTTCAATGCTTCATC |
| | Reverse | | GAACTCCGCC |
| RrUGT3 | pHis8-4 | 121 | GAAAACTTGTACTTCCAGGCCCATGGCATGTCAG |
| | Forward | | GCACACCACACATCG |
| RrUGT3 | pHis8-4 | 122 | CTCGAATTCGGATCCGCCATGGTCAATGCTTCAT |
| | Reverse | | CGAACTCCGCC |
| RrUGT3 | pEAQ-HT | 123 | GTATATTCTGCCCAAATTCGCGACCGGTATGTCA |
| | Forward | | GGCACACCACACATCG |
| RrUGT3 | pEAQ-HT | 124 | GAAAATTTAATGAAACCAGAGTTAAAGGCCTCG |
| | Reverse | | AGTCAATGCTTCATCGAACTCCGCC |
| RrUGT4 | p426TEF | 125 | CAATCTAATCTAAGTTTTCTAGAACTAGTATGGG |
| | Forward | | TTCACAAGCCTCTCCAAAACC |
| RrUGT4 | p426TEF | 126 | CAGCCCGGGGGATCCACTAGTTCATTCCTTGAAC |
| | Reverse | | TGGAGAATATCTTTCACAAGCC |
| RrUGT5 | p426TEF | 127 | CAATCTAATCTAAGTTTTCTAGAACTAGTATGGA |
| | Forward | | ACCGAGACCTCACGCAG |
| RrUGT5 | p426TEF | 128 | CAGCCCGGGGGATCCACTAGTTTAATTAGTGTCA |
| | Reverse | | CCAAGATGAGTTTTCTTTAGTAAG |
| RrUGT6 | p426TEF | 129 | CAATCTAATCTAAGTTTTCTAGAACTAGTATGGA |
| | Forward | | ATCTGTACAAGGTGTTCAAGAAAAGC |
| RrUGT6 | p426TEF | 130 | CAGCCCGGGGGATCCACTAGTTCAGTTTGAATTC |
| | Reverse | | CTCGACAGGAGCAC |
| RrUGT7 | p426TEF | 131 | CAATCTAATCTAAGTTTTCTAGAACTAGTATGGC |
| | Forward | | TGAAAACACTCATGCTCATGC |
| RrUGT7 | p426TEF | 132 | CAGCCCGGGGGATCCACTAGTTCATTTCTTGAAG |
| | Reverse | | ATTTGTAGGTCGTGGATG |
| RrUGT8 | p426TEF | 133 | CAATCTAATCTAAGTTTTCTAGAACTAGTATGGC |
| | Forward | | TTCCTCCTCTTTAGCTTGTGATTC |
| RrUGT8 | p426TEF | 134 | CAGCCCGGGGGATCCACTAGTTTATTTAACTGTT |
| | Reverse | | TCTTGTTTTTGCAGGACAGAATGAATG |
| RrUGT9 | p426TEF | 135 | CAATCTAATCTAAGTTTTCTAGAACTAGTATGGG |
| | Forward | | GTCTGAGCCACTAGTCC |
| RrUGT9 | p426TEF | 136 | CAGCCCGGGGGATCCACTAGTTTATGCTGAAATT |
| | Reverse | | GCATCCTTAGCAACTGG |
| RrUGT10 | p426TEF | 137 | CAATCTAATCTAAGTTTTCTAGAACTAGTATGAC |
| | Forward | | GAGGCGCCACCAC |
| RrUGT10 | p426TEF | 138 | CAGCCCGGGGGATCCACTAGTTCATCCAAGGCC |
| | Reverse | | ATTGACAAAACGAC |
| RrUGT11 | p426TEF | 139 | CAATCTAATCTAAGTTTTCTAGAACTAGTATGGC |
| | Forward | | AGGCGAGATTCTAATACTTCCG |
| RrUGT11 | p426TEF | 140 | CAGCCCGGGGGATCCACTAGTTCACTTGTGGGA |
| | Reverse | | GATAATGAAGTCCCTG |
| RrUGT12 | p426TEF | 141 | CAATCTAATCTAAGTTTTCTAGAACTAGTATGGA |
| | Forward | | GGAGGCGGCCAG |
| RrUGT12 | p426TEF | 142 | CAGCCCGGGGGATCCACTAGTTTAACACAGAGT |
| | Reverse | | CCAAATGTCCAGCAAC |
| RrUGT13 | p426TEF | 143 | CAATCTAATCTAAGTTTTCTAGAACTAGTATGCT |
| | Forward | | ACCTCTCTTACATGTTACACTAAC |
| RrUGT13 | p426TEF | 144 | CAGCCCGGGGGATCCACTAGTTTACAAGCCAAT |
| | Reverse | | GTTGGTCCTGAGATCAC |
| RrUGT14 | p426TEF | 145 | CAATCTAATCTAAGTTTTCTAGAACTAGTATGGA |
| | Forward | | CACCACCGCCGC |
| RrUGT14 | p426TEF | 146 | CAGCCCGGGGGATCCACTAGTTTATCCCCTTCCA |
| | Reverse | | AGTTGAGTCAACGAC |
| RrUGT15 | p426TEF | 147 | CAATCTAATCTAAGTTTTCTAGAACTAGTATGGC |
| | Forward | | TGATGCTGCTCAACATGTC |
| RrUGT15 | p426TEF | 148 | CAGCCCGGGGGATCCACTAGTTTATTGAACTTTG |
| | Reverse | | TGAAATTGAAGATGACTCAAAAGG |
| RrUGT16 | p426TEF | 149 | CAATCTAATCTAAGTTTTCTAGAACTAGTATGGC |
| | Forward | | AGAGGAAAACAGAACCAGC |
| RrUGT16 | p426TEF | 150 | CAGCCCGGGGGATCCACTAGTTCATACAGCTGA |
| | Reverse | | AGATATTTTGGATATGAATTGGTC |
| RrUGT17 | p426TEF | 151 | CAATCTAATCTAAGTTTTCTAGAACTAGTATGGG |
| | Forward | | CTCACTTCCTTCCAC |
| RrUGT17 | p426TEF | 152 | CAGCCCGGGGGATCCACTAGTTCAGACGCTAAA |
| | Reverse | | CTGGACCACTTTTTCC |
| RrUGT18 | p426TEF | 153 | CAATCTAATCTAAGTTTTCTAGAACTAGTATGGG |
| | Forward | | CTCCCGAGGAAAGCCACATG |
| RrUGT18 | p426TEF | 154 | CAGCCCGGGGGATCCACTAGTTCATTTTGGGGA |
| | Reverse | | ATTAGACAGCAGG |
| RrUGT19 | p426TEF | 155 | CAATCTAATCTAAGTTTTCTAGAACTAGTATGAC |
| | Forward | | GTCATCAACACCTCCTCCTC |
| RrUGT19 | p426TEF | 156 | CAGCCCGGGGGATCCACTAGTCTAAAAAAATGC |
| | Reverse | | TTTAACATAGCTAGCGTCCG |
| RrUGT20 | p426TEF | 157 | CAATCTAATCTAAGTTTTCTAGAACTAGTATGGG |
| | Forward | | TTCACTCGACGTCGTC |
| RrUGT20 | p426TEF | 158 | CAGCCCGGGGGATCCACTAGTTCATTTCATAATA |
| | Reverse | | GCTTCATCAATCAACTCGG |
| RrUGT21 | p426TEF | 159 | CAATCTAATCTAAGTTTTCTAGAACTAGTATGAA |
| | Forward | | GTCCAACACTCATCTATTCCTC |
| RrUGT21 | p426TEF | 160 | CAGCCCGGGGGATCCACTAGTTCATACAACCGG |
| | Reverse | | CTCCAGTTGAC |
| RrUGT22 | p426TEF | 161 | CAATCTAATCTAAGTTTTCTAGAACTAGTATGAA |
| | Forward | | AACTCCTCAAAATCCACACGTAG |
| RrUGT22 | p426TEF | 162 | CAGCCCGGGGGATCCACTAGTTCAATCCTGATA |
| | Reverse | | AATCTTTGAACTCATCTTGCTC |
| RrUGT23 | p426TEF | 163 | CAATCTAATCTAAGTTTTCTAGAACTAGTATGGA |
| | Forward | | AAGGCAGAGTGATCACCAAG |
| RrUGT23 | p426TEF | 164 | CAGCCCGGGGGATCCACTAGTTCATTTGGTGGAT |
| | Reverse | | ATCACATCTCTAACAAACTG |
| RrUGT24 | p426TEF | 165 | CAATCTAATCTAAGTTTTCTAGAACTAGTATGAG |
| | Forward | | CAACGCCGCCG |
| RrUGT24 | p426TEF | 166 | CAGCCCGGGGGATCCACTAGTTTAGTTTATGACT |
| | Reverse | | TCATTCACTTGCTCCAACAAC |
| RrUGT25 | p426TEF | 167 | CAATCTAATCTAAGTTTTCTAGAACTAGTATGGC |
| | Forward | | GCGCCACCACTTTG |
| RrUGT25 | p426TEF | 168 | CAGCCCGGGGGATCCACTAGTTTAGCAGGTAAC |
| | Reverse | | AAGGTTATTAACCAAATCCTTGAG |
| RrUGT26 | p426TEF | 169 | CAATCTAATCTAAGTTTTCTAGAACTAGTATGTC |
| | Forward | | ATCAGATTCCGGCCACATTATCC |
| RrUGT26 | p426TEF | 170 | CAGCCCGGGGGATCCACTAGTCTATATTATTTTT |
| | Reverse | | CTTAATGCCATGACTTGTCGGACC |
| RrUGT27 | p426TEF | 171 | CAATCTAATCTAAGTTTTCTAGAACTAGTATGAG |
| | Forward | | TTCAGTCAATGCTCAAAAGCC |
| RrUGT27 | p426TEF | 172 | CAGCCCGGGGGATCCACTAGTTCAAAAGTGCAT |
| | Reverse | | TAGTAGTCCTTCCACAAATC |
| RrUGT28 | p426TEF | 173 | CAATCTAATCTAAGTTTTCTAGAACTAGTATGGA |
| | Forward | | CTCGGTTGATCTGAACAAG |
| RrUGT28 | p426TEF | 174 | CAGCCCGGGGGATCCACTAGTCTAGTTGGCACTT |
| | Reverse | | GGCAACACAATCG |
| RrUGT29 | p426TEF | 175 | CAATCTAATCTAAGTTTTCTAGAACTAGTATGGG |
| | Forward | | ATCTCTAGGAAAGAAGATTCAAC |
| RrUGT29 | p426TEF | 176 | CAGCCCGGGGGATCCACTAGTTTAGGTTGTAACT |
| | Reverse | | ACAATTTTTTTTTTGGAC |
| RrUGT29 | pHis8-4 | 177 | GAAAACTTGTACTTCCAGGCCCATGGCATGGGA |
| | Forward | | TCTCTAGGAAAGAAGATTCAAC |
| RrUGT29 | pHis8-4 | 178 | CTCGAATTCGGATCCGCCATGGTTAGGTTGTAAC |
| | Reverse | | TACAATTTTTTTTTTGGAC |
| RrUGT29 | pEAQ-HT | 179 | GTATATTCTGCCCAAATTCGCGACCGGTATGGGA |
| | Forward | | TCTCTAGGAAAGAAGATTCAAC |
| RrUGT29 | pEAQ-HT | 180 | GAAAATTTAATGAAACCAGAGTTAAAGGCCTCG |
| | Reverse | | AGTTAGGTTGTAACTACAATTTTTTTTTTGGAC |
| RrUGT30 | p426TEF | 181 | CAATCTAATCTAAGTTTTCTAGAACTAGTATGGG |
| | Forward | | CTCCCGAGGAAAGCCACATG |
| RrUGT30 | p426TEF | 182 | CAGCCCGGGGGATCCACTAGTTCATTTTGGGGA |
| | Reverse | | ATTAGACAGCAGG |
| RrUGT31 | p426TEF | 183 | CAATCTAATCTAAGTTTTCTAGAACTAGTATGGA |
| | Forward | | ATCTGTACAAGGTGTTCAAGAAAAG |
| RrUGT31 | p426TEF | 184 | CAGCCCGGGGGATCCACTAGTTCAGTTTGAATTC |
| | Reverse | | CTCGACAGGAGCAC |
| RrUGT32 | p426TEF | 185 | CAATCTAATCTAAGTTTTCTAGAACTAGTATGGA |
| | Forward | | CTCGGTTGATCTGAACAAGAAACC |
| RrUGT32 | p426TEF | 186 | CAGCCCGGGGGATCCACTAGTCTACAATTTTTTT |
| | Reverse | | TTGGACAGAAGTACGTCATTTATAAGTC |
| RrUGT33 | p426TEF | 187 | CAATCTAATCTAAGTTTTCTAGAACTAGTATGAG |
| | Forward | | CTTAATTGAAAAACCACTCACG |
| RrUGT33 | p426TEF | 188 | CAGCCCGGGGGATCCACTAGTCTAACGGATATG |
| | Reverse | | TTTTGTTTTTGAGAGCAGGAC |
| RrUGT33 | pHis8-4 | 189 | GAAAACTTGTACTTCCAGGCCCATGGCATGAGCT |
| | Forward | | TAATTGAAAAACCACTCACG |
| RrUGT33 | pHis8-4 | 190 | CTCGAATTCGGATCCGCCATGGCTAACGGATATG |
| | Reverse | | TTTTGTTTTTGAGAGCAGGAC |
| RrUGT33 | pEAQ-HT | 191 | GTATATTCTGCCCAAATTCGCGACCGGTATGAGC |
| | Forward | | TTAATTGAAAAACCACTCACG |
| RrUGT33 | pEAQ-HT | 192 | GAAAATTTAATGAAACCAGAGTTAAAGGCCTCG |
| | Reverse | | AGCTAACGGATATGTTTTGTTTTTGAGAGCAGGA |
| | | | C |
| RrUGT34 | p426TEF | 193 | GCATAGCAATCTAATCTAAGTTTTCTAGAACTAG |
| | Forward | | TTGGACCCTGACGACAGCGTTTTG |
| RrUGT34 | p426TEF | 194 | CAGCCCGGGGGATCCACTAGTTTAGTTTTTGTTC |
| | Reverse | | TCGTACAAATAATGCACAAACTCATC |
| Pc4HPAAS | pHis8-4 | 195 | GAAAACTTGTACTTCCAGGCCCATGGCATGGGCT |
| | Forward | | CCATCGATAATC |
| Pc4HPAAS | pHis8-4 | 196 | CTCGAATTCGGATCCGCCATGGTTAGGATAAAAT |
| | Reverse | | ATTCACGATCTTCT |
| Pc4HPAAS | pEAQ-HT | 197 | GTATATTCTGCCCAAATTCGCGACCGGTATGGGC |
| | Forward | | TCCATCGATAATC |
| Pc4HPAAS | pEAQ-HT | 198 | GAAAATTTAATGAAACCAGAGTTAAAGGCCTCG |
| | Reverse | | AGTTAGGATAAAATATTCACGATCTTC |
| PsTyDC | pHis8-4 | 199 | GAAAACTTGTACTTCCAGGCCCATGGCATGGGA |
| | Forward | | AGCCTTCCGACTAATAACCTTG |
| PsTyDC | pHis8-4 | 200 | CTCGAATTCGGATCCGCCATGGCTAGGCACCAA |
| | Reverse | | GTATGGCATCTGTATG |
| PsTyDC | pEAQ-HT | 201 | GTATATTCTGCCCAAATTCGCGACCGGTATGGGA |
| | Forward | | AGCCTTCCGACTAATAACCTTG |
| PsTyDC | pEAQ-HT | 202 | GAAAATTTAATGAAACCAGAGTTAAAGGCCTCG |
| | Reverse | | AGCTAGGCACCAAGTATGGCATCTGTATG |
| AAS55083 | p426TEF | 203 | CAATCTAATCTAAGTTTTCTAGAACTAGTATGGC |
| | Forward | | AGGCAGTGGGACTG |
| AAS55083 | p426TEF | 204 | CAGCCCGGGGGATCCACTAGTTCAGTGTTTAACT |
| | Reverse | | GAGGATCTCCACTTTTTAGC |
| EU567325 | p426TEF | 205 | GCATAGCAATCTAATCTAAGTTTTCTAGAACTAG |
| | Forward | | TATGGGTTCTGAAACTCGGCCTTTG |
| EU567325 | p426TEF | 206 | CAGCCCGGGGGATCCACTAGTCTAGACTTTCTTT |
| | Reverse | | AACTTGAGTTCCTGAAGCAG |

### Example #2: Results

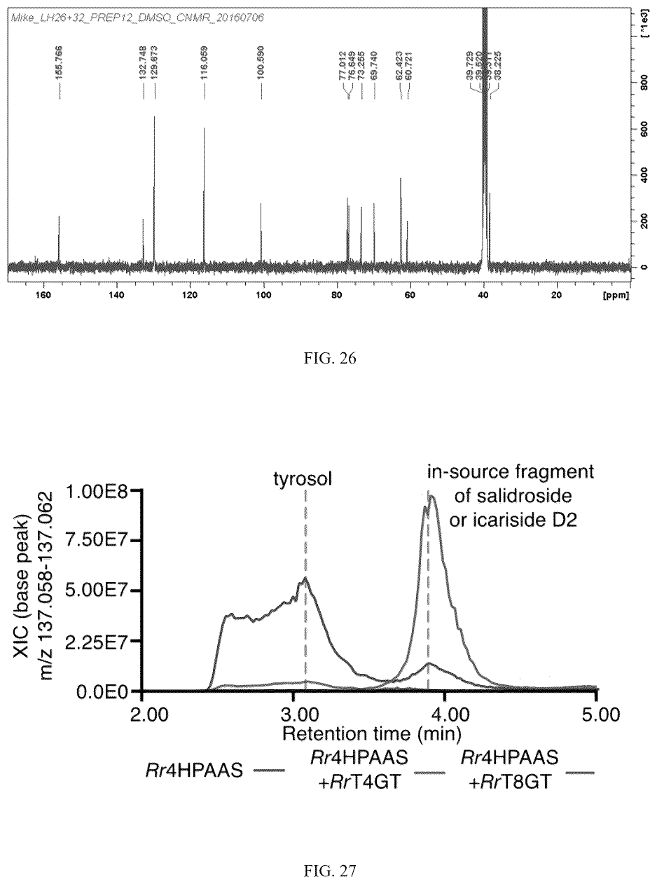

Enzymes of the plant aromatic amino acid decarboxylases (AAAD) family that can be used in the production of one or more of tyrosol, salidroside, and icariside D2 were identified. These plant AAAD-family enzymes contain substitutions in one of two active site residues responsible for influencing aldehyde synthase chemistry. These activity-influencing residues are boxed in the multiple sequence alignment of biochemically characterized plant AAADs show in FIG. 26.

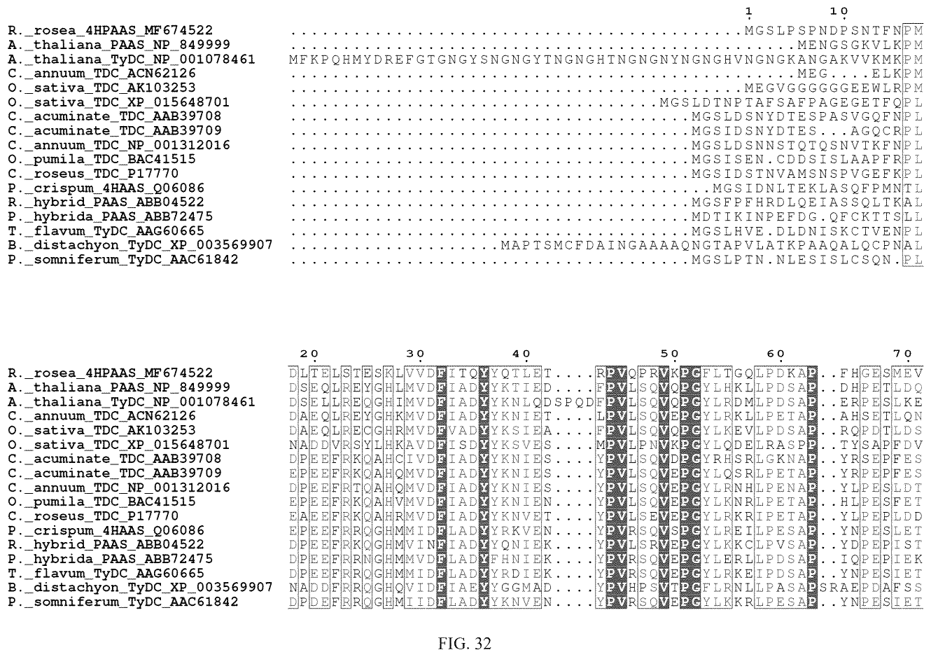

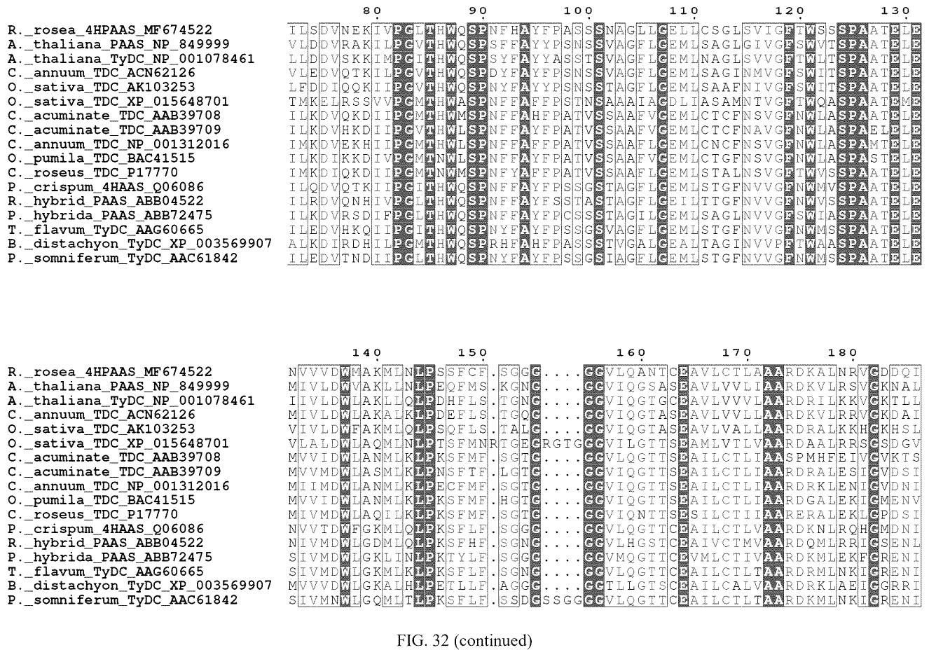

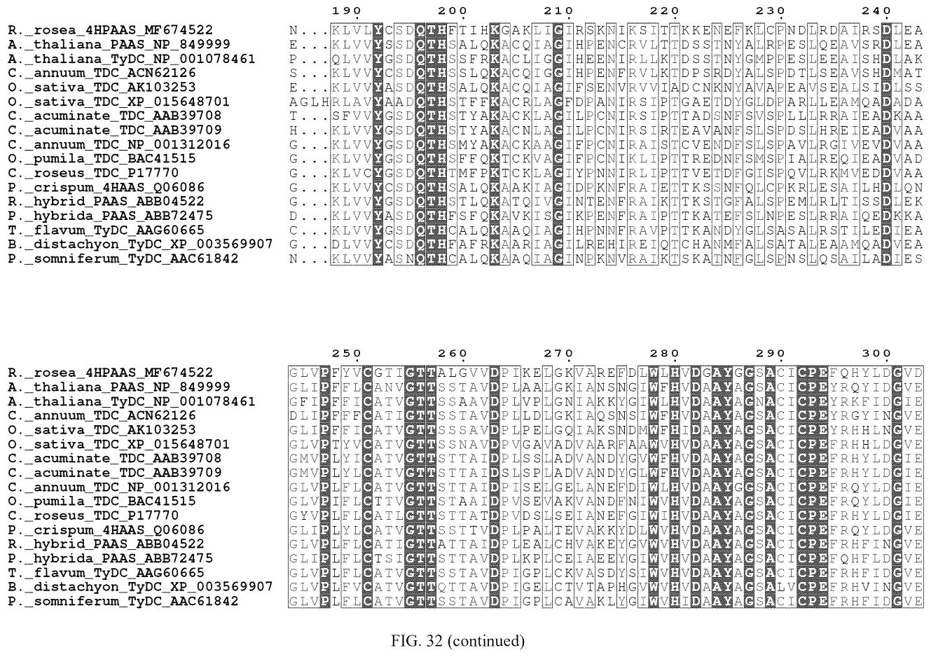

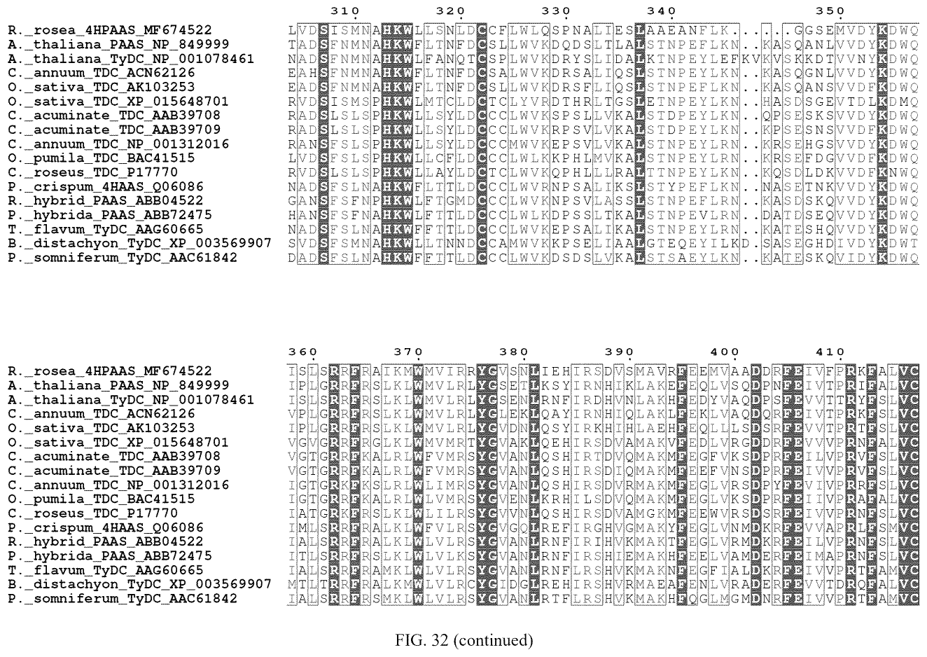

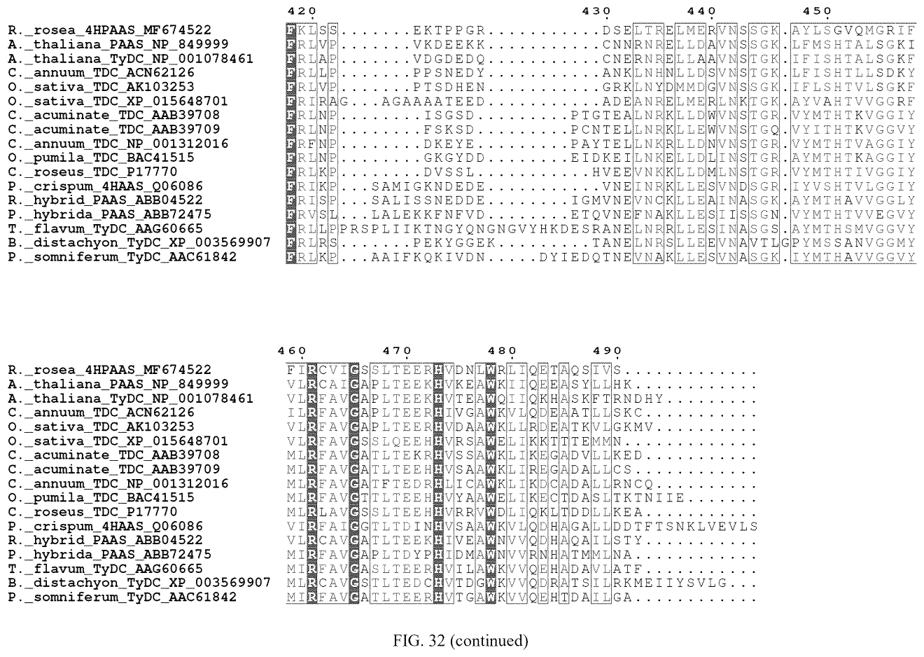

Plant AAAD enzymes that contain an active site histidine to asparagine or aspartic acid substitution have an aldehyde synthase activity. This active site substitution is represented by the Rr4HPAAS MF674522 histidine 198 in FIGS. 31 and 32.

Plant AAAD enzymes that contain an active site tyrosine to leucine, isoleucine, phenylalanine, methionine or valine substitution have an aldehyde synthase activity. This active site substitution is represented by the Rr4HPAAS MF674522 phenylalanine 343 in FIGS. 31 and 32.

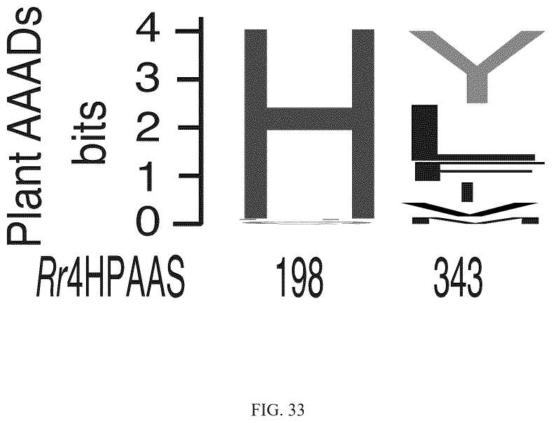

These active site substitutions at positions 198 and 343 were selected from natural variation, shown in FIG. 33, found within all plant AAAD sequences available on Phytozome V12.1. We have curated 226 plant AAAD sequences from Phytozome 12. The list was queried for sequences containing substitution in one of the two activity dictating residues to make a list of 73 enzymes that likely have some 41PAAS activity. These 73 AAS enzymes are identified as SEQ ID NOS: 21-93. In some embodiments, any of the enzymes of SEQ ID NOS: 21-93 can provide 4HPAAS activity in a host cell or method described herein.

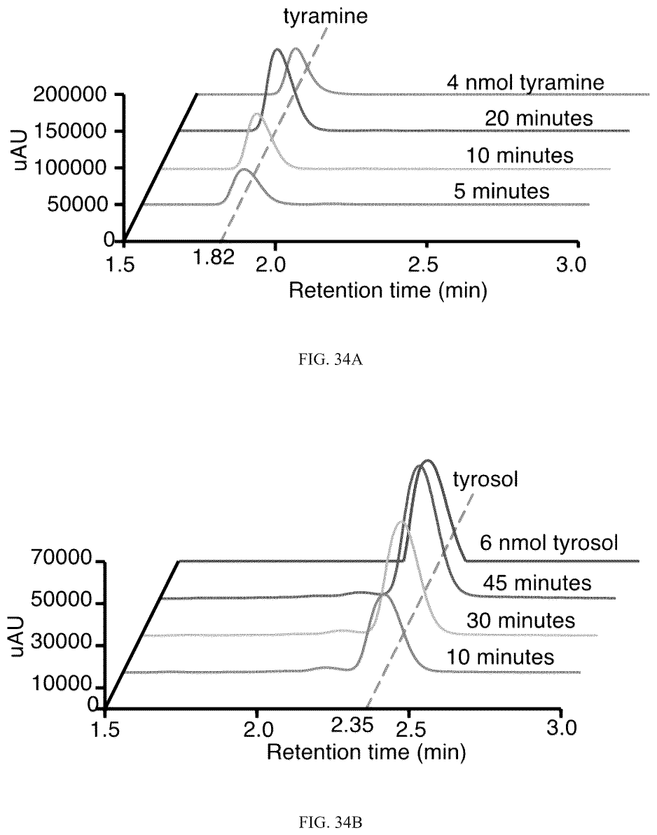

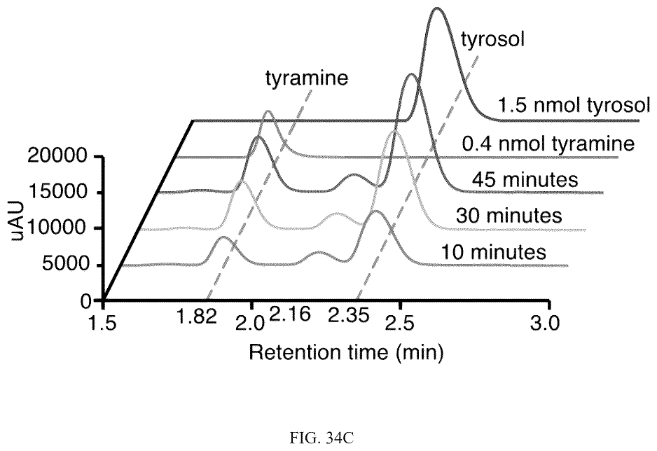

To demonstrate the roles of these residues in aldehyde synthase chemistry, the biochemical activity of wild type and mutant Papaver somniferum tyrosine decarboxylase (PsTyDC) enzymes were characterized. The substitution of the active site histidine (Rr4HPAAS MF674522 histidine 198) or the active site tyrosine (Rr4HPAAS MF674522 phenylalanine 343) within a Papaver somniferum tyrosine decarboxylase (PsTyDC) results in aldehyde synthase chemistry. FIGS. 34A-C are chromatograms showing product formation of PsTyDC and mutants.

### Sequences

TABLE 2
Summary of Sequences.

| SEQ | |
|---|---|
| ID | |
| NO.: | Description |
| 1 | Rr4HPAAS DNA |
| 2 | Rr4HPAAS GenBank accession MF674522 |
| 3 | Rr4HPAR1 DNA |
| 4 | Rr4HPAR1 amino acid GenBank accession MF674524 |
| 5 | RrUGT2 DNA |
| 6 | RrUGT2 amino acid GenBank accession MF674527 |
| 7 | RrUGT3 DNA |
| 8 | RrUGT3 amino acid GenBank accession MF674528 |
| 9 | RrUGT7 DNA |
| 10 | RrUGT7 amino acid GenBank accession MF674532 |
| 11 | RrUGT13 DNA |
| 12 | RrUGT13 amino acid GenBank accession MF674538 |
| 13 | RrUGT17DNA |
| 14 | RrUGT17 amino acid GenBank accession MF674542 |
| 15 | RrUGT29 DNA |
| 16 | RrUGT29 amino acid GenBank accession MF674554 |
| 17 | RrUGT32 DNA |
| 18 | RrUGT32 amino acid GenBank accession MF674557 |
| 19 | RrUGT33 DNA |
| 20 | RrUGT33 amino acid GenBank accession MF674558 |
| 21 | Arabidopsis thaliana AT2G20340.1 |
| 22 | Brachypodium distachyon 1g28960.3 |
| 23 | Carica papaya 16427710 |
| 24 | Ricinus communis 16804377 |
| 25 | Cucumis sativus 16963476 |
| 26 | Vitis vinifera 17835588 |
| 27 | Citrus sinensis 18113817 |
| 28 | Capsella rubella 20900667 |
| 29 | Malus domestica 22636618 |
| 30 | Linum usitatissimum 23178995 |
| 31 | Eutrema salsugineum 20200788 |
| 32 | Populus trichocarpa 27022899 |
| 33 | Brachypodium stacei 06G160800.1 |
| 34 | Physcomitrella patens Pp3c4_30790V3.1 |
| 35 | Ananas comosus 33033299 |
| 36 | Zostera marina 33182387 |
| 37 | Daucus carota subsp. sativus 36055203 |
| 38 | Trifolium pratense 35974269 |
| 39 | Arabidopsis lyrata 35943929 |
| 40 | Sorghum bicolor 002G120700.1 |
| 41 | Sphagnum fallax 0166s0011.1 |
| 42 | Kalanchoe laxiflora 1398s0003.1 |
| 43 | Manihot esculenta 12G038600.1 |
| 44 | Prunus persica 8G214500.1 |
| 45 | Eucalyptus grandis K01418.1 |
| 46 | Amborella trichopoda 31565185 |
| 47 | Salix purpurea 0252s0200.1 |
| 48 | Medicago truncatula 31080941 |
| 49 | Brassica rapa I01156.1 |
| 50 | Brassica rapa I04706.1 |
| 51 | Brassica rapa G00043.1 |
| 52 | Glycine max 03G167900.1 |
| 53 | Fragaria vesca 27261550 |
| 54 | Kalanchoe fedtschenkoi 0172s0035.1 |
| 55 | Capsella grandiflora 22666s0001.1 |
| 56 | Selaginella moellendorffii 15420188 |
| 57 | Setaria italica 3G188200.1 |
| 58 | Kalanchoe fedtschenkoi 0033s0078.1 |
| 59 | Daucus carota subsp. sativus 36068870 |
| 60 | Daucus carota subsp. sativus 36056758 |
| 61 | Solanum tuberosum 3DMP400026166 |
| 62 | Solanum tuberosum 3DMP400024738 |
| 63 | Solanum lycopersicum 36137005 |
| 64 | Daucus carota subsp. sativus 36065781 |
| 65 | Oropetium thomaeum 35995617 |
| 66 | Oryza sativa 33157740 |
| 67 | Brachypodium stacei 01G392300.1 |
| 68 | Amaranthus hypochondriacus 32828676 |
| 69 | Brachypodium distachyon 5g21770.1 |
| 70 | Brachypodium distachyon 2g02360.1 |
| 71 | Sorghum bicolor 009G192600.1 |
| 72 | Kalanchoe laxiflora 0994s0009.1 |
| 73 | Kalanchoe laxiflora 0003s0173.1 |
| 74 | Panicum hallii 32512198 |
| 75 | Prunus persica 6G202600.1 |
| 76 | Prunus persica 4G086700.1 |
| 77 | Prunus persica 4G087100.1 |
| 78 | Medicago truncatula 31073039 |
| 79 | Zea mays GRMZM2G009400 |
| 80 | Glycine max 07G059000.1 |
| 81 | Panicum virgatum Ca01381.1 |
| 82 | Theobroma cacao 27425420 |
| 83 | Fragaria vesca 27274768 |
| 84 | Gossypium raimondii 26786642 |
| 85 | Populus trichocarpa 26994989 |
| 86 | Malus domestica 22679008 |
| 87 | Citrus Clementina 20801973 |
| 88 | Citrus Clementina 20818150 |
| 89 | Vitis vinifera 17834108 |
| 90 | Petunia hybrida ABB72475.1 |
| 91 | Carica papaya 16421889 |
| 92 | Sphagnum fallax 0042s0024.1 |
| 93 | Eucalyptus grandis E01788.1 |
| 94 | pHis8-4 |
| 95 | pEAQ-HT |
| 96 | pJKW 1410 |

Rr4HPAAS DNA (SEQ ID NO: 1):
ATGGGCAGCTTGCCTTCTCCTAATGATCCATCAAACACCTTCAACCCCATGGACCTC
ACCGAGTTATCCACCGAGTCGAAACTCGTCGTAGATTTCATAACTCAGTACTACCAA
ACCCTAGAGACCCGACCCGTCCAGCCACGGGTCAAGCCAGGTTTCTTAACGGGCCA
GCTTCCAGATAAAGCACCCTTTCATGGTGAATCAATGGAAGTAATATTGTCTGATGT
AAATGAGAAGATTGTCCCTGGCCTCACTCATTGGCAAAGCCCTAATTTCCATGCATA
CTTTCCAGCCAGTTCCAGCAACGCAGGGCTGTTGGGAGAGTTACTATGCTCCGGACT
CAGTGTCATTGGGTTCACATGGAGCTCCTCCCCTGCCGCGACGGAGCTTGAGAATGT
CGTGGTTGACTGGATGGCCAAGATGCTTAACCTTCCATCCTCTTTCTGCTTCTCCGGC
GGAGGCGGTGGCGTTCTGCAAGCAAACACTTGCGAGGCTGTGTTGTGCACTTTAGCC
GCTGCGAGGGACAAGGCTCTTAACCGGGTGGGAGATGATCAGATCAATAAACTGGT
CCTCTACTGCTCCGACCAAACACATTTCACAATCCACAAGGGCGCAAAGTTGATAGG
AATCCGATCAAAGAACATAAAATCAATCACTACTAAGAAAGAGAACGAGTTTAAAC
TCTGTCCTAACGACCTACGCGACGCGATAAGGAGTGATCTGGAAGCAGGACTAGTT
CCGTTTTACGTATGCGGAACGATTGGAACGACCGCGTTAGGAGTTGTGGATCCGATT
AAAGAGCTGGGTAAGGTGGCAAGAGAGTTTGATTTGTGGTTACATGTTGATGGAGC
TTATGGTGGCAGTGCATGCATATGCCCTGAGTTTCAGCATTACCTTGATGGAGTTGA
CCTTGTTGACTCGATCAGCATGAATGCACATAAATGGCTTTTATCCAATCTAGATTG
CTGCTTCCTGTGGCTTCAATCTCCTAACGCCCTAATCGAATCCCTGGCCGCAGAAGC
TAACTTTCTGAAAGGTGGTAGTGAGATGGTGGATTACAAGGACTGGCAGATATCGTT
GAGTCGTCGATTTAGAGCGATCAAGATGTGGATGGTGATAAGGCGATACGGTGTGA
GTAATCTCATTGAGCATATTCGATCCGACGTGAGCATGGCGGTGAGATTCGAAGAG
ATGGTGGCGGCGGACGACCGGTTTGAAATCGTGTTTCCTAGAAAGTTTGCGCTTGTT
TGCTTCAAGCTTAGTAGCGAGAAGACACCACCGGGCCGCGACTCGGAGTTAACTCG
TGAGCTGATGGAGAGAGTCAACTCGAGTGGGAAGGCTTACTTGAGTGGAGTTCAAA
TGGGTCGGATCTTCTTCATCAGGTGTGTGATCGGGTCGAGTTTGACTGAGGAGAGAC
ACGTCGATAATCTGTGGAGGCTCATTCAAGAAACAGCTCAAAGCATCGTGTCTTAG
Rr4HPAAS GenBank accession MF674522 (SEQ ID NO: 2):
MGSLPSPNDPSNTFNPMDLTELSTESKLVVDFITQYYQTLETRPVQPRVKPGFLTGQLPD
KAPFHGESMEVILSDVNEKIVPGLTHWQSPNFHAYFPASSSNAGLLGELLCSGLSVIGFT
WSSSPAATELENVVVDWMAKMLNLPSSFCFSGGGGGVLQANTCEAVLCTLAAARDKA
LNRVGDDQINKLVLYCSDQTHFTIHKGAKLIGIRSKNIKSITTKKENEFKLCPNDLRDAIR
SDLEAGLVPFYVCGTIGTTALGVVDPIKELGKVAREFDLWLHVDGAYGGSACICPEFQH
YLDGVDLVDSISMNAHKWLLSNLDCCFLWLQSPNALIESLAAEANFLKGGSEMVDYKD
WQISLSRRFRAIKMWMVIRRYGVSNLIEHIRSDVSMAVRFEEMVAADDRFEIVFPRKFA
LVCFKLSSEKTPPGRDSELTRELMERVNSSGKAYLSGVQMGRIFFIRCVIGSSLTEERHVD
NLWRLIQETAQSIVS
Rr4HPAR1 DNA (SEQ ID NO: 3):
ATGAGTTTAAGCGGAGCGGGGAAGGTGGTTTGCGTTACCGGCGCGTCTGGCTACAT
AGCGTCCTGGCTCGTCAAGCTTCTTCTCCAGCGCGGTTATACCGTCAAGGCCTCCGT
TCGCGATCCTAATGATCCGAAAAAGACTCAGCACTTGACGGCACTTGATGGAGCTA
AGGAGAGGCTGCAGTTGTACAAAGCCAATTTGCTTGAACAAGGCTCGTTTGATCCCA
TAGTTGAAGGATGTGAAGGTGTTTTCCACACCGCGTCTCCCTTTTATCATGCAGTGG
ATGATCCGCAGGCCGAGTTAATTGACCCTGCTGTCAAGGGAACACTCAATGTTCTTT
CTTCATGTGCTAAAGTTGCGTCTCTTAAAAGAGTAGTCCTGACTTCTTCGATTGCTGC
TGTTGCATATAATGGGAAACCCCGTACTCCGGAGGTTGTAGTTGACGAGACTTGGTT
TTCTAACCCAGATGTTTGTAAGGAGATGAAGCTTTGGTATGTCATATCCAAGACACT
CGCTGAAGAAGCAGCATGGAAGTTTGTGAAAGAGAAAGGAATAGACATGGTTACCA
TAAATCCGGCCATGGTGATTGGTCCCCTTCTGCAACCAACACTCAATACCAGTGCTG
CTGCTATTCTGAACTTGATCAATGGATCGGAGACATACCCAAATGCTTCTTTTGGAT
GGGTCAATGTGAAAGATGTTGCAGAAGCACACGTTCTTGCATTTGAGGTTCCTTCAG
CTAATGGTAGATACTGCTTGGTGGAAAGAGTTGCCCACAGTTCTGAAGTGGTGAACA
TGCTCCATGAGCTCTACCCTGATATCAAACTTCCCGCCAAGTGTGCAGATGACAAAC
CATTTGTGCCAATTTATCAAGTTTCAAAAGAAAAGGCACATACTTTAGGGGTAAAAT
TCATTCCTTTAGAGGTAAGCCTCAAGGAAACAGTTGAAAGCTTGAAGGAAAAGGGT
TTCGCCAAACTCTGA
Rr4HPAR1 amino acid GenBank accession MF674524 (SEQ ID NO: 4):
MSLSGAGKVVCVTGASGYIASWLVKLLLQRGYTVKASVRDPNDPKKTQHLTALDGAK
ERLQLYKANLLEQGSFDPIVEGCEGVFHTASPFYHAVDDPQAELIDPAVKGTLNVLSSC
AKVASLKRVVLTSSIAAVAYNGKPRTPEVVVDETWFSNPDVCKEMKLWYVISKTLAEE
AAWKFVKEKGIDMVTINPAMVIGPLLQPTLNTSAAAILNLINGSETYPNASFGWVNVKD
VAEAHVLAFEVPSANGRYCLVERVAHSSEVVNMLHELYPDIKLPAKCADDKPFVPIYQ
VSKEKAHTLGVKFIPLEVSLKETVESLKEKGFAKL
RrUGT2 DNA (SEQ ID NO: 5):
ATGGGTTCTGATTCACGGCCTCTACGCGTCTTCTTCTTTCCCTTCATGGCTCACGGCC
ATCTGATTCCGATGGTCGACATCGCCAGACTCTTCTCTTCTCAAGGAGTCCACTCCA
CCATCATCACCACCCCACTAAACGCCAATTACATCTCCAAAACGACGTCTCTATCCA
TCAAAACGATACCGTTTCCTGCTGCGGAAGTTGGGCTTCCGGACGGCTGCGAGAATA
TCGACATGCTTCCTTCGCCCGATCTCTTCTTCAAATTTTTCCAAGCCGCCAATTTACT
CCAAGCGCCGTTCGAGAACCTTCTAGAACTCGAAAGGCCCGATTGCTTAATCTCCGA
CATCTTCTTCCCCTGGTCAGTCGACTCCGCCGAGAAATTCAACATCCCGAGACTCGT
TTTCCACGGCACGAGCTTCTTCGCCATGTGCGCCATGGAGAGCTTGAAGACCCACAA
GCCCTATAAATCGGTAAGCACCGACTCTGAACCGTTCTTAATCCCGAATCTCCCTGA
TGAAATCAAAATGACTAAAAGTCAGTTCACGGTTGACGCTTGGGAAGACACCGAAA
AGGGCCTTGGGAAGCTGTTGGCTGATGCGAGAGCTTCAGGGCTGAGGAGCTTCGGC
ATGATCGTAAACAGCTTCCACGAGCTCGAACCGGCTTACGCGGATTATTACAAGAAT
GTGTTGAACATGAAAGCGTGGTGTGTCGGGCCTGTTTCGTTATATAACCGAAACGAT
GACGAGAAAATTGCAAGAGGGAAGAAATCAGCAATCGATGATCATGAGTGTTTAAA
ATGGCTGGAGGGAAAGCAGCCAGACTCCGTCGTGTACGTTTGTTTCGGGAGCAGCG
CGAGCTTCCCTGATGAGCAGTTGCGCGATATCGCATTGGGGCTGGAAGAATCTGGA
GTAAATTTCATCTGGGTGATCAGGAGAAGTTCCGAGTCAGGATCAGAAGATTACTTG
CCGGAGGGGTTTGAGGACCGGGTGAAGGACAGAGGGCTCGTGATCCGAGGTTGGGC
GCCACAGGTACTGATTTTGGACCATCCGTCGGTTGGGGGATTTGTGACTCACTGCGG
ATGGAATTCGGCATTGGAGGGGATTTCAGCTGGCTTGCCGATGGTGACTTGGCCACT
GTTCGCAGAGCAGTTTTTCAACCAGAAATTGATTACGGATGTGTTGAAAGTTGGGGT
TGAGGTTGGAGTGCAGAAATGGTCTCGGAACGGGGAGGATCGCGTGACGAAGGAG
AAGGTTGAGAAGGCGGTGAGGGCTGTTATGGTTGGGGAGGACGCTGAGGAGAGGC
GTGGCAGAGCTCGTCAGCTTGGGAAATTGGCAAAGAAAGCTGTGGCGAAAGATGGG
TCTTCGTACATTGATCTCCACAATTTGCTTGATGAATTGAAGTTGAGAAGAGAGACT
TTGTCCTAG
RrUGT2 amino acid GenBank accession MF674527 (SEQ ID NO: 6):
MGSDSRPLRVFFFPFMAHGHLIPMVDIARLFSSQGVHSTIITTPLNANYISKTTSLSIKTIPF
PAAEVGLPDGCENIDMLPSPDLFFKFFQAANLLQAPFENLLELERPDCLISDIFFPWSVDS
AEKFNIPRLVFHGTSFFAMCAMESLKTHKPYKSVSTDSEPFLIPNLPDEIKMTKSQFTVD
AWEDTEKGLGKLLADARASGLRSFGMIVNSFHELEPAYADYYKNVLNMKAWCVGPVS
LYNRNDDEKIARGKKSAIDDHECLKWLEGKQPDSVVYVCFGSSASFPDEQLRDIALGLE
ESGVNFIWVIRRSSESGSEDYLPEGFEDRVKDRGLVIRGWAPQVLILDHPSVGGFVTHCG
WNSALEGISAGLPMVTWPLFAEQFFNQKLITDVLKVGVEVGVQKWSRNGEDRVTKEK
VEKAVRAVMVGEDAEERRGRARQLGKLAKKAVAKDGSSYIDLHNLLDELKLRRETLS
RrUGT3 DNA (SEQ ID NO: 7):
ATGTCAGGCACACCACACATCGCCATCCTCCCCAGCCCCGGCATGGGCCACCTCATC
CCCATGGCCGAGTTCGCCAAGCGCCTAGTCCACCACCACAACTTCAGTATCACCTTC
GTCATCCCTACCGACGGCCCACCTTCCTCCGCCTACCAACAAGTCCTCACCTCCCTCC
CATCTTCCATAGATCACATCTTCCTTCCACAAGTCGACTTAACCGACGTCGTATCAC
AATCACCAGCTCATCCCAGAATCGAAACCCTAATCTCCCTCACCGTCGCTCGCTCCC
TCTCCTCCCTCCGCACCACCTTATCCTCTCTCCAATCGTCTAAAAACCTCGTCTCGCT
CGTCGTTGATCTTTTCGGCACTGATGCATTCGACCCGGCCATCGAGCTCGGCATCTC
GCCCTACATTTTCTTCCCTTCCACAGCCATGACGCTCTCGCTCTTCCTATACATGCCT
CAGCTTGACAAATCAGTCACGTGCGAATTTCGTCACATGACGGATTTGGTTCGAATT
CCTGGATGCGTTCCTGTCCGTGGATCGGATTTATTCGACCCGGTTCAAGACAGGACC
GACGAGGCTTATAAATGGGTCATACATCACTCCAACAGGTACCCTATGGCGGAGGG
TGTTATAGAGAATAGCTTCATGGAGTTGGAACATGGTGCGTTAAAGTATTTGCAAAC
GGTTCAATCGGGTAAGCCGCCTGTCTACGCGGTCGGACCGTTGATTAAAATGGATTA
TGATGTTGACGATTCCGGGTCGAAGATAATCGAGTGGCTCGATGATCAACCGGTTGG
TTCGGTTTTATTTGTTTCGTTTGGAAGCGGCGGAACGCTCTCGTATGAGCAAATGAC
CGAGCTGGCTCACGGTTTGGAATCGAGCCAGCAACGGTTCTTATGGGTGGTTCGGAG
TCCGAATCAAATCCCCAACAGCACGTATTTCAGTGTACAAAGCCAAAAAGACCCGT
TGGCTTACTTGCCAGAAGGATTTTTAAACCGAACCGAGGGTAGGGGTCTGGTCGTAT
CGAATTGGGCCCCACAGGCTCAAATTTTGAGTCACGGTTCGACCGGTGGGTTCATGA
GCCACTGTGGTTGGAATTCGATTTTGGAGAGTGTGGTGCACGGCGTGCCGATCATAG
CGTGGCCGTTGTACGCCGAGCAGAAGATGAATTCGATAATCGTGGTGGAGGACGTT
AAGGTGGCGCTGAGGCCGGCGGGGGTAGGGGAGAGGGTGGTGGAGAGGTCGGAGA
TAACCGCAGTGGTGAAGGCGTTGATGGAGGGTGAGGAGGGGAAGAAGGTAAGGAA
TAGGATGAAGGAACTCAAGGAAGCGGCGGCACGTGCGGTTAGTGATGACGGTGCGT
CGACCATAGCGATTGCGGACTTGGCGCAAAAATGGCGGAGTTCGATGAAGCATTGA
RrUGT3 amino acid GenBank accession MF674528 (SEQ ID NO: 8):
MSGTPHIAILPSPGMGHLIPMAEFAKRLVHHHNFSITFVIPTDGPPSSAYQQVLTSLPSSID
HIFLPQVDLTDVVSQSPAHPRIETLISLTVARSLSSLRTTLSSLQSSKNLVSLVVDLFGTDA
FDPAIELGISPYIFFPSTAMTLSLFLYMPQLDKSVTCEFRHMTDLVRIPGCVPVRGSDLFD
PVQDRTDEAYKWVIHHSNRYPMAEGVIENSFMELEHGALKYLQTVQSGKPPVYAVGPL
IKMDYDVDDSGSKIIEWLDDQPVGSVLFVSFGSGGTLSYEQMTELAHGLESSQQRFLWV
VRSPNQIPNSTYFSVQSQKDPLAYLPEGFLNRTEGRGLVVSNWAPQAQILSHGSTGGFM
SHCGWNSILESVVHGVPIIAWPLYAEQKMNSIIVVEDVKVALRPAGVGERVVERSEITAV
VKALMEGEEGKKVRNRMKELKEAAARAVSDDGASTIAIADLAQKWRSSMKH-
RrUGT7 DNA (SEQ ID NO: 9):
ATGGCTGAAAACACTCATGCTCATGCCATAGTGGTACCATTTCCAGTTCAAGGACAC
ATAAAGCCCTCGCTGAATCTAGCCCTCAAGCTAGCATCTCAAGGCTTCACCATCACT
TTTGTCACCACTCATTTCACCCACCAGCAAATCTCCCAAGCTCACAAAAACAGTACA
AATACAAACCATGACATGTTTTTCCAGGCACGAAACTCCAGTCTCGATATCCGCCAT
GTAACGGTGACAGACACTTTTCCTTTGGGATTCGATCGCGCAGGGAATCAGGATCAG
TTTTGGGAGGGCATGCTTCACGTATTCCCTGCACATGTTGATGAACTGGTGGATCAG
TTAATGAATTCTTCGAAGCCGAGACCAACTTGTTTGATTCTGGATACATTTTATAACT
GGGGTTCCAAAATTGCTAACAAGTTTAATTTAGTGCATATTTCATTTTGGACTCAGTC
TGCTCTTTCTTTCACTTTGTTTTACCATTGGGAACTTTTAAAGAAAAATGGTCACTTT
GGCTCTCCAGATAATCGCACGGATGTCATCGATTATATTCCCGGTGTGCAAGAGATC
AAGCCCGCAGACTTAATATCCTACCTTCAGATGAGTGATACAACTACTGTGGCTCAC
AGGACTTGTTTCACAGCATTTGAAGATGTCAGGAAGGCAGATTTCATCCTGGCTAAT
ACAATCCAAGAATTTGAAACTGATACAATTTCTTCTATCCGATTTCACCAGCCATTTT
TCTACCCAATTGGACCTGTTTTTTTAACAAAGTCTGAACAACAAGCTAGCTCAGCTTT
GTGGTCTGAGTCAGACTGTGAGCAGTGGCTAAGTACAAAACCAAAAGGGTCTGTTC
TCTATGCCTCATTTGGGAGCTATGCTCGTGTAACTAGGCATGATATCGCAGAGATAG
CCTACGGATTGATGCAAAGTGAGGTGAATTTTATTTGGGTGATTCGCGACGATATTG
TGGGTGCACACGAGACTGATTTTTTACCAACAGAATTCATAAATGGAATCAAACTCA
AAGATCAGGGACTACTAGTTTCCTGGTGCTCTCAAACTGAAGTTTTGTCCAATGCGG
CGATTGGAGGATTTCTGACTCATTGTGGATGGAACTCGATACTCGAAAGCGTATGGT
GTGAAGTTCCATTATTGTGTTTTCCAATAATGACTGATCAGCCTAGTAACAGGAAAC
TGGTGGTGGATGACTGGAGGATCGGCGTCAACCTATCTGCGGCGGAGGAGGTCAGT
AGAGAAGAAGTGTCAATGAAGGTCAGGAACTTGATTTCTGGAGAATTGGGGAATGA
GTTGAGAGTGCAGATTCAAAAGTACAAAAAGTTGATGGAGAATGGTATAATGGAAG
GTGGATCATCACATTCCAATTGGAACAAGTTCATCCACGACCTACAAATCTTCAAGA
AATGA
RrUGT7 amino acid GenBank accession MF674532 (SEQ ID NO: 10):
MAENTHAHAIVVPFPVQGHIKPSLNLALKLASQGFTITFVTTHFTHQQISQAHKNSTNTN
HDMFFQARNSSLDIRHVTVTDTFPLGFDRAGNQDQFWEGMLHVFPAHVDELVDQLMN
SSKPRPTCLILDTFYNWGSKIANKFNLVHISFWTQSALSFTLFYHWELLKKNGHFGSPDN
RTDVIDYIPGVQEIKPADLISYLQMSDTTTVAHRTCFTAFEDVRKADFILANTIQEFETDTI
SSIRFHQPFFYPIGPVFLTKSEQQASSALWSESDCEQWLSTKPKGSVLYASFGSYARVTR
HDIAEIAYGLMQSEVNFIWVIRDDIVGAHETDFLPTEFINGIKLKDQGLLVSWCSQTEVLS
NAAIGGFLTHCGWNSILESVWCEVPLLCFPIMTDQPSNRKLVVDDWRIGVNLSAAEEVS
REEVSMKVRNLISGELGNELRVQIQKYKKLMENGIMEGGSSHSNWNKFIHDLQIFKK-
RrUGT13 DNA (SEQ ID NO: 11):
ATGGCAGAAATAAGTCTCATCTTCATCCCTTTTCCCGTAATCAGCCATCTCACTCCCA
CAATCGAAATCGCCAAAATCCTCCTCAGCAGAGACCACCGCCTTTCCATCACCTTCC
TCGTCATCGACATCCCCCAACGAGACGCCTCACTCGCCTCCCTCACCACCTCCATCA
TCTCCGATCGCCTCCACTTCCTCGATGTCGTACTTCCTCCCAACCAACACTCCCAATC
ATCCAAGCCATCAGGCATCGCGGCTATCGAGTCCGCCAAACCCGCAGTCAAGAAAA
CGATCAGCGATCTTGTTGTACGATCTCAGTCCGCCGCATCTGGTCCGCGGATAGCTG
GCTTCGTGCTGGACATGTTCTGCACGGCCATGATCGACATCGCAACTGAGTTTAACC
TTCCTTCGTATATTTACTACACTTGCGGCTCTTCGTTTCTTTCAATCGTGCTCCACGTC
CAGAAGCTCTGCGATGACGACGCTCTCGATATCGCCGATTTCAAAAACTCGAGTGTG
GAGTTTTCGTTACCTGAGTTTTCAAACTTGATTCCGGCTAGGCTGCTTCCATCCATGG
CGCTCGATAAGGACTTCTCGGCTTCATTCGTCGGCAAAGCTAGAGCGTTCAGGAAGA
CGAAGGGCATTTTGGTCAACTCGCTTGTAGAGTTGGAGCCTCACGCAATCGAGTCGA
TGAAATTAGACCGGTCTGTTCCTCCGATTTACTCGGTCGGACCAGTGCTCAACATGA
ATAGCAACACTGCATTTATCAGACAGGAGCAGGAGAAGGAGATCATGGAGTGGCTG
GACCAACAGCCTCCAGCATCTGTAGTTTTCTTGTGTTTTGGCAGCAGGGGAGCGTTC
AAGCCGGACCAGGTGAAGGAAATCGCACGGGGGTTGGAGTCGAGCGGCTGCCGGTT
CCTCTGGGCGCTTCGGCAGCCTTCATCAAGCAATGTGAGGTTTTCACCTCCTACAGA
TTATGAAGATTTCTCTGAGGTTCTGCCTGAAGGGTTTTTGCAGCGGACATATGGTGTT
GGGAAAGTGATTGGTTGGGCACCCCAGACAGCTGTTTTAGACCACCCTTCGGTGGGT
GGATTCGTATCGCATTGCGGTTGGAACTCGATACTGGAATCTCTTTGGTTTGGTGTGC
CGATTGCGACTTGGCCTCTGTATGCTGAGCAGCAGATGAATGCGTTTGAGGTTGTGA
AGGAGATGAAGATTGGAGTGGAGATAAGTTTGGATTATCGGCTTGAAATGGGCGGT
AAACAAGCAGAAGGTTCTGGGATTATAAGTGGTGAACAGATTGAGAGAGGGATTAG
AGATGTGATGCAGGAGGATAGTGAAGTGAGGAAGAAGGTGAAGCTGATGATGGAA
AAGAGTAGAGAGGCAGTTGTGGAGGGAGGCTCCTCTTATAATTATATCCAAAACTTC
ATCAGTGATCTCAGGACCAACATTGGCTTGTAA
RrUGT13 amino acid GenBank accession MF674538 (SEQ ID NO: 12):
MAEISLIFIPFPVISHLTPTIEIAKILLSRDHRLSITFLVIDIPQRDASLASLTTSIISDRLHFLD
VVLPPNQHSQSSKPSGIAAIESAKPAVKKTISDLVVRSQSAASGPRIAGFVLDMFCTAMID
IATEFNLPSYIYYTCGSSFLSIVLHVQKLCDDDALDIADFKNSSVEFSLPEFSNLIPARLLPS
MALDKDFSASFVGKARAFRKTKGILVNSLVELEPHAIESMKLDRSVPPIYSVGPVLNMN
SNTAFIRQEQEKEIMEWLDQQPPASVVFLCFGSRGAFKPDQVKEIARGLESSGCRFLWAL
RQPSSSNVRFSPPTDYEDFSEVLPEGFLQRTYGVGKVIGWAPQTAVLDHPSVGGFVSHC
GWNSILESLWFGVPIATWPLYAEQQMNAFEVVKEMKIGVEISLDYRLEMGGKQAEGSGI
ISGEQIERGIRDVMQEDSEVRKKVKLMMEKSREAVVEGGSSYNYIQNFISDLRTNIGL-
RrUGT17 DNA (SEQ ID NO: 13):
ATGGGCTCACTTCCTTCCACAAAATCCCATGCAGTCCTCGTCCCATACCCTGCCCAA
GGCCACATCAACCCTTTCATGCAACTTGCCAAGCTCCTACACTCAAAAGGTTTCCAC
ATAACCTTCGTCAACAATGACCACAACCATCGCCGTTTGCTCAGAACAAAAGGGCA
TGATTTTGTTCAAGGGTTGGAAGGTTTAAGGTTTGAAGCTGTGCCGGATGGCCTACC
TCCATCTGACCGTGATGCCACTCAGGATGTCCCTAAGCTGACTGAATCTATTTACAA
TAAGAGCATGAACCAACCGTTCAGTGATCTGCTTCAGAGGCTAAACTCAACGCCCG
GTTCCCCTCCGGTCACTTGTGTCATATCCGATGTTGCCATGTTTTTTGCTTGGGACGT
GGCGGATGAGCTTGGCATCCCTAATGTTCAGTTTTGGACAGCTTCAGCTTGTGGCCT
TTTGGGATACTTACAGTATGATGAGCTCCTAAGAAGAGCCATAGTCCCATTCAAAGA
TGAAAATTTCATGACGGATGGTTCGTTGGAGGCTTTGATTGACTGGATTCCTGGCAT
GCCTAACATGAGGCTGAAGGACTTGCCAAGCTTCATGCGGACCACAAGCCCTGACG
ACGTGTTGTTCAATTACTTGCGTACAATAACCACGAAAGCTCTAAAATCCTCGGCCT
TGTTGCTGAACACATTTGATGATTTTGAACATGAAGTAGTTGAAGAGATGAAGAAA
ATGCAACCAAACATATTCCTAGGAGGTCCACTCAACATGCTTCTCAGGCACACATCA
AAAACTGAAATCACATCCTTAACAACAAGTTTATGGAAAGAGGACACTCATTGTTTA
GAATGGCTGGACAAGCAAGAACCGGAGTCAGTGGTATACATCAATTACGGATCGGT
GACGATAATGTCTGATCACCATTTAAATGAGTTTGCTTGGGGTTTGGCTAACAGCAA
GCACCCTTTTTTGTGGATCGTGAGGCCGGATGTTGTGAGGGGCGAGTCGGGGACTTT
GCCCAAGGAGTTTTATGATGAGATCAAGGACAGGGGATTGATAACGAGCTGGTGTC
CGCAACCAGAGGTGCTTAAACATCCATCCGTAGGTGTATACTTGACGCATTGTGGTT
GGAACTCTATCACGGAGAGTGTGGCCGGAGGAGTGCCATTGATGTGCTGGCCGTTTT
TCGCTGAGCAACAGACGAATAGCCGATTCGCGTGTACGGTGTGGGGCACTGGAGTG
GAGGTGAATGCGGATGTGAAGAGGGAGGAGCTAGCGGAACAAGTGATGGAGATGT
TGGAAGGAAAGAGGGGGCAAGAGTTGAGGAAAAATGCTAAGGAGTGGAGGAGGAA
GGCGGAGGAGGCGACGGACATTGGCGGTTCTGCCTATGCTGATTTCGATAGGTTTAT
GGAAAAAGTGGTCCAGTTTAGCGTCTGA
RrUGT17 amino acid GenBank accession MF674542 (SEQ ID NO: 14):
MGSLPSTKSHAVLVPYPAQGHINPFMQLAKLLHSKGFHITFVNNDHNHRRLLRTKGHDF
VQGLEGLRFEAVPDGLPPSDRDATQDVPKLTESIYNKSMNQPFSDLLQRLNSTPGSPPVT
CVISDVAMFFAWDVADELGIPNVQFWTASACGLLGYLQYDELLRRAIVPFKDENFMTD
GSLEALIDWIPGMPNMRLKDLPSFMRTTSPDDVLFNYLRTITTKALKSSALLLNTFDDFE
HEVVEEMKKMQPNIFLGGPLNMLLRHTSKTEITSLTTSLWKEDTHCLEWLDKQEPESVV
YINYGSVTIMSDHHLNEFAWGLANSKHPFLWIVRPDVVRGESGTLPKEFYDEIKDRGLIT
SWCPQPEVLKHPSVGVYLTHCGWNSITESVAGGVPLMCWPFFAEQQTNSRFACTVWGT
GVEVNADVKREELAEQVMEMLEGKRGQELRKNAKEWRRKAEEATDIGGSAYADFDRF
MEKVVQFSV-
RrUGT29 DNA (SEQ ID NO: 15):
ATGGGATCTCTAGGAAAGAAGATTCAACAAAAGCCACATGCAATATGCACCCCATA
CCCAGCACAAGGCCATATTAATCCCATGCTTAAACTAGCCAAGCTCCTACACCACTC
AGGCTTCTACATAACCTTTGTTCACACAACCTACAACTACAATCGCCTTCTCAAGAC
CCACGGGTCTGATTCCTTAAGTGGTCTACCAGATTTCCAATTTGAGACCATCCCTGAT
GGACTACCACCATCAGATGCAGCTGATGTCACACAAGACATCCCTGCCTTGTGTAAA
TCAACCACCGAAACCTGCTTAGTCCCATTCAAAGAGCTCCTGGCTAAGCTGCATAAC
AAGTCAATGGCGTCACCGGAGGAAGTTCCTCCAGTGACATGCATAGTTTCTGATGGT
TGCATGTCATTTACTGTGGATGCTGCAGAAGAGGCAGGGGTTCCTAATGTGCTTCTT
TGGACTACCAGTGCATGCGGATTTTTAGGATATGCTAATTACCCGAAACTTATTGAC
AGAGGCATAATTCCACTCAAAGATGAGAGCTACTTTACGAATGGGTACCTAGACAA
GACAGTAGATGGAATACCTGGAATGAAAGGCATACGGCTACGAGACTTCCCAAACT
TTGTATGCACCACAAACCCAGATGAGTTTATGGTGAAATATGCAATTCAAGAGATCA
CTAGAGCTGCCAGAGCAGATGCTGTTATTTTGAACACCTTTGACGCTTTGGAACATG
ATTTCTTAGATGGCCTATCAAACATATACCCAAAGGTCCTCCCTATTGGCCCGCTCC
AGCTTCCGCTCAACCAAATCCCAGAGAGCTCACCTCTACATTCAATCTGTTCTAGTC
TCTGGAAAGATGAACCACAGTGCATTACCTGGTTAAACTCCCAAAAACCAAAATCA
GTCGTTTATGTTAACTACGGAAGTATCACAGTTATGACTCCGCAACAAATGGTGGAG
TTCGCATGGGGACTGGCTAATACAAAATACCCTTTTCTGTGGATTATTAGACCTGAT
TTGGTTGCTGGTGAGACAGCTGTCCTACCTCCAGATTTTTTGGAAGTGACAAAAGGA
AGGAGCTGCTTGGCTAGTTGGTGCCCACAGGAACAAGTTCTTAGTCACACATCCATA
GGAGGGTTCTTAACCCATTGTGGGTGGAACTCAATGCTAGAAAGCGTGGTCGAAGG
AGTTCCAATGGTATGCTGGCCGTTTTTTGCTGAGCAACAGACTAATTGCTGGGCTGC
TCGGACAAAATGGGGTATAGGTATGGAAATTGACAATGATGTTAAGAGGGATAAGG
TTCAGAAAATGGTGACAGAGCTTATGGAGGGCGAAAAGGGAAAGGAGATGAAGAG
GAAGGGCGGAGAATGGAAGAAGCTTGGGGCAGAAGCTGCCGGTCCTAATGGCTCAG
CTACCTTAAACTTCAGCAGACTTATAAATGACGTACTTCTGTCCAAAAAAAAAATTG
TAGTTACAACCTAA
RrUGT29 amino acid GenBank accession MF674554 (SEQ ID NO: 16):
MGSLGKKIQQKPHAICTPYPAQGHINPMLKLAKLLHHSGFYITFVHTTYNYNRLLKTHG
SDSLSGLPDFQFETIPDGLPPSDAADVTQDIPALCKSTTETCLVPFKELLAKLHNKSMASP
EEVPPVTCIVSDGCMSFTVDAAEEAGVPNVLLWTTSACGFLGYANYPKLIDRGIIPLKDE
SYFTNGYLDKTVDGIPGMKGIRLRDFPNFVCTTNPDEFMVKYAIQEITRAARADAVILNT
FDALEHDFLDGLSNIYPKVLPIGPLQLPLNQIPESSPLHSICSSLWKDEPQCITWLNSQKPK
SVVYVNYGSITVMTPQQMVEFAWGLANTKYPFLWIIRPDLVAGETAVLPPDFLEVTKGR
SCLASWCPQEQVLSHTSIGGFLTHCGWNSMLESVVEGVPMVCWPFFAEQQTNCWAAR
TKWGIGMEIDNDVKRDKVQKMVTELMEGEKGKEMKRKGGEWKKLGAEAAGPNGSAT
LNFSRLINDVLLSKKKIVVTT
RrUGT32 DNA (SEQ ID NO: 17):
ATGGGATCTCTAGGAAAGAAGATTCAACAAAAGCCACATGCAATATGCACCCCATA
CCCAGCACAAGGCCATATTAATCCCATGCTTAAACTAGCCAAGCTCCTACACCACTC
AGGCTTCTACATAACCTTTGTTCACACAACCTACAACTACAATCGCCTTCTCAAGAC
CCACGGGTCTGATTCCTTAAGTGGTCTACCAGATTTCCAATTTGAGACCATCCCTGAT
GGACTACCACCATCAGATGCAGCTGATGTCACACAAGACATCCCTGCCTTGTGTAAA
TCAACCACCGAAACCTGCTTAGTCCCATTCAAAGAGCTCCTGGCTAAGCTGCATAAC
AAGTCAATGGCGTCACCGGAGGAAGTTCCTCCAGTGACATGCATAGTTTCTGATGGT
TGCATGTCATTTACTGTGGATGCTGCAGAAGAGGCAGGGGTTCCTAATGTGCTTCTT
TGGACTACCAGTGCATGCGGATTTTTAGGATATGCTAATTACCCGAAACTTATTGAC
AGAGGCATAATTCCACTCAAAGATGAGAGCTACTTTACGAATGGGTACCTAGACAA
GACAGTAGATGGAATACCTGGAATGAAAGGCATACGGCTACGAGACTTCCCAAACT
TTGTATGCACCACAAACCCAGATGAGTTTATGGTGAAATATGCAATTCAAGAGATCA
CTAGAGCTGCCAGAGCAGATGCTGTTATTTTGAACACCTTTGACGCTTTGGAACATG
ATTTCTTAGATGGCCTATCAAACATATACCCAAAGGTCCTCCCTATTGGCCCGCTCC
AGCTTCCGCTCAACCAAATCCCAGAGAGCTCACCTCTACATTCAATCTGTTCTAGTC
TCTGGAAAGATGAACCACAGTGCATTACCTGGTTAAACTCCCAAAAACCAAAATCA
GTCGTTTATGTTAACTACGGAAGTATCACAGTTATGACTCCGCAACAAATGGTGGAG
TTCGCATGGGGACTGGCTAATACAAAATACCCTTTTCTGTGGATTATTAGACCTGAT
TTGGTTGCTGGTGAGACAGCTGTCCTACCTCCAGATTTTTTGGAAGTGACAAAAGGA
AGGAGCTGCTTGGCTAGTTGGTGCCCACAGGAACAAGTTCTTAGTCACACATCCATA
GGAGGGTTCTTAACCCATTGTGGGTGGAACTCAATGCTAGAAAGCGTGGTCGAAGG
AGTTCCAATGGTATGCTGGCCGTTTTTTGCTGAGCAACAGACTAATTGCTGGGCTGC
TCGGACAAAATGGGGTATAGGTATGGAAATTGACAATGATGTTAAGAGGGATAAGG
TTCAGAAAATGGTGACAGAGCTTATGGAGGGCGAAAAGGGAAAGGAGATGAAGAG
GAAGGGCGGAGAATGGAAGAAGCTTGGGGCAGAAGCTGCCGGTCCTAATGGCTCAG
CTACCTTAAACTTCAGCAGACTTATAAATGACGTACTTCTGTCCAAAAAAAAATTGT
AG
RrUGT32 amino acid GenBank accession MF674557 (SEQ ID NO: 18):
MGSLGKKIQQKPHAICTPYPAQGHINPMLKLAKLLHHSGFYITFVHTTYNYNRLLKTHG
SDSLSGLPDFQFETIPDGLPPSDAADVTQDIPALCKSTTETCLVPFKELLAKLHNKSMASP
EEVPPVTCIVSDGCMSFTVDAAEEAGVPNVLLWTTSACGFLGYANYPKLIDRGIIPLKDE
SYFTNGYLDKTVDGIPGMKGIRLRDFPNFVCTTNPDEFMVKYAIQEITRAARADAVILNT
FDALEHDFLDGLSNIYPKVLPIGPLQLPLNQIPESSPLHSICSSLWKDEPQCITWLNSQKPK
SVVYVNYGSITVMTPQQMVEFAWGLANTKYPFLWIIRPDLVAGETAVLPPDFLEVTKGR
SCLASWCPQEQVLSHTSIGGFLTHCGWNSMLESVVEGVPMVCWPFFAEQQTNCWAAR
TKWGIGMEIDNDVKRDKVQKMVTELMEGEKGKEMKRKGGEWKKLGAEAAGPNGSAT
LNFSRLINDVLLSKKKL-
RrUGT33 DNA (SEQ ID NO: 19):
ATGAGCTTAATTGAAAAACCACTCACGGCCATAGAGACTCGTGAAAAACCACACGC
TGTGTGCATCCCATACCCAGCTCAAGGCCATATCAATCCCATGATGCAACTTGCAAA
GCTCCTCCACCACTCTGGTTTCCACATAACGTTTGTCCACACTGAGTATAATTATGAC
CGTCTAGTGAAGTCTCAAGGTTCAGCTTGTGTGGCTGGTTTACCGGATTTCCGCTTTG
AAGCCATCCCAGATGGCTTGCCCTCGACGAATGGTGATGTTACTCAAGACATTCCTC
TGTTGAGTAGCTCTACTTCTAAAACCTGCTTGAAGCCGTTTAAGGAGTTATTGAAGA
GGTTGCAGGACAAATGCAAAGAGTTACCTGATGATGTTCCGCCTCTGTCGTGCATCG
TGTCTGATGCAGCCATGTCGTTTACGATCGATGCATCTGAGGAGTTTGGAGTGCCCA
TAGCGCTTCTTTGGACTGCAAGTGCCTGCGGGTTCTTGGGTTACACGCATTACCCAT
ATCTAATTGACAGAGGTGTCATCCCATTGAAAGATGAGAGCCAATTAACAAACGGA
TACCTAGATATGAGCATAGATGGCATACCTTGTATGGAAGGTATCCGCTTACGAGAC
CTCCCAAGCTTTCTACGCACAACTGATTTAGATGATATGATGTTTAGTTATATACTGC
ACGAAATAAAACAAGTTTCAAGAGGCAGTGCTATCATTCTGAACACCTTTGAAGCTT
TGGACCATGATGTCTTGGATAGTCTCTCCAAAATTTACCAAAATGTCATCCTGCCAG
TTGGCCCTCTACATGTCTCGCTCAACAAGATCCCAAAACACTACCCACTTCAATCTTT
AAGCTCGAATTTATGGAAAGATGACACAGACTGCATTCCCTGGCTGAGCTCTAAGGC
TTCAAAATCAGTTATATACGTTAACTTTGGGAGCATCACGACGGTATCACCAAAACA
AATTGTGGAGTTTGCGTGGGGATTGGCTAACAGCAAACACCCTTTCCTTTGGATAAT
CAGACCGGACTTGGTGGCAGGTGAGGCATCCATCATTCCGCAGGACTTCATGGATG
AAACAAAAGGAAGAGGTTTGTTGGCTGGTTGGTGTGACCAAGAGCTTGTTCTCAACC
ATCCATCCATTGGAGGGTTTCTTACGCACTGTGGCTGGAACTCAATTATTGAAAGCA
TTAGCGCAGGAGTCCCTACGGTCTGCTGGCCATTTTTTGCTGAGCAGCAAACAAATT
GTTGGTTTGCTTGCAAAAAATGGTGCATTGGGATGGAGATGCATACTGATGTAAAGA
GGGATGAGGTTGACAAGCTGTTGAGAGAGCTAATGGAAGGTGACAAAGGGGAGGA
GTTGAAGAGGAAGGCAACCAACTGGAAGAGGCTGGCAGAAGAAGCTGTTTCCTCCA
CTGGCTTATCAACCTTAAACTTCAGGACGTTAGTGAATCAAGTCCTGCTCTCAAAAA
CAAAACATATCCGTTAG
RrUGT33 amino acid GenBank accession MF674558 (SEQ ID NO: 20):
MSLIEKPLTAIETREKPHAVCIPYPAQGHINPMMQLAKLLHHSGFHITFVHTEYNYDRLV
KSQGSACVAGLPDFRFEAIPDGLPSTNGDVTQDIPLLSSSTSKTCLKPFKELLKRLQDKCK
ELPDDVPPLSCIVSDAAMSFTIDASEEFGVPIALLWTASACGFLGYTHYPYLIDRGVIPLK
DESQLTNGYLDMSIDGIPCMEGIRLRDLPSFLRTTDLDDMMFSYILHEIKQVSRGSAIILN
TFEALDHDVLDSLSKIYQNVILPVGPLHVSLNKIPKHYPLQSLSSNLWKDDTDCIPWLSS
KASKSVIYVNFGSITTVSPKQIVEFAWGLANSKHPFLWIIRPDLVAGEASIIPQDFMDETK
GRGLLAGWCDQELVLNHPSIGGFLTHCGWNSIIESISAGVPTVCWPFFAEQQTNCWFAC
KKWCIGMEMHTDVKRDEVDKLLRELMEGDKGEELKRKATNWKRLAEEAVSSTGLSTL
NFRTLVNQVLLSKTKHIR
Arabidopsis thaliana AT2G20340.1 (SEQ ID NO: 21):
MENGSGKVLKPMDSEQLREYGHLMVDFIADYYKTIEDFPVLSQVQPGYLHKLLPDSAP
DHPETLDQVLDDVRAKILPGVTHWQSPSFFAYYPSNSSVAGFLGEMLSAGLGIVGFSWV
TSPAATELEMIVLDWVAKLLNLPEQFMSKGNGGGVIQGSASEAVLVVLIAARDKVLRSV
GKNALEKLVVYSSDQTHSALQKACQIAGIHPENCRVLTTDSSTNYALRPESLQEAVSRD
LEAGLIPFFLCANVGTTSSTAVDPLAALGKIANSNGIWFHVDAAYAGSACICPEYRQYID
GVETADSFNMNAHKWFLTNFDCSLLWVKDQDSLTLALSTNPEFLKNKASQANLVVDY
KDWQIPLGRRFRSLKLWMVLRLYGSETLKSYIRNHIKLAKEFEQLVSQDPNFEIVTPRIF
ALVCFRLVPVKDEEKKCNNRNRELLDAVNSSGKLFMSHTALSGKIVLRCAIGAPLTEEK
HVKEAWKIIQEEASYLLHK
Brachypodium distachyon 1g28960.3 (SEQ ID NO: 22):
MDGSTTSNGGGGWMRPMDEEQLRECGHRMVDFIADYYKSIETYPVLSQVQPGYLKEL
LPDSAPNQPDTLDALFDDIREKIVPGVTHWQSPNYFAYYPSNSSTAGFLGEMLSAAFNIV
GFSWITSPAATELEVIVLDWVAKMLKLPSEFLSAALGGGVIQGTASEAILVVLLSARDRT
LRKHGKKSLEKIVVYASDQTHSALKKACQIAGIFPENIRIVKADCSMNYAVTPGAVSEAI
SIDLSAGLIPFFICATVGTTSSSAVDPLHELGQIAQAHDMWFHIDAAYAGSACICPEYRKY
LNGVEEADSFNMNAHKWFLTNFDCSLLWVKDRNYLIQALSTNPEFLKNKASQENSVID
FKDWQIPLGRRFRSLKLWMVLRLYGVENLQSYIRKHIQLAQHFEQLVISDPRFEVVTPR
NFSLVCFCLVPPTCEVDNGHKLNYDLMDSANSSGKIFISHTVLSGKFVLRFVVGAPLTEE
QHVDAAWKLLQDEATKLLGNVVQ
Carica papaya 16427710 (SEQ ID NO: 23):
MDAEQLRENGHKMVDFIADYYKTIENFPVLSQVEPGYLRDLIPDSAPNSPESFQQLLDD
VRTKILPGVTHWQSPNYFAYYPSNSSVAGFLGEMLSAGLNIVGFSWITSPAATELEMIVL
DWLAKLLKLPEDFHSTGNGGGVIQGTASEAILVVLLAARDKVLKRVGKNALEKLVVYT
SDQTHSAFQKACQIGGIHPENCRVLKTDSSTNYALSPDLLKEAISCDVAAGLIPFFFCATV
GTTSSTAVDPLMALGKIATSNEIWFHVDAAYAGSACICPEYRPYIDGVEEADSFNMNAH
KWFLTNFDCSVLWVKDKYSLIQSLSTNPEFLKNKASQADMVVDYKDWQIPLGRRFRSL
KLWMVLRLYGVENLKSYIRNHIKLAKHFEELVTQDPRFEVVTPRIFSLVCFRLLPPGNDE
NHGNKLNQDLLETVNSTGKLFISHTVLSGKYILRFAVGAPLTEERHVNEAWKILQDEAS
TLLENP
Ricinus communis 16804377 (SEQ ID NO: 24):
MFREGELRPMDAEQLREHGHKMVDFIADYYKTIENFPVLSQVEPGYLRKLLPDSAPNQP
ESLQNVLDDVQAKILPGVTHWQSPNYFAYYPSNSSVAGFLGEMLSAGINMVGFSWITSP
AATELEMIVLDWLGKMLKLPEEFLSTGQGGGVIQGTASEAVLVALVAARDKVLRRVGK
DALRKLVVYGSDQTHSALQKACQIGGIHPVNCRLLETDSSTNYALAPDLLSRAISEDISL
GLIPFFLCATVGTTSSTAVDPLLALGKIAKSNGMWFHVDAAYAGSACVCPEYRCYMDG
VEEADSFNMNAHKWFLTNFDCSALWVKDRNALIQSLSTSPEFLQNKPSQTNTVVDYKD
WQIPLGRRFRSLKLWMVLRLYGVEKLQCYIRNHINLAKYFEGLIAEDTRFEVVSPPIFAL
VCFRLLPPDNNVDHGNKLSHDLLDAVNSTGKIFISHTVLSGKYILRFAVGAPLTEERHVT
AAWKVLQDEACALLETSRIS
Cucumis sativus 16963476 (SEQ ID NO: 25):
MDNELKPMDAEQLREHAHKMVDFIADYYKNIEDFPVLSQVEPGYLQNLLPESAPLNPES
LQSVLDDVQKKIFPGVTHWQSPNYFAYYPSNSSIAGFLGEMLSAAFNVIGFSWVTSPAA
TELEMIVLDWLAKLLKLPDDFLSSGNGGGVIQGTASEAVLVVLLAARDRALRRFGKDY
LKKLVVYASDQTHSALQKACQIGGIHPENCRWLKADISTNYALSPDVLSEELSRDTARG
LIPFFLCATVGTTSSTAVDPLPELGTIAKRHEMWFHVDAAYAGSACVCPEYRQYIDGVE
EADSFNMNLHKWFLTNFDCSALWIKDRHALIRSLSTNPEFLKNKASEAELVVDYKDWQI
PLGRRFRSLKVWMVLRLYGTENLQKYIRNHISLAERFEALVREDPRFEIVTPRIFSLVCFR
LLPSRKNEDGGNRLNQSLLDAVNASGNIFISHTVLSGKYILRFAVGAPLTEEKHINSAWK
LLQDVASTLLAI
Vitis vinifera 17835588 (SEQ ID NO: 26):
MDAEQLRENGHKMVDFIADYYKSIENFPVLSQVEPGYLRELLPDSAPNQPESLQQVFDD
LQAKILPGVTHWQSPNFFAYYPSNSSTAGFLGEMLSAGLNIVGFSWITSPAATELEMIVL
DWLAKLLNLPDDFLSAGNGGGVIQGTASEAVLVVLLAARDRVLRTVGKTALEKLVVY
GSDQTHSALQKACQIGGIHPENCKLLKADSSTGYALSPDLLSEAVSHDITNGLIPFFLCAN
VGTTSSTAVDPLLELGKVTKSNGIWFHVDAAYAGSACVCPEYRHYIDGVEEADSFNMN
AHKWFLTNFDCSVLWVKDRNALVQALSTNPVFLKNKASDANMVVDYKDWQVPLGRR
FRSLKLWMVLRLYGVENLQRYIRNHIKLAKQFEELVAQDPRFEIVAPRKFALVCFRLLPP
HRNEDFSNKLNHNLLDTVNSTGKVYISHTALSGKYTLRLAVGAPLTEERHVNAAWKVI
QEKASVLLSEFGMNGLFDNINLKFILNHQIDISILLNYN
Citrus sinensis 18113817 (SEQ ID NO: 27):
MDAEQLRENAHKMVDFIADYYKSIENFPVLSQVQPGYLHNLIPDSAPHHPESLQNVLDG
YIDIQEKILPGVTHWQSPNYFAYYPSNSSVAGFLGEMLSAGLNIVGFSWITSPAATELEMI
VLDWLAKLLKLPEDFLSSGQGGGVIQGTASEAVLVVLLAARDKALKRVGKNSLEKLVV
YASDQTHSALQKACQIGGIHPQNFRVLKTDSSTNYSLSPDSLAEAISRDLTIGLIPFFLCAT
VGTTSSTAVDPLLALGNIAKSNGMWFHVDAAYAGSACICPEYRQYIDGVEEADSFNMN
AHKWFLTNFDCSALWVKDRNTLIQSLSTNPEFLKNKASQANMVVDYKDWQIPLGRRFR
SLKLWMVLRLYGLENLQGYIRNHIQLAKHFEGLVAQDLRFEVVTPRIFSLVCFRLLPPHN
DEDHGNKLNHKLLDDINSTGKIFISHTVLSGKYILRFAVGAPLTEWRHVNAAWEVMQD
KASALLARLSIE
Capsella rubella 20900667 (SEQ ID NO: 28):
MGFCQIELLRHINKHNMQNGSGKNVLKPMDSEQLREYGHRMVDFIADYYKTIEDFPVL
SQVQPGYLHQLLPDSAPDHPETLDQVLDDVRAKILPGVTHWQSPGFFAYYPSNSSVAGF
LGEMLSAGLGIVGFSWVTSPAATELEMIVLDWLAKLLNLPKEFLSKGNGGGVIQGSASE
AVLVVLIAARDKVLRSAGKNALGKLVVYSSDQTHSALQKACQIAGIHPENCRVLETDAS
TNYALRPELLQEAVSKDLKAGLIPFFLCANVGTTSSTAVDPLAALGKIANSNEIWFHVDA
AYAGSACICPEYRKYIDGVETADSFNMNAHKWFLTNFDCSLLWVKEQDSLTEALSTNP
EFLKNKASQANLVVDYKDWQIPLGRRFRSLKLWMVLRLYGAETLKSYIRNHIKLAKYF
EKLVSQDPNFEIVTPRIFSLVCFRLVPKNDDEKKCNNQNRKLLEAANSSGKLFMSHTALS
GKIVLRCAIGAPLTEEKHMKEAWKVIQDEASFLLHK
Malus domestica 22636618 (SEQ ID NO: 29):
MSGLKPMDAEQLRENAHKMVDFIADYYKTIEDFPVLSQVQPGYLRDLLPDSAPTHPESL
QQVFDDIQAKILPGVTHWQSPNFFGYYPSNSSVAGFLGEMLSAGLNIVGFSWITSPAATE
LEMIVLDWFAKMLKLPEEFLSAGQGGGVIQGTASEAVLVVLLAARDRILRAEGKKALE
KLVVYASDQTHSALQKACQIGGIHPENCRVLSTDSSTNYALSPNVLNEAISNDIASGLVP
FFLCATVGTTSSTAVDPLLELGKITKSNGMWFHVDAAYAGSACICPEYRHHIDGVEEAD
SFNMNAHKWFLTNFDCSLLWIKDRNALVQALSTNPEFLKNKASQANLVVDYKDWQIPL
GRRFRSLKLWMVLRLYGLENLQSYIRNHIDLAKCFEDLVAQDSRFEIVTPRIFSLVCFRL
LPPHNDETYATKLNHDLLDTVNSTGKIFVSHTVLSGKYVLRFAVGAPLTEERHVLAAW
KLLQEEASALLAPL
Linum usitatissimum 23178995 (SEQ ID NO: 30):
MGGYRSLNLIFIFISFVADIRDLGYNTKEGDDGGGALKPMDAEQLRQNAHQMVDFIADY
YKNIETYPVLSQVEPGYLRELLPDSAPNRPESLQSVLDDVQSKIMPGVTHWQSPNYFAY
YPSNSSVAGFLGEMLSAGINMVGFSWITSPAATELEMIVLDWLGKLLKLPEEFLSSGHG
GGVIQGTASEAILVVLLAARDKMLRKFGKSALEKLVVYASDQTHSALQKACQIGGIYPE
NCRLLKTDSSVNYSLTPELVSEAVSQDISAGLIPFFLCGTVGTTSSATVDPLGTLGKIAKN
NDMWFHVDAAYAGSACICPEYRQYLDGVEEADSFNMNAHKWFLTNFDCSTLWVKDK
SALIQALSTNPEFLKNKASQANLVVDYKDWQIPLGRRFRSLKLWMVLRLYGVENLQQY
LRNHIELARHFEECVNHDPRFEALSGKYTLRVAIGAPLTEKRHVAAALKVLQDEATSLL
VATSPLLENGNSS
Eutrema salsugineum 20200788 (SEQ ID NO: 31):
MENGNKNVLKPMDSEQLREYGHRMVDFIADYYKTIEDFPVLSQVQPGYLHNLLPDSAP
DQPETLEEVLDDVKGKILPGVTHWQSPSFFAYYPSNSSVAGFLGEMLSAGLGIVGFSWIT
SPAATELEMIVLDWLAKLLNLPEQFLSRGNGGGVIQGSASEAELVVLIAARDKVLRSVG
KKALEKLVVYSSDQTHSALQKACQIAGIHPENCRVLKADYSTNYALRPETLQEAVSKDL
EAGLIPFFLCANVGTTSSTAVDPLAALGEIAKSNEMWFHVDAAYAGSACICPEYRQYID
GVETADSFNMNAHKWFLTNFDCSLLWVKDQYALTEARSTNPEFLKNKASQANLVVDY
KDWQIPLGRRFRSLKLWMVLRLYGSENLKSYIRNHIKLAKDFEQLVSEDPNFEIVTPRIFS
LVCFRIVPAENDEKKCNNQNRNLLDAVNSSGKLFLSHTALSGKIVLRCAIGAPLTEEKHV
KEAWKVIQEEASYLLRK
Populus trichocarpa 27022899 (SEQ ID NO: 32):
MESKGLQPMDSEQLRENAHKMVDFIADYYKSIENFPVLSQVEPGYLRELLPDSAPNQPE
TLQNVLDDVQAKILPGVTHWQSPSYFAYYPSNSSVAGFLGEMLSAGINMVGFSWITSPA
ATELEMIVLDWLGKLLKLPEDFLSTGQGGGVIQGTASEAVLVVLLAARDRVLRKLGKN
ALEKLVVYASDQTHSALQKACQIGGIHPENCKLLKTGSSTNYALSPDLLGKAISDDISTG
LVPFFLCATVGTTSSTAVDPLLSLGKIAKNNGIWFHVDAAYAGSACICPEYRCYIDGVEE
ADSFNMNAHKWFLTNFDCSALWVKDRNALIQSLSTNPEFLKNKASQANMVVDYKDW
QIPLGRRFRSLKLWMVLRLYGLENLQCYIRNHINLAKYFEGLVAADSRFEVVTPRIFSLV
CFRLLPPNNNEDHGNNLNHDLLDAVNSTGKIFISHTVLSGKYILRFAVGAPLTEERHVTA
AWKVLQDEASALLGSL
Brachypodium stacei 06G160800.1 (SEQ ID NO: 33):
MDGSTTSNGDGGGGWMRPMDEEQLRECGHRMVDFIADYYKSIETYPVLSQVQPGYLK
ELLPDSAPNQPDTLDALFDDIQEKIVPGVTHWQSPNYFAYYPSNSSTAGFLGEMLSAAFN
IVGFSWITSPAATELEVIVLDWVAKMLKLPSQFLSAGLGGGVIQGTASEAILVVLLSARD
RTLRKHGKKSLEKLVVYASDQTHSALQKACQIAGIFSDNIRIVKADCSMNYAVTPGSVS
EAISIDLSSGLIPFFICATLGTTSSSAVDPLHELGQIAQAHDMWFHIDAAYAGSACICPEYQ
QYLNGVEEADSFNMNAHKWFLTNFDCSLLWVKDRNYLIQALSTNPEFLKNKASQENSV
IDFKDWQIPLGRRFRSLKLWMVLRLYGVENLQSYIRKHIQLAQRFEQLVISDSRFEVVTP
RNFSLVCFCLVPPTSEVDNGHKLNYDLMDSVNSSGKIFISHTVLSGKFVLRFAVGAPLTE
EQHVNAAWKLLQDEATKLLGSVVV
Physcomitrella patens Pp3c4_30790V3.1 (SEQ ID NO: 34):
MGSEAGSRSSLTKPFDPEEFRKHAHRMVDFIADYHRDIENFPVQSQVEPGYLQKLLPEN
APDEPESLDDILADVQSKIVPGVTHWQSPNFYGYYPSNGSTAGFLGEMLSGGFNIIGFSW
ITSPAATELEIIVMDWLGKLLKLPNEFLSSGKGGGVIQGTASEAVLVVMLAARKRAVEK
LTKEQGISEFEALAKLVAYTSDQAHSCVNKASQIAGISIENLRLIPTDVSTNYAMSSKVLA
NTLANDVKAGLVPFFLCGVIGSTSSAAVDPLSELGDLAQEYGMWFHVDGAYAGNACIC
PEFRPYLNGVEKADSFDMNPHKWLLTNFDCSTLWVKNPSLLVDALSTNPVFLRNKQSD
NNLVVDYKDWQIPLGRRFRSLKLWMVLRMYGSNGLRSYITNHCNLAKHFEELLRTDSR
FEVVAPRVFSLVCFRLKSPANDADNSCSLSAKLVDALNSDGNILITNTVLGGRYTIRFTV
GASRTELRHVDAAWKVIQQLASKLLKECSS
Ananas comosus 33033299 (SEQ ID NO: 35):
MESELKPMDSEQLREYAHKMVDFIADYYKMIESFPVLSQVKPGYLKELLPDSAPCKPEN
LEDVFDDIRQKIIPGITHWQSPDYFAYYPSNSSTAGFLGEMLSAGFNIIGFSWIASPAATEL
EMIVLDWFAKMLKLPEQFLSTGQGGGVIQGTASEAVLVVLLAARDKILLKAGRKSLEKL
VVYCSDQTHSAMQKACQIAGIFPENFRVLKTDSSSNYALLPEVLSEAISKDLSFGLIPFFL
CATVGTTSSAAVDPLLKLGNISKVHDMWFHVDAAHAGSACICPEYRHHIDGVEEADSF
CMNAHKWFLTNFDCSLLWVKDRSALIQSLSTNPEFLKNKASQENSVVDFKDWQIPLGR
RFRSLKLWMVLRLYGLENLQSYIREHIKLAEQFEQLISSDSRFEIVAPRTFSLVCFRLLPPL
YDQDDGYKLNYNLLDAVNRSGKIFMSHTVLSGKFVLRFAIGAPLTEERHVVAAWKVLQ
DEATILLRGS
Zostera marina 33182387 (SEQ ID NO: 36):
MLNGNMGENEPFKPMDSEQLREYGHKMVDFIADYYKSIEKFPVLSQVQPYYLKDLLPD
AAPDQPEKFQDVLDDITKKIIPGVTHWQSPNFFGYYPGNSSIAGFLGEMICSGLNVIGFS
WITSPASTELEVIVLDWLAKLLNLPDQFLSSGHGGGVIQGTASEAILVVLLAARDKILGRI
GRNSLDKLVVYSSDQVHAAFKKACQIAGIYTENFRVLKTDASSGYGIDPKKFDQAIHDD
MEAGLIPFFLCSTVGTTSSASVDPLVEIGQITEENDMWFHVDAAYAGSACICPEYRHYLD
GVEYADSFCMNAHKWLLTNFDCSALWVKDSSALVNSLSTNPEFLKNKMSEQKKVVDF
KDWQIPLGRRFRSLKLWMVLRLYGAENLREYIRNHIKLANLFEQLVRSDSRFEIVCPTLF
SLVCFRFLPSNDDNDGYELNSMLLDAVNSTGQLFFTHTIISDKYILRFAVGAALTEERHV
RESWKVIQNQATIISRQHILSKTNMKSKCEGMIANE
Daucus carota subsp. sativus 36055203 (SEQ ID NO: 37):
MDGVLKPMDAEQLRENAHKMVDFIADYYKNIETFPVLSQVEPGYLRDLLPHSAPDQPE
SLQNILDDIQAKILPGVTHWQSPNYFAYFPSNSSVAGFLGEMLSAGINMVGFSWITSPAA
TELEMIVLDWLAKLLKLPDHFLSTGQGGGVIQGTASEAVLVVLLAARDKVLRITGKDAL
GKLVVYCSDQTHSALQKACQIAGIHPGNCRVLKTESCNDYSLSPETFEQAISTDVASGLI
PLLLCATVGTTSSTAVDPLLELGKITKMKGIWLHVDAAYAGSACVCPEFRHYIDGVEEA
DSFNMNAHKWFLTNFDCSALWVKDRSALIHSLSTNPEFLKNKASQENLVVDYKDWQIP
LGRRFRSLKLWMVLRLYGLENLQSYIRNHIQLAATFESFVTEDPRFEVVAPRKFALVCFR
LLPPSHKDEDCSNQLNRDLLDAVNATGKAFVSHTALSGRYVVRFAIGAPLTEESHIIEAW
KIFQEVATVLLKSLKMNHTRPLN
Trifolium pratense 35974269 (SEQ ID NO: 38):
MVDFIADYYKTIENFPVLSQVEPGYLGKLLPDSAPTYPTTLEHVLNDVQHKILPGVTHW
QSPNYFAYFPSNSSIAGFLGEMLSAGINIVGFSWITSPAATELESIVLDWLAKALFLPQDF
LSNGKGGGVIQGTASEAVLVVLLAARDKILRTVGRSALPKLVTYASDHVHSSLLKACQI
GGLDPELCRLLKTDSSTNFALSPDVLSEAISNDIASGLIPFFLCANVGTTSSTAVDPLPALA
KVTKTNNIWLHVDAAYAGSACICPEYRHFIDGVEEADSFNMNAHKWFLTNFDCSLLWV
KDRSALIQSLSTNPEFLKNKASEGNMVIDYKDWQIPLGRRFRSLKLWMVLRLYGLEGLR
SHIRNHIALAASFEELVVQDARFKVVTPRTFSLVCFRLLPPPNSEDNGNKLNHDLLDLVN
STGSVFITHTVLSGEYILRLAVGAPLTEVRHVNAAWQILQEKATALLENL
Arabidopsis lyrata 35943929 (SEQ ID NO: 39):
MDSEQLREYGHRMVDFIADYYKTIEDFPVLSQVQPGYLHKLLPDSAPDHPETLDQVLDD
VRAKILPGVTHWQSPSFFAYYPSNSSVAGFLGEMLSAGLGIVGFSWVTSPAATELEMIVL
DWLAKLLNLPEQFMSKGNGGGVIQGSASEAVLVVLIAARDKVLRSVGKNALQKLVVYS
SDQTHSALQKACQIAGIHPENCRVLKTDSSTNYALRPELLQEAVSQDLDAGLIPFFLCAN
VGTTSSTAVDPLAALGKIANRNEMWFHVDAAYAGSACICPEYRQYIDGVETADSFNMN
AHKWFLTNFDCSLLWVKDQDSLTLALSTNPEFLKNKASQANLVVDYKDWQIPLGRRFR
SLKLWMVLRLYGSETLKSYIRNHIKLAKEFEQLVSQDPNFEIVTPRIFSLVCFRLVPVKNE
EKKCNNRNRELLDAVNSSGKLFISHTVSDFSSFFLLFFLLDNVLNLRGNRLCRGKSYCVA
Q
Sorghum bicolor 002G120700.1 (SEQ ID NO: 40):
MDGSGSSGGTNGGSGGDGAGWLRPMDAEQLRECGHRMVDFVADYYKSIETFPVLSQV
QPGYLKELLPDTAPNKPDTLEALFDDIREKIVPGVTHWQSPNYFAYYPSNSSTAGFLGE
MLSAAFNIVGFSWITSPAATELEVIVLDWFAKMLRLPSQFLSTALGGGVIQGTASEAVLV
VLLAARDRTLRKHGKTSLEKLVVYASDQTHSALQKACQIAGIFPENVRLVKADCNRNY
AVAPLAISDAIATDLSSGLIPFFICATVGTTSSSAVDPLPELGQIAKANDMWLHIDAAYAG
SACICPEYRHHLNGVEEADSFNMNAHKWFLTNFDCSLLWVKDRSYLIQSLSTNPEFLKN
KASEANSVFDFKDWQIPLGRRFRSLKLWMVLRLYGVENLQSYIRKHIELAKEFEQLVIS
DSRFEVVTPRTFSLVCFRLVPLASDQDNGRKLNYDLMDAANSSGKIFISHTVLSGKFVLR
FAVGAPLTEGQHIFSAWKILQDLATKQLLESS
Sphagnum fallax 0166s0011.1 (SEQ ID NO: 41):
MGSEAGEGSRLSKPLDVEEFRKHAHQMVDFVADYHRDIESFPVRSQVKPGYLRPLLPDS
APAEPETVEDVFADLWSKILPGLTHWQSPKFFGYYPCNVSTAGMLGEMLCGGLNVNGF
SWITSPAATELETIVLDWLGKLLHLPEEFLSTSGKGGGVIQGTASEAVLVVMLAARKRA
LKQVSSAAQGMSEAEALSKLVVYSSDQTHSCVIKACQVASIATENFRPLPTDASTNFALS
PAVVRKAIATDVEAGLIPFFLCGTLGTTSSAAVDPLEELGDIAKEYGMWYHIDAAYAGN
ACICPEFRHYLNGVEKADSYNMNPHKWLLTNFDCSTLWMKDSEFLLAALSNKPVFLRN
EATDNNLVVDYKDWQIPLGRRFRALKLWMVMRLYGTSGLQSFIRSHVSSAKHFESLVR
ADSRFEVMAPMTFSLVCFRLRTLPGSQDNSNSLNSKLVDALNRKGNILVTHTELSGIYTV
RFAVGATHTELQHVQAAWEVIQAEASHLLNGKQ
Kalanchoe laxiflora 1398s0003.1 (SEQ ID NO: 42):
MILSIHPFPFTLSARFSGAAAANILSKASCWLRCLRSMEGELKPMDAEQLREYGHRMVD
FVADYYKTIEDHPVLSQVEPGYLRKLLPDSAPDKPESFENVLSDVKTKIIPGVTHWQSPN
YFAYFPSNSSTAGFLGEMLSACFNIVGFSWITSPAATELEMIVLDWFAKMLKLPDFFLST
GQGGGVIQGTASEAVLVVLLAARDIFLRKLGKGFLEKLVVYASDQTHSALQKACQIAGI
HPENVRALKTDSSTNYGLSPDLLSKEICHDIANGLVPFFACASVGTTSSTAVDPILELANV
TKSYNIWLHVDSAYAGSACVCPEYRHHIDGVEEVDSFNMNAHKWFLTNFDCSLLWVK
DRNALIQSLSTNPEFLKNKASQSNSVLDYKDWQIPLGRRFRSLKLWLVLRLYGVENLQA
YIRNHIELALNFEELVSQDMRFEIVAPRTFALVCFRLLLPCGFEDHTNDVNSDLLQAVNS
TGKIFISHTVLSGTYVLRFAVGAPLTEERHIDAAWKLIQDQASSLLEKL
Manihot esculenta 12G038600.1 (SEQ ID NO: 43):
MEGELRPMDAEQLREYGHQMVDFIADYYKTIENFPVLSQVEPGYLHKLLPDSAPNQPE
ALQNVLDDVRVKILPGVTHWQSPNYFAYYPSNSSVAGFLGEMLSAGINMIGFSWITSPA
ATELEMIVLDWLGKMLKLPEEFLSSGQGGGVIQGTASEAVLVVLLAARDKVLTRVGKD
SLKKLVVYGSDQTHSALQKACQIAGVHLDNCRLLKTDSSKNYALSPDILCDAISQDMSN
GLIPFFLCATVGTTSSATVDPLLALGKIAKKYGMWFHVDAAYAGSACICPEYRCYIDGV
EEADSFNMNAHKWFLTNFDCSALWVKDRNALIQSLSTNPEFL1<NKASQANMVVDYKD
WQIPLGRRFRSLKLWMVLRLYGVANLQSYIRNHINLAKYFEGLVAGDSRFEVVAPRLFS
LVCFRLLPPDNDENHGNKLNHDLLDAANSTGKIFISHTVLSGKYILRFAVGAPLTEERHV
TAAWKVLQDEASALLGSL
Prunus persica 8G214500.1 (SEQ ID NO: 44):
MESGLKPMDAEQLRENAHKMVDFIADYYKTIENFPVLSQVQPGYLRELLPDSAPTHPEP
LQHIFDDIQAKILPGVTHWQSPNFFGYYPSNSSIAGFLGEMMSAGLNIVGFSWITSPAATE
LEMIVLDWFGKMLKLPEEFLSAGKGGGVIQGTASEAVLVVLLAARDKILRRVGKNSLE
KLVVYASDQTHSALQKACQIGGIHPENCRLLRTDSSTNYALSPNVLNEAISNDVTSGLIP
FFLCATVGTTSSTAVDPLLELGKIAKSNDMWFHVDAAYAGSACICPEYRHYIDGVEEAD
SFNTNAHKWFLTNFDCSVLWIKDRNALIQALSTNPEFLKNKASQANLVVDYKDWQIPL
GRRFRSLKLWMVLRLYGLENLQSYIRNHINLAKHFKELVAQDPRFEIVTPRLFSLVCFRL
LPPHNDETCATKLNHGLLDAVNATGKIFISHTVLSGKYLLRLAVGAPLTEERHVNAAWK
LLQDEASALLATL
Eucalyptus grandis K01418.1 (SEQ ID NO: 45):
MEERLKPMDAEQLRESAHRMVDFIADYYKSIESFPVLSQVEPGYLRKLLPDSAPDHPESL
QQVLEDVQAKILPGVTHWQSPNYFAYYPSNSSIAGFMGEMLSAGLNIVGFSWITSPAAT
ELEIIVLDWLAKLLNLPDDFLSTGPGGGVIQGTASEAVLVVLLAARDKFLSRIGKSSLDK
LVVYSSDQTHSALQKACQIGGIYPENCRVLKTDASTNYALSPDLLNEVISQDISTGLVPFL
LCATVGTTSSTAVDPLPALATVAKRNGMWFHIDAAYAGSACICPEYRPYIDGVEEADSF
NMNAHKWFLTNFDCSALWIKDRKALIQALSTNPEFLKNKASQANMVVDYRDWQIPLG
RRFRSLKLWMVLRLYGVQNLQQYIRNHIELARQFEDLVIQDPRFEVVTPRIFSLVCFRLL
SPDNDGDKGNKLNRDLLDTVNSTGKIFISHTVLSGTYILRFAVGAPLTEERHVNEAWKV
LQDEASKLLATIQNN
Amborella trichopoda 31565185 (SEQ ID NO: 46):
MDAEELREHGHRMVDFISDYYKEIESYPVRSQVQPGYLRNLIPDSAPDMPESFESILEDIR
HKIIPGVTHWQSPKYFAYYPSNSSTAGFLGEMLSAGFNIVGFSWVTSPAATELEVIVLDW
LAKVLKLPEQFLSTGKGGGVIQGTASEAMLVALLAARDKALRRVGQNLLENLVVYGSD
QTHSALIKACKIAGINPMNCRLLQATFMTNYALSPEVASESISNDIAAGLLPIFLCATVGT
TSSTAVDPLAALGRLAKANDMWFHIDAAYAGSACICPEYRHYIDGVEEADSFNMNPHK
WLLTNFDCSTLWVKDSSNLIQSLSTNPEFLRNKASEEDLVVDYKDWQIPLGRRFRSLKL
WMVLRMYGVANLQNHIRTHINLAKHFEELIATDTRFEIIVPRVFALVCFALKPMPNGQD
DASKLNLKLLEAVNNSGAMFLTHTVLSGRFVLRFVVGAPLTEERHVNTAWKVLQDHA
NLILGTV
Salix purpurea 0252s0200.1 (SEQ ID NO: 47):
MESKGLKPMDSEQLRENAHKMVDFIADYYKSIENFPVLSQVEPGYLRELLPDSAPNQPE
TLQNVLDDVQAKILPGVTHWQSPSYFAYYPSNSSVAGFLGEMLSAGINMVGFSWITSPA
ATELEMIVLEWLGKLLKLPEDFLSTGQGGGVIQGTASESVLVVLLAARDRVLTKLGKNA
LEKLVVYASDQTHSALQKACKIGGIHPENCKLLKTDSSTNYALSPDLLSKAISDDISTGLI
PFFLCATVGTTSSTAVDPLHALGKIAKNNGIWFHVDAAYAGSACICPEYRCYIDGVEEA
DSFNMNAHKWLLTNFDCSALWVKDRNALIQALSTNPEFLKNKASQANMVVDYKDWQI
PLGRRFRSLKLWMVLRLYGLENLQCYIRNHINLAKYFEGLVAADSRFEVVTPRIFSLVCF
RLLPPSNNEDHGNNLNRDLLDAVNSSGKIFISHTVLSGKYILRFAVGAPLTEERHVIAAW
KVLQDESTSLLGSL
Medicago truncatula 31080941 (SEQ ID NO: 48):
MVLQIWCLTHDSDKKLGGGYLLFPVIKVAYTVHTLTEWCCVTEEGGGSELKAMDAEQ
LREQGHMMVDFIADYYKTIENFPVLSQVQPGYLGKLLPDSAPTHPESLQHVLNDVQEKI
LPGVTHWQSPNYFAYFPSNSSIAGFLGEMLSAGLSIVGFSWISSPAATELETIVLDWLAK
ALLLPHDFFSTGQGGGVIQGTASEAVLVVLVAARDKILRTVGRSALPKLVTYASDQTHS
SLQKACQIAGLNPELCRLLKTDSSTNFALSPDVLSEAISNDIASGLTPFFLCATVGTTSSTA
VDPLPALAKVTKPNNIWLHVDAAYAGSACICPEYRHFIDGVEEADSFNMNAHKWFLTN
FDCSVLWVKDRSALIQSLSTNPEFLKNKASQENTVIDYKDWQIPLGRRFRSLKLWMVM
RLYGLEGLRTHIRSHIALAVYFEELVVQDTRFKVVAPRTFSLVCFRLLPPQNSEDNGNKL
NHDLLDAVNSTGDVFITHTVLSGEYILRLAVGAPLTEVRHVHAAWQILQEKATALLESL
Brassica rapa I01156.1 (SEQ ID NO: 49):
MQIRAKIPVFGRENGSRHVLKPMDSEQLREYGHRMVDFIADYYKTIESFPVLSQVQPGY
LHNLLPDSAPDHPETVEQVLDDVKTKILPGVTHWQSPNFFAYYPSNSSVAGFLGEMLSA
GVGIVGFSWVTSPAATELEMIVLDWLAKLLNLPEHFLSKGNGGGVIQGSASEAILVVMI
AARDKVLRSAGKNALGKLVVYSSDQTHSALQKACQIAGIHPENCRVLKADSSTNYALR
PELLQEAVSRDLEAGLIPFFLCGNVGTTSSAAVDPLAALGKIAKSNEIWFHVDAAYAGS
ACICPEYRQYIDGVETADSFNMNAHKWFLTNFDCSLLWVKDQHALTEALSTNPEFLKN
KASQANLVVDYKDWQIPLGRRFRSLKLWMVLRLYGAEALKNYIRNHIKLAKDLEQLVS
QDPNFEVITPRIFSLVCFRIVPTDNDEKKCNSRNLELLEAVNSSGKLFISHTALSGKIVLRC
AIGAPLTEEKHVKETWKVIQEKVSYLLRK
Brassica rapa I04706.1 (SEQ ID NO: 50):
MDSEQLREYGHRMVDFIADYYKTIETFPVLSQVQPGYLHNLLPDSAPDQPETVEQVLDD
VKTKILPGITHWQSPTFYAYYPSNSSVAGFLGEMLSAGLGIVGFSWVTSPAATELEMIVL
DWLAKLLNLPEQFLSKGNGGGVIQGSASEAILVVMIGAREKVLRRVGKNALGKLVVYS
SDQTHSALQKACQIAGIHPENCRVLKADSSTNYALRPELLQEAVSKDIEAGLIPFFLCGN
VGTTSSTAVDPLAALGKIAKSNEIWFHVDAAYAGSACICPEYRQYIDGVETADSFNMNA
HKWFLTNFDCSLLWVKDQYVLTEALSTNPEFLKNKASQANLVVDYKDWQIPLGRRFRS
LKLWMVLRLYGAETLKSYIRNHIKLAKDLEQLVSQDPNFEVVTPRIFSLVCFRILPVDND
EKECNNRNRNLLDAVNSSGKLFLSHTALSGKIVLRCAIGAPLTEERHVKETWKVIQEEAS
RLLGK
Brassica rapa G00043.1 (SEQ ID NO: 51):
MDSEQLREYGHRMVDFIADYYKTIETFPVLSQVQPGYLHNLLPDSAPDQPETLEQVLDD
VKEKILPGVTHWQSPSFFAYYPANSSVAGFLGEMLSAALNIVGFSWVSSPAATELEMIVL
DWFAKLLNLPEQFLSRGNGGGVIQGTASEAILVVMIAARDKVLRSLGKKALEKLVVYSS
DQTHSSLLKACQIAGIHLENCRMLKTDSSTNYALRPESLQEAVSGDLEAGLIPFFLCGTV
GTTSSTAVDPLAELGKIAKSNEMWFHVDAAYAGSACICPEYRQYIDGVETADSFNMNA
HKWFLTNFDCSLLWVKDRYALTEALSTNPEFLKNKASQANLVVDYKDWQIPLGRRFRS
LKLWMVLRLYGAETLKSYIKNHIKLAKDLEQLVSQDPNFEVVTPRIFSLVCFRIVPVDND
EKTCNNLNRSLLDAVNSSGKLFISHTTLSGKFVLRLAIGAPLTEEKHVMDAWKVIQEEAS
FLLASQVK
Glycine max 03G167900.1 (SEQ ID NO: 52):
MEEESALRPMDAEQLREQAHKMVDFIADYYKTIEDFPVLSQVQPGYLGKLLPDSAPDSP
ESLQNVLDDVQEKILPGVTHWQSPNYFAYFPSNSSIAGFLGEMLSAGLNIVGFSWITSPA
ATELETIVLDWLAKAFQLPDYFYSSGKGGGVIQGTASEAVLVVLLAARDKILRRVGRNA
LPKLVMYASDQTHSALLKACQIAGINPELCRLLKTDSSTNYALSPDVLSEAISNDIAGGL
VPFFLCATVGTTSSTAVDPLPALGKIAKTNKLWFHVDAAYAGSACVCPEYRHCIDGVEE
ADSFNMNAHKWFLTNFDCSLLWVKDRSSLIQSLSTNPEFLKNKASQGNMVIDYKDWQI
PLGRRFRSLKLWMVLRLYGLDGLRSHIRNHIELAANFEELVRQDTRFKVVAPRTFSLVC
FRLLPHPNSADHGNKLNSDLLDSVNSTGNAFITHTVLSGEYILRFAVGAPLTERRHVNM
AWQILQDKATALLESL
Fragaria vesca 27261550 (SEQ ID NO: 53):
MDAEQLRENAHKMVDFIADYYKTIEDFPVLSQVQPGYLRELLPDSAPTQPESLQHIFDDI
QAKILPGVTHWQSPNFFAYYPSNSSIAGFLGEMLSAGLNIVGFSWVTSPAATELEMIVLD
WLAKLIKLPDEFLSAGQGGGVIQGTASEAILVVMLAARDKILRRVGKNALEKLVVYASD
QTHSALQKACQIAGIHPENCRILSTNSTTNYALSPSVGTTSSTAVDPLGELGKIAKNNEM
WFHVDAAYAGSACICPEYRHYIDGVEKADSFNMNAHKWFLTNFDCSVLWIKDRNALV
QSLSTNPEFLKNKASQANMVVDYKDWQVPLGRRFRSLKLWMVLRLYGLENLQSYIRT
HINLAKHFEELVAQDPRFEIVTPRLYSLVCFRLLPPHGNEACASKLNHDLLDAVNSTGKI
YISHTVLSGAYILRFAVGAPLTEEKHVTAAWKKLKSVIRDVLALANSFVSITFSHMYREA
NFLTDALASVGHSLSSSMCWFDGIPPQAQMALLMDSSCIGHLRGSSL
Kalanchoe fedtschenkoi 0172s0035.1 (SEQ ID NO: 54):
MEGELKPMDAEQLREYGHRMVDFVADYYKTIEDHPVLSQVEPGYLRKLLPDSAPDKPE
SFENVLSDVKTKIIPGVTHWQSPNYFAYFPSNSSTAGFLGEMLSACFNIVGFSWITSPAAT
ELEMIVLDWFAKMLKLPDFFLSTGQGGGVIQGTASEAVLVVLLAARDIFLRKLGKGFLE
KLVVYASDQTHSALQKACQIAGIHPENVKALKTDSSTNYGLSPDLLSKEICHDIANGLVP
FFACASVGTTSSTAIDPILELANVTKSYNIWLHVDSAYAGSACVCPEYRHHIDGVEEVDS
FNMNAHKWFLTNFDCSLLWVKDRNALIQSLSTNPEFLKNKASQSKSVLDYKDWQIPLG
RRFRSLKLWLVLRLYGVENLQAYIRNHIELAIHFEELVSQDMRFEIVAPRTFALVCFRLL
LPCGFEDRTNDVNGDLLQAVNSTGKIFISHTVLSGTYVMRFAVGAPLTEERHIDAAWKL
IQDQASSLLEKL
Capsella grandiflora 22666s0001.1 (SEQ ID NO: 55):
MDSEQLREYGHRMVDFIADYYKTIEDFPVLSQVQPGYLHKLLPDSAPDQPETLDQVLDD
VRAKILPGVTHWQSPGFFAYYPSNSSVAGFLGEMLSAGLGIVGFSWVTSPAATELEMIV
LDWLAKLLNLPKEFLSKGNGGGVIQGSASEAVLVVLIAARDKVLRSAGKNALGKLVVY
SSDQTHSALQKACQIAGIHPENCRVLETDASTNYALRPELLQEAVSKDLKAGLIPFFLCA
NVGTTSSTAVDPLAALGKIANSNEIWFHVDAAYAGSACICPEYRKYIDGVETADSFNMN
AHKWFLTNFDCSLLWVKEQDSLTEALSTNPEFLKNKASQANLVVDYKDWQIPLGRRFR
SLKLWMVLRLYGAETLKSYIRNHIKLAKYYEKLVSQDPNFEIVTPRIFSLVCFRLVPKNE
DEKKCNNQNRKLLEAANSSGKLFMSHTALSGKIVLRCAIGAPLTEEKHMKEAWKVIQD
EASFLLHK
Selaginella moellendorffii 15420188 (SEQ ID NO: 56):
MGEANIGPKPIDAEEFRKHAHEMVDFIADYYRDIESFPVRSQVSQPGYLKTLLPPAAPED
PEALEEVFADIQSKIIPGVTHWQSPNFFGYYPSNSSTAGLLGEMLSAGLNIVGFSWITSPA
ATELEIIVLDWLAKLLKLPDEFLFGGNGGGVIQGTASEAVSVVLLAARTRAISENKRKGL
SEAEILSKLAVYTSDQTHSCLQKGCAIAGIPLENLVIVPTDSSTNYAVSPAAMRQALEDG
VKQGLLPFFLCGTVGTTSSSAVDPLSALGDIAKDFGMWFHVDAAYAGSACICPEFRHHL
DGVEKADSFNMNAHKWLLTNFDCSALWVKESSHLVSALSTTPEFLRNKASDLNQVVD
YKDWQIPLGRRFRSLKLWFVMRMNGASGLRSYIRNHVRLAKRFEGFVREDPRFQLLVP
RTFGLICFRLKPESDDPDNGRTLNSTLLEAVNSSGRMFITHTVLSGVYTLRMAIGGPLTQ
DKHVDAAWKLIQEEATTLLVKGPSHILANNLRLSPILANNLRLSPILANNRI
Setaria italica 3G188200.1 (SEQ ID NO: 57):
MDILNHADTTTANGTSPAAAAAAAVVAPATPSSLVTPPLDADEFRRQGRLVVDFIADYY
TRINEYPVRPAVAPGFLARQLPETAPARPERDALAAALRDVRDLILPGVTHWQSPRHFA
HFAATASNVGALGEALAAGLNINPFTWAASPAATELEVVVTDWLGKALHLPERLLFSG
GGGGTLLGTSCEAMLCTIVAARDRKLAEIGEERIGDLVVYFSDQTHFSFQKAARIAGIRR
GNCREIPTSRESGFTLSPKALRAAVRADEASGRVPLFLCATVGTTPTAAIDPLRELCAAV
SGHGVWVHVDAAYAGAACVCPEFRHAIAGAEAVDSFSTNPHKWLLANMDCCALWVT
RPAALVAALGTDHDVILKDPSAAAQDGHDVVVDYKDWQVALSRRFRALKLWLVLRC
HGVEGLRGFVRAHVRMAAAFEAMVRADTRFEVPVPRQFALVCFRLRPASAGEKRTRG
GEVVEPNELNRRLLEAVNATGRAYISSAVVGGVYVLRCAIGNSLTEERHVREAWSVVQ
EQANVVLAAATATCPDERAVHRARCVETDAADAPASVPPVQMRFPSAQS
Kalanchoe fedtschenkoi 0033s0078.1 (SEQ ID NO: 58):
MGSLPSPHDPSNAFNPMDVAELSIESRLVMDFITQYYQTLETRPVQPRVKPGFLTGQLPE
KAPFHAESMEEILSDVSEKIVPGLTHWQSPNFHAYFPASSSNAGLLGEMLCSGLSVIGFT
WNSSPAATELENVVVDWLADMLNLPPSFRFSGGGGGGGVLQSNTCEAVLCTLAAARD
KVLERIGDDKINKLVAYCSDQTHFTLHKGAKLIGIRRANIKSIGTRRENGFGLCPNDLRN
AITGDLEAGLVPFYLCGTIGTTALGAVDPIKELGKVAREFDLWFHIDAAYGGSACICPEF
RHYLDGVELVDSISMNAHKWLLSNLDCCFLWLQNPKCLIQCLAAEAEFLKGSGEMVDY
KDWQISLSRRFRAIKMWMVFRRYGVSNLMEHIRSDVSMAARFEEMVSADDRFEIVFPR
KFALVCFKLNTKGSVQHGEDDGEDGLDGDSVLTRELMGRVNSSGKAYLSGVEMGRIFF
IRCVIGSSLTEERHVDNLWNLIQEKTQSIMPCRA
Daucus carota subsp. sativus 36068870 (SEQ ID NO: 59):
MGSLSTQKFNPLNLDFFSSESNKVIEFITAYYKNVEKYPVRSQVEPGFLLNMYPKKAPSQ
PVSLDTILQELEADIIPGMTHWQSPNFYAYFRTTTSNAAFQGEMLCNALNVAGFNWICSP
AATELEMIVMDWLGKMLSLPQSFLFAGNGGGVLQGSTSEALICVLSAARDRALKQYGE
DSITKLVVYASDQTHFVVKKAAKLVGIPTKNFRVIPTSIATCFALKPNDIKMAIERDLESG
LVPLFVCATVGATPSGSVDPVEGLGLLAKNYGLWLHIEAAYAGSAFICPELTHYLRGIEH
AHSISINLHKWLLTNMDCSCLWVKSPDVLLESLSMTDEILRNEASESKKVVDFMDWQIA
TSKLFRALKLWFVLRRYGVDNLMAHIRSDIELAKHFEALVNSDKRFEVVVPVNFSLVCF
RLKPNEEGEESLKVLMNWNLMEAVNSSGRAYMTHAVLGDIFVIRCAIGTSLTEERHVNE
LWKLILEKTEVILKRDQ
Daucus carota subsp. sativus 36056758 (SEQ ID NO: 60):
MNTFDTEDFRKQAHLIIDFLADYYQNIEKFPVRSQVSPGYLGEILPDSAPHDPEPIEKILED
VRSNIIPGITHWQSPNFFAYFPSCGSTAGFLGEMLANGFNVVGFNWISSPAATELETIVM
DWLGKMLQLPEAFLFSGGGGGVLQGTTCEAMLCTLVAARDRTLREQGMENFDKLLCP
VQLELEILSDVQNGLIPLFLCVTIGTTPSTAVDPLATLSEVAKKYKLWVHVDAAYAGSA
CICPEFRHFLDGLENVNSFSMNAHKWFLTTLDCCCLWVNDPSALIKSLSTYPEFLRNHAS
ESNKVVDYKDWQIMLSRRFRALKLWFVLRSYGVEKLREFIRVHVEMAKYFEGLVAMD
QRFEVVVPRLFAMVCFRVVCCGENDVNEINEKLLESVNQSGRIYVSHAVLDGVYVIRFA
IGATLTDYSHVSAAWEVVQEHADALLA
Solanum tuberosum 3DMP400026166 (SEQ ID NO: 61):
MGTLNINHELDDQIFNTINPLDPEEFRRQGHKIVNFLADYYQNIEQYPVCSQVNPGYLQK
IVPNSAPNNSESLEKILKDVERDIIPGLTHWQSPNFFAYFPSSGSTAGFLGEMLSVGFNVV
GFNWISSPAATELESIVMDWFGKMLNLPNCFLFASGGGGVLQGTTCEAMLCTIVAARD
QMLRKISRENFGKLVVYASDQTHFSLKKAAHIAGIDPGNFRVIPTIKANEYTLCPKSLRL
AILNDLKEGNVPLFLCATIGTTATTSVDPLRLLCEIAKEFGIWVHVDAAYAGSACICPEFQ
VFLDGVENANSFSLNAHKWFFSTLDCCCLWVKDPSALTNALSTNPECLRNKATELNQVI
DYKDWQIALSKRFRALKLWLVLRSYGVTNLRNLIRSHVNMAKHFEGLVATDKRFEIFV
PRKFAMVCFRISPLVLSQVSTKFDDEKEVNMFNTKLVESINSCGKLYLTHGVVGGTYIIR
FAIGASLTHYRHVDVAWKVIQDHANALLNQGYV
Solanum tuberosum 3DMP400024738 (SEQ ID NO: 62):
MGTMKINPEHEFDGQFSINTSSSRLLDPEEFRRQGHMMVDFLADYFQNIEKYPVRSQVE
PGYLKKLLPDSAPYKPEPIAKILEDVERDIFPGLTHWQSPNFFAYFPCTSSTAGILGEMLS
AGLNVVGFSLIASPAATELESIVMDWLGKMISLPKTYLFSGGHGGGGVIQGTTCEAMLC
TIVAAREQMLEKVGREKVDKLVVYASDQTHFSFEKAVKISGIKLENFRVIPTTKDTEFAL
DPKSLSRTIEQDIKSGFIPLFMCATIGTTSTTVVDPLKLLCEITKDYGIWVHVDAAYAGGA
CICPEFQHFLDGIENANSFSFNAHKWLFSNLDCCCLWVKDPSALTNALSTRPECLRNKAT
DTKQVVDYKDWQLSLSRRFRALKLWLVLRSYGIDNLRNFIRSHVKMAKHFEQLVSMD
ERFEIVAPRNFSMVCFRVSPLALGNKQVNKFNMELLESINSCGNIHMTHALVGGVYMIR
FAIAAPLTEYKHIDMAWEVICNHANAMLDVN
Solanum lycopersicum 36137005 (SEQ ID NO: 63):
MGTLNINHELDDQIFNTINPLDPEEFRRQGHKIVNFLADYYQNIEQYPVCSQVNPGYLQN
IVPNSAPNNPESLDKILKDVQNDIIPGLTHWQSPNFFAYFPSSGSTVGFVGEMLSVGFNV
VGFNWISSPAATELESIVMDWFGKMLNLPNCFLFASGGGGVLQGTTCEAILCTIVAARD
QMLRKISRENFGKLVVYASGQTHFSLKKSAHIAGIDPGNFRVIPTIKAKEYTLCPKSLRLA
ILNDLKEGNVPLFLCATIGTTSTTSVDPLRLLCDISKEFGIWVHVDAAYVGSACICPEFQV
FLDGVENANSFSLNDPSALTNALSTNLEFLRNKATELNQVIDYKDWQIALSRRFRALKL
WLVLRSYGVTNLRNLIRSHVNMTKHFEGLIAMDKRFEIFVPRKFAMVCFRISPLVLSQVS
IKFDDEKEVNMFNTKLLESINSCSKLYLTHGIVGGTYIIRFAIGASLTHYRHVDIA
Daucus carota subsp. sativus 36065781 (SEQ ID NO: 64):
MCKPKSSPASHINWQSPNFFAYFPSSGSTAGFLGEMLSTGFNVVGFHWMASPAATELEN
VVTDWFGKMLQLPKSFLFSGGGGGVLQGTTCEAMLCTLVAARDKNLRQHGMENIGKL
VVYCSDQTHSAMQKAAKIAGIDPKNFRTVETSRASNFQLCPRRLESAILTDIQNGLIPLYL
CATVGTTSSTAVDPLPALTEVAKKYDLWVHVDAAYAGSACICPELRQYLNGVENADSF
SLNAHKWFLTTLDCCCLWVKNPSALIKSLSTYPEFLRNNASETNKVVDYKDWQIMLSR
RFRALKLWFVLRSYGVGQLREFIRGHVDMAKYFEGLVGKDKRFEVVVPRLFSMVCIRV
RPSAMTGKSCGNDVNELNRKLLESLNESGRIYVSHTVLDGIYIIRFAIGATLTDINHVSAA
WKVVQDHATALLDDTNFLAKKVADIILS
Oropetium thomaeum 35995617 (SEQ ID NO: 65):
MAILNHADDASPANDDNPATAPAMAPATNPRPLDADEFRRQGRLVVDFIADYYARVEE
YPVRPSVTPGFLSRKLPETAPEQPEPGHGDAFASALRDVRDLILPGITHWQSPNHFAHFA
ATASNVGALGEALAAGLNINPFTWAASSAATELEVVVTDWLGKALHLPQELLFSGGGG
GTLLGTSCEAMLCTVVAARDRKLGEIGEHRIGDLVVYCSDQTHFSFRKAARVAGIRRAN
CREIPTSLESDFALSPSALLAAVRADEAAGLVPLYLCVTVGTTPTAAVDPVRELCAAVA
GRGVWVHVDAAYAGAARVCPELLRHAGAIVDGVDSFSTNPHKWLLANMDCCALWVQ
QPDALVAALGTDHDVILKDPAAAAAGDVVVDYKDWQVALSRRFRALKLWLLLRCHG
VEGLRAHVRDGLRMAEAFEAMVRADARFEVPVRRQLSLVCFRLRPTAVIREKQQQQRG
RRRDHDDDTAAANELNRRLLEAVNATGRTYMSCAVVGGVYMLRCAIGNSLTEDRHVE
EAWNVVQEQASAILDAAMVVRADECTVCTAAHCVQMGMVDDILAASFPTGNEVTIR
Oryza sativa 33157740 (SEQ ID NO: 66):
MAILNHSDAAFPVAATTPLLGRRPLDAGEFRRQGRQVVDFIADYYAGINDYPVRPAVAP
GFLAGKLPATAPSTPEPDALTAGLRDVRELMLPGLTHWQSPRHFAHFSATASNVGALGE
ALAAGLNVNPFTWEASPAATELEVVVTDWLGKALHLPERLLFAGGGGGTLLGTSCEAM
LCTIVAARDEKLAEIGEERIGDLVVYCSDQTHFSFQKAARIAGIRRGNCREIPTCRESGFV
LTATALQAAVAADEAAGRVPLFLCATVGTTPTAAVDPLRELCAAVEGRGVWVHVDAA
YAGAACVCPEFRHAIAGAEAVDSFSTNPHKWLLANMDCCALWVARPAALVAALGTDD
DVILKDAAAAARPARGDHHHHAAVDYKDWQVALSRRFRALKLWLVLRCHGVDGLRA
VVRSHVRMAAALERMVRADARFEVPVPRQFALVCFRLRGGGAAAQLVGGDELTASNE
LNRRLLEAVNATGRAYMSSAVVGGMYVLRCAVGNSLTEEHHVREAWSVVQGQAAAV
LATAGAAADTARTKDHAAGDDHGADQPHAMTTTTTMGCRSGPWEL
Brachypodium stacei 01G392300.1 (SEQ ID NO: 67):
MAPASSTRQVITDHKTQKENSSCTVINHLLDADEFRRQGHKVIDFIADYYSGIADYPVHP
SVTPGFLLNQLPADPPEDPDTFASALQDVRDLILPGMTHWQSPRHLAHFPASSSVTGALG
EALAAGINAVPFMWSASPAATELEMVAVDWLGKALHLPKTLLFSGAGGGTLLGTSYRK
LAETGAGRIGDLVVYGSDQTHFALRKAARIAGIRHGRCRELRTCIADMFALSPAALSAA
MDADAGAGLVPLFLCATVGTTQTKAVDPIGALCAEAAPHGVWVHVDAAYGGSALVCP
ELARDAIDGVEAVDSFSMNAHKWLLVNTDCCALWVKRPALLVSALGTQDEDEVILRD
AAAQGHDVVDYKDWAVTLTRRFRALKLWLVLRCYGVEGLREHIRGHVRMAALFEGM
VNADPRFEVVTERRFALVCFRLRPDQLPDEGNKKKTMAAANELNRRLLQEVNAAALGP
YMSAANVGGIYVLRCAVGSTLTEKRHVRQAWEVVQEKATSILRA
Amaranthus hypochondriacus 32828676 (SEQ ID NO: 68):
SLHDETLQGIKYVTQYYKNVEKYPVVSKVKWGYLRQILPENAPSLPESIDQILEDVDTKI
VPGLTHWQSPNFFAYFPATASNAAMLGDIVCSGLNVVGFSWISSPAATELEAIVMDWM
AKLLMLPPTFLFSGGGGGVIHGSTCEAIVCTQAAARDVALNIHGEEKITKLVVYASDQT
HISFQKAAKLIGIPPRNFRVLPTSSATDFALSPTTLRASIEVDLSQGLVPFYICATIGATPSG
AVDPIDGLGQIARDYGAWLHVDAAFAGNACICPEYRHYLDGVELADSISMNPHKWLLT
NMECSCLWLKNPKLMVDSLSTKPEILNNKATQSGDVIDYKDWQIALSRRFRALKLWIVI
RRYGSTYLMNHVRSDIELAKYFESLIKQDERFELVVPRKFSLVCFRMKLVGREDVETLT
NQKLLEDVNSSGKAYMTHAVIGGKFVIRCAIGGTLTEKRHIDSLWKLIIEKVPLTTCEL
Brachypodium distachyon 5g21770.1 (SEQ ID NO: 69):
MSSNSCPAAAAATFTTPPGAHPLPLDADAFRRQGRQVADFIADYYDRIEDYPVRPNVSP
GFLAAQLPDAAPSWPEEPDALASALRDVRDLILPGLTHWQSPRHFAHFAATASNAGAL
GEFLAAGLNVNPFTWAASPAAAELEVVVTDWLGQALGLPEKLLFRGGSGGGGTLLGTS
CEAMLCTIVAARDQKLLKIGEDRIGDLVVYCSDQTHFSFKKAARVAGIRRGNCRVIPTRF
EDGFALSPAALAAAVRDDVARGKVPLFLCATVGTTATGAVDPVRELCAAVGAGHGSG
VWVHVDAAYAGGACVCPEFRHVAAGAEEADSFSTNPHKWLLANMDCCALWIRRPGL
LVAALGAGEDEDAILNKAPPAARGMQADLMVDYKDWQVPLSRRFRALKLWLVLRCH
GVEGLRGVVRGHVRMAAAFEAMVRADPRFEVPVPPAFALVCFRLRPLAAHPGSSSGID
EVNGRLLEAVNGTGRAYMSGAVVGGAYVLRCAVGNSLTEDRHVREAWSVVQEQADA
ILAPSDDEDRCCTDQIQTEMELQRRPLGAAADVFA
Brachypodium distachyon 2g02360.1 (SEQ ID NO: 70):
MAPASSKLHAITDDKTQQQNSSCPAASNGAIEPSNAKCAASSNHLLDADEFRRQGHKVI
DFIADYYAGIADYPVHPSVTPGFLLNQLPADPPSRPEDHPDGAFGPALQDVRDVILPGMT
HWQSPRHFAHFPASSSVAGVLGEALAAGINAVPFTWAASPAAAELEMVAVDWLGKAL
HLPESLLFSGAGGGTLLGTSCEAILCALVAARDRKLADIGTDRIGDLVVYGSDQTHFALR
KAARIAGIRHDRCRELQTCLADMFALSPAALSAAMDADAGAGLVPLFLCATVGTTQTT
AVDQVGALCAAAAPHGVWVHVDAAYAGSALVCPELARDAIDGIEVVDSFSMNAHKW
LLANTDCCALWVKQPKLLVVSLGTQNEELILRDAAAEGHDVVDYKDWAITLTRRFRAL
KLWLVFRCYGVEGLREHIRAHVRMAALFEGLVKDDPRFEVVTERRFALVCFRLRAPDQ
LMDEGNEKKKTTAAANELNRRLLREVNGVALGPYMSAAVVGGIYILRCAVGSTLTEER
HVRQAWEVVQERATSILRG
Sorghum bicolor 009G192600.1 (SEQ ID NO: 71):
MGVAVTAEVVHARSCKGTPPVGAAASVMVWDGAGQGYSCQPVGTTTANGGTTPAAP
VAIAMPSLPHPLLDADEFRRQGRLVVDFIADYYARIDEYPVRPAVAPGFLARQLPETAPA
RPEPDALAAALRDVRDLILPGVTHWQSPRHFAHFAATASNVGALGEALAAGLNINPFT
WAASPAATELEVVVTDWLGKALHLPESLLFSGGGGGTLLGTSCEAMLCTIVAARDRKL
AEVGEERMGDLVVYCSDQTHFSFQKAARIAGIRRGNCREIPTSMEAGFTLSPKALAAAV
RADEAAGRVPLFLCATVGTTPTAAVDPVRELCAAVAGRGVWVHVDAAYAGAASVCPE
LRHAVAGVERVDSFSTNPHKWLLANMDCCALWVRRPAALTAALGTDHDVILKDPSAQ
AAQEGGAVVDYKDWQVALSRRFRALKLWLVLRCHGVEGLRGLVRAHVRMAAAFEA
MVRTDARFEVPVPRQFALVCFRLRAAAVLVVGEKRARDGDDEVVTAGNELNRRLLEA
VNATGRVYMSSAVVGGTYILRCAIGNSLTEERHVREAWSVVQEQATAILAAARRPTAR
TNRRTVRRAHAAL
Kalanchoe laxiflora 0994s0009.1 (SEQ ID NO: 72):
MGSLQSPHDPNAFNPMDVAELSIESRLVMDFITQYYQTLETRPVQPRVKPGFLTGQLPE
KPPFHAESMEEILSDVSEKIVPGLTHWQSPNFHAYFPASSSNAGLLGEMLCSGLSVIGFT
WNSSPAATELENVVVDWLADMLNLPPSFRFSGGGGGVLQSNTCEAVLCTLAAARDKV
LERIGDDKINKLVVYCSDQTHFTLHKGAKLIGIRRANIKSISTRRENGFGLCPNDLRNAIK
SDLEAGLVPFYLCGTIGTTALGAVDPIKELGKVAREFDLWFHIDAAYGGSACICPEFRHY
LDGVELVDSISMNAHKWLLSNLDCCFLWLQNPKCLIQCLAAEGEFLKGSGEMVDYKD
WQISLSRRFRAIKMWMVFRRYGVSNLMEHIRSDVSMAARFEEMVAADDRFEIVFPRKF
ALVCFKLNTKGSVQHGEVDGEDGLDGDSVLTRELMGRVNSSGKAYLSGVEMGRIFFIR
CVIGSSLTEERHVDNLWNLIQEKTQSIMPRRA
Kalanchoe laxiflora 0003s0173.1 (SEQ ID NO: 73):
MGSLSSPRDLTKPFNPLDPTELAVESSLVTDFIAEYYRTVEQRPVQPHVTPGFLTSQLPSA
APFASESVESILQDVYDKILPGLVQWQSPNFHAYYPATCSNAGLLGEMLCSGLNVVGFT
WSASPAAAELEQVVVDWMGKMMGLPQSFLFSGGGGGVLQGSTCEAVVCTLAAARDR
ALERVGDDMFNKLVVYCSDQTHFTLKKGSKLVGIRPANVKAIKTTKNNEYGLCPTDLR
NLVASDVKAGFIPIYLCGTIGTTAFGAVDPIRELGKVAREFNMWFHVDAAYAGSAFICPE
FRHYMDGVELADSFSTNPHKWLLSNMDCCVLWLKFPKRVIKSLAAEGVFLEGGSETMV
DYKDWQIALSRRFRAIKLWMVIKRYGLKNLISHIRSDVSMAKRFEELLLSDRRFEVVFPR
KFSLVCFKLDVMKNVPEVVDEDDGELSHDSKLTRELMASVNVTGKAFLTGVRLGRIFFI
RCAIGSTLTEDRHIQDLWKLIQEKAHKICANHDLKFRV
Panicum hallii 32512198 (SEQ ID NO: 74):
MAILNHGDTTTANGSSPADAAAVAPAMPSLVQPPLDADEFRRQGRLVVDFIADYYTRID
EHPVRPAVAPGFLARQLPDTAPARPEPGDDALAAALRDVRDLILPGVTHWQSPRHFAHF
AATASNVGALGEALTAGLNINPFTWAASPAATELEVVVTDWLGKALHLPESLLFSGGG
GATLLGTSCEAMLCTLVAARDRKLAEIGEERIGDLVVYCSDQTHFSFQKAARIAGIRRG
NYREIPTSRESGFTLSPKVLRAAVRADEAAGRVPLFLCATVGTTPTAAVDPLRELCATVA
GHGVWVHVDAAYAGAACVCPEFRHAIAGAEAVDSFSTNPHKWLLANMDCCALWVRR
PEALTAALGTDHDVILKDPSSERDCGRGVVDYKDWQVALSRRFRALKLWLVLRCHGV
EGLRGFVRAHVRMAAAFEDMVRADARFEVPVPRQFALVCFRLRSAAAGEKRARDGDD
AEPNELNRRLLEAVNATGRAYMSSAVVGGIYVLRCAIGNSLTEERHVREAWCVVQEQA
TVVLAAAACTEERAVHSARCADAPAAVPPVQNEGYGEPTSIAAKIFGTSIARCSIKSEAS
TYHSWSTLWRTLMFKLLTWIISRL
Prunus persica 6G202600.1 (SEQ ID NO: 75):
MTSALDPVEFRRQGHMMVDFIADYYQNIDKYPVLSQVDPGYLRKRLPESAPDNPEPIETI
LQDVQEHIVPGLTHWQSPSFFAYFASNVSIAGFLGEMLSTGFNVVGFNWVSSPAATELE
SIVMDWLGNLLSLPKSFLFSGNGGGVIHGSTCEAIVCTMAASRDQMLSRIGGDNIGKLV
VYGSDQTHSALQKASQIVGINPKNFRAIEATRSTTFALSPESLKLAISSDIEAGLVPLFLCA
TVGTTATTAVDPLGPLCDVAKHHGMWVHVDAAYAGSACICPEFRHFIDGIEGVDSFSFN
AHKWFFTGLDCCCLWVKNPGALISSLSANPEFLRNKPTDSKQVVDYKDWQIALSRRFR
AMKLWLVLRSYGVVNLRNFLRSHVKMAKLFEGLVAMDQRFEIVVPRNFSMVPPTTPTS
NSFHQNGIEINVEKCTNEVNCKLLEAINASGRVFMTHAMVGGMYVIRCAVGVTQTEEK
HIAMAWKVVQEHADVILKNNGDDGDANLKLPLLDKIA
Prunus persica 4G086700.1 (SEQ ID NO: 76):
MGSLNFDHPQENNSAHMSGPLDLVELRRQGHMIIDFITDYYQNIEKHPVLSQVQPGYLK
QRLPESAPYNPEPIETILRDVQDHIVPGLTHWQSPNHFAYFPATISTAGFLGEMLTTCFNV
VGFNWMASPAATELETIVMDWLGDMLKLPNSFLFSGTGGGVLHGSTHESVVCTMAAA
RDQILSRIGEENIGKLVVYGSDQTHSVIQKVSQIVGIPSKNFRAIETTISSSFTLSPETLRLT
VCSDMEAGLVPFYLCATVGTTATTAVDPLGPLCDVAKDYGMWVHVDAAYAGSACICP
EFRQYIDGIEGANSFSFNAQKWFFTALDCCCLWVKNPSALTKSMSTDLEVLRNKASESK
RVVDFKDWQIALTRRFRAIKLWLVLRSYGVANLRNFLRSHVKMAKRFEGLVRTDERFE
VVVPRIFALVCFRISPSAISKANPTPSDEKCVNEVNCKLLEAINGSGWVYMTHAVVGGM
YVLRCAIGASLTKEKHVAMAWKVVQEHVDAILPLTMY
Prunus persica 4G087100.1 (SEQ ID NO: 77):
MMGSVEFEHPQENNSAHMTTSPLDPEEFRRQGHMVIDFIADYYKTIEKYPVLSQVQPGY
LKKRLPESAPYDPEPIETILQDVQDHLVPGLTHWLSPNHFGYFPAAISTAAFLGEMLTTG
FNVVGFNWMASPAATELENIVMDWLGDMLKLPKSFLFSGNGGGVLQGTTCEAIVCTM
AAARDQMLRQIGRENIGKLVVYGSDQTHSALQKASQIVGIHPKNFRAIETTTSTSFALSP
EVLKSTICSDIEAGLVPLFLCATVGTTAITAVDPLGPLCEVAKEHDMWVHVDAAYAGSA
FICPEFQYFIDGVEGADSFSLNAHKWFFTTLDCCCLWVKNPSALVSSLSTNPEFLRNKAT
DSKQVVDYKDWQIALSRRFKAIKLWLVLRSYGVGNLRNFLRSHVKMAKIFEGLVGMD
KRFEIVAPRHFSLVCFRVSPSAISKANPSLSDHDNGKLKAHNYELLNGVKCVVNEVNSK
LLEAINGSGLVYMSHAVVGGMYVLRCAIGASLTEEKHVAMAWKVVQEHADAILGTKII
VDQT
Medicago truncatula 31073039 (SEQ ID NO: 78):
MNTSSSNPPQSDPQKTMNPLDLEEFKRQGYMMIDFLTDYYKNIENYPVLSKVEPGYLAK
ILPSSAPFQPESIESILEDVQQHIIPGITHWMSPNYYAYFPSSGSIAGFIGEMLSTGFNVVGF
NWLSSPAATELETIVMNWLGKLLNLPKSFIFSSNIKGGGEIKKLSQIGKDNIGKLVVYCSD
QTHSALQKATQIVGIHSENFRVIKTKGSNLFALSPDSLLSTILLDVDNGLIPYFLCATIGTT
STNAVDPIKLLCNVTKEYDIWVHVDAAYAGSVCICPEFRHCIDGIEELNSFSFNAHKWFL
TNLACCCLWVKDHNALTTSLSTNPEFLRNKKSDSKEVIDYKDWQIPLSRKFNALKLWIV
LRSYGVENLKNFLRNHVEMAKIFEGLVRKDERFEIVVPSKFSLVCFRISPFAISIANDSEG
YYVGKMMNDAYLVNEMNHKLLDLINSSGKAYMSHGEVEGSFVIRCAIGATLTEEHHVT
MTWKLVQQIASFLLGTPLN
Zea mays GRMZM2G009400 (SEQ ID NO: 79):
MAILNRADTSHTTTASNGSATPAAPVAIAMPSLPHPPLDADEFRRQGRLVVDFIADYYA
RIDGYPVRPAVAPGFLIRQLPEAAPARPEPDALAAALRDVRDLILPGVTHWQSPRHFAHF
AATASNVGALGEALAAGLNVNPFTWAASPAATELEVVVTDWLGKALHLPESLLFSGGG
GGTLLGTSCEAMLCTIVAARDRKLAEVGEERIGDLVVYCSDQTHFSFQKAARIAGIRRG
NCREIPTSRESGFTLSPKALAAAVRADEAAGRVPLFLCATVGTTPTAAVDPLRELCAAV
AGHDVWVHVDAAYAGAACVCPEFSHVVAGVEAAESFSTNPHKWLLANMDCCALWV
RRPAALTAALGTDHDVILKDPAAAQAQAQQQQCSDGGVVDYKDWQVALSRRFRALKL
WLVLRCHGVEGLRGLVRAHVRMAAAFEAMVRGDARFEVHVPRQFALVCFRLRAVAV
AVAGEKRAGDYDGVAAGNELNRRLLEAVNATGRVYMSSAVVGGAYILRCAIGNSLTE
ERHVREAWSVVQEQATAILSAATATARTNGLTVRRARCDAEADVSDVPTPQQPLPLG
Glycine max 07G059000.1 (SEQ ID NO: 80):
MEMKNTMNRNPQSDAPIIKPLDPEEFKRQGYMMVDFLADYIRNVSHYPVLSKVEPGYL
KQRLPTSAPCGPEPIESILKDVQDHIIPGLTHWQSPNFYGYFPSSGSIAGFMGEMLSAGLN
VVGFNWVSSPSATELESIVMDWLGQVLNLPKSFLFCGDHGGGVVLGTTCEAILCTLVAA
REKKLSQVGKENIGKLVVYGSDQTHSALQKAAQIAGIHPANFRVIKTKRSNSFALSPDSL
LSTILLDVERGLIPCFLCATVGTTAIATIDPIGPLCNVAKDYGIWVHVDAAYAGSACICPE
FRHCIDGVEEVNSFSLNAHKWFLTNLTCCCLWVKDHIALTKSLTVNPQFLRNKASESKR
VIDYKDWQIPLSRKFNALKLWLVLRSYGVENIRNFLRNHVQMAKTFEGLVRLDKRFEIV
VPPKFSLVCFRIAPSAIIANGLSKGVEACYNGKLVNDEYMVNEVNRKLLDSVNSSGDAF
MTHGEVEGAFMIRCAIGGTLTEEHHVIMAWKLVQEHANSLLGL
Panicum virgatum Ca01381.1 (SEQ ID NO: 81):
MAILNHGDTTAASGTSPAAAAVNVAPPMHSLVQPVLDADEFRRQGRLVVDFIADYYTR
IDEYPVRPAVAPGFLARQLPEAAPARPEPGGDALAAALRDVRDLILPGVTHWQSPRHFA
HFATTGSNVGALGEALAAGLNINPFTWAASPAATELEVVVTDWLGKALHLPERLLFSG
GGGGTLLGTSCEAMLCTLVAARDRKLAEIGEERMGDLVVYCSDQTHFSFRKAARIAGIR
RGNCREIPTSRESGFALQPRTLLAAVRADEAAGRVPMFLCATVGTTPTAAVDPLRELCA
AVAGRGVWVHVDAAYAGAACVCPEFRGATAGAEAVDSFSTNPHKWLLANMDCCAL
WVRRPEALTAALGTDHDVILKDPSSERGGGVVDYKDWQVALSRRFRALKLWLVLRCH
GVEGLRGLVRADARFEVPVPRQFALVCFRLRAAAAAAVGEKRGRDRDNDAEPNELNR
RLLEAVNATGRAYMSSAVVGGIYVLRCAIGNSLTEERHVREAWRVVQEQATAVLAAA
ACTEERAVRSAR
Theobroma cacao 27425420 (SEQ ID NO: 82):
MSSASRKTFLPLEPTSFTNESKAVIDFIADYYKNIEEYPVQSGVEPGYLSAKLPDSAPYCP
ESLEDILKDVNDCIIPGLTHWQSPNFFAYFQANASTAGFLGEMLCSGFNVVGFNWISSPA
ATELESIVLDWMGKLLKLPSSFLFSGTGGGVLHGSTCEAAVCTLAAARDKALKELGGW
ENITKLMVYASDQTHFTFQKAAKLVGIPPSNFRFIETSLSTGFSMSSDQVRLAIEHDIKSG
LVPLFLCATIGTTACGAIDPIAELGQVAREYKLWLHIDAAYAGSACICPELRHFLDGVEL
ANSVSMNPHKWFLTNMDCCCLWITEPRLLVDSLSTDPEILRNKASEFKAVLDYKDWQV
ALSRRFRALKLWIVIRRHGLANLVYHIRSDISMAERFEAFVAKDDRFDIVVPRKFALVCF
RLKPKQELEGLELNSRLLEAINSSGRAFMTHAVVGGIYVIRCAIGTTMTEERHVDALWK
LIQEKAQGLLME
Fragaria vesca 27274768 (SEQ ID NO: 83):
MGSLDFHHVPEKTNSDPPMANPMDPEEFRRQGHIMIDFIADYYKNIEKYPVLSQVQPGY
LKKLLPESAPYNPEPIETILQDVQDHIVPGITHWQSPSYFAYFPSSGSIAGFLGEMLSTGFN
VVGFNWMSSPAATELERTTCEAIVCTMAAARDQMLSRIGKDNIGKLVVYGSDQTHSAL
KKASQIVGIHPNNFRAIKTTKSTEFALSPELLRSTICSDIDKGLVPLFLCATMGTTATTSVD
PLRGLCDVAKDYDLWVHVDAAYAGSICICPEFRHFIEGVDGANSFSFNAHKWFFTTLDC
CCLWVKNPTALINSLSTNPEFLRNKASDSKQVVDYKDWQVALSRRFRALKLWLVLRSY
GVANLRSFLRSHVKMAEVFEKLVRENKWFEVVVPRNFAMVCFRISPSAIRKAPTDDDGI
DVVINEVNSKLLEAMNTSGSVYMTHAVVGGMYVLRCAIGATMTEEKHVLMAWKCGS
ALERKDVAANETLSFNFQRRFDRRARQRRGHVGFRLAITMLDLKTSERDGARRWSIGA
YANQITTISQANSSVAWTMEFHSCFIFFCGSIKLDTQVPNDDFVLSARWPPSFPVSGWSTI
NFHETIKIYVGSLDSLDSWTMEFHSCFTFFCGS
Gossypium raimondii 26786642 (SEQ ID NO: 84):
MVSASRKTFLPLDPVTFSNESKAVIDFIADYYENVEKYPVQSTVEPGYLSAMLPESAPYC
PEPLQDILEDVSNCIIPGLTHWQSPNFFAYFHANASTAGFFGEMLCSGFNVVGFNWISSP
AATELESIVLDWMGKMLKLPSSFLFSGTGGGVLHGSSCEAAVCVLAAARDKALKELGG
WENITKLVVYASDQAHFTFQKAAKLVGIPPSNFRLIETSFSTGFSLSPENLRFVIEDNIRSG
LVPLFLCATIGTTPSGAVDPIAELGKVAMEFKLWLHIDAAYAGSGCICPELRHYLDGVEL
ANSISMNPHKWFLTNMDCCCLWIKEPKLLVDSLSTDPEILRNNASKSKAVVDCKDWQI
ALSRRFRALKLWVVIRRHGLANLMCHIRSDIAMAKRFEALVGEDERFEIVVPRKFALVC
FRLKPKVEEEDLNCKLVEAINSSGRAFMSHAVLSGIYVIRCAIGTTLTQQHHVDALWKLI
QDKAQSLLM
Populus trichocarpa 26994989 (SEQ ID NO: 85):
MGSLSTNTFSPLDPNGFTNDSKMVIDFIADYYKNIENNPVQSQVKPGYLLTQLPDTAPYC
EESLEDVLKDVTDSIIPGLTHWQSPNFFAYFQANASTAGFVGEMLCTGLNVVGFNWIAS
PAATELESIVMDWMGKMLKLPSTFLFSGNGGGVLHGSTCEAIVCTLVAARDETLRMIGA
ENITKLVVYASDQTHSTLLKGVKLVGIPSSNFRCLSTSFSSEFSLSPQALEDAIENDIKAGL
VPLFLCATVGTTACGAVDPVMDLGEIARKYNLWFHIDAAYAGSACICPEFRHYLDGVEL
ADSLSMNPHKWLLTNMDCCCLWVKQPRLLIESLSSDAEFLRNNASESSDVVDYKDWQI
ALSRRFRALKLWIVIRRHGLANLMCHIRSDVNLAKRFESLVAKDSRFEVVVRRRFSLVC
FRLKHNDECQGLELNRKLLAAVNESGRAFMTHAVVGGLFIIRCAIGSTLTEERHVDDLW
KLIQEKAADLLSKKQVLLDN
Malus domestica 22679008 (SEQ ID NO: 86):
MSLLAFYSNSGERSKRVHLSASTYGNSTPNSYISLPYALFSSATQLINIHSNSSNFQMGSLI
SQENNSPNVPTNPLDPEEFRRQGHLVIDFIADYYKSIEKHPVLSQVQPGYLKKRLPDTAP
YNPEPLETILQDVQDHIVPGITHWQSPNYFAYFPSSGSVAGFLGEMLSSGFNVVGFNWM
SSPAATELESTVRDWFGNMLKLPKSFLFSGNGGDVIQGTTCEALVCAMVAARDQKLSK
FGRHNIGKLVVYGSDQTHSALQKASQIVGIHPENFRSIETTRSTSFALSPESLKVIIYSDIEA
GLVPLFLCATVGTTAIATVDPLGPLCGVAGDYGMWVHVDAAYAGSACICPSFDISLMA
SRVQIHSVSTRTNGSSPLSTVVAFGLRIPTRWNKATELKQVVDYKDWQIALSRRFRSMK
LWLVLRSYGVANLRNFLRSHVKMAKIFEGLVAMDKRFEIVAPRNFSLVCFRVSPSSISN
KASSDQNGKTDYCCDANGDENSVIINEVNRKLLESINVSGHVYMTHGVVGGLYMLRFA
VGATLTEEHHIALAWKVVQEHADQILTKY
Citrus Clementina 20801973 (SEQ ID NO: 87):
MRAGEASIIKMGSFGLSANNITHGSSFSADLEPKSFSDESKAVIDFIADYYKNIEKYPVQS
KVEPGYLSARLPDTAPHSPESLDDILKDVTDCILPGLTHWQSPNFFGYFQANASTAGFLG
EMLCSGFNVVGFNWLASPVATELESIVMDWMGKMLKLPSSFLFSGTGGGVLHGSTCES
LVCTLAAARDKALEKLGGGFDNITKLAVYASDQTHFALQKSAKLIGIPPANFRPLRTSFS
TEFSLSPDTVRAAIEDDIKSGHVPLYLCATVGTTGAGAVDPIEELGKIANEYKLWLHIDA
AYAGSACICPEYRHYLNGVELADSISLNPHKWFLTNMDCCCLWVKHPSFLVDSLSTESD
IMRNRSPASNTSTNAAPVIDYKDWQIALSRRFKALKLWTVIRKHGYSGLMYHIRSDVSM
AKRFAAMVAKDERFEIVVPRKFALVCFRLKPKRESEGSELNRELVDALNGSGRAFLTQA
MLGGVYVIRCSIGTTLTQDRHVDDLWKLIQEKADRLLSLQEPEHASR
Citrus Clementina 20818150 (SEQ ID NO: 88):
MGSLNSDHELKTNSASFNNPMDSEEFRRQGHMIIDFIADYYRDVEKYPVLSQVEPGYLQ
KRLPESAPYNPEPIETILQDVQQHIVPGITHWQSPYYFAYFPSSGSIAGFLGEMLSSGFNV
VGFNWMSSPAATELENIVMDWLGEMLKLPKSFLFSGTGGGVIQGTTCEAILCTLAAARD
QILNEIGRENISRLVVYGSDQTHSALQKAAQIAGIDPKNFRAIKTTKSSSFTLTPESLQAAI
DLDIQSGLIPLFLCATVGTTAITTVDPLGPLCDIAKRYSIWIHVDAAYAGSACICPEFRHFI
DGIESADSFSLNAHKWFFTTLDCCCMWVKNPNALIKALSTNPEFLRNKASDSKQVVDY
KDWQISLSRRFRALKLWLVLRSFGVANLRNFLRSHVGMAQLFQELVGGDNRFEIVAPR
NFAVVCFRVLPSASGLGNGKANEGANELNRKLLESINASGQLYVSHGMVAGIYFIRFAV
GATLTEDRHVIAAWKVVQEKLDGILATS
Vitis vinifera 17834108 (SEQ ID NO: 89):
MGSLSFNTFSPLDPQSFSEESKMVVDFIADYYKNVEKYPVQSQVDPGYLMHHCPDTAPY
CPEPLETILKDVSDGIIPGLTHWQSPNFFGYFQANASTAGFLGEMLCTGLNVVGFNWIAS
PAATELESIAIICSLAAARDKVLKKLGHHKITKLVVYGSDQTHSTLQKASKLVGIPASNFR
SLPTSFSNYFALCPDDVRTAMEEDIGAGLVPLFLCATVGTTSSGAVDPLEALGHVAKDF
KVHHLNGVELAHSISMNPHKWLLTNMDCCCLWIKEPKLFVDSLSTAPEFLRNNASESK
KVIDYKDWQIALSRRFRAIKVWAVVPRRFALVCFRLRPREEGESTELNSRLLMAVNGSG
AAFMTHAVVGGIYIIRCAIGSTLTETRHVDSLWKLIQEKAQLVLQEPGLALEEDYIDPCIG
VSATSLHAVVRWYCNYSSEINAHLVFIAFFVVVCKENRENYVLGVNGPPN
Petunia hybrida ABB72475.1 (SEQ ID NO: 90):
MDTIKINPEFDGQFCKTTSLLDPEEFRRNGHMMVDFLADYFHNIEKYPVRSQVEPGYLE
RLLPDSAPIQPEPIEKILKDVRSDIFPGLTHWQSPNFFAYFPCSSSTAGILGEMLSAGLNVV
GFSWIASPAATELESIVMDWLGKLINLPKTYLFSGGGGGVMQGTTCEVMLCTIVAARDK
MLEKFGRENIDKLVVYASDQTHFSFQKAVKISGIKPENFRAIPTTKATEFSLNPESLRRAI
QEDKKAGLIPLFLCTSIGTTSTTAVDPLKPLCEIAEEYGIWVHVDAAYAGSACICPEFQHF
LDGVEHANSFSFNAHKWLFTTLDCCCLWLKDPSSLTKALSTNPEVLRNDATDSEQVVD
YKDWQITLSRRFRSLKLWLVLKSYGVANLRNFIRSHIEMAKHFEELVAMDERFEIMAPR
NFSLVCFRVSLLALEKKFNFVDETQVNEFNAKLLESIISSGNVYMTHTVVEGVYMIRFAV
GAPLTDYPHIDMAWNVVRNHATMMLNA
Carica papaya 16421889 (SEQ ID NO: 91):
MSSLSRDLNASPLEPENFRVESKRVIDFIADYYKNIETYPVQSRVKPGYLAGRLPSSAPFS
PESLETILQDIAENISPGLTHWQSPNFFGYFQANASTAGFHGEMLCSGLNVVGFNWISSP
AATELESLVMDWMGNMLKLPSSFLFSGSGGGVLHGSTCEAVVCTLAAARDKTLNQLG
GNYQNITKFVVYASDQTHFTLQKAAKLIGIPPSNFRSLTTSFPSGFSLSPEKLQSAIKDDIK
SGYVPLYVCATVGTTAAGAVDPILELGKVAQEYNLWFHIDAAYAGSACICTEFRHYLN
GVELADSISTNPHKWLLTNMECSCLWVKSPSSLVDSLSTKSEIMRNAATDSNQVIDYKD
WQIALSRRFRALKLWIVIRRHGLSGLTSHIHKDIKMAELFESLVAKDKRFEIVVPRKFAL
VCFRFKPEKENQDLSELNSKLLNAVNSSGCAFMTHAVLEGVYTIRCAIGTTLTEEHHVV
NLWKLIQEKAQSLIINEY
Sphagnum fallax 0042s0024.1 (SEQ ID NO: 92):
MSSKVAPWSRLSKPLDVEEFRTHAHRMVDFIADYHHNIQSFPVHSQLKPGYLRPLLPDT
APTEPEVVEDVFADVWNKILPGITHWQSPKFFGYYPFNVSTAGILGEILSGGVNVTGFSW
ITSPVVTELEIIVLDWLGKLLHLPEEFLSSGKGGGVIQGTSSEAVVCTSQHMSEAEALTKL
VVYTSDQAQSCVLRACQIAGIATANFRPLPTDASSHFSLSPAVLIKAAATDVAAGLFPFF
LCGKVGTTSSSAVDPLLELGDIAKRYGMWYHIDAAYAGSACICPEFRHYLNGVEKADS
YNMNPHDWMLTNFDCSTLWVKNSELLVAALSNKPVYLQNEATDNNLVDCSHIRNHISI
AKHFESLVRADFRFEMIVPTNFSLVCFRLRTPAGSKDNSRTLNSKLVEALNRKGDILVTH
TELSGRYTLRFAVGGTHMELHHVQAAWNLRLQRQVF
Eucalyptus grandis E01788.1 (SEQ ID NO: 93):
MNPLDPGEFRRQGHMVVDFLAKYYENIEKYPVLSQVEPGYLSKRLPSSAPQDEEPMEAI
LDDVHQHIFPGLTHWQSPNFFAYYQTNTSTAAILGEMLCAGFNVAGFNWVSSPAATELE
SLVMDWLGKMLDLPRPFLPFGNGGGVIEGNTSEAIICTLTAARDRVLRKLGHNSIAKLV
VYGSDQTNCSFQKAARVVGIDPRNFRALKMTRSTLFGLSPDSLEKAIRLDINAGLIPLYL
CATVGTTSCAAVDPLEPLCKVASKFSMWIHVDAAYAGASCICPEYRKFINGVEFADSFS
FNAHKWLLTPLDCCCLWVKDPNALVKSLSTDPEYLKNEATESKQVIDYADWQLSLSRR
FRALKLWLVLRSHGVQNLRSHIKNHCRLAKLFEELVEEDPQFEVVFPRNFALVCFRIHPS
GVAGMLNAQLLHAINASGRVFMSHTTVGGVYVLRFAVGATLVTEKHVIMAWKVVQE
HANSLLSMPASEQHSA
pHis8-4 (SEQ ID NO: 94):
TGGCGAATGGGACGCGCCCTGTAGCGGCGCATTAAGCGCGGCGGGTGTGGTGGTTA
CGCGCAGCGTGACCGCTACACTTGCCAGCGCCCTAGCGCCCGCTCCTTTCGCTTTCTT
CCCTTCCTTTCTCGCCACGTTCGCCGGCTTTCCCCGTCAAGCTCTAAATCGGGGGCTC
CCTTTAGGGTTCCGATTTAGTGCTTTACGGCACCTCGACCCCAAAAAACTTGATTAG
GGTGATGGTTCACGTAGTGGGCCATCGCCCTGATAGACGGTTTTTCGCCCTTTGACG
TTGGAGTCCACGTTCTTTAATAGTGGACTCTTGTTCCAAACTGGAACAACACTCAAC
CCTATCTCGGTCTATTCTTTTGATTTATAAGGGATTTTGCCGATTTCGGCCTATTGGTT
AAAAAATGAGCTGATTTAACAAAAATTTAACGCGAATTTTAACAAAATATTAACGTT
TACAATTTCAGGTGGCACTTTTCGGGGAAATGTGCGCGGAACCCCTATTTGTTTATTT
TTCTAAATACATTCAAATATGTATCCGCTCATGAATTAATTCTTAGAAAAACTCATC
GAGCATCAAATGAAACTGCAATTTATTCATATCAGGATTATCAATACCATATTTTTG
AAAAAGCCGTTTCTGTAATGAAGGAGAAAACTCACCGAGGCAGTTCCATAGGATGG
CAAGATCCTGGTATCGGTCTGCGATTCCGACTCGTCCAACATCAATACAACCTATTA
ATTTCCCCTCGTCAAAAATAAGGTTATCAAGTGAGAAATCACCATGAGTGACGACTG
AATCCGGTGAGAATGGCAAAAGTTTATGCATTTCTTTCCAGACTTGTTCAACAGGCC
AGCCATTACGCTCGTCATCAAAATCACTCGCATCAACCAAACCGTTATTCATTCGTG
ATTGCGCCTGAGCGAGACGAAATACGCGATCGCTGTTAAAAGGACAATTACAAACA
GGAATCGAATGCAACCGGCGCAGGAACACTGCCAGCGCATCAACAATATTTTCACC
TGAATCAGGATATTCTTCTAATACCTGGAATGCTGTTTTCCCGGGGATCGCAGTGGT
GAGTAACCATGCATCATCAGGAGTACGGATAAAATGCTTGATGGTCGGAAGAGGCA
TAAATTCCGTCAGCCAGTTTAGTCTGACCATCTCATCTGTAACATCATTGGCAACGCT
ACCTTTGCCATGTTTCAGAAACAACTCTGGCGCATCGGGCTTCCCATACAATCGATA
GATTGTCGCACCTGATTGCCCGACATTATCGCGAGCCCATTTATACCCATATAAATC
AGCATCCATGTTGGAATTTAATCGCGGCCTAGAGCAAGACGTTTCCCGTTGAATATG
GCTCATAACACCCCTTGTATTACTGTTTATGTAAGCAGACAGTTTTATTGTTCATGAC
CAAAATCCCTTAACGTGAGTTTTCGTTCCACTGAGCGTCAGACCCCGTAGAAAAGAT
CAAAGGATCTTCTTGAGATCCTTTTTTTCTGCGCGTAATCTGCTGCTTGCAAACAAAA
AAACCACCGCTACCAGCGGTGGTTTGTTTGCCGGATCAAGAGCTACCAACTCTTTTT
CCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTG
CTAATCCTGTTACCAGTGGCTGCTGCCAGTGGCGATAAGTCGTGTCTTACCGGGTTG
GACTCAAGACGATAGTTACCGGATAAGGCGCAGCGGTCGGGCTGAACGGGGGGTTC
GTGCACACAGCCCAGCTTGGAGCGAACGACCTACACCGAACTGAGATACCTACAGC
GTGAGCTATGAGAAAGCGCCACGCTTCCCGAAGGGAGAAAGGCGGACAGGTATCCG
GTAAGCGGCAGGGTCGGAACAGGAGAGCGCACGAGGGAGCTTCCAGGGGGAAACG
CCTGGTATCTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGGCCTTTT
TACGGTTCCTGGCCTTTTGCTGGCCTTTTGCTCACATGTTCTTTCCTGCGTTATCCCCT
GATTCTGTGGATAACCGTATTACCGCCTTTGAGTGAGCTGATACCGCTCGCCGCAGC
CGAACGACCGAGCGCAGCGAGTCAGTGAGCGAGGAAGCGGAAGAGCGCCTGATGC
GGTATTTTCTCCTTACGCATCTGTGCGGTATTTCACACCGCATATATGGTGCACTCTC
AGTACAATCTGCTCTGATGCCGCATAGTTAAGCCAGTATACACTCCGCTATCGCTAC
GTGACTGGGTCATGGCTGCGCCCCGACACCCGCCAACACCCGCTGACGCGCCCTGA
CGGGCTTGTCTGCTCCCGGCATCCGCTTACAGACAAGCTGTGACCGTCTCCGGGAGC
TGCATGTGTCAGAGGTTTTCACCGTCATCACCGAAACGCGCGAGGCAGCTGCGGTAA
AGCTCATCAGCGTGGTCGTGAAGCGATTCACAGATGTCTGCCTGTTCATCCGCGTCC
AGCTCGTTGAGTTTCTCCAGAAGCGTTAATGTCTGGCTTCTGATAAAGCGGGCCATG
TTAAGGGCGGTTTTTTCCTGTTTGGTCACTGATGCCTCCGTGTAAGGGGGATTTCTGT
TCATGGGGGTAATGATACCGATGAAACGAGAGAGGATGCTCACGATACGGGTTACT
GATGATGAACATGCCCGGTTACTGGAACGTTGTGAGGGTAAACAACTGGCGGTATG
GATGCGGCGGGACCAGAGAAAAATCACTCAGGGTCAATGCCAGCGCTTCGTTAATA
CAGATGTAGGTGTTCCACAGGGTAGCCAGCAGCATCCTGCGATGCAGATCCGGAAC
ATAATGGTGCAGGGCGCTGACTTCCGCGTTTCCAGACTTTACGAAACACGGAAACCG
AAGACCATTCATGTTGTTGCTCAGGTCGCAGACGTTTTGCAGCAGCAGTCGCTTCAC
GTTCGCTCGCGTATCGGTGATTCATTCTGCTAACCAGTAAGGCAACCCCGCCAGCCT
AGCCGGGTCCTCAACGACAGGAGCACGATCATGCGCACCCGTGGGGCCGCCATGCC
GGCGATAATGGCCTGCTTCTCGCCGAAACGTTTGGTGGCGGGACCAGTGACGAAGG
CTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTG
TCCTACGAGTTGCATGATAAAGAAGACAGTCATAAGTGCGGCGACGATAGTCATGC
CCCGCGCCCACCGGAAGGAGCTGACTGGGTTGAAGGCTCTCAAGGGCATCGGTCGA
GATCCCGGTGCCTAATGAGTGAGCTAACTTACATTAATTGCGTTGCGCTCACTGCCC
GCTTTCCAGTCGGGAAACCTGTCGTGCCAGCTGCATTAATGAATCGGCCAACGCGCG
GGGAGAGGCGGTTTGCGTATTGGGCGCCAGGGTGGTTTTTCTTTTCACCAGTGAGAC
GGGCAACAGCTGATTGCCCTTCACCGCCTGGCCCTGAGAGAGTTGCAGCAAGCGGT
CCACGCTGGTTTGCCCCAGCAGGCGAAAATCCTGTTTGATGGTGGTTAACGGCGGGA
TATAACATGAGCTGTCTTCGGTATCGTCGTATCCCACTACCGAGATATCCGCACCAA
CGCGCAGCCCGGACTCGGTAATGGCGCGCATTGCGCCCAGCGCCATCTGATCGTTGG
CAACCAGCATCGCAGTGGGAACGATGCCCTCATTCAGCATTTGCATGGTTTGTTGAA
AACCGGACATGGCACTCCAGTCGCCTTCCCGTTCCGCTATCGGCTGAATTTGATTGC
GAGTGAGATATTTATGCCAGCCAGCCAGACGCAGACGCGCCGAGACAGAACTTAAT
GGGCCCGCTAACAGCGCGATTTGCTGGTGACCCAATGCGACCAGATGCTCCACGCC
CAGTCGCGTACCGTCTTCATGGGAGAAAATAATACTGTTGATGGGTGTCTGGTCAGA
GACATCAAGAAATAACGCCGGAACATTAGTGCAGGCAGCTTCCACAGCAATGGCAT
CCTGGTCATCCAGCGGATAGTTAATGATCAGCCCACTGACGCGTTGCGCGAGAAGAT
TGTGCACCGCCGCTTTACAGGCTTCGACGCCGCTTCGTTCTACCATCGACACCACCA
CGCTGGCACCCAGTTGATCGGCGCGAGATTTAATCGCCGCGACAATTTGCGACGGC
GCGTGCAGGGCCAGACTGGAGGTGGCAACGCCAATCAGCAACGACTGTTTGCCCGC
CAGTTGTTGTGCCACGCGGTTGGGAATGTAATTCAGCTCCGCCATCGCCGCTTCCAC
TTTTTCCCGCGTTTTCGCAGAAACGTGGCTGGCCTGGTTCACCACGCGGGAAACGGT
CTGATAAGAGACACCGGCATACTCTGCGACATCGTATAACGTTACTGGTTTCACATT
CACCACCCTGAATTGACTCTCTTCCGGGCGCTATCATGCCATACCGCGAAAGGTTTT
GCGCCATTCGATGGTGTCCGGGATCTCGACGCTCTCCCTTATGCGACTCCTGCATTA
GGAAGCAGCCCAGTAGTAGGTTGAGGCCGTTGAGCACCGCCGCCGCAAGGAATGGT
GCATGCAAGGAGATGGCGCCCAACAGTCCCCCGGCCACGGGGCCTGCCACCATACC
CACGCCGAAACAAGCGCTCATGAGCCCGAAGTGGCGAGCCCGATCTTCCCCATCGG
TGATGTCGGCGATATAGGCGCCAGCAACCGCACCTGTGGCGCCGGTGATGCCGGCC
ACGATGCGTCCGGCGTAGAGGATCGAGATCTCGATCCCGCGAAATTAATACGACTC
ACTATAGGGGAATTGTGAGCGGATAACAATTCCCCTCTAGAAATAATTTTGTTTAAC
TTTAAGAAGGAGATATACCATGAAACACCACCACCACCACCACCACCACGGTGGTG
AAAACTTGTACTTCCAGGCCCATGGCGGATCCGAATTCGAGCTCCGTCGACAAGCTT
GCGGCCGCACTCGAGCACCACCACCACCACCACTGAGATCCGGCTGCTAACAAAGC
CCGAAAGGAAGCTGAGTTGGCTGCTGCCACCGCTGAGCAATAACTAGCATAACCCC
TTGGGGCCTCTAAACGGGTCTTGAGGGGTTTTTTGCTGAAAGGAGGAACTATATCCG
GAT
pEAQ-HT (SEQ ID NO: 95):
CCTGTGGTTGGCATGCACATACAAATGGACGAACGGATAAACCTTTTCACGCCCTTT
TAAATATCCGATTATTCTAATAAACGCTCTTTTCTCTTAGGTTTACCCGCCAATATAT
CCTGTCAAACACTGATAGTTTGTGAACCATCACCCAAATCAAGTTTTTTGGGGTCGA
GGTGCCGTAAAGCACTAAATCGGAACCCTAAAGGGAGCCCCCGATTTAGAGCTTGA
CGGGGAAAGCCGGCGAACGTGGCGAGAAAGGAAGGGAAGAAAGCGAAAGGAGCG
GGCGCCATTCAGGCTGCGCAACTGTTGGGAAGGGCGATCGGTGCGGGCCTCTTCGCT
ATTACGCCAGCTGGCGAAAGGGGGATGTGCTGCAAGGCGATTAAGTTGGGTAACGC
CAGGGTTTTCCCAGTCACGACGTTGTAAAACGACGGCCAGTGAATTGTTAATTAAGA
ATTCGAGCTCCACCGCGGAAACCTCCTCGGATTCCATTGCCCAGCTATCTGTCACTTT
ATTGAGAAGATAGTGGAAAAGGAAGGTGGCTCCTACAAATGCCATCATTGCGATAA
AGGAAAGGCCATCGTTGAAGATGCCTCTGCCGACAGTGGTCCCAAAGATGGACCCC
CACCCACGAGGAGCATCGTGGAAAAAGAAGACGTTCCAACCACGTCTTCAAAGCAA
GTGGATTGATGTGATATCTCCACTGACGTAAGGGATGACGCACAATCCCACTATCCT
TCGCAAGACCCTTCCTCTATATAAGGAAGTTCATTTCATTTGGAGAGGTATTAAAAT
CTTAATAGGTTTTGATAAAAGCGAACGTGGGGAAACCCGAACCAAACCTTCTTCTAA
ACTCTCTCTCATCTCTCTTAAAGCAAACTTCTCTCTTGTCTTTCTTGCGTGAGCGATCT
TCAACGTTGTCAGATCGTGCTTCGGCACCAGTACAACGTTTTCTTTCACTGAAGCGA
AATCAAAGATCTCTTTGTGGACACGTAGTGCGGCGCCATTAAATAACGTGTACTTGT
CCTATTCTTGTCGGTGTGGTCTTGGGAAAAGAAAGCTTGCTGGAGGCTGCTGTTCAG
CCCCATACATTACTTGTTACGATTCTGCTGACTTTCGGCGGGTGCAATATCTCTACTT
CTGCTTGACGAGGTATTGTTGCCTGTACTTCTTTCTTCTTCTTCTTGCTGATTGGTTCT
ATAAGAAATCTAGTATTTTCTTTGAAACAGAGTTTTCCCGTGGTTTTCGAACTTGGAG
AAAGATTGTTAAGCTTCTGTATATTCTGCCCAAATTCGCGACCGGTATGCATCACCA
TCACCATCATCCCGGGCATCACCATCACCATCACTAGCTCGAGGCCTTTAACTCTGG
TTTCATTAAATTTTCTTTAGTTTGAATTTACTGTTATTCGGTGTGCATTTCTATGTTTG
GTGAGCGGTTTTCTGTGCTCAGAGTGTGTTTATTTTATGTAATTTAATTTCTTTGTGA
GCTCCTGTTTAGCAGGTCGTCCCTTCAGCAAGGACACAAAAAGATTTTAATTTTATT
AAAAAAAAAAAAAAAAAAGACCGGGAATTCGATATCAAGCTTATCGACCTGCAGAT
CGTTCAAACATTTGGCAATAAAGTTTCTTAAGATTGAATCCTGTTGCCGGTCTTGCG
ATGATTATCATATAATTTCTGTTGAATTACGTTAAGCATGTAATAATTAACATGTAAT
GCATGACGTTATTTATGAGATGGGTTTTTATGATTAGAGTCCCGCAATTATACATTTA
ATACGCGATAGAAAACAAAATATAGCGCGCAAACTAGGATAAATTATCGCGCGCGG
TGTCATCTATGTTACTAGATCTCTAGAGTCTCAAGCTTGGCGCGCCAGCTTGGCGTA
ATCATGGTCATAGCTGTTGCGATTAAGAATTCGAGCTCGGTACCCCCCTACTCCAAA
AATGTCAAAGATACAGTCTCAGAAGACCAAAGGGCTATTGAGACTTTTCAACAAAG
GGTAATTTCGGGAAACCTCCTCGGATTCCATTGCCCAGCTATCTGTCACTTCATCGA
AAGGACAGTAGAAAAGGAAGGTGGCTCCTACAAATGCCATCATTGCGATAAAGGAA
AGGCTATCATTCAAGATGCCTCTGCCGACAGTGGTCCCAAAGATGGACCCCCACCCA
CGAGGAGCATCGTGGAAAAAGAAGACGTTCCAACCACGTCTTCAAAGCAAGTGGAT
TGATGTGACATCTCCACTGACGTAAGGGATGACGCACAATCCCACTATCCTTCGCAA
GACCCTTCCTCTATATAAGGAAGTTCATTTCATTTGGAGAGGACAGCCCAAGCTTCG
ACTCTAGAGGATCCCCTTAAATCGATATGGAACGAGCTATACAAGGAAACGACGCT
AGGGAACAAGCTAACAGTGAACGTTGGGATGGAGGATCAGGAGGTACCACTTCTCC
CTTCAAACTTCCTGACGAAAGTCCGAGTTGGACTGAGTGGCGGCTACATAACGATGA
GACGAATTCGAATCAAGATAATCCCCTTGGTTTCAAGGAAAGCTGGGGTTTCGGGA
AAGTTGTATTTAAGAGATATCTCAGATACGACAGGACGGAAGCTTCACTGCACAGA
GTCCTTGGATCTTGGACGGGAGATTCGGTTAACTATGCAGCATCTCGATTTTTCGGTT
TCGACCAGATCGGATGTACCTATAGTATTCGGTTTCGAGGAGTTAGTATCACCGTTT
CTGGAGGGTCTCGAACTCTTCAGCATCTCTGTGAGATGGCAATTCGGTCTAAGCAAG
AACTGCTACAGCTTGCCCCAATCGAAGTGGAAAGTAATGTATCAAGAGGATGCCCT
GAAGGTACTGAGACCTTCGAAAAAGAAAGCGAGTAAGGGGAGCTCGAATTCGCTGA
AATCACCAGTCTCTCTCTACAAATCTATCTCTCTCTATTTTCTCCATAAATAATGTGT
GAGTAGTTTCCCGATAAGGGAAATTAGGGTTCTTATAGGGTTTCGCTCATGTGTTGA
GCATATAAGAAACCCTTAGTATGTATTTGTATTTGTAAAATACTTCTATCAATAAAA
TTTCTAATTCCTAAAACCAAAATCCAGTACTAAAATCCAGATCTCCTAAAGTCCCTA
TAGATCTTTGTCGTGAATATAAACCAGACACGAGACGACTAAACCTGGAGCCCAGA
CGCCGTTCGAAGCTAGAAGTACCGCTTAGGCAGGAGGCCGTTAGGGAAAAGATGCT
AAGGCAGGGTTGGTTACGTTGACTCCCCCGTAGGTTTGGTTTAAATATGATGAAGTG
GACGGAAGGAAGGAGGAAGACAAGGAAGGATAAGGTTGCAGGCCCTGTGCAAGGT
AAGAAGATGGAAATTTGATAGAGGTACGCTACTATACTTATACTATACGCTAAGGG
AATGCTTGTATTTATACCCTATACCCCCTAATAACCCCTTATCAATTTAAGAAATAAT
CCGCATAAGCCCCCGCTTAAAAATTGGTATCAGAGCCATGAATAGGTCTATGACCAA
AACTCAAGAGGATAAAACCTCACCAAAATACGAAAGAGTTCTTAACTCTAAAGATA
AAAGATGGCGCGTGGCCGGCCTACAGTATGAGCGGAGAATTAAGGGAGTCACGTTA
TGACCCCCGCCGATGACGCGGGACAAGCCGTTTTACGTTTGGAACTGACAGAACCG
CAACGTTGAAGGAGCCACTCAGCCGCGGGTTTCTGGAGTTTAATGAGCTAAGCACAT
ACGTCAGAAACCATTATTGCGCGTTCAAAAGTCGCCTAAGGTCACTATCAGCTAGCA
AATATTTCTTGTCAAAAATGCTCCACTGACGTTCCATAAATTCCCCTCGGTATCCAAT
TAGAGTCTCATATTCACTCTCAATCCAAATAATCTGCACCGGATCTGGATCGTTTCGC
ATGATTGAACAAGATGGATTGCACGCAGGTTCTCCGGCCGCTTGGGTGGAGAGGCT
ATTCGGCTATGACTGGGCACAACAGACAATCGGCTGCTCTGATGCCGCCGTGTTCCG
GCTGTCAGCGCAGGGGCGCCCGGTTCTTTTTGTCAAGACCGACCTGTCCGGTGCCCT
GAATGAACTGCAGGACGAGGCAGCGCGGCTATCGTGGCTGGCCACGACGGGCGTTC
CTTGCGCAGCTGTGCTCGACGTTGTCACTGAAGCGGGAAGGGACTGGCTGCTATTGG
GCGAAGTGCCGGGGCAGGATCTCCTGTCATCTCACCTTGCTCCTGCCGAGAAAGTAT
CCATCATGGCTGATGCAATGCGGCGGCTGCATACGCTTGATCCGGCTACCTGCCCAT
TCGACCACCAAGCGAAACATCGCATCGAGCGAGCACGTACTCGGATGGAAGCCGGT
CTTGTCGATCAGGATGATCTGGACGAAGAGCATCAGGGGCTCGCGCCAGCCGAACT
GTTCGCCAGGCTCAAGGCGCGCATGCCCGACGGCGATGATCTCGTCGTGACCCATG
GCGATGCCTGCTTGCCGAATATCATGGTGGAAAATGGCCGCTTTTCTGGATTCATCG
ACTGTGGCCGGCTGGGTGTGGCGGACCGCTATCAGGACATAGCGTTGGCTACCCGTG
ATATTGCTGAAGAGCTTGGCGGCGAATGGGCTGACCGCTTCCTCGTGCTTTACGGTA
TCGCCGCTCCCGATTCGCAGCGCATCGCCTTCTATCGCCTTCTTGACGAGTTCTTCTG
AGCGGGACTCTGGGGTTCGAAATGACCGACCAAGCGACGCCCAACCTGCCATCACG
AGATTTCGATTCCACCGCCGCCTTCTATGAAAGGTTGGGCTTCGGAATCGTTTTCCG
GGACGCCGGCTGGATGATCCTCCAGCGCGGGGATCTCATGCTGGAGTTCTTCGCCCA
CGGGATCTCTGCGGAACAGGCGGTCGAAGGTGCCGATATCATTACGACAGCAACGG
CCGACAAGCACAACGCCACGATCCTGAGCGACAATATGATCGCGGCGTCCACATCA
ACGGCGTCGGCGGCGACTGCCCAGGCAAGACCGAGATGCACCGCGATATCTTGCTG
CGTTCGGATATTTTCGTGGAGTTCCCGCCACAGACCCGGATGATCCCCGATCGTTCA
AACATTTGGCAATAAAGTTTCTTAAGATTGAATCCTGTTGCCGGTCTTGCGATGATT
ATCATATAATTTCTGTTGAATTACGTTAAGCATGTAATAATTAACATGTAATGCATG
ACGTTATTTATGAGATGGGTTTTTATGATTAGAGTCCCGCAATTATACATTTAATACG
CGATAGAAAACAAAATATAGCGCGCAAACTAGGATAAATTATCGCGCGCGGTGTCA
TCTATGTTACTAGATCGGGACTGTAGGCCGGCCCTCACTGGTGAAAAGAAAAACCA
CCCCAGTACATTAAAAACGTCCGCAATGTGTTATTAAGTTGTCTAAGCGTCAATTTG
TTTACACCACAATATATCCTGCCACCAGCCAGCCAACAGCTCCCCGACCGGCAGCTC
GGCACAAAATCACCACTCGATACAGGCAGCCCATCAGTCCGGGACGGCGTCAGCGG
GAGAGCCGTTGTAAGGCGGCAGACTTTGCTCATGTTACCGATGCTATTCGGAAGAAC
GGCAACTAAGCTGCCGGGTTTGAAACACGGATGATCTCGCGGAGGGTAGCATGTTG
ATTGTAACGATGACAGAGCGTTGCTGCCTGTGATCAAATATCATCTCCCTCGCAGAG
ATCCGAATTATCAGCCTTCTTATTCATTTCTCGCTTAACCGTGACAGAGTAGACAGG
CTGTCTCGCGGCCGAGGGGCGCAGCCCCTGGGGGGGATGGGAGGCCCGCGTTAGCG
GGCCGGGAGGGTTCGAGAAGGGGGGGCACCCCCCTTCGGCGTGCGCGGTCACGCGC
ACAGGGCGCAGCCCTGGTTAAAAACAAGGTTTATAAATATTGGTTTAAAAGCAGGT
TAAAAGACAGGTTAGCGGTGGCCGAAAAACGGGCGGAAACCCTTGCAAATGCTGGA
TTTTCTGCCTGTGGACAGCCCCTCAAATGTCAATAGGTGCGCCCCTCATCTGTCAGC
ACTCTGCCCCTCAAGTGTCAAGGATCGCGCCCCTCATCTGTCAGTAGTCGCGCCCCT
CAAGTGTCAATACCGCAGGGCACTTATCCCCAGGCTTGTCCACATCATCTGTGGGAA
ACTCGCGTAAAATCAGGCGTTTTCGCCGATTTGCGAGGCTGGCCAGCTCCACGTCGC
CGGCCGAAATCGAGCCTGCCCCTCATCTGTCAACGCCGCGCCGGGTGAGTCGGCCCC
TCAAGTGTCAACGTCCGCCCCTCATCTGTCAGTGAGGGCCAAGTTTTCCGCGAGGTA
TCCACAACGCCGGCGGCCGCGGTGTCTCGCACACGGCTTCGACGGCGTTTCTGGCGC
GTTTGCAGGGCCATAGACGGCCGCCAGCCCAGCGGCGAGGGCAACCAGCCCGGTGA
GCGTCGGAAAGGCGCTCGGTCTTGCCTTGCTCGTCGGTGATGTACACTAGTCGCTGG
CTGCTGAACCCCCAGCCGGAACTGACCCCACAAGGCCCTAGCGTTTGCAATGCACC
AGGTCATCATTGACCCAGGCGTGTTCCACCAGGCCGCTGCCTCGCAACTCTTCGCAG
GCTTCGCCGACCTGCTCGCGCCACTTCTTCACGCGGGTGGAATCCGATCCGCACATG
AGGCGGAAGGTTTCCAGCTTGAGCGGGTACGGCTCCCGGTGCGAGCTGAAATAGTC
GAACATCCGTCGGGCCGTCGGCGACAGCTTGCGGTACTTCTCCCATATGAATTTCGT
GTAGTGGTCGCCAGCAAACAGCACGACGATTTCCTCGTCGATCAGGACCTGGCAAC
GGGACGTTTTCTTGCCACGGTCCAGGACGCGGAAGCGGTGCAGCAGCGACACCGAT
TCCAGGTGCCCAACGCGGTCGGACGTGAAGCCCATCGCCGTCGCCTGTAGGCGCGA
CAGGCATTCCTCGGCCTTCGTGTAATACCGGCCATTGATCGACCAGCCCAGGTCCTG
GCAAAGCTCGTAGAACGTGAAGGTGATCGGCTCGCCGATAGGGGTGCGCTTCGCGT
ACTCCAACACCTGCTGCCACACCAGTTCGTCATCGTCGGCCCGCAGCTCGACGCCGG
TGTAGGTGATCTTCACGTCCTTGTTGACGTGGAAAATGACCTTGTTTTGCAGCGCCTC
GCGCGGGATTTTCTTGTTGCGCGTGGTGAACAGGGCAGAGCGGGCCGTGTCGTTTGG
CATCGCTCGCATCGTGTCCGGCCACGGCGCAATATCGAACAAGGAAAGCTGCATTTC
CTTGATCTGCTGCTTCGTGTGTTTCAGCAACGCGGCCTGCTTGGCCTCGCTGACCTGT
TTTGCCAGGTCCTCGCCGGCGGTTTTTCGCTTCTTGGTCGTCATAGTTCCTCGCGTGT
CGATGGTCATCGACTTCGCCAAACCTGCCGCCTCCTGTTCGAGACGACGCGAACGCT
CCACGGCGGCCGATGGCGCGGGCAGGGCAGGGGGAGCCAGTTGCACGCTGTCGCGC
TCGATCTTGGCCGTAGCTTGCTGGACCATCGAGCCGACGGACTGGAAGGTTTCGCGG
GGCGCACGCATGACGGTGCGGCTTGCGATGGTTTCGGCATCCTCGGCGGAAAACCC
CGCGTCGATCAGTTCTTGCCTGTATGCCTTCCGGTCAAACGTCCGATTCATTCACCCT
CCTTGCGGGATTGCCCCGACTCACGCCGGGGCAATGTGCCCTTATTCCTGATTTGAC
CCGCCTGGTGCCTTGGTGTCCAGATAATCCACCTTATCGGCAATGAAGTCGGTCCCG
TAGACCGTCTGGCCGTCCTTCTCGTACTTGGTATTCCGAATCTTGCCCTGCACGAATA
CCAGCGACCCCTTGCCCAAATACTTGCCGTGGGCCTCGGCCTGAGAGCCAAAACACT
TGATGCGGAAGAAGTCGGTGCGCTCCTGCTTGTCGCCGGCATCGTTGCGCCACATCT
AGGTACTAAAACAATTCATCCAGTAAAATATAATATTTTATTTTCTCCCAATCAGGC
TTGATCCCCAGTAAGTCAAAAAATAGCTCGACATACTGTTCTTCCCCGATATCCTCC
CTGATCGACCGGACGCAGAAGGCAATGTCATACCACTTGTCCGCCCTGCCGCTTCTC
CCAAGATCAATAAAGCCACTTACTTTGCCATCTTTCACAAAGATGTTGCTGTCTCCC
AGGTCGCCGTGGGAAAAGACAAGTTCCTCTTCGGGCTTTTCCGTCTTTAAAAAATCA
TACAGCTCGCGCGGATCTTTAAATGGAGTGTCTTCTTCCCAGTTTTCGCAATCCACAT
CGGCCAGATCGTTATTCAGTAAGTAATCCAATTCGGCTAAGCGGCTGTCTAAGCTAT
TCGTATAGGGACAATCCGATATGTCGATGGAGTGAAAGAGCCTGATGCACTCCGCA
TACAGCTCGATAATCTTTTCAGGGCTTTGTTCATCTTCATACTCTTCCGAGCAAAGGA
CGCCATCGGCCTCACTCATGAGCAGATTGCTCCAGCCATCATGCCGTTCAAAGTGCA
GGACCTTTGGAACAGGCAGCTTTCCTTCCAGCCATAGCATCATGTCCTTTTCCCGTTC
CACATCATAGGTGGTCCCTTTATACCGGCTGTCCGTCATTTTTAAATATAGGTTTTCA
TTTTCTCCCACCAGCTTATATACCTTAGCAGGAGACATTCCTTCCGTATCTTTTACGC
AGCGGTATTTTTCGATCAGTTTTTTCAATTCCGGTGATATTCTCATTTTAGCCATTTAT
TATTTCCTTCCTCTTTTCTACAGTATTTAAAGATACCCCAAGAAGCTAATTATAACAA
GACGAACTCCAATTCACTGTTCCTTGCATTCTAAAACCTTAAATACCAGAAAACAGC
TTTTTCAAAGTTGTTTTCAAAGTTGGCGTATAACATAGTATCGACGGAGCCGATTTTG
AAACCACAATTATGGGTGATGCTGCCAACTTACTGATTTAGTGTATGATGGTGTTTTT
GAGGTGCTCCAGTGGCTTCTGTTTCTATCAGCTGTCCCTCCTGTTCAGCTACTGACGG
GGTGGTGCGTAACGGCAAAAGCACCGCCGGACATCAGCGCTATCTCTGCTCTCACTG
CCGTAAAACATGGCAACTGCAGTTCACTTACACCGCTTCTCAACCCGGTACGCACCA
GAAAATCATTGATATGGCCATGAATGGCGTTGGATGCCGGGCAACAGCCCGCATTA
TGGGCGTTGGCCTCAACACGATTTTACGTCACTTAAAAAACTCAGGCCGCAGTCGGT
AACTATGCGGTGTGAAATACCGCACAGATGCGTAAGGAGAAAATACCGCATCAGGC
GCTCTTCCGCTTCCTCGCTCACTGACTCGCTGCGCTCGGTCGTTCGGCTGCGGCGAGC
GGTATCAGCTCACTCAAAGGCGGTAATACGGTTATCCACAGAATCAGGGGATAACG
CAGGAAAGAACATGTGAGCAAAAGGCCAGCAAAAGGCCAGGAACCGTAAAAAGGC
CGCGTTGCTGGCGTTTTTCCATAGGCTCCGCCCCCCTGACGAGCATCACAAAAATCG
ACGCTCAAGTCAGAGGTGGCGAAACCCGACAGGACTATAAAGATACCAGGCGTTTC
CCCCTGGAAGCTCCCTCGTGCGCTCTCCTGTTCCGACCCTGCCGCTTACCGGATACCT
GTCCGCCTTTCTCCCTTCGGGAAGCGTGGCGCTTTCTCATAGCTCACGCTGTAGGTAT
CTCAGTTCGGTGTAGGTCGTTCGCTCCAAGCTGGGCTGTGTGCACGAACCCCCCGTT
CAGCCCGACCGCTGCGCCTTATCCGGTAACTATCGTCTTGAGTCCAACCCGGTAAGA
CACGACTTATCGCCACTGGCAGCAGGTAACCTCGCGCATACAGCCGGGCAGTGACG
TCATCGTCTGCGCGGAAATGGACGGGCCCCCGGCGCCAGATCTGGGGAAC
pJKW 1410 (SEQ ID NO: 96):
CCCTGAATTCGCATCTAGACTGATGAGACGTGGTAGAGCCACAAACAGCCGGTACA
AGCAACGATCTCCAGGACCATCTGAATCATGCGCGGATGACACGAACTCACGACGG
CGATCACAGACATTAACCCACAGTACAGACACTGCGACAACGTGGCAATTCGTCGC
AATACAACGTGAGACCGAAAGTGAAACGTGATTTCATGCGTCATTTTGAACATTTTG
TAAATCTTATTTAATAATGTGTGCGGCAATTCACATTTAATTTATGAATGTTTTCTTA
ACATCGCGGCAACTCAAGAAACGGCAGGTTCGGATCTTAGCTACTAGAGAAAGAGG
AGAAATACTAGATGCGTAAAGGCGAAGAGCTGTTCACTGGTGTCGTCCCTATTCTGG
TGGAACTGGATGGTGATGTCAACGGTCATAAGTTTTCCGTGCGTGGCGAGGGTGAA
GGTGACGCAACTAATGGTAAACTGACGCTGAAGTTCATCTGTACTACTGGTAAACTG
CCGGTTCCTTGGCCGACTCTGGTAACGACGCTGACTTATGGTGTTCAGTGCTTTGCTC
GTTATCCGGACCATATGAAGCAGCATGACTTCTTCAAGTCCGCCATGCCGGAAGGCT
ATGTGCAGGAACGCACGATTTCCTTTAAGGATGACGGCACGTACAAAACGCGTGCG
GAAGTGAAATTTGAAGGCGATACCCTGGTAAACCGCATTGAGCTGAAAGGCATTGA
CTTTAAAGAGGACGGCAATATCCTGGGCCATAAGCTGGAATACAATTTTAACAGCC
ACAATGTTTACATCACCGCCGATAAACAAAAAAATGGCATTAAAGCGAATTTTAAA
ATTCGCCACAACGTGGAGGATGGCAGCGTGCAGCTGGCTGATCACTACCAGCAAAA
CACTCCAATCGGTGATGGTCCTGTTCTGCTGCCAGACAATCACTATCTGAGCACGCA
AAGCGTTCTGTCTAAAGATCCGAACGAGAAACGCGATCATATGGTTCTGCTGGAGTT
CGTAACCGCAGCGGGCATCACGCATGGTATGGATGAACTGTACAAATGACCAGGCA
TCAAATAAAACGAAAGGCTCAGTCGAAAGACTGGGCCTTTCGTTTTATCTGTTGTTT
GTCGGTGAACGCTCTCTACTAGAGTCACACTGGCTCACCTTCGGGTGGGCCTTTCTG
CGTTTATAGGTCTCAGCTGGAAATCTGCTCGTCAGTGGTGCTCACACTGACGAATCA
TGTACAGATCATACCGATGACTGCCTGGCGACTCACAACTAAGCAAGACAGCCGGA
ACCAGCGCCGGCGAACACCACTGCATATATGGCATATCACAACAGTCCACGTCTCA
AGCAGTTACAGAGATGTTACGAACCACTAGTGCACTGCAGTACAAACACAGTCCTTT
CCCGCAATTTTCTTTTTCTATTACTCTTGGCCTCCTCTAGTACACTCTATATTTTTTTA
TGCCTCGGTAATGATTTTCATTTTTTTTTTTCCACCTAGCGGATGACTCTTTTTTTTTC
TTAGCGATTGGCATTATCACATAATGAATTATACATTATATAAAGTAATGTGATTTCT
TCGAAGAATATACTAAAAAATGAGCAGGCAAGATAAACGAAGGCAAAGATGACAG
AGCAGAAAGCCCTAGTAAAGCGTATTACAAATGAAACCAAGATTCAGATTGCGATC
TCTTTAAAGGGTGGTCCCCTAGCGATAGAGCACTCGATCTTCCCAGAAAAAGAGGC
AGAAGCAGTAGCAGAACAGGCCACACAATCGCAAGTGATTAACGTCCACACAGGTA
TAGGGTTTCTGGACCATATGATACATGCTCTGGCCAAGCATTCCGGCTGGTCGCTAA
TCGTTGAGTGCATTGGTGACTTACACATAGACGACCATCACACCACTGAGGACTGCG
GGATTGCTCTCGGTCAAGCTTTTAAAGAGGCCCTAGGGGCCGTGCGTGGAGTAAAA
AGGTTTGGATCAGGATTTGCGCCTTTGGATGAGGCACTTTCCAGAGCGGTGGTTGAT
CTTTCGAACAGGCCGTACGCAGTTGTCGAACTTGGTTTGCAAAGGGAGAAAGTAGG
TGATCTCTCTTGCGAGATGATCCCGCATTTTCTTGAAAGCTTTGCAGAGGCTAGCAG
AATTACCCTCCACGTTGATTGTCTGCGAGGCAAGAATGATCATCACCGTAGTGAGAG
TGCGTTCAAGGCTCTTGCGGTTGCCATAAGAGAAGCCACCTCGCCCAATGGTACCAA
CGATGTTCCCTCCACCAAAGGTGTTCTTATGTAGTGACACCGATTATTTAAAGCTGCT
GCATACGATATATATACATGTGTATATATGTATACCTATGAATGTCAGTAAGTATGT
ATACGAACAGTATGATACTGAAGATGACAAGGTAATGCATCATTCTATACGTGTCAT
TCTGAACGAGGCGCGCTTTCCTTTTTTCTTTTTGCTTTTTCTTTTTTTTTCTCTTGAACT
CGACGGATCATAGAGTAACGAAGCATCTGTGCTTCATTTTGTAGAACAAAAATGCA
ACGCGAGAGCGCTAATTTTTCAAACAAAGAATCTGAGCTGCATTTTTACAGAACAGA
AATGCAACGCGAAAGCGCTATTTTACCAACGAAGAATCTGTGCTTCATTTTTGTAAA
ACAAAAATGCAACGCGAGAGCGCTAATTTTTCAAACAAAGAATCTGAGCTGCATTTT
TACAGAACAGAAATGCAACGCGAGAGCGCTATTTTACCAACAAAGAATCTATACTT
CTTTTTTGTTCTACAAAAATGCATCCCGAGAGCGCTATTTTTCTAACAAAGCATCTTA
GATTACTTTTTTTCTCCTTTGTGCGCTCTATAATGCAGTCTCTTGATAACTTTTTGCAC
TGTAGGTCCGTTAAGGTTAGAAGAAGGCTACTTTGGTGTCTATTTTCTCTTCCATAAA
AAAAGCCTGACTCCACTTCCCGCGTTTACTGATTACTAGCGAAGCTGCGGGTGCATT
TTTTCAAGATAAAGGCATCCCCGATTATATTCTATACCGATGTGGATTGCGCATACTT
TGTGAACAGAAAGTGATAGCGTTGATGATTCTTCATTGGTCAGAAAATTATGAACGG
TTTCTTCTATTTTGTCTCTATATACTACGTATAGGAAATGTTTACATTTTCGTATTGTT
TTCGATTCACTCTATGAATAGTTCTTACTACAATTTTTTTGTCTAAAGAGTAATACTA
GAGATAAACATAAAAAATGTAGAGGTCGAGTTTAGATGCAAGTTCAAGGAGCGAAA
GGTGGATGGGTAGGTTATATAGGGATATAGCACAGAGATATATAGCAAAGAGATAC
TTTTGAGCAATGTTTGTGGAAGCGGTATTCGCAATATTTTAGTAGCTCGTTACAGTCC
GGTGCGTTTTTGGTTTTTTGAAAGTGCGTCATCAGAGCGCTTTTGGTTTTCAAAAGCG
CTCTGAAGTTCCTATACTTTCTAGCTAGAGAATAGGAACTTCCCGAGCGGCCGCGTG
TTACAACCAATTAACCAATTCTGATTAGAAAAACTCATCGAGCATCAAATGAAACTG
CAATTTATTCATATCAGGATTATCAATACCATATTTTTGAAAAAGCCGTTTCTGTAAT
GAAGGAGAAAACTCACCGAGGCAGTTCCATAGGATGGCAAGATCCTGGTATCGGTC
TGCGATTCCGACTCGTCCAACATCAATACAACCTATTAATTTCCCCTCGTCAAAAAT
AAGGTTATCAAGTGAGAAATCACCATGAGTGACGACTGAATCCGGTGAGAATGGCA
AAAGCTTATGCATTTCTTTCCAGACTTGTTCAACAGGCCAGCCATTACGCTCGTCATC
AAAATCACTCGCATCAACCAAACCGTTATTCATTCGTGATTGCGCCTGAGCGAGGCG
AAATACGCGATCGCTGTTAAAAGGACAATTACAAACAGGAATCGAATGCAACCGGC
GCAGGAACACTGCCAGCGCATCAACAATATTTTCACCTGAATCAGGATATTCTTCTA
ATACCTGGAATGCTGTTTTCCCGGGGATCGCAGTGGTGAGTAACCATGCATCATCAG
GAGTACGGATAAAATGCTTGATGGTCGGAAGAGGCATAAATTCCGTCAGCCAGTTT
AGTCTGACCATCTCATCTGTAACATCATTGGCAACGCTACCTTTGCCATGTTTCAGAA
ACAACTCTGGCGCATCGGGCTTCCCATACAATCGATAGATTGTCGCACCTGATTGCC
CGACATTATCGCGAGCCCATTTATACCCATATAAATCAGCATCCATGTTGGAATTTA
ATCGCGGCCTGGAGCAAGACGTTTCCCGTTGAATATGGCTCATAACACCCCTTGTAT
TACTGTTTATGTAAGCAGACAGTTTTATTGTTCATGATGATATATTTTTATCTTGTGC
AATGTAACATCAGAGATTTTGAGACACAACGTGGCTTTGTTGAATAAATCGAACTTT
TGCTGAGTTGAAGGATCAGTCATGACCAAAATCCCTTAACGTGAGTTTTCGTTCCAC
TGAGCGTCAGACCCCGTAGAAAAGATCAAAGGATCTTCTTGAGATCCTTTTTTTCTG
CGCGTAATCTGCTGCTTGCAAACAAAAAAACCACCGCTACCAGCGGTGGTTTGTTTG
CCGGATCAAGAGCTACCAACTCTTTTTCCGAAGGTAACTGGCTTCAGCAGAGCGCAG
ATACCAAATACTGTTCTTCTAGTGTAGCCGTAGTTAGGCCACCACTTCAAGAACTCT
GTAGCACCGCCTACATACCTCGCTCTGCTAATCCTGTTACCAGTGGCTGCTGCCAGT
GGCGATAAGTCGTGTCTTACCGGGTTGGACTCAAGACGATAGTTACCGGATAAGGC
GCAGCGGTCGGGCTGAACGGGGGGTTCGTGCACACAGCCCAGCTTGGAGCGAACGA
CCTACACCGAACTGAGATACCTACAGCGTGAGCTATGAGAAAGCGCCACGCTTCCC
GAAGGGAGAAAGGCGGACAGGTATCCGGTAAGCGGCAGGGTCGGAACAGGAGAGC
GCACGAGGGAGCTTCCAGGGGGAAACGCCTGGTATCTTTATAGTCCTGTCGGGTTTC
GCCACCTCTGACTTGAGCGTCGATTTTTGTGATGCTCGTCAGGGGGGCGGAGCCTAT
GGAAAAACGCCAGCAACGCGGCCTTTTTACGGTTCCTGGCCTTTTGCTGGCCTTTTG
CTCACATGTTCTTTCCTGCGTTATCCCCTGATTCTGTGGATAACCGTGCGGCCGC

### REFERENCES

Altschul, S., Gish, W., Miller, W., Myers, E., and Lipman, D. (1990). Basic local alignment search tool. Journal of Molecular Biology. 215 (3): 403-4.
Anurag Priyam, B. J. W., Vivek Rai, Alekhya Munagala, Ismail Moghul, Filip Ter, Mark Anthony Gibbins, HongKee Moon, Guy Leonard, Wolfgang Rumpf, View ORCID ProfileYannick Wurm. (2015). Sequenceserver: a modern graphical user interface for custom BLAST databases. bioRxiv 033142.
Bai, Y. F., Bi, H. P., Zhuang, Y. B., Liu, C., Cai, T., Liu, X. N., Zhang, X. L., Liu, T., and Ma, Y. H. (2014). Production of salidroside in metabolically engineered Escherichia coli. Sci Rep-Uk 4.
Bolger, A. M., Lohse, M., and Usadel, B. (2014). Trimmomatic: a flexible trimmer for Illumina sequence data. Bioinformatics 30, 2114-2120.
Booker, A., Zhai, L., Gkouva, C., Li, S., and Heinrich, M. (2016). From Traditional Resource to Global Commodities:—A Comparison of Rhodiola Species Using NMR Spectroscopy-Metabolomics and HPTLC. Frontiers in pharmacology 7, 254.
Burkhard, P., Dominici, P., Borri-Voltattorni, C., Jansonius, J. N., and Malashkevich, V. N. (2001). Structural insight into Parkinson's disease treatment from drug-inhibited DOPA decarboxylase. Nature structural biology 8, 963-967.
Chambers, M. C., Maclean, B., Burke, R., Amodei, D., Ruderman, D. L., Neumann, S., Gatto, L., Fischer, B., Pratt, B., Egertson, J., et al. (2012). A cross-platform toolkit for mass spectrometry and proteomics. Nat Biotechnol 30, 918-920.
Chapple, C. C., Walker, M. A., and Ellis, B. E. (1986). Plant tyrosine decarboxylase can be strongly inhibited by L-alpha-aminooxy-beta-phenylpropionate. Planta 167, 101-105.
Chung, D., Kim, S. Y., and Ahn, J. H. (2017). Production of three phenylethanoids, tyrosol, hydroxytyrosol, and salidroside, using plant genes expressing in Escherichia coli. Sci Rep 7, 2578.
Cifani, C., Micioni Di, B. M., Vitale, G., Ruggieri, V., Ciccocioppo, R., and Massi, M. (2010). Effect of salidroside, active principle of Rhodiola rosea extract, on binge eating. Physiology & behavior 101, 555-562.
De Luca, V., Marineau, C., and Brisson, N. (1989). Molecular cloning and analysis of cDNA encoding a plant tryptophan decarboxylase: comparison with animal dopa decarboxylases. Proc Natl Acad Sci USA 86, 2582-2586.
Dorji, L. a. K. (2016). Ecological status of high altitude medicinal plants and their sustainability: Lingshi, Bhutan. BMC Ecology.
Du, M., and Xie, J. M. (1995). Flavonol Glycosides from Rhodiola-Crenulata. Phytochemistry 38, 809-810.
Ehrlich, F. (1907). Über die Bedingungen der Fuselölbildung und über ihren Zusammenhang mit dem Eiweissaufbau der Hefe. European journal of inorganic chemistry 40, 1027-1047.
Facchini, P. J., Huber-Allanach, K. L., and Tari, L. W. (2000). Plant aromatic L-amino acid decarboxylases: evolution, biochemistry, regulation, and metabolic engineering applications. Phytochemistry 54, 121-138.
Fan, B., Chen, T., Zhang, S., Wu, B., and He, B. (2017). Mining of efficient microbial UDP-glycosyltransferases by motif evolution cross plant kingdom for application in biosynthesis of salidroside. Scientific Reports 7:463, 1-9.
Fu, K. J., Ohba, H., Gilbert, M. G. (2009). Rhodiola. Flora China 8.
Gachon, C. M., Langlois-Meurinne, M. & Saindrenan, P. (2005) Plant secondary metabolism glycosyltransferases: the emerging functional analysis. Trends in plant science 10, 542-549.
Gantt, R. W., Peltier-Pain, P., and Thorson, J. S. (2011). Enzymatic methods for glyco(diversification/randomization) of drugs and small molecules. Natural product reports 28, 1811-1853.
Gauger, K. J., Rodriguez-Cortes, A., Hartwich, M., and Schneider, S. S. (2010). Rhodiola crenulata inhibits the tumorigenic properties of invasive mammary epithelial cells with stem cell characteristics. J Med Plants Res 4, 446-454.
Gold, N. D., Gowen, C. M., Lussier, F. X., Cautha, S. C., Mahadevan, R., and Martin, V. J. J. (2015). Metabolic engineering of a tyrosine-overproducing yeast platform using targeted metabolomics. Microb Cell Fact 14.
Gouet, P., Robert, X., and Courcelle, E. (2003). ESPript/ENDscript: Extracting and rendering sequence and 3D information from atomic structures of proteins. Nucleic acids research 31, 3320-3323.
Grabherr, M. G., Haas, B. J., Yassour, M., Levin, J. Z., Thompson, D. A., Amit, I., Adiconis, X., Fan, L., Raychowdhury, R., Zeng, Q. D., et al. (2011). Full-length transcriptome assembly from RNA-Seq data without a reference genome. Nat. Biotechnol. 29, 644-U130.
Guan, S., Xiong, Y., Song, B., Song, Y., Wang, D., Chu, X., Chen, N., Huo, M., Deng, X., and Lu, J. (2012). Protective effects of salidroside from Rhodiola rosea on LPS-induced acute lung injury in mice. Immunopharmacology and immunotoxicology 34, 667-672.
Gutensohn, M., Klempien, A., Kaminaga, Y., Nagegowda, D. A., Negre-Zakharov, F., Huh, J. H., Luo, H., Weizbauer, R., Mengiste, T., Tholl, D., et al. (2011). Role of aromatic aldehyde synthase in wounding/herbivory response and flower scent production in different Arabidopsis ecotypes. The Plant journal: for cell and molecular biology 66, 591-602.
Haas, B. J., Papanicolaou, A., Yassour, M., Grabherr, M., Blood, P. D., Bowden, J., Couger, M. B., Eccles, D., Li, B., Lieber, M., et al. (2013). De novo transcript sequence reconstruction from RNA-seq using the Trinity platform for reference generation and analysis. Nat Protoc 8, 1494-1512.
Hagel, J. M., and Facchini, P. J. (2013). Benzylisoquinoline alkaloid metabolism: a century of discovery and a brave new world. Plant & cell physiology 54, 647-672.
Han, Q., Ding, H., Robinson, H., Christensen, B. M., and Li, J. (2010). Crystal structure and substrate specificity of Drosophila 3,4-dihydroxyphenylalanine decarboxylase. PloS one 5, e8826.
Jones, P., and Vogt, T. (2001). Glycosyltransferases in secondary plant metabolism: tranquilizers and stimulant controllers. Planta 213, 164-174.
Kaminaga, Y., Schnepp, J., Peel, G., Kish, C. M., Ben-Nissan, G., Weiss, D., Orlova, I., Lavie, O., Rhodes, D., Wood, K., et al. (2006). Plant phenylacetaldehyde synthase is a bifunctional homotetrameric enzyme that catalyzes phenylalanine decarboxylation and oxidation. The Journal of biological chemistry 281, 23357-23366.
Kawalleck, P., Keller, H., Hahlbrock, K., Scheel, D., and Somssich, I. E. (1993). A pathogen-responsive gene of parsley encodes tyrosine decarboxylase. J. Biol. Chem. 268, 2189-2194.
Khanum, F., Bawa, A. S., and Singh, B. (2005). Rhodiola rosea: A versatile adaptogen. Compr Rev Food Sci F 4, 55-62.
Kumar, S., Stecher, G., and Tamura, K. (2016). MEGA7: Molecular Evolutionary Genetics Analysis Version 7.0 for Bigger Datasets. Mol Biol Evol 33, 1870-1874.
L, Z. E. a. P. (1965). Evolutionary divergence and convergence in proteins. Evolving Genes and Proteins.
Lan, X., Chang, K., Zeng, L., Liu, X., Qiu, F., Zheng, W., Quan, H., Liao, Z., Chen, M., Huang, W., et al. (2013). Engineering salidroside biosynthetic pathway in hairy root cultures of Rhodiola crenulata based on metabolic characterization of tyrosine decarboxylase. PloS one 8, e75459.
Landtag, J., Baumert, A., Degenkolb, T., Schmidt, J., Wray, V., Scheel, D., Strack, D., and Rosahl, S. (2002). Accumulation of tyrosol glucoside in transgenic potato plants expressing a parsley tyrosine decarboxylase. Phytochemistry 60, 683-689.
Lee, M. E., DeLoache, W. C., Cervantes, B., and Dueber, J. E. (2015). A Highly Characterized Yeast Toolkit for Modular, Multipart Assembly. ACS synthetic biology 4, 975-986.
Lei, Y. D., Gao, H., Tsering, T., Shi, S. H., and Zhong, Y. (2006). Determination of genetic variation in Rhodiola crenulata from the Hengduan Mountains Region, China using inter-simple sequence repeats. Genet Mol Biol 29, 339-344.
Li, B., and Dewey, C. N. (2011). RSEM: accurate transcript quantification from RNA-Seq data with or without a reference genome. Bmc Bioinformatics 12.
Li, B., Ruotti, V., Stewart, R. M., Thomson, J. A., and Dewey, C. N. (2010). RNA-Seq gene expression estimation with read mapping uncertainty. Bioinformatics 26, 493-500.
Li, Y., Baldauf, S., Lim, E. K., and Bowles, D. J. (2001). Phylogenetic analysis of the UDP-glycosyltransferase multigene family of Arabidopsis thaliana. J. Biol. Chem. 276, 4338-4343.
Ma, L. Q., Liu, B. Y., Gao, D. Y., Pang, X. B., Lu, S. Y., Yu, H. S., Wang, H., Yan, F., Li, Z. Q., Li, Y. F., et al. (2007). Molecular cloning and overexpression of a novel UDP-glucosyltransferase elevating salidroside levels in Rhodiola sachalinensis. Plant cell reports 26, 989-999.
Mumberg, D., Muller, R., and Funk, M. (1995). Yeast Vectors for the Controlled Expression of Heterologous Proteins in Different Genetic Backgrounds. Gene 156, 119-122.
Nelissen, H., Clarke, J. H., De Block, M., De Block, S., Vanderhaeghen, R., Zielinski, R. E., Dyer, T., Lusta, S., Inze, D., and Van Lijsebettens, M. (2003). DRL1, a homolog of the yeast TOT4/KT112 protein, has a function in meristem activity and organ growth in plants. The Plant cell 15, 639-654.
O'Connor, S. E. (2015). Engineering of Secondary Metabolism. Annual review of genetics 49, 71-94.
Panossian, A., Hamm, R., Wikman, G., and Efferth, T. (2014). Mechanism of action of Rhodiola, salidroside, tyrosol and triandrin in isolated neuroglial cells: an interactive pathway analysis of the downstream effects using RNA microarray data. Phytomedicine: international journal of phytotherapy and phytopharmacology 21, 1325-1348.
Peyret, H., and Lomonossoff, G. P. (2013). The pEAQ vector series: the easy and quick way to produce recombinant proteins in plants. Plant Mol. Biol. 83, 51-58.
Pluskal, T., Castillo, S., Villar-Briones, A., and Oresic, M. (2010). MZmine 2: Modular framework for processing, visualizing, and analyzing mass spectrometry-based molecular profile data. Bmc Bioinformatics 11.
Rohloff, J. (2002). Volatiles from rhizomes of Rhodiola rosea L. Phytochemistry 59, 655-661.
Ross, J., Li, Y., Lim, E., and Bowles, D. J. (2001). Higher plant glycosyltransferases. Genome Biol 2, REVIEWS3004.
Sainsbury, F., Thuenemann, E. C., and Lomonossoff, G. P. (2009). pEAQ: versatile expression vectors for easy and quick transient expression of heterologous proteins in plants. Plant Biotechnol J 7, 682-693.
Samanani, N., Liscombe, D. K., and Facchini, P. J. (2004). Molecular cloning and characterization of norcoclaurine synthase, an enzyme catalyzing the first committed step in benzylisoquinoline alkaloid biosynthesis. The Plant journal: for cell and molecular biology 40, 302-313.
Sanderson, M. J., and Wojciechowski, M. F. (2000). Improved bootstrap confidence limits in large-scale phylogenies, with an example from Neo-Astragalus (Leguminosae). Systematic biology 49, 671-685.
Schneider, C. A., Rasband, W. S., and Eliceiri, K. W. (2012). NIH Image to ImageJ: 25 years of image analysis. Nat Methods 9, 671-675.
Simao, F. A., Waterhouse, R. M., Ioannidis, P., Kriventseva, E. V., and Zdobnov, E. M. (2015). BUSCO: assessing genome assembly and annotation completeness with single-copy orthologs. Bioinformatics 31, 3210-3212.
Skopinska-Rozewska, E., Malinowski, M., Wasiutynski, A., Sommer, E., Furmanowa, M., Mazurkiewicz, M., and Siwicki, A. K. (2008). The influence of Rhodiola quadrifida 50% hydro-alcoholic extract and salidroside on tumor-induced angiogenesis in mice. Pol J Vet Sci 11, 97-104.
Strommer, J. (2011). The plant ADH gene family. Plant Journal 66, 128-142.
Thompson, J. D., Gibson, T. J., and Higgins, D. G. (2002). Multiple sequence alignment using ClustalW and ClustalX. Current protocols in bioinformatics Chapter 2, Unit 2 3.
Tieman, D. M., Loucas, H. M., Kim, J. Y., Clark, D. G., and Klee, H. J. (2007). Tomato phenylacetaldehyde reductases catalyze the last step in the synthesis of the aroma volatile 2-phenylethanol. Phytochemistry 68, 2660-2669.
Torrens-Spence, M. P., Fallon, T. R., and Weng, J. K. (2016). A Workflow for Studying Specialized Metabolism in Nonmodel Eukaryotic Organisms. Methods Enzymol. 576, 69-97.
Torrens-Spence, M. P., Gillaspy, G., Zhao, B., Harich, K., White, R. H., and Li, J. (2012). Biochemical evaluation of a parsley tyrosine decarboxylase results in a novel 4-hydroxyphenylacetaldehyde synthase enzyme. Biochemical and biophysical research communications 418, 211-216.
Torrens-Spence, M. P., Lazear, M., von Guggenberg, R., Ding, H., and Li, J. (2014). Investigation of a substrate-specifying residue within Papaver somniferum and Catharanthus roseus aromatic amino acid decarboxylases. Phytochemistry 106, 37-43.
Torrens-Spence, M. P., Liu, P., Ding, H., Harich, K., Gillaspy, G., and Li, J. (2013). Biochemical evaluation of the decarboxylation and decarboxylation-deamination activities of plant aromatic amino acid decarboxylases. The Journal of biological chemistry 288, 2376-2387.
Tu, Y., Roberts, L., Shetty, K., and Schneider, S. S. (2008). Rhodiola crenulata induces death and inhibits growth of breast cancer cell lines. Journal of medicinal food 11, 413-423.
Wang, H., Fan, W., Li, H., Yang, J., Huang, J., and Zhang, P. (2013). Functional characterization of Dihydroflavonol-4-reductase in anthocyanin biosynthesis of purple sweet potato underlies the direct evidence of anthocyanins function against abiotic stresses. PloS one 8, e78484.
Wang, M., and Maeda, H. A. (2017). Aromatic amino acid aminotransferases in plants. Phytochemistry Reviews.
Weng, J. K., Ye, M., Li, B., and Noel, J. P. (2016). Co-evolution of Hormone Metabolism and Signaling Networks Expands Plant Adaptive Plasticity. Cell 166, 881-893.
Wyk, H.S.L.A.l.o.t.a.w.P.M.T.A.l.o.t.a.w.B.-E.V. (2010). The ethnobotany and pharmacognosy of Olea europaea subsp. africana (Oleaceae). South African Journal of Botany 76, 324-331.
Xia, J., and Wishart, D. S. (2016). Using MetaboAnalyst 3.0 for Comprehensive Metabolomics Data Analysis. Current protocols in bioinformatics 55, 14 10 11-14 10 91.
Xie, D. Y., Sharma, S. B., Paiva, N. L., Ferreira, D., and Dixon, R. A. (2003). Role of anthocyanidin reductase, encoded by BANYULS in plant flavonoid biosynthesis. Science 299, 396-399.
Yang, Y. N., Liu, Z. Z., Feng, Z. M., Jiang, J. S., and Zhang, P. C. (2012). Lignans from the Root of Rhodiola crenulata. J Agr Food Chem 60, 964-972.
Yousef, G. G., Grace, M. H., Cheng, D. M., Belolipov, I. V., Raskin, I., and Lila, M. A. (2006). Comparative phytochemical characterization of three Rhodiola species. Phytochemistry 67, 2380-2391.
Yu, H. S., Ma, L. Q., Zhang, J. X., Shi, G. L., Hu, Y. H., and Wang, Y. N. (2011). Characterization of glycosyltransferases responsible for salidroside biosynthesis in Rhodiola sachalinensis. Phytochemistry 72, 862-870.
Yuan, T., Fujioka, S., Takatsuto, S., Matsumoto, S., Gou, X., He, K., Russell, S. D., and Li, J. (2007). BEN1, a gene encoding a dihydroflavonol 4-reductase (DFR)-like protein, regulates the levels of brassinosteroids in Arabidopsis thaliana. The Plant journal: for cell and molecular biology 51, 220-233.
Zhang, L., Yu, H. X., Sun, Y., Lin, X. F., Chen, B., Tan, C., Cao, G. X., and Wang, Z. W. (2007). Protective effects of salidroside on hydrogen peroxide-induced apoptosis in SH-SY5Y human neuroblastoma cells. Eur J Pharmacol 564, 18-25.

### INCORPORATION BY REFERENCE AND EQUIVALENTS

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1-21. (canceled)

22. A host cell comprising:
a transgene encoding a heterologous 4-hydroxyphenylacetaldehyde synthase (4HPAAS) and operably linked to a first promoter; and
a transgene encoding a heterologous tyrosol:UDP-glucose 8-O-glucosyltransferase (T8GT) and operably linked to the first promoter or to a second promoter.

23. The host cell of claim 22, wherein the host cell does not comprise a transgene encoding a heterologous 4-hydroxyphenylacetaldehyde reductase (4HPAR).

24. The host cell of claim 22, wherein the host cell does not comprise a transgene encoding a tyrosol:UDP-glucose 4-O-glucosyltransferase (T4GT).

25. The host cell of claim 22, wherein the host cell does not comprise a transgene encoding a heterologous 4-hydroxyphenylacetaldehyde reductase (4HPAR), and does not comprise a transgene encoding a tyrosol:UDP-glucose 4-O-glucosyltransferase (T4GT).

26. The host cell of claim 22, wherein the 4HPAAS comprises an amino acid sequence having at least 90% amino acid sequence identity to SEQ ID NO: 2.

27. The host cell of claim 26, wherein the amino acid sequence of the 4HPAAS comprises: a) an amino acid residue selected from the group consisting of F, L, I, M and V at a position corresponding to the F residue at position 343 in SEQ ID NO: 2; b) an amino acid residue selected from the group consisting of N and D at a position corresponding to the H residue at position 198 in SEQ ID NO: 2; or c) a combination thereof.

28. The host cell of claim 22, wherein the T8GT comprises an amino acid sequence having at least 90% identity to one or more of SEQ ID NO: 14, SEQ ID NO: 16, SEQ ID NO: 18, and SEQ ID NO: 20.

29. The host cell of claim 22, wherein the host cell is a fungi cell, a bacterial cell, or a plant cell.

30. The host cell of claim 29, wherein the fungi cell is a yeast cell.

31. The host cell of claim 30, wherein the yeast cell is a Saccharomyces cerevisiae cell.

32. The host cell of claim 29, wherein the bacterial cell is a cell from a genus selected from Agrobacterium, Escherichia, Bacillus, Pseudomonas and Streptomyces.

33. The host cell of claim 32, wherein the bacterial cell is an Agrobacterium tumefaciens cell, an Escherichia co/i cell, a Bacillus subtilis cell, a Pseudomonas aeruginosa cell, or a Streptomyces griseus cell.

34. The host cell of claim 29, wherein the plant cell is a Nicotiana benthamiana cell, or a cell from a genus selected from the group consisting of Arabidopsis, Beta, Glycine, Helianthus, Solanum, Triticum, Oryza, Brassica, Medicago, Prunus, Malus, Hordeum, Musa, Phaseolus, Citrus, Piper, Sorghum, Daucus, Manihot, Capsicum, and Zea.

35. A method of producing the host cell of claim 22, wherein the method comprises introducing into a cell:
(i) a vector comprising a transgene encoding a heterologous 4-hydroxyphenylacetaldehyde synthase (4HPAAS); and
(ii) a vector comprising a transgene encoding a heterologous tyrosol:UDP-glucose 8-O-glucosyltransferase (T8GT).

36. A method of producing the host cell of claim 22, wherein the method comprises introducing into a cell a vector comprising:
(i) a transgene encoding a heterologous 4-hydroxyphenylacetaldehyde synthase (4HPAAS); and
(ii) a transgene encoding a heterologous tyrosol:UDP-glucose 8-O-glucosyltransferase (T8GT).

37. A method of producing tyrosol 8-O-glucoside (salidroside), wherein the method comprises:
(i) introducing into a host cell:
(a) a transgene encoding a 4-hydroxyphenylacetaldehyde synthase (4HPAAS) and operably linked to a first promoter, and
(b) a transgene encoding a tyrosol:UDP-glucose 8-O-glucosyltransferase (T8GT) and operably linked to the first promoter or to a second promoter; and
(ii) culturing the host cell in a culture media comprising tyrosol.

38. The method of claim 37, wherein the host cell is a fungi cell, a bacterial cell, or a plant cell.

39. The method of claim 38, wherein the fungi cell is a yeast cell.

40. The method of claim 39, wherein the yeast cell is a Saccharomyces cerevisiae cell.

41. The method of claim 38, wherein the bacterial cell is a cell from a genus selected from Agrobacterium, Escherichia, Bacillus, Pseudomonas and Streptomyces.

42. The method of claim 41, wherein the bacterial cell is an Agrobacterium tumefaciens cell, an Escherichia coli cell, a Bacillus subtilis cell, a Pseudomonas aeruginosa cell, or a Streptomyces griseus cell.

43. The method of claim 38, wherein the plant cell is a Nicotiana benthamiana cell, or a cell from a genus selected from the group consisting of Arabidopsis, Beta, Glycine, Helianthus, Solanum, Triticum, Oryza, Brassica, Medicago, Prunus, Malus, Hordeum, Musa, Phaseolus, Citrus, Piper, Sorghum, Daucus, Manihot, Capsicum, and Zea.

44. The method of claim 37, wherein the 4HPAAS comprises an amino acid sequence having at least 90% amino acid sequence identity to SEQ ID NO: 2.

45. The method of claim 37, wherein the T8GT comprises an amino acid sequence having at least 90% identity to one or more of SEQ ID NO: 14, SEQ ID NO: 16, SEQ ID NO: 18, and SEQ ID NO: 20.


SEQUENCE LISTING (291 sequences)

SEQ ID NO: 1 - DNA, 1473 bp, Rhodiola rosea
  source 1..1473: mol_type = other DNA
atgggcagcttgccttctcctaatgatccatcaaacaccttcaaccccatggacctcaccgagttatccaccgagtcgaaactcgtcgtagatttcataactcagtactaccaaaccctagagacccgacccgtccagccacgggtcaagccaggtttcttaacgggccagcttccagataaagcaccctttcatggtgaatcaatggaagtaatattgtctgatgtaaatgagaagattgtccctggcctcactcattggcaaagccctaatttccatgcatactttccagccagttccagcaacgcagggctgttgggagagttactatgctccggactcagtgtcattgggttcacatggagctcctcccctgccgcgacggagcttgagaatgtcgtggttgactggatggccaagatgcttaaccttccatcctctttctgcttctccggcggaggcggtggcgttctgcaagcaaacacttgcgaggctgtgttgtgcactttagccgctgcgagggacaaggctcttaaccgggtgggagatgatcagatcaataaactggtcctctactgctccgaccaaacacatttcacaatccacaagggcgcaaagttgataggaatccgatcaaagaacataaaatcaatcactactaagaaagagaacgagtttaaactctgtcctaacgacctacgcgacgcgataaggagtgatctggaagcaggactagttccgttttacgtatgcggaacgattggaacgaccgcgttaggagttgtggatccgattaaagagctgggtaaggtggcaagagagtttgatttgtggttacatgttgatggagcttatggtggcagtgcatgcatatgccctgagtttcagcattaccttgatggagttgaccttgttgactcgatcagcatgaatgcacataaatggcttttatccaatctagattgctgcttcctgtggcttcaatctcctaacgccctaatcgaatccctggccgcagaagctaactttctgaaaggtggtagtgagatggtggattacaaggactggcagatatcgttgagtcgtcgatttagagcgatcaagatgtggatggtgataaggcgatacggtgtgagtaatctcattgagcatattcgatccgacgtgagcatggcggtgagattcgaagagatggtggcggcggacgaccggtttgaaatcgtgtttcctagaaagtttgcgcttgtttgcttcaagcttagtagcgagaagacaccaccgggccgcgactcggagttaactcgtgagctgatggagagagtcaactcgagtgggaaggcttacttgagtggagttcaaatgggtcggatcttcttcatcaggtgtgtgatcgggtcgagtttgactgaggagagacacgtcgataatctgtggaggctcattcaagaaacagctcaaagcatcgtgtcttag

SEQ ID NO: 2 - protein, 490 aa, Rhodiola rosea
MGSLPSPNDPSNTFNPMDLTELSTESKLVVDFITQYYQTLETRPVQPRVKPGFLTGQLPDKAPFHGESMEVILSDVNEKIVPGLTHWQSPNFHAYFPASSSNAGLLGELLCSGLSVIGFTWSSSPAATELENVVVDWMAKMLNLPSSFCFSGGGGGVLQANTCEAVLCTLAAARDKALNRVGDDQINKLVLYCSDQTHFTIHKGAKLIGIRSKNIKSITTKKENEFKLCPNDLRDAIRSDLEAGLVPFYVCGTIGTTALGVVDPIKELGKVAREFDLWLHVDGAYGGSACICPEFQHYLDGVDLVDSISMNAHKWLLSNLDCCFLWLQSPNALIESLAAEANFLKGGSEMVDYKDWQISLSRRFRAIKMWMVIRRYGVSNLIEHIRSDVSMAVRFEEMVAADDRFEIVFPRKFALVCFKLSSEKTPPGRDSELTRELMERVNSSGKAYLSGVQMGRIFFIRCVIGSSLTEERHVDNLWRLIQETAQSIVS

SEQ ID NO: 3 - DNA, 981 bp, Rhodiola rosea
  source 1..981: mol_type = other DNA
atgagtttaagcggagcggggaaggtggtttgcgttaccggcgcgtctggctacatagcgtcctggctcgtcaagcttcttctccagcgcggttataccgtcaaggcctccgttcgcgatcctaatgatccgaaaaagactcagcacttgacggcacttgatggagctaaggagaggctgcagttgtacaaagccaatttgcttgaacaaggctcgtttgatcccatagttgaaggatgtgaaggtgttttccacaccgcgtctcccttttatcatgcagtggatgatccgcaggccgagttaattgaccctgctgtcaagggaacactcaatgttctttcttcatgtgctaaagttgcgtctcttaaaagagtagtcctgacttcttcgattgctgctgttgcatataatgggaaaccccgtactccggaggttgtagttgacgagacttggttttctaacccagatgtttgtaaggagatgaagctttggtatgtcatatccaagacactcgctgaagaagcagcatggaagtttgtgaaagagaaaggaatagacatggttaccataaatccggccatggtgattggtccccttctgcaaccaacactcaataccagtgctgctgctattctgaacttgatcaatggatcggagacatacccaaatgcttcttttggatgggtcaatgtgaaagatgttgcagaagcacacgttcttgcatttgaggttccttcagctaatggtagatactgcttggtggaaagagttgcccacagttctgaagtggtgaacatgctccatgagctctaccctgatatcaaacttcccgccaagtgtgcagatgacaaaccatttgtgccaatttatcaagtttcaaaagaaaaggcacatactttaggggtaaaattcattcctttagaggtaagcctcaaggaaacagttgaaagcttgaaggaaaagggtttcgccaaactctga

SEQ ID NO: 4 - protein, 326 aa, Rhodiola rosea
MSLSGAGKVVCVTGASGYIASWLVKLLLQRGYTVKASVRDPNDPKKTQHLTALDGAKERLQLYKANLLEQGSFDPIVEGCEGVFHTASPFYHAVDDPQAELIDPAVKGTLNVLSSCAKVASLKRVVLTSSIAAVAYNGKPRTPEVVVDETWFSNPDVCKEMKLWYVISKTLAEEAAWKFVKEKGIDMVTINPAMVIGPLLQPTLNTSAAAILNLINGSETYPNASFGWVNVKDVAEAHVLAFEVPSANGRYCLVERVAHSSEVVNMLHELYPDIKLPAKCADDKPFVPIYQVSKEKAHTLGVKFIPLEVSLKETVESLKEKGFAKL

SEQ ID NO: 5 - DNA, 1425 bp, Rhodiola rosea
  source 1..1425: mol_type = other DNA
atgggttctgattcacggcctctacgcgtcttcttctttcccttcatggctcacggccatctgattccgatggtcgacatcgccagactcttctcttctcaaggagtccactccaccatcatcaccaccccactaaacgccaattacatctccaaaacgacgtctctatccatcaaaacgataccgtttcctgctgcggaagttgggcttccggacggctgcgagaatatcgacatgcttccttcgcccgatctcttcttcaaatttttccaagccgccaatttactccaagcgccgttcgagaaccttctagaactcgaaaggcccgattgcttaatctccgacatcttcttcccctggtcagtcgactccgccgagaaattcaacatcccgagactcgttttccacggcacgagcttcttcgccatgtgcgccatggagagcttgaagacccacaagccctataaatcggtaagcaccgactctgaaccgttcttaatcccgaatctccctgatgaaatcaaaatgactaaaagtcagttcacggttgacgcttgggaagacaccgaaaagggccttgggaagctgttggctgatgcgagagcttcagggctgaggagcttcggcatgatcgtaaacagcttccacgagctcgaaccggcttacgcggattattacaagaatgtgttgaacatgaaagcgtggtgtgtcgggcctgtttcgttatataaccgaaacgatgacgagaaaattgcaagagggaagaaatcagcaatcgatgatcatgagtgtttaaaatggctggagggaaagcagccagactccgtcgtgtacgtttgtttcgggagcagcgcgagcttccctgatgagcagttgcgcgatatcgcattggggctggaagaatctggagtaaatttcatctgggtgatcaggagaagttccgagtcaggatcagaagattacttgccggaggggtttgaggaccgggtgaaggacagagggctcgtgatccgaggttgggcgccacaggtactgattttggaccatccgtcggttgggggatttgtgactcactgcggatggaattcggcattggaggggatttcagctggcttgccgatggtgacttggccactgttcgcagagcagtttttcaaccagaaattgattacggatgtgttgaaagttggggttgaggttggagtgcagaaatggtctcggaacggggaggatcgcgtgacgaaggagaaggttgagaaggcggtgagggctgttatggttggggaggacgctgaggagaggcgtggcagagctcgtcagcttgggaaattggcaaagaaagctgtggcgaaagatgggtcttcgtacattgatctccacaatttgcttgatgaattgaagttgagaagagagactttgtcctag

SEQ ID NO: 6 - protein, 474 aa, Rhodiola rosea
MGSDSRPLRVFFFPFMAHGHLIPMVDIARLFSSQGVHSTIITTPLNANYISKTTSLSIKTIPFPAAEVGLPDGCENIDMLPSPDLFFKFFQAANLLQAPFENLLELERPDCLISDIFFPWSVDSAEKFNIPRLVFHGTSFFAMCAMESLKTHKPYKSVSTDSEPFLIPNLPDEIKMTKSQFTVDAWEDTEKGLGKLLADARASGLRSFGMIVNSFHELEPAYADYYKNVLNMKAWCVGPVSLYNRNDDEKIARGKKSAIDDHECLKWLEGKQPDSVVYVCFGSSASFPDEQLRDIALGLEESGVNFIWVIRRSSESGSEDYLPEGFEDRVKDRGLVIRGWAPQVLILDHPSVGGFVTHCGWNSALEGISAGLPMVTWPLFAEQFFNQKLITDVLKVGVEVGVQKWSRNGEDRVTKEKVEKAVRAVMVGEDAEERRGRARQLGKLAKKAVAKDGSSYIDLHNLLDELKLRRETLS

SEQ ID NO: 7 - DNA, 1419 bp, Rhodiola rosea
  source 1..1419: mol_type = other DNA
atgtcaggcacaccacacatcgccatcctccccagccccggcatgggccacctcatccccatggccgagttcgccaagcgcctagtccaccaccacaacttcagtatcaccttcgtcatccctaccgacggcccaccttcctccgcctaccaacaagtcctcacctccctcccatcttccatagatcacatcttccttccacaagtcgacttaaccgacgtcgtatcacaatcaccagctcatcccagaatcgaaaccctaatctccctcaccgtcgctcgctccctctcctccctccgcaccaccttatcctctctccaatcgtctaaaaacctcgtctcgctcgtcgttgatcttttcggcactgatgcattcgacccggccatcgagctcggcatctcgccctacattttcttcccttccacagccatgacgctctcgctcttcctatacatgcctcagcttgacaaatcagtcacgtgcgaatttcgtcacatgacggatttggttcgaattcctggatgcgttcctgtccgtggatcggatttattcgacccggttcaagacaggaccgacgaggcttataaatgggtcatacatcactccaacaggtaccctatggcggagggtgttatagagaatagcttcatggagttggaacatggtgcgttaaagtatttgcaaacggttcaatcgggtaagccgcctgtctacgcggtcggaccgttgattaaaatggattatgatgttgacgattccgggtcgaagataatcgagtggctcgatgatcaaccggttggttcggttttatttgtttcgtttggaagcggcggaacgctctcgtatgagcaaatgaccgagctggctcacggtttggaatcgagccagcaacggttcttatgggtggttcggagtccgaatcaaatccccaacagcacgtatttcagtgtacaaagccaaaaagacccgttggcttacttgccagaaggatttttaaaccgaaccgagggtaggggtctggtcgtatcgaattgggccccacaggctcaaattttgagtcacggttcgaccggtgggttcatgagccactgtggttggaattcgattttggagagtgtggtgcacggcgtgccgatcatagcgtggccgttgtacgccgagcagaagatgaattcgataatcgtggtggaggacgttaaggtggcgctgaggccggcgggggtaggggagagggtggtggagaggtcggagataaccgcagtggtgaaggcgttgatggagggtgaggaggggaagaaggtaaggaataggatgaaggaactcaaggaagcggcggcacgtgcggttagtgatgacggtgcgtcgaccatagcgattgcggacttggcgcaaaaatggcggagttcgatgaagcattga

SEQ ID NO: 8 - protein, 472 aa, Rhodiola rosea
MSGTPHIAILPSPGMGHLIPMAEFAKRLVHHHNFSITFVIPTDGPPSSAYQQVLTSLPSSIDHIFLPQVDLTDVVSQSPAHPRIETLISLTVARSLSSLRTTLSSLQSSKNLVSLVVDLFGTDAFDPAIELGISPYIFFPSTAMTLSLFLYMPQLDKSVTCEFRHMTDLVRIPGCVPVRGSDLFDPVQDRTDEAYKWVIHHSNRYPMAEGVIENSFMELEHGALKYLQTVQSGKPPVYAVGPLIKMDYDVDDSGSKIIEWLDDQPVGSVLFVSFGSGGTLSYEQMTELAHGLESSQQRFLWVVRSPNQIPNSTYFSVQSQKDPLAYLPEGFLNRTEGRGLVVSNWAPQAQILSHGSTGGFMSHCGWNSILESVVHGVPIIAWPLYAEQKMNSIIVVEDVKVALRPAGVGERVVERSEITAVVKALMEGEEGKKVRNRMKELKEAAARAVSDDGASTIAIADLAQKWRSSMKH

SEQ ID NO: 9 - DNA, 1431 bp, Rhodiola rosea
  source 1..1431: mol_type = other DNA
atggctgaaaacactcatgctcatgccatagtggtaccatttccagttcaaggacacataaagccctcgctgaatctagccctcaagctagcatctcaaggcttcaccatcacttttgtcaccactcatttcacccaccagcaaatctcccaagctcacaaaaacagtacaaatacaaaccatgacatgtttttccaggcacgaaactccagtctcgatatccgccatgtaacggtgacagacacttttcctttgggattcgatcgcgcagggaatcaggatcagttttgggagggcatgcttcacgtattccctgcacatgttgatgaactggtggatcagttaatgaattcttcgaagccgagaccaacttgtttgattctggatacattttataactggggttccaaaattgctaacaagtttaatttagtgcatatttcattttggactcagtctgctctttctttcactttgttttaccattgggaacttttaaagaaaaatggtcactttggctctccagataatcgcacggatgtcatcgattatattcccggtgtgcaagagatcaagcccgcagacttaatatcctaccttcagatgagtgatacaactactgtggctcacaggacttgtttcacagcatttgaagatgtcaggaaggcagatttcatcctggctaatacaatccaagaatttgaaactgatacaatttcttctatccgatttcaccagccatttttctacccaattggacctgtttttttaacaaagtctgaacaacaagctagctcagctttgtggtctgagtcagactgtgagcagtggctaagtacaaaaccaaaagggtctgttctctatgcctcatttgggagctatgctcgtgtaactaggcatgatatcgcagagatagcctacggattgatgcaaagtgaggtgaattttatttgggtgattcgcgacgatattgtgggtgcacacgagactgattttttaccaacagaattcataaatggaatcaaactcaaagatcagggactactagtttcctggtgctctcaaactgaagttttgtccaatgcggcgattggaggatttctgactcattgtggatggaactcgatactcgaaagcgtatggtgtgaagttccattattgtgttttccaataatgactgatcagcctagtaacaggaaactggtggtggatgactggaggatcggcgtcaacctatctgcggcggaggaggtcagtagagaagaagtgtcaatgaaggtcaggaacttgatttctggagaattggggaatgagttgagagtgcagattcaaaagtacaaaaagttgatggagaatggtataatggaaggtggatcatcacattccaattggaacaagttcatccacgacctacaaatcttcaagaaatga

SEQ ID NO: 10 - protein, 476 aa, Rhodiola rosea
MAENTHAHAIVVPFPVQGHIKPSLNLALKLASQGFTITFVTTHFTHQQISQAHKNSTNTNHDMFFQARNSSLDIRHVTVTDTFPLGFDRAGNQDQFWEGMLHVFPAHVDELVDQLMNSSKPRPTCLILDTFYNWGSKIANKFNLVHISFWTQSALSFTLFYHWELLKKNGHFGSPDNRTDVIDYIPGVQEIKPADLISYLQMSDTTTVAHRTCFTAFEDVRKADFILANTIQEFETDTISSIRFHQPFFYPIGPVFLTKSEQQASSALWSESDCEQWLSTKPKGSVLYASFGSYARVTRHDIAEIAYGLMQSEVNFIWVIRDDIVGAHETDFLPTEFINGIKLKDQGLLVSWCSQTEVLSNAAIGGFLTHCGWNSILESVWCEVPLLCFPIMTDQPSNRKLVVDDWRIGVNLSAAEEVSREEVSMKVRNLISGELGNELRVQIQKYKKLMENGIMEGGSSHSNWNKFIHDLQIFKK

SEQ ID NO: 11 - DNA, 1458 bp, Rhodiola rosea
  source 1..1458: mol_type = other DNA
atggcagaaataagtctcatcttcatcccttttcccgtaatcagccatctcactcccacaatcgaaatcgccaaaatcctcctcagcagagaccaccgcctttccatcaccttcctcgtcatcgacatcccccaacgagacgcctcactcgcctccctcaccacctccatcatctccgatcgcctccacttcctcgatgtcgtacttcctcccaaccaacactcccaatcatccaagccatcaggcatcgcggctatcgagtccgccaaacccgcagtcaagaaaacgatcagcgatcttgttgtacgatctcagtccgccgcatctggtccgcggatagctggcttcgtgctggacatgttctgcacggccatgatcgacatcgcaactgagtttaaccttccttcgtatatttactacacttgcggctcttcgtttctttcaatcgtgctccacgtccagaagctctgcgatgacgacgctctcgatatcgccgatttcaaaaactcgagtgtggagttttcgttacctgagttttcaaacttgattccggctaggctgcttccatccatggcgctcgataaggacttctcggcttcattcgtcggcaaagctagagcgttcaggaagacgaagggcattttggtcaactcgcttgtagagttggagcctcacgcaatcgagtcgatgaaattagaccggtctgttcctccgatttactcggtcggaccagtgctcaacatgaatagcaacactgcatttatcagacaggagcaggagaaggagatcatggagtggctggaccaacagcctccagcatctgtagttttcttgtgttttggcagcaggggagcgttcaagccggaccaggtgaaggaaatcgcacgggggttggagtcgagcggctgccggttcctctgggcgcttcggcagccttcatcaagcaatgtgaggttttcacctcctacagattatgaagatttctctgaggttctgcctgaagggtttttgcagcggacatatggtgttgggaaagtgattggttgggcaccccagacagctgttttagaccacccttcggtgggtggattcgtatcgcattgcggttggaactcgatactggaatctctttggtttggtgtgccgattgcgacttggcctctgtatgctgagcagcagatgaatgcgtttgaggttgtgaaggagatgaagattggagtggagataagtttggattatcggcttgaaatgggcggtaaacaagcagaaggttctgggattataagtggtgaacagattgagagagggattagagatgtgatgcaggaggatagtgaagtgaggaagaaggtgaagctgatgatggaaaagagtagagaggcagttgtggagggaggctcctcttataattatatccaaaacttcatcagtgatctcaggaccaacattggcttgtaa

SEQ ID NO: 12 - protein, 485 aa, Rhodiola rosea
MAEISLIFIPFPVISHLTPTIEIAKILLSRDHRLSITFLVIDIPQRDASLASLTTSIISDRLHFLDVVLPPNQHSQSSKPSGIAAIESAKPAVKKTISDLVVRSQSAASGPRIAGFVLDMFCTAMIDIATEFNLPSYIYYTCGSSFLSIVLHVQKLCDDDALDIADFKNSSVEFSLPEFSNLIPARLLPSMALDKDFSASFVGKARAFRKTKGILVNSLVELEPHAIESMKLDRSVPPIYSVGPVLNMNSNTAFIRQEQEKEIMEWLDQQPPASVVFLCFGSRGAFKPDQVKEIARGLESSGCRFLWALRQPSSSNVRFSPPTDYEDFSEVLPEGFLQRTYGVGKVIGWAPQTAVLDHPSVGGFVSHCGWNSILESLWFGVPIATWPLYAEQQMNAFEVVKEMKIGVEISLDYRLEMGGKQAEGSGIISGEQIERGIRDVMQEDSEVRKKVKLMMEKSREAVVEGGSSYNYIQNFISDLRTNIGL

SEQ ID NO: 13 - DNA, 1443 bp, Rhodiola rosea
  source 1..1443: mol_type = other DNA
atgggctcacttccttccacaaaatcccatgcagtcctcgtcccataccctgcccaaggccacatcaaccctttcatgcaacttgccaagctcctacactcaaaaggtttccacataaccttcgtcaacaatgaccacaaccatcgccgtttgctcagaacaaaagggcatgattttgttcaagggttggaaggtttaaggtttgaagctgtgccggatggcctacctccatctgaccgtgatgccactcaggatgtccctaagctgactgaatctatttacaataagagcatgaaccaaccgttcagtgatctgcttcagaggctaaactcaacgcccggttcccctccggtcacttgtgtcatatccgatgttgccatgttttttgcttgggacgtggcggatgagcttggcatccctaatgttcagttttggacagcttcagcttgtggccttttgggatacttacagtatgatgagctcctaagaagagccatagtcccattcaaagatgaaaatttcatgacggatggttcgttggaggctttgattgactggattcctggcatgcctaacatgaggctgaaggacttgccaagcttcatgcggaccacaagccctgacgacgtgttgttcaattacttgcgtacaataaccacgaaagctctaaaatcctcggccttgttgctgaacacatttgatgattttgaacatgaagtagttgaagagatgaagaaaatgcaaccaaacatattcctaggaggtccactcaacatgcttctcaggcacacatcaaaaactgaaatcacatccttaacaacaagtttatggaaagaggacactcattgtttagaatggctggacaagcaagaaccggagtcagtggtatacatcaattacggatcggtgacgataatgtctgatcaccatttaaatgagtttgcttggggtttggctaacagcaagcacccttttttgtggatcgtgaggccggatgttgtgaggggcgagtcggggactttgcccaaggagttttatgatgagatcaaggacaggggattgataacgagctggtgtccgcaaccagaggtgcttaaacatccatccgtaggtgtatacttgacgcattgtggttggaactctatcacggagagtgtggccggaggagtgccattgatgtgctggccgtttttcgctgagcaacagacgaatagccgattcgcgtgtacggtgtggggcactggagtggaggtgaatgcggatgtgaagagggaggagctagcggaacaagtgatggagatgttggaaggaaagagggggcaagagttgaggaaaaatgctaaggagtggaggaggaaggcggaggaggcgacggacattggcggttctgcctatgctgatttcgataggtttatggaaaaagtggtccagtttagcgtctga

SEQ ID NO: 14 - protein, 480 aa, Rhodiola rosea
MGSLPSTKSHAVLVPYPAQGHINPFMQLAKLLHSKGFHITFVNNDHNHRRLLRTKGHDFVQGLEGLRFEAVPDGLPPSDRDATQDVPKLTESIYNKSMNQPFSDLLQRLNSTPGSPPVTCVISDVAMFFAWDVADELGIPNVQFWTASACGLLGYLQYDELLRRAIVPFKDENFMTDGSLEALIDWIPGMPNMRLKDLPSFMRTTSPDDVLFNYLRTITTKALKSSALLLNTFDDFEHEVVEEMKKMQPNIFLGGPLNMLLRHTSKTEITSLTTSLWKEDTHCLEWLDKQEPESVVYINYGSVTIMSDHHLNEFAWGLANSKHPFLWIVRPDVVRGESGTLPKEFYDEIKDRGLITSWCPQPEVLKHPSVGVYLTHCGWNSITESVAGGVPLMCWPFFAEQQTNSRFACTVWGTGVEVNADVKREELAEQVMEMLEGKRGQELRKNAKEWRRKAEEATDIGGSAYADFDRFMEKVVQFSV

SEQ ID NO: 15 - DNA, 1488 bp, Rhodiola rosea
  source 1..1488: mol_type = other DNA
atgggatctctaggaaagaagattcaacaaaagccacatgcaatatgcaccccatacccagcacaaggccatattaatcccatgcttaaactagccaagctcctacaccactcaggcttctacataacctttgttcacacaacctacaactacaatcgccttctcaagacccacgggtctgattccttaagtggtctaccagatttccaatttgagaccatccctgatggactaccaccatcagatgcagctgatgtcacacaagacatccctgccttgtgtaaatcaaccaccgaaacctgcttagtcccattcaaagagctcctggctaagctgcataacaagtcaatggcgtcaccggaggaagttcctccagtgacatgcatagtttctgatggttgcatgtcatttactgtggatgctgcagaagaggcaggggttcctaatgtgcttctttggactaccagtgcatgcggatttttaggatatgctaattacccgaaacttattgacagaggcataattccactcaaagatgagagctactttacgaatgggtacctagacaagacagtagatggaatacctggaatgaaaggcatacggctacgagacttcccaaactttgtatgcaccacaaacccagatgagtttatggtgaaatatgcaattcaagagatcactagagctgccagagcagatgctgttattttgaacacctttgacgctttggaacatgatttcttagatggcctatcaaacatatacccaaaggtcctccctattggcccgctccagcttccgctcaaccaaatcccagagagctcacctctacattcaatctgttctagtctctggaaagatgaaccacagtgcattacctggttaaactcccaaaaaccaaaatcagtcgtttatgttaactacggaagtatcacagttatgactccgcaacaaatggtggagttcgcatggggactggctaatacaaaatacccttttctgtggattattagacctgatttggttgctggtgagacagctgtcctacctccagattttttggaagtgacaaaaggaaggagctgcttggctagttggtgcccacaggaacaagttcttagtcacacatccataggagggttcttaacccattgtgggtggaactcaatgctagaaagcgtggtcgaaggagttccaatggtatgctggccgttttttgctgagcaacagactaattgctgggctgctcggacaaaatggggtataggtatggaaattgacaatgatgttaagagggataaggttcagaaaatggtgacagagcttatggagggcgaaaagggaaaggagatgaagaggaagggcggagaatggaagaagcttggggcagaagctgccggtcctaatggctcagctaccttaaacttcagcagacttataaatgacgtacttctgtccaaaaaaaaaattgtagttacaacctaa

SEQ ID NO: 16 - protein, 495 aa, Rhodiola rosea
MGSLGKKIQQKPHAICTPYPAQGHINPMLKLAKLLHHSGFYITFVHTTYNYNRLLKTHGSDSLSGLPDFQFETIPDGLPPSDAADVTQDIPALCKSTTETCLVPFKELLAKLHNKSMASPEEVPPVTCIVSDGCMSFTVDAAEEAGVPNVLLWTTSACGFLGYANYPKLIDRGIIPLKDESYFTNGYLDKTVDGIPGMKGIRLRDFPNFVCTTNPDEFMVKYAIQEITRAARADAVILNTFDALEHDFLDGLSNIYPKVLPIGPLQLPLNQIPESSPLHSICSSLWKDEPQCITWLNSQKPKSVVYVNYGSITVMTPQQMVEFAWGLANTKYPFLWIIRPDLVAGETAVLPPDFLEVTKGRSCLASWCPQEQVLSHTSIGGFLTHCGWNSMLESVVEGVPMVCWPFFAEQQTNCWAARTKWGIGMEIDNDVKRDKVQKMVTELMEGEKGKEMKRKGGEWKKLGAEAAGPNGSATLNFSRLINDVLLSKKKIVVTT

SEQ ID NO: 17 - DNA, 1476 bp, Rhodiola rosea
  source 1..1476: mol_type = other DNA
atgggatctctaggaaagaagattcaacaaaagccacatgcaatatgcaccccatacccagcacaaggccatattaatcccatgcttaaactagccaagctcctacaccactcaggcttctacataacctttgttcacacaacctacaactacaatcgccttctcaagacccacgggtctgattccttaagtggtctaccagatttccaatttgagaccatccctgatggactaccaccatcagatgcagctgatgtcacacaagacatccctgccttgtgtaaatcaaccaccgaaacctgcttagtcccattcaaagagctcctggctaagctgcataacaagtcaatggcgtcaccggaggaagttcctccagtgacatgcatagtttctgatggttgcatgtcatttactgtggatgctgcagaagaggcaggggttcctaatgtgcttctttggactaccagtgcatgcggatttttaggatatgctaattacccgaaacttattgacagaggcataattccactcaaagatgagagctactttacgaatgggtacctagacaagacagtagatggaatacctggaatgaaaggcatacggctacgagacttcccaaactttgtatgcaccacaaacccagatgagtttatggtgaaatatgcaattcaagagatcactagagctgccagagcagatgctgttattttgaacacctttgacgctttggaacatgatttcttagatggcctatcaaacatatacccaaaggtcctccctattggcccgctccagcttccgctcaaccaaatcccagagagctcacctctacattcaatctgttctagtctctggaaagatgaaccacagtgcattacctggttaaactcccaaaaaccaaaatcagtcgtttatgttaactacggaagtatcacagttatgactccgcaacaaatggtggagttcgcatggggactggctaatacaaaatacccttttctgtggattattagacctgatttggttgctggtgagacagctgtcctacctccagattttttggaagtgacaaaaggaaggagctgcttggctagttggtgcccacaggaacaagttcttagtcacacatccataggagggttcttaacccattgtgggtggaactcaatgctagaaagcgtggtcgaaggagttccaatggtatgctggccgttttttgctgagcaacagactaattgctgggctgctcggacaaaatggggtataggtatggaaattgacaatgatgttaagagggataaggttcagaaaatggtgacagagcttatggagggcgaaaagggaaaggagatgaagaggaagggcggagaatggaagaagcttggggcagaagctgccggtcctaatggctcagctaccttaaacttcagcagacttataaatgacgtacttctgtccaaaaaaaaattgtag

SEQ ID NO: 18 - protein, 491 aa, Rhodiola rosea
MGSLGKKIQQKPHAICTPYPAQGHINPMLKLAKLLHHSGFYITFVHTTYNYNRLLKTHGSDSLSGLPDFQFETIPDGLPPSDAADVTQDIPALCKSTTETCLVPFKELLAKLHNKSMASPEEVPPVTCIVSDGCMSFTVDAAEEAGVPNVLLWTTSACGFLGYANYPKLIDRGIIPLKDESYFTNGYLDKTVDGIPGMKGIRLRDFPNFVCTTNPDEFMVKYAIQEITRAARADAVILNTFDALEHDFLDGLSNIYPKVLPIGPLQLPLNQIPESSPLHSICSSLWKDEPQCITWLNSQKPKSVVYVNYGSITVMTPQQMVEFAWGLANTKYPFLWIIRPDLVAGETAVLPPDFLEVTKGRSCLASWCPQEQVLSHTSIGGFLTHCGWNSMLESVVEGVPMVCWPFFAEQQTNCWAARTKWGIGMEIDNDVKRDKVQKMVTELMEGEKGKEMKRKGGEWKKLGAEAAGPNGSATLNFSRLINDVLLSKKKL

SEQ ID NO: 19 - DNA, 1497 bp, Rhodiola rosea
  source 1..1497: mol_type = other DNA
atgagcttaattgaaaaaccactcacggccatagagactcgtgaaaaaccacacgctgtgtgcatcccatacccagctcaaggccatatcaatcccatgatgcaacttgcaaagctcctccaccactctggtttccacataacgtttgtccacactgagtataattatgaccgtctagtgaagtctcaaggttcagcttgtgtggctggtttaccggatttccgctttgaagccatcccagatggcttgccctcgacgaatggtgatgttactcaagacattcctctgttgagtagctctacttctaaaacctgcttgaagccgtttaaggagttattgaagaggttgcaggacaaatgcaaagagttacctgatgatgttccgcctctgtcgtgcatcgtgtctgatgcagccatgtcgtttacgatcgatgcatctgaggagtttggagtgcccatagcgcttctttggactgcaagtgcctgcgggttcttgggttacacgcattacccatatctaattgacagaggtgtcatcccattgaaagatgagagccaattaacaaacggatacctagatatgagcatagatggcataccttgtatggaaggtatccgcttacgagacctcccaagctttctacgcacaactgatttagatgatatgatgtttagttatatactgcacgaaataaaacaagtttcaagaggcagtgctatcattctgaacacctttgaagctttggaccatgatgtcttggatagtctctccaaaatttaccaaaatgtcatcctgccagttggccctctacatgtctcgctcaacaagatcccaaaacactacccacttcaatctttaagctcgaatttatggaaagatgacacagactgcattccctggctgagctctaaggcttcaaaatcagttatatacgttaactttgggagcatcacgacggtatcaccaaaacaaattgtggagtttgcgtggggattggctaacagcaaacaccctttcctttggataatcagaccggacttggtggcaggtgaggcatccatcattccgcaggacttcatggatgaaacaaaaggaagaggtttgttggctggttggtgtgaccaagagcttgttctcaaccatccatccattggagggtttcttacgcactgtggctggaactcaattattgaaagcattagcgcaggagtccctacggtctgctggccattttttgctgagcagcaaacaaattgttggtttgcttgcaaaaaatggtgcattgggatggagatgcatactgatgtaaagagggatgaggttgacaagctgttgagagagctaatggaaggtgacaaaggggaggagttgaagaggaaggcaaccaactggaagaggctggcagaagaagctgtttcctccactggcttatcaaccttaaacttcaggacgttagtgaatcaagtcctgctctcaaaaacaaaacatatccgttag

SEQ ID NO: 20 - protein, 498 aa, Rhodiola rosea
MSLIEKPLTAIETREKPHAVCIPYPAQGHINPMMQLAKLLHHSGFHITFVHTEYNYDRLVKSQGSACVAGLPDFRFEAIPDGLPSTNGDVTQDIPLLSSSTSKTCLKPFKELLKRLQDKCKELPDDVPPLSCIVSDAAMSFTIDASEEFGVPIALLWTASACGFLGYTHYPYLIDRGVIPLKDESQLTNGYLDMSIDGIPCMEGIRLRDLPSFLRTTDLDDMMFSYILHEIKQVSRGSAIILNTFEALDHDVLDSLSKIYQNVILPVGPLHVSLNKIPKHYPLQSLSSNLWKDDTDCIPWLSSKASKSVIYVNFGSITTVSPKQIVEFAWGLANSKHPFLWIIRPDLVAGEASIIPQDFMDETKGRGLLAGWCDQELVLNHPSIGGFLTHCGWNSIIESISAGVPTVCWPFFAEQQTNCWFACKKWCIGMEMHTDVKRDEVDKLLRELMEGDKGEELKRKATNWKRLAEEAVSSTGLSTLNFRTLVNQVLLSKTKHIR

SEQ ID NO: 21 - protein, 490 aa, Arabidopsis thaliana
MENGSGKVLKPMDSEQLREYGHLMVDFIADYYKTIEDFPVLSQVQPGYLHKLLPDSAPDHPETLDQVLDDVRAKILPGVTHWQSPSFFAYYPSNSSVAGFLGEMLSAGLGIVGFSWVTSPAATELEMIVLDWVAKLLNLPEQFMSKGNGGGVIQGSASEAVLVVLIAARDKVLRSVGKNALEKLVVYSSDQTHSALQKACQIAGIHPENCRVLTTDSSTNYALRPESLQEAVSRDLEAGLIPFFLCANVGTTSSTAVDPLAALGKIANSNGIWFHVDAAYAGSACICPEYRQYIDGVETADSFNMNAHKWFLTNFDCSLLWVKDQDSLTLALSTNPEFLKNKASQANLVVDYKDWQIPLGRRFRSLKLWMVLRLYGSETLKSYIRNHIKLAKEFEQLVSQDPNFEIVTPRIFALVCFRLVPVKDEEKKCNNRNRELLDAVNSSGKLFMSHTALSGKIVLRCAIGAPLTEEKHVKEAWKIIQEEASYLLHK

SEQ ID NO: 22 - protein, 498 aa, Brachypodium distachyon
MDGSTTSNGGGGWMRPMDEEQLRECGHRMVDFIADYYKSIETYPVLSQVQPGYLKELLPDSAPNQPDTLDALFDDIREKIVPGVTHWQSPNYFAYYPSNSSTAGFLGEMLSAAFNIVGFSWITSPAATELEVIVLDWVAKMLKLPSEFLSAALGGGVIQGTASEAILVVLLSARDRTLRKHGKKSLEKIVVYASDQTHSALKKACQIAGIFPENIRIVKADCSMNYAVTPGAVSEAISIDLSAGLIPFFICATVGTTSSSAVDPLHELGQIAQAHDMWFHIDAAYAGSACICPEYRKYLNGVEEADSFNMNAHKWFLTNFDCSLLWVKDRNYLIQALSTNPEFLKNKASQENSVIDFKDWQIPLGRRFRSLKLWMVLRLYGVENLQSYIRKHIQLAQHFEQLVISDPRFEVVTPRNFSLVCFCLVPPTCEVDNGHKLNYDLMDSANSSGKIFISHTVLSGKFVLRFVVGAPLTEEQHVDAAWKLLQDEATKLLGNVVQ

SEQ ID NO: 23 - protein, 480 aa, Carica papaya
MDAEQLRENGHKMVDFIADYYKTIENFPVLSQVEPGYLRDLIPDSAPNSPESFQQLLDDVRTKILPGVTHWQSPNYFAYYPSNSSVAGFLGEMLSAGLNIVGFSWITSPAATELEMIVLDWLAKLLKLPEDFHSTGNGGGVIQGTASEAILVVLLAARDKVLKRVGKNALEKLVVYTSDQTHSAFQKACQIGGIHPENCRVLKTDSSTNYALSPDLLKEAISCDVAAGLIPFFFCATVGTTSSTAVDPLMALGKIATSNEIWFHVDAAYAGSACICPEYRPYIDGVEEADSFNMNAHKWFLTNFDCSVLWVKDKYSLIQSLSTNPEFLKNKASQADMVVDYKDWQIPLGRRFRSLKLWMVLRLYGVENLKSYIRNHIKLAKHFEELVTQDPRFEVVTPRIFSLVCFRLLPPGNDENHGNKLNQDLLETVNSTGKLFISHTVLSGKYILRFAVGAPLTEERHVNEAWKILQDEASTLLENP

SEQ ID NO: 24 - protein, 492 aa, Ricinus communis
MFREGELRPMDAEQLREHGHKMVDFIADYYKTIENFPVLSQVEPGYLRKLLPDSAPNQPESLQNVLDDVQAKILPGVTHWQSPNYFAYYPSNSSVAGFLGEMLSAGINMVGFSWITSPAATELEMIVLDWLGKMLKLPEEFLSTGQGGGVIQGTASEAVLVALVAARDKVLRRVGKDALRKLVVYGSDQTHSALQKACQIGGIHPVNCRLLETDSSTNYALAPDLLSRAISEDISLGLIPFFLCATVGTTSSTAVDPLLALGKIAKSNGMWFHVDAAYAGSACVCPEYRCYMDGVEEADSFNMNAHKWFLTNFDCSALWVKDRNALIQSLSTSPEFLQNKPSQTNTVVDYKDWQIPLGRRFRSLKLWMVLRLYGVEKLQCYIRNHINLAKYFEGLIAEDTRFEVVSPPIFALVCFRLLPPDNNVDHGNKLSHDLLDAVNSTGKIFISHTVLSGKYILRFAVGAPLTEERHVTAAWKVLQDEACALLETSRIS

SEQ ID NO: 25 - protein, 486 aa, Cucumis sativus
MDNELKPMDAEQLREHAHKMVDFIADYYKNIEDFPVLSQVEPGYLQNLLPESAPLNPESLQSVLDDVQKKIFPGVTHWQSPNYFAYYPSNSSIAGFLGEMLSAAFNVIGFSWVTSPAATELEMIVLDWLAKLLKLPDDFLSSGNGGGVIQGTASEAVLVVLLAARDRALRRFGKDYLKKLVVYASDQTHSALQKACQIGGIHPENCRWLKADISTNYALSPDVLSEELSRDTARGLIPFFLCATVGTTSSTAVDPLPELGTIAKRHEMWFHVDAAYAGSACVCPEYRQYIDGVEEADSFNMNLHKWFLTNFDCSALWIKDRHALIRSLSTNPEFLKNKASEAELVVDYKDWQIPLGRRFRSLKVWMVLRLYGTENLQKYIRNHISLAERFEALVREDPRFEIVTPRIFSLVCFRLLPSRKNEDGGNRLNQSLLDAVNASGNIFISHTVLSGKYILRFAVGAPLTEEKHINSAWKLLQDVASTLLAI

SEQ ID NO: 26 - protein, 508 aa, Vitis vinifera
MDAEQLRENGHKMVDFIADYYKSIENFPVLSQVEPGYLRELLPDSAPNQPESLQQVFDDLQAKILPGVTHWQSPNFFAYYPSNSSTAGFLGEMLSAGLNIVGFSWITSPAATELEMIVLDWLAKLLNLPDDFLSAGNGGGVIQGTASEAVLVVLLAARDRVLRTVGKTALEKLVVYGSDQTHSALQKACQIGGIHPENCKLLKADSSTGYALSPDLLSEAVSHDITNGLIPFFLCANVGTTSSTAVDPLLELGKVTKSNGIWFHVDAAYAGSACVCPEYRHYIDGVEEADSFNMNAHKWFLTNFDCSVLWVKDRNALVQALSTNPVFLKNKASDANMVVDYKDWQVPLGRRFRSLKLWMVLRLYGVENLQRYIRNHIKLAKQFEELVAQDPRFEIVAPRKFALVCFRLLPPHRNEDFSNKLNHNLLDTVNSTGKVYISHTALSGKYTLRLAVGAPLTEERHVNAAWKVIQEKASVLLSEFGMNGLFDNINLKFILNHQIDISILLNYN

SEQ ID NO: 27 - protein, 486 aa, Citrus sinensis
MDAEQLRENAHKMVDFIADYYKSIENFPVLSQVQPGYLHNLIPDSAPHHPESLQNVLDGYIDIQEKILPGVTHWQSPNYFAYYPSNSSVAGFLGEMLSAGLNIVGFSWITSPAATELEMIVLDWLAKLLKLPEDFLSSGQGGGVIQGTASEAVLVVLLAARDKALKRVGKNSLEKLVVYASDQTHSALQKACQIGGIHPQNFRVLKTDSSTNYSLSPDSLAEAISRDLTIGLIPFFLCATVGTTSSTAVDPLLALGNIAKSNGMWFHVDAAYAGSACICPEYRQYIDGVEEADSFNMNAHKWFLTNFDCSALWVKDRNTLIQSLSTNPEFLKNKASQANMVVDYKDWQIPLGRRFRSLKLWMVLRLYGLENLQGYIRNHIQLAKHFEGLVAQDLRFEVVTPRIFSLVCFRLLPPHNDEDHGNKLNHKLLDDINSTGKIFISHTVLSGKYILRFAVGAPLTEWRHVNAAWEVMQDKASALLARLSIE

SEQ ID NO: 28 - protein, 507 aa, Capsella rubella
MGFCQIELLRHINKHNMQNGSGKNVLKPMDSEQLREYGHRMVDFIADYYKTIEDFPVLSQVQPGYLHQLLPDSAPDHPETLDQVLDDVRAKILPGVTHWQSPGFFAYYPSNSSVAGFLGEMLSAGLGIVGFSWVTSPAATELEMIVLDWLAKLLNLPKEFLSKGNGGGVIQGSASEAVLVVLIAARDKVLRSAGKNALGKLVVYSSDQTHSALQKACQIAGIHPENCRVLETDASTNYALRPELLQEAVSKDLKAGLIPFFLCANVGTTSSTAVDPLAALGKIANSNEIWFHVDAAYAGSACICPEYRKYIDGVETADSFNMNAHKWFLTNFDCSLLWVKEQDSLTEALSTNPEFLKNKASQANLVVDYKDWQIPLGRRFRSLKLWMVLRLYGAETLKSYIRNHIKLAKYFEKLVSQDPNFEIVTPRIFSLVCFRLVPKNDDEKKCNNQNRKLLEAANSSGKLFMSHTALSGKIVLRCAIGAPLTEEKHMKEAWKVIQDEASFLLHK

SEQ ID NO: 29 - protein, 486 aa, Malus domestica
MSGLKPMDAEQLRENAHKMVDFIADYYKTIEDFPVLSQVQPGYLRDLLPDSAPTHPESLQQVFDDIQAKILPGVTHWQSPNFFGYYPSNSSVAGFLGEMLSAGLNIVGFSWITSPAATELEMIVLDWFAKMLKLPEEFLSAGQGGGVIQGTASEAVLVVLLAARDRILRAEGKKALEKLVVYASDQTHSALQKACQIGGIHPENCRVLSTDSSTNYALSPNVLNEAISNDIASGLVPFFLCATVGTTSSTAVDPLLELGKITKSNGMWFHVDAAYAGSACICPEYRHHIDGVEEADSFNMNAHKWFLTNFDCSLLWIKDRNALVQALSTNPEFLKNKASQANLVVDYKDWQIPLGRRFRSLKLWMVLRLYGLENLQSYIRNHIDLAKCFEDLVAQDSRFEIVTPRIFSLVCFRLLPPHNDETYATKLNHDLLDTVNSTGKIFVSHTVLSGKYVLRFAVGAPLTEERHVLAAWKLLQEEASALLAPL

SEQ ID NO: 30 - protein, 483 aa, Linum usitatissimum
MGGYRSLNLIFIFISFVADIRDLGYNTKEGDDGGGALKPMDAEQLRQNAHQMVDFIADYYKNIETYPVLSQVEPGYLRELLPDSAPNRPESLQSVLDDVQSKIMPGVTHWQSPNYFAYYPSNSSVAGFLGEMLSAGINMVGFSWITSPAATELEMIVLDWLGKLLKLPEEFLSSGHGGGVIQGTASEAILVVLLAARDKMLRKFGKSALEKLVVYASDQTHSALQKACQIGGIYPENCRLLKTDSSVNYSLTPELVSEAVSQDISAGLIPFFLCGTVGTTSSATVDPLGTLGKIAKNNDMWFHVDAAYAGSACICPEYRQYLDGVEEADSFNMNAHKWFLTNFDCSTLWVKDKSALIQALSTNPEFLKNKASQANLVVDYKDWQIPLGRRFRSLKLWMVLRLYGVENLQQYLRNHIELARHFEECVNHDPRFEALSGKYTLRVAIGAPLTEKRHVAAALKVLQDEATSLLVATSPLLENGNSS

SEQ ID NO: 31 - protein, 490 aa, Eutrema salsugineum
MENGNKNVLKPMDSEQLREYGHRMVDFIADYYKTIEDFPVLSQVQPGYLHNLLPDSAPDQPETLEEVLDDVKGKILPGVTHWQSPSFFAYYPSNSSVAGFLGEMLSAGLGIVGFSWITSPAATELEMIVLDWLAKLLNLPEQFLSRGNGGGVIQGSASEAELVVLIAARDKVLRSVGKKALEKLVVYSSDQTHSALQKACQIAGIHPENCRVLKADYSTNYALRPETLQEAVSKDLEAGLIPFFLCANVGTTSSTAVDPLAALGEIAKSNEMWFHVDAAYAGSACICPEYRQYIDGVETADSFNMNAHKWFLTNFDCSLLWVKDQYALTEARSTNPEFLKNKASQANLVVDYKDWQIPLGRRFRSLKLWMVLRLYGSENLKSYIRNHIKLAKDFEQLVSEDPNFEIVTPRIFSLVCFRIVPAENDEKKCNNQNRNLLDAVNSSGKLFLSHTALSGKIVLRCAIGAPLTEEKHVKEAWKVIQEEASYLLRK

SEQ ID NO: 32 - protein, 488 aa, Populus trichocarpa
MESKGLQPMDSEQLRENAHKMVDFIADYYKSIENFPVLSQVEPGYLRELLPDSAPNQPETLQNVLDDVQAKILPGVTHWQSPSYFAYYPSNSSVAGFLGEMLSAGINMVGFSWITSPAATELEMIVLDWLGKLLKLPEDFLSTGQGGGVIQGTASEAVLVVLLAARDRVLRKLGKNALEKLVVYASDQTHSALQKACQIGGIHPENCKLLKTGSSTNYALSPDLLGKAISDDISTGLVPFFLCATVGTTSSTAVDPLLSLGKIAKNNGIWFHVDAAYAGSACICPEYRCYIDGVEEADSFNMNAHKWFLTNFDCSALWVKDRNALIQSLSTNPEFLKNKASQANMVVDYKDWQIPLGRRFRSLKLWMVLRLYGLENLQCYIRNHINLAKYFEGLVAADSRFEVVTPRIFSLVCFRLLPPNNNEDHGNNLNHDLLDAVNSTGKIFISHTVLSGKYILRFAVGAPLTEERHVTAAWKVLQDEASALLGSL

SEQ ID NO: 33 - protein, 500 aa, Brachypodium stacei
MDGSTTSNGDGGGGWMRPMDEEQLRECGHRMVDFIADYYKSIETYPVLSQVQPGYLKELLPDSAPNQPDTLDALFDDIQEKIVPGVTHWQSPNYFAYYPSNSSTAGFLGEMLSAAFNIVGFSWITSPAATELEVIVLDWVAKMLKLPSQFLSAGLGGGVIQGTASEAILVVLLSARDRTLRKHGKKSLEKLVVYASDQTHSALQKACQIAGIFSDNIRIVKADCSMNYAVTPGSVSEAISIDLSSGLIPFFICATLGTTSSSAVDPLHELGQIAQAHDMWFHIDAAYAGSACICPEYQQYLNGVEEADSFNMNAHKWFLTNFDCSLLWVKDRNYLIQALSTNPEFLKNKASQENSVIDFKDWQIPLGRRFRSLKLWMVLRLYGVENLQSYIRKHIQLAQRFEQLVISDSRFEVVTPRNFSLVCFCLVPPTSEVDNGHKLNYDLMDSVNSSGKIFISHTVLSGKFVLRFAVGAPLTEEQHVNAAWKLLQDEATKLLGSVVV

SEQ ID NO: 34 - protein, 503 aa, Physcomitrella patens
MGSEAGSRSSLTKPFDPEEFRKHAHRMVDFIADYHRDIENFPVQSQVEPGYLQKLLPENAPDEPESLDDILADVQSKIVPGVTHWQSPNFYGYYPSNGSTAGFLGEMLSGGFNIIGFSWITSPAATELEIIVMDWLGKLLKLPNEFLSSGKGGGVIQGTASEAVLVVMLAARKRAVEKLTKEQGISEFEALAKLVAYTSDQAHSCVNKASQIAGISIENLRLIPTDVSTNYAMSSKVLANTLANDVKAGLVPFFLCGVIGSTSSAAVDPLSELGDLAQEYGMWFHVDGAYAGNACICPEFRPYLNGVEKADSFDMNPHKWLLTNFDCSTLWVKNPSLLVDALSTNPVFLRNKQSDNNLVVDYKDWQIPLGRRFRSLKLWMVLRMYGSNGLRSYITNHCNLAKHFEELLRTDSRFEVVAPRVFSLVCFRLKSPANDADNSCSLSAKLVDALNSDGNILITNTVLGGRYTIRFTVGASRTELRHVDAAWKVIQQLASKLLKECSS

SEQ ID NO: 35 - protein, 487 aa, Ananas comosus
MESELKPMDSEQLREYAHKMVDFIADYYKMIESFPVLSQVKPGYLKELLPDSAPCKPENLEDVFDDIRQKIIPGITHWQSPDYFAYYPSNSSTAGFLGEMLSAGFNIIGFSWIASPAATELEMIVLDWFAKMLKLPEQFLSTGQGGGVIQGTASEAVLVVLLAARDKILLKAGRKSLEKLVVYCSDQTHSAMQKACQIAGIFPENFRVLKTDSSSNYALLPEVLSEAISKDLSFGLIPFFLCATVGTTSSAAVDPLLKLGNISKVHDMWFHVDAAHAGSACICPEYRHHIDGVEEADSFCMNAHKWFLTNFDCSLLWVKDRSALIQSLSTNPEFLKNKASQENSVVDFKDWQIPLGRRFRSLKLWMVLRLYGLENLQSYIREHIKLAEQFEQLISSDSRFEIVAPRTFSLVCFRLLPPLYDQDDGYKLNYNLLDAVNRSGKIFMSHTVLSGKFVLRFAIGAPLTEERHVVAAWKVLQDEATILLRGS

SEQ ID NO: 36 - protein, 511 aa, Zostera marina
MLNGNMGENEPFKPMDSEQLREYGHKMVDFIADYYKSIEKFPVLSQVQPYYLKDLLPDAAPDQPEKFQDVLDDITKKIIPGVTHWQSPNFFGYYPGNSSIAGFLGEMICSGLNVIGFSWITSPASTELEVIVLDWLAKLLNLPDQFLSSGHGGGVIQGTASEAILVVLLAARDKILGRIGRNSLDKLVVYSSDQVHAAFKKACQIAGIYTENFRVLKTDASSGYGIDPKKFDQAIHDDMEAGLIPFFLCSTVGTTSSASVDPLVEIGQITEENDMWFHVDAAYAGSACICPEYRHYLDGVEYADSFCMNAHKWLLTNFDCSALWVKDSSALVNSLSTNPEFLKNKMSEQKKVVDFKDWQIPLGRRFRSLKLWMVLRLYGAENLREYIRNHIKLANLFEQLVRSDSRFEIVCPTLFSLVCFRFLPSNDDNDGYELNSMLLDAVNSTGQLFFTHTIISDKYILRFAVGAALTEERHVRESWKVIQNQATIISRQHILSKTNMKSKCEGMIANE

SEQ ID NO: 37 - protein, 497 aa, Daucus carota subsp. sativus
MDGVLKPMDAEQLRENAHKMVDFIADYYKNIETFPVLSQVEPGYLRDLLPHSAPDQPESLQNILDDIQAKILPGVTHWQSPNYFAYFPSNSSVAGFLGEMLSAGINMVGFSWITSPAATELEMIVLDWLAKLLKLPDHFLSTGQGGGVIQGTASEAVLVVLLAARDKVLRITGKDALGKLVVYCSDQTHSALQKACQIAGIHPGNCRVLKTESCNDYSLSPETFEQAISTDVASGLIPLLLCATVGTTSSTAVDPLLELGKITKMKGIWLHVDAAYAGSACVCPEFRHYIDGVEEADSFNMNAHKWFLTNFDCSALWVKDRSALIHSLSTNPEFLKNKASQENLVVDYKDWQIPLGRRFRSLKLWMVLRLYGLENLQSYIRNHIQLAATFESFVTEDPRFEVVAPRKFALVCFRLLPPSHKDEDCSNQLNRDLLDAVNATGKAFVSHTALSGRYVVRFAIGAPLTEESHIIEAWKIFQEVATVLLKSLKMNHTRPLN

SEQ ID NO: 38 - protein, 468 aa, Trifolium pratense
MVDFIADYYKTIENFPVLSQVEPGYLGKLLPDSAPTYPTTLEHVLNDVQHKILPGVTHWQSPNYFAYFPSNSSIAGFLGEMLSAGINIVGFSWITSPAATELESIVLDWLAKALFLPQDFLSNGKGGGVIQGTASEAVLVVLLAARDKILRTVGRSALPKLVTYASDHVHSSLLKACQIGGLDPELCRLLKTDSSTNFALSPDVLSEAISNDIASGLIPFFLCANVGTTSSTAVDPLPALAKVTKTNNIWLHVDAAYAGSACICPEYRHFIDGVEEADSFNMNAHKWFLTNFDCSLLWVKDRSALIQSLSTNPEFLKNKASEGNMVIDYKDWQIPLGRRFRSLKLWMVLRLYGLEGLRSHIRNHIALAASFEELVVQDARFKVVTPRTFSLVCFRLLPPPNSEDNGNKLNHDLLDLVNSTGSVFITHTVLSGEYILRLAVGAPLTEVRHVNAAWQILQEKATALLENL

SEQ ID NO: 39 - protein, 475 aa, Arabidopsis lyrata
MDSEQLREYGHRMVDFIADYYKTIEDFPVLSQVQPGYLHKLLPDSAPDHPETLDQVLDDVRAKILPGVTHWQSPSFFAYYPSNSSVAGFLGEMLSAGLGIVGFSWVTSPAATELEMIVLDWLAKLLNLPEQFMSKGNGGGVIQGSASEAVLVVLIAARDKVLRSVGKNALQKLVVYSSDQTHSALQKACQIAGIHPENCRVLKTDSSTNYALRPELLQEAVSQDLDAGLIPFFLCANVGTTSSTAVDPLAALGKIANRNEMWFHVDAAYAGSACICPEYRQYIDGVETADSFNMNAHKWFLTNFDCSLLWVKDQDSLTLALSTNPEFLKNKASQANLVVDYKDWQIPLGRRFRSLKLWMVLRLYGSETLKSYIRNHIKLAKEFEQLVSQDPNFEIVTPRIFSLVCFRLVPVKNEEKKCNNRNRELLDAVNSSGKLFISHTVSDFSSFFLLFFLLDNVLNLRGNRLCRGKSYCVAQ

SEQ ID NO: 40 - protein, 505 aa, Sorghum bicolor
MDGSGSSGGTNGGSGGDGAGWLRPMDAEQLRECGHRMVDFVADYYKSIETFPVLSQVQPGYLKELLPDTAPNKPDTLEALFDDIREKIVPGVTHWQSPNYFAYYPSNSSTAGFLGEMLSAAFNIVGFSWITSPAATELEVIVLDWFAKMLRLPSQFLSTALGGGVIQGTASEAVLVVLLAARDRTLRKHGKTSLEKLVVYASDQTHSALQKACQIAGIFPENVRLVKADCNRNYAVAPLAISDAIATDLSSGLIPFFICATVGTTSSSAVDPLPELGQIAKANDMWLHIDAAYAGSACICPEYRHHLNGVEEADSFNMNAHKWFLTNFDCSLLWVKDRSYLIQSLSTNPEFLKNKASEANSVFDFKDWQIPLGRRFRSLKLWMVLRLYGVENLQSYIRKHIELAKEFEQLVISDSRFEVVTPRTFSLVCFRLVPLASDQDNGRKLNYDLMDAANSSGKIFISHTVLSGKFVLRFAVGAPLTEGQHIFSAWKILQDLATKQLLESS

SEQ ID NO: 41 - protein, 504 aa, Sphagnum fallax
MGSEAGEGSRLSKPLDVEEFRKHAHQMVDFVADYHRDIESFPVRSQVKPGYLRPLLPDSAPAEPETVEDVFADLWSKILPGLTHWQSPKFFGYYPCNVSTAGMLGEMLCGGLNVNGFSWITSPAATELETIVLDWLGKLLHLPEEFLSTSGKGGGVIQGTASEAVLVVMLAARKRALKQVSSAAQGMSEAEALSKLVVYSSDQTHSCVIKACQVASIATENFRPLPTDASTNFALSPAVVRKAIATDVEAGLIPFFLCGTLGTTSSAAVDPLEELGDIAKEYGMWYHIDAAYAGNACICPEFRHYLNGVEKADSYNMNPHKWLLTNFDCSTLWMKDSEFLLAALSNKPVFLRNEATDNNLVVDYKDWQIPLGRRFRALKLWMVMRLYGTSGLQSFIRSHVSSAKHFESLVRADSRFEVMAPMTFSLVCFRLRTLPGSQDNSNSLNSKLVDALNRKGNILVTHTELSGIYTVRFAVGATHTELQHVQAAWEVIQAEASHLLNGKQ

SEQ ID NO: 42 - protein, 524 aa, Kalanchoe laxiflora
MILSIHPFPFTLSARFSGAAAANILSKASCWLRCLRSMEGELKPMDAEQLREYGHRMVDFVADYYKTIEDHPVLSQVEPGYLRKLLPDSAPDKPESFENVLSDVKTKIIPGVTHWQSPNYFAYFPSNSSTAGFLGEMLSACFNIVGFSWITSPAATELEMIVLDWFAKMLKLPDFFLSTGQGGGVIQGTASEAVLVVLLAARDIFLRKLGKGFLEKLVVYASDQTHSALQKACQIAGIHPENVRALKTDSSTNYGLSPDLLSKEICHDIANGLVPFFACASVGTTSSTAVDPILELANVTKSYNIWLHVDSAYAGSACVCPEYRHHIDGVEEVDSFNMNAHKWFLTNFDCSLLWVKDRNALIQSLSTNPEFLKNKASQSNSVLDYKDWQIPLGRRFRSLKLWLVLRLYGVENLQAYIRNHIELALNFEELVSQDMRFEIVAPRTFALVCFRLLLPCGFEDHTNDVNSDLLQAVNSTGKIFISHTVLSGTYVLRFAVGAPLTEERHIDAAWKLIQDQASSLLEKL

SEQ ID NO: 43 - protein, 487 aa, Manihot esculenta
MEGELRPMDAEQLREYGHQMVDFIADYYKTIENFPVLSQVEPGYLHKLLPDSAPNQPEALQNVLDDVRVKILPGVTHWQSPNYFAYYPSNSSVAGFLGEMLSAGINMIGFSWITSPAATELEMIVLDWLGKMLKLPEEFLSSGQGGGVIQGTASEAVLVVLLAARDKVLTRVGKDSLKKLVVYGSDQTHSALQKACQIAGVHLDNCRLLKTDSSKNYALSPDILCDAISQDMSNGLIPFFLCATVGTTSSATVDPLLALGKIAKKYGMWFHVDAAYAGSACICPEYRCYIDGVEEADSFNMNAHKWFLTNFDCSALWVKDRNALIQSLSTNPEFLKNKASQANMVVDYKDWQIPLGRRFRSLKLWMVLRLYGVANLQSYIRNHINLAKYFEGLVAGDSRFEVVAPRLFSLVCFRLLPPDNDENHGNKLNHDLLDAANSTGKIFISHTVLSGKYILRFAVGAPLTEERHVTAAWKVLQDEASALLGSL

SEQ ID NO: 44 - protein, 487 aa, Prunus persica
MESGLKPMDAEQLRENAHKMVDFIADYYKTIENFPVLSQVQPGYLRELLPDSAPTHPEPLQHIFDDIQAKILPGVTHWQSPNFFGYYPSNSSIAGFLGEMMSAGLNIVGFSWITSPAATELEMIVLDWFGKMLKLPEEFLSAGKGGGVIQGTASEAVLVVLLAARDKILRRVGKNSLEKLVVYASDQTHSALQKACQIGGIHPENCRLLRTDSSTNYALSPNVLNEAISNDVTSGLIPFFLCATVGTTSSTAVDPLLELGKIAKSNDMWFHVDAAYAGSACICPEYRHYIDGVEEADSFNTNAHKWFLTNFDCSVLWIKDRNALIQALSTNPEFLKNKASQANLVVDYKDWQIPLGRRFRSLKLWMVLRLYGLENLQSYIRNHINLAKHFKELVAQDPRFEIVTPRLFSLVCFRLLPPHNDETCATKLNHGLLDAVNATGKIFISHTVLSGKYLLRLAVGAPLTEERHVNAAWKLLQDEASALLATL

SEQ ID NO: 45 - protein, 490 aa, Eucalyptus grandis
MEERLKPMDAEQLRESAHRMVDFIADYYKSIESFPVLSQVEPGYLRKLLPDSAPDHPESLQQVLEDVQAKILPGVTHWQSPNYFAYYPSNSSIAGFMGEMLSAGLNIVGFSWITSPAATELEIIVLDWLAKLLNLPDDFLSTGPGGGVIQGTASEAVLVVLLAARDKFLSRIGKSSLDKLVVYSSDQTHSALQKACQIGGIYPENCRVLKTDASTNYALSPDLLNEVISQDISTGLVPFLLCATVGTTSSTAVDPLPALATVAKRNGMWFHIDAAYAGSACICPEYRPYIDGVEEADSFNMNAHKWFLTNFDCSALWIKDRKALIQALSTNPEFLKNKASQANMVVDYRDWQIPLGRRFRSLKLWMVLRLYGVQNLQQYIRNHIELARQFEDLVIQDPRFEVVTPRIFSLVCFRLLSPDNDGDKGNKLNRDLLDTVNSTGKIFISHTVLSGTYILRFAVGAPLTEERHVNEAWKVLQDEASKLLATIQNN

SEQ ID NO: 46 - protein, 480 aa, Amborella trichopoda
MDAEELREHGHRMVDFISDYYKEIESYPVRSQVQPGYLRNLIPDSAPDMPESFESILEDIRHKIIPGVTHWQSPKYFAYYPSNSSTAGFLGEMLSAGFNIVGFSWVTSPAATELEVIVLDWLAKVLKLPEQFLSTGKGGGVIQGTASEAMLVALLAARDKALRRVGQNLLENLVVYGSDQTHSALIKACKIAGINPMNCRLLQATFMTNYALSPEVASESISNDIAAGLLPIFLCATVGTTSSTAVDPLAALGRLAKANDMWFHIDAAYAGSACICPEYRHYIDGVEEADSFNMNPHKWLLTNFDCSTLWVKDSSNLIQSLSTNPEFLRNKASEEDLVVDYKDWQIPLGRRFRSLKLWMVLRMYGVANLQNHIRTHINLAKHFEELIATDTRFEIIVPRVFALVCFALKPMPNGQDDASKLNLKLLEAVNNSGAMFLTHTVLSGRFVLRFVVGAPLTEERHVNTAWKVLQDHANLILGTV

SEQ ID NO: 47 - protein, 488 aa, Salix purpurea
MESKGLKPMDSEQLRENAHKMVDFIADYYKSIENFPVLSQVEPGYLRELLPDSAPNQPETLQNVLDDVQAKILPGVTHWQSPSYFAYYPSNSSVAGFLGEMLSAGINMVGFSWITSPAATELEMIVLEWLGKLLKLPEDFLSTGQGGGVIQGTASESVLVVLLAARDRVLTKLGKNALEKLVVYASDQTHSALQKACKIGGIHPENCKLLKTDSSTNYALSPDLLSKAISDDISTGLIPFFLCATVGTTSSTAVDPLHALGKIAKNNGIWFHVDAAYAGSACICPEYRCYIDGVEEADSFNMNAHKWLLTNFDCSALWVKDRNALIQALSTNPEFLKNKASQANMVVDYKDWQIPLGRRFRSLKLWMVLRLYGLENLQCYIRNHINLAKYFEGLVAADSRFEVVTPRIFSLVCFRLLPPSNNEDHGNNLNRDLLDAVNSSGKIFISHTVLSGKYILRFAVGAPLTEERHVIAAWKVLQDESTSLLGSL

SEQ ID NO: 48 - protein, 532 aa, Medicago truncatula
MVLQIWCLTHDSDKKLGGGYLLFPVIKVAYTVHTLTEWCCVTEEGGGSELKAMDAEQLREQGHMMVDFIADYYKTIENFPVLSQVQPGYLGKLLPDSAPTHPESLQHVLNDVQEKILPGVTHWQSPNYFAYFPSNSSIAGFLGEMLSAGLSIVGFSWISSPAATELETIVLDWLAKALLLPHDFFSTGQGGGVIQGTASEAVLVVLVAARDKILRTVGRSALPKLVTYASDQTHSSLQKACQIAGLNPELCRLLKTDSSTNFALSPDVLSEAISNDIASGLTPFFLCATVGTTSSTAVDPLPALAKVTKPNNIWLHVDAAYAGSACICPEYRHFIDGVEEADSFNMNAHKWFLTNFDCSVLWVKDRSALIQSLSTNPEFLKNKASQENTVIDYKDWQIPLGRRFRSLKLWMVMRLYGLEGLRTHIRSHIALAVYFEELVVQDTRFKVVAPRTFSLVCFRLLPPQNSEDNGNKLNHDLLDAVNSTGDVFITHTVLSGEYILRLAVGAPLTEVRHVHAAWQILQEKATALLESL

SEQ ID NO: 49 - protein, 501 aa, Brassica rapa
MQIRAKIPVFGRENGSRHVLKPMDSEQLREYGHRMVDFIADYYKTIESFPVLSQVQPGYLHNLLPDSAPDHPETVEQVLDDVKTKILPGVTHWQSPNFFAYYPSNSSVAGFLGEMLSAGVGIVGFSWVTSPAATELEMIVLDWLAKLLNLPEHFLSKGNGGGVIQGSASEAILVVMIAARDKVLRSAGKNALGKLVVYSSDQTHSALQKACQIAGIHPENCRVLKADSSTNYALRPELLQEAVSRDLEAGLIPFFLCGNVGTTSSAAVDPLAALGKIAKSNEIWFHVDAAYAGSACICPEYRQYIDGVETADSFNMNAHKWFLTNFDCSLLWVKDQHALTEALSTNPEFLKNKASQANLVVDYKDWQIPLGRRFRSLKLWMVLRLYGAEALKNYIRNHIKLAKDLEQLVSQDPNFEVITPRIFSLVCFRIVPTDNDEKKCNSRNLELLEAVNSSGKLFISHTALSGKIVLRCAIGAPLTEEKHVKETWKVIQEKVSYLLRK

SEQ ID NO: 50 - protein, 479 aa, Brassica rapa
MDSEQLREYGHRMVDFIADYYKTIETFPVLSQVQPGYLHNLLPDSAPDQPETVEQVLDDVKTKILPGITHWQSPTFYAYYPSNSSVAGFLGEMLSAGLGIVGFSWVTSPAATELEMIVLDWLAKLLNLPEQFLSKGNGGGVIQGSASEAILVVMIGAREKVLRRVGKNALGKLVVYSSDQTHSALQKACQIAGIHPENCRVLKADSSTNYALRPELLQEAVSKDIEAGLIPFFLCGNVGTTSSTAVDPLAALGKIAKSNEIWFHVDAAYAGSACICPEYRQYIDGVETADSFNMNAHKWFLTNFDCSLLWVKDQYVLTEALSTNPEFLKNKASQANLVVDYKDWQIPLGRRFRSLKLWMVLRLYGAETLKSYIRNHIKLAKDLEQLVSQDPNFEVVTPRIFSLVCFRILPVDNDEKECNNRNRNLLDAVNSSGKLFLSHTALSGKIVLRCAIGAPLTEERHVKETWKVIQEEASRLLGK

SEQ ID NO: 51 - protein, 482 aa, Brassica rapa
MDSEQLREYGHRMVDFIADYYKTIETFPVLSQVQPGYLHNLLPDSAPDQPETLEQVLDDVKEKILPGVTHWQSPSFFAYYPANSSVAGFLGEMLSAALNIVGFSWVSSPAATELEMIVLDWFAKLLNLPEQFLSRGNGGGVIQGTASEAILVVMIAARDKVLRSLGKKALEKLVVYSSDQTHSSLLKACQIAGIHLENCRMLKTDSSTNYALRPESLQEAVSGDLEAGLIPFFLCGTVGTTSSTAVDPLAELGKIAKSNEMWFHVDAAYAGSACICPEYRQYIDGVETADSFNMNAHKWFLTNFDCSLLWVKDRYALTEALSTNPEFLKNKASQANLVVDYKDWQIPLGRRFRSLKLWMVLRLYGAETLKSYIKNHIKLAKDLEQLVSQDPNFEVVTPRIFSLVCFRIVPVDNDEKTCNNLNRSLLDAVNSSGKLFISHTTLSGKFVLRLAIGAPLTEEKHVMDAWKVIQEEASFLLASQVK

SEQ ID NO: 52 - protein, 489 aa, Glycine max
MEEESALRPMDAEQLREQAHKMVDFIADYYKTIEDFPVLSQVQPGYLGKLLPDSAPDSPESLQNVLDDVQEKILPGVTHWQSPNYFAYFPSNSSIAGFLGEMLSAGLNIVGFSWITSPAATELETIVLDWLAKAFQLPDYFYSSGKGGGVIQGTASEAVLVVLLAARDKILRRVGRNALPKLVMYASDQTHSALLKACQIAGINPELCRLLKTDSSTNYALSPDVLSEAISNDIAGGLVPFFLCATVGTTSSTAVDPLPALGKIAKTNKLWFHVDAAYAGSACVCPEYRHCIDGVEEADSFNMNAHKWFLTNFDCSLLWVKDRSSLIQSLSTNPEFLKNKASQGNMVIDYKDWQIPLGRRFRSLKLWMVLRLYGLDGLRSHIRNHIELAANFEELVRQDTRFKVVAPRTFSLVCFRLLPHPNSADHGNKLNSDLLDSVNSTGNAFITHTVLSGEYILRFAVGAPLTERRHVNMAWQILQDKATALLESL

SEQ ID NO: 53 - protein, 520 aa, Fragaria vesca
MDAEQLRENAHKMVDFIADYYKTIEDFPVLSQVQPGYLRELLPDSAPTQPESLQHIFDDIQAKILPGVTHWQSPNFFAYYPSNSSIAGFLGEMLSAGLNIVGFSWVTSPAATELEMIVLDWLAKLIKLPDEFLSAGQGGGVIQGTASEAILVVMLAARDKILRRVGKNALEKLVVYASDQTHSALQKACQIAGIHPENCRILSTNSTTNYALSPSVGTTSSTAVDPLGELGKIAKNNEMWFHVDAAYAGSACICPEYRHYIDGVEKADSFNMNAHKWFLTNFDCSVLWIKDRNALVQSLSTNPEFLKNKASQANMVVDYKDWQVPLGRRFRSLKLWMVLRLYGLENLQSYIRTHINLAKHFEELVAQDPRFEIVTPRLYSLVCFRLLPPHGNEACASKLNHDLLDAVNSTGKIYISHTVLSGAYILRFAVGAPLTEEKHVTAAWKKLKSVIRDVLALANSFVSITFSHMYREANFLTDALASVGHSLSSSMCWFDGIPPQAQMALLMDSSCIGHLRGSSL

SEQ ID NO: 54 - protein, 487 aa, Kalanchoe fedtschenkoi
MEGELKPMDAEQLREYGHRMVDFVADYYKTIEDHPVLSQVEPGYLRKLLPDSAPDKPESFENVLSDVKTKIIPGVTHWQSPNYFAYFPSNSSTAGFLGEMLSACFNIVGFSWITSPAATELEMIVLDWFAKMLKLPDFFLSTGQGGGVIQGTASEAVLVVLLAARDIFLRKLGKGFLEKLVVYASDQTHSALQKACQIAGIHPENVKALKTDSSTNYGLSPDLLSKEICHDIANGLVPFFACASVGTTSSTAIDPILELANVTKSYNIWLHVDSAYAGSACVCPEYRHHIDGVEEVDSFNMNAHKWFLTNFDCSLLWVKDRNALIQSLSTNPEFLKNKASQSKSVLDYKDWQIPLGRRFRSLKLWLVLRLYGVENLQAYIRNHIELAIHFEELVSQDMRFEIVAPRTFALVCFRLLLPCGFEDRTNDVNGDLLQAVNSTGKIFISHTVLSGTYVMRFAVGAPLTEERHIDAAWKLIQDQASSLLEKL

SEQ ID NO: 55 - protein, 479 aa, Capsella grandiflora
MDSEQLREYGHRMVDFIADYYKTIEDFPVLSQVQPGYLHKLLPDSAPDQPETLDQVLDDVRAKILPGVTHWQSPGFFAYYPSNSSVAGFLGEMLSAGLGIVGFSWVTSPAATELEMIVLDWLAKLLNLPKEFLSKGNGGGVIQGSASEAVLVVLIAARDKVLRSAGKNALGKLVVYSSDQTHSALQKACQIAGIHPENCRVLETDASTNYALRPELLQEAVSKDLKAGLIPFFLCANVGTTSSTAVDPLAALGKIANSNEIWFHVDAAYAGSACICPEYRKYIDGVETADSFNMNAHKWFLTNFDCSLLWVKEQDSLTEALSTNPEFLKNKASQANLVVDYKDWQIPLGRRFRSLKLWMVLRLYGAETLKSYIRNHIKLAKYYEKLVSQDPNFEIVTPRIFSLVCFRLVPKNEDEKKCNNQNRKLLEAANSSGKLFMSHTALSGKIVLRCAIGAPLTEEKHMKEAWKVIQDEASFLLHK

SEQ ID NO: 56 - protein, 527 aa, Selaginella moellendorffii
MGEANIGPKPIDAEEFRKHAHEMVDFIADYYRDIESFPVRSQVSQPGYLKTLLPPAAPEDPEALEEVFADIQSKIIPGVTHWQSPNFFGYYPSNSSTAGLLGEMLSAGLNIVGFSWITSPAATELEIIVLDWLAKLLKLPDEFLFGGNGGGVIQGTASEAVSVVLLAARTRAISENKRKGLSEAEILSKLAVYTSDQTHSCLQKGCAIAGIPLENLVIVPTDSSTNYAVSPAAMRQALEDGVKQGLLPFFLCGTVGTTSSSAVDPLSALGDIAKDFGMWFHVDAAYAGSACICPEFRHHLDGVEKADSFNMNAHKWLLTNFDCSALWVKESSHLVSALSTTPEFLRNKASDLNQVVDYKDWQIPLGRRFRSLKLWFVMRMNGASGLRSYIRNHVRLAKRFEGFVREDPRFQLLVPRTFGLICFRLKPESDDPDNGRTLNSTLLEAVNSSGRMFITHTVLSGVYTLRMAIGGPLTQDKHVDAAWKLIQEEATTLLVKGPSHILANNLRLSPILANNLRLSPILANNRI

SEQ ID NO: 57 - protein, 572 aa, Setaria italica
MDILNHADTTTANGTSPAAAAAAAVVAPATPSSLVTPPLDADEFRRQGRLVVDFIADYYTRINEYPVRPAVAPGFLARQLPETAPARPERDALAAALRDVRDLILPGVTHWQSPRHFAHFAATASNVGALGEALAAGLNINPFTWAASPAATELEVVVTDWLGKALHLPERLLFSGGGGGTLLGTSCEAMLCTIVAARDRKLAEIGEERIGDLVVYFSDQTHFSFQKAARIAGIRRGNCREIPTSRESGFTLSPKALRAAVRADEASGRVPLFLCATVGTTPTAAIDPLRELCAAVSGHGVWVHVDAAYAGAACVCPEFRHAIAGAEAVDSFSTNPHKWLLANMDCCALWVTRPAALVAALGTDHDVILKDPSAAAQDGHDVVVDYKDWQVALSRRFRALKLWLVLRCHGVEGLRGFVRAHVRMAAAFEAMVRADTRFEVPVPRQFALVCFRLRPASAGEKRTRGGEVVEPNELNRRLLEAVNATGRAYISSAVVGGVYVLRCAIGNSLTEERHVREAWSVVQEQANVVLAAATATCPDERAVHRARCVETDAADAPASVPPVQMRFPSAQS

SEQ ID NO: 58 - protein, 505 aa, Kalanchoe fedtschenkoi
MGSLPSPHDPSNAFNPMDVAELSIESRLVMDFITQYYQTLETRPVQPRVKPGFLTGQLPEKAPFHAESMEEILSDVSEKIVPGLTHWQSPNFHAYFPASSSNAGLLGEMLCSGLSVIGFTWNSSPAATELENVVVDWLADMLNLPPSFRFSGGGGGGGVLQSNTCEAVLCTLAAARDKVLERIGDDKINKLVAYCSDQTHFTLHKGAKLIGIRRANIKSIGTRRENGFGLCPNDLRNAITGDLEAGLVPFYLCGTIGTTALGAVDPIKELGKVAREFDLWFHIDAAYGGSACICPEFRHYLDGVELVDSISMNAHKWLLSNLDCCFLWLQNPKCLIQCLAAEAEFLKGSGEMVDYKDWQISLSRRFRAIKMWMVFRRYGVSNLMEHIRSDVSMAARFEEMVSADDRFEIVFPRKFALVCFKLNTKGSVQHGEDDGEDGLDGDSVLTRELMGRVNSSGKAYLSGVEMGRIFFIRCVIGSSLTEERHVDNLWNLIQEKTQSIMPCRA

SEQ ID NO: 59 - protein, 493 aa, Daucus carota subsp. sativus
MGSLSTQKFNPLNLDFFSSESNKVIEFITAYYKNVEKYPVRSQVEPGFLLNMYPKKAPSQPVSLDTILQELEADIIPGMTHWQSPNFYAYFRTTTSNAAFQGEMLCNALNVAGFNWICSPAATELEMIVMDWLGKMLSLPQSFLFAGNGGGVLQGSTSEALICVLSAARDRALKQYGEDSITKLVVYASDQTHFVVKKAAKLVGIPTKNFRVIPTSIATCFALKPNDIKMAIERDLESGLVPLFVCATVGATPSGSVDPVEGLGLLAKNYGLWLHIEAAYAGSAFICPELTHYLRGIEHAHSISINLHKWLLTNMDCSCLWVKSPDVLLESLSMTDEILRNEASESKKVVDFMDWQIATSKLFRALKLWFVLRRYGVDNLMAHIRSDIELAKHFEALVNSDKRFEVVVPVNFSLVCFRLKPNEEGEESLKVLMNWNLMEAVNSSGRAYMTHAVLGDIFVIRCAIGTSLTEERHVNELWKLILEKTEVILKRDQ

SEQ ID NO: 60 - protein, 441 aa, Daucus carota subsp. sativus
MNTFDTEDFRKQAHLIIDFLADYYQNIEKFPVRSQVSPGYLGEILPDSAPHDPEPIEKILEDVRSNIIPGITHWQSPNFFAYFPSCGSTAGFLGEMLANGFNVVGFNWISSPAATELETIVMDWLGKMLQLPEAFLFSGGGGGVLQGTTCEAMLCTLVAARDRTLREQGMENFDKLLCPVQLELEILSDVQNGLIPLFLCVTIGTTPSTAVDPLATLSEVAKKYKLWVHVDAAYAGSACICPEFRHFLDGLENVNSFSMNAHKWFLTTLDCCCLWVNDPSALIKSLSTYPEFLRNHASESNKVVDYKDWQIMLSRRFRALKLWFVLRSYGVEKLREFIRVHVEMAKYFEGLVAMDQRFEVVVPRLFAMVCFRVVCCGENDVNEINEKLLESVNQSGRIYVSHAVLDGVYVIRFAIGATLTDYSHVSAAWEVVQEHADALLA

SEQ ID NO: 61 - protein, 510 aa, Solanum tuberosum
MGTLNINHELDDQIFNTINPLDPEEFRRQGHKIVNFLADYYQNIEQYPVCSQVNPGYLQKIVPNSAPNNSESLEKILKDVERDIIPGLTHWQSPNFFAYFPSSGSTAGFLGEMLSVGFNVVGFNWISSPAATELESIVMDWFGKMLNLPNCFLFASGGGGVLQGTTCEAMLCTIVAARDQMLRKISRENFGKLVVYASDQTHFSLKKAAHIAGIDPGNFRVIPTIKANEYTLCPKSLRLAILNDLKEGNVPLFLCATIGTTATTSVDPLRLLCEIAKEFGIWVHVDAAYAGSACICPEFQVFLDGVENANSFSLNAHKWFFSTLDCCCLWVKDPSALTNALSTNPECLRNKATELNQVIDYKDWQIALSKRFRALKLWLVLRSYGVTNLRNLIRSHVNMAKHFEGLVATDKRFEIFVPRKFAMVCFRISPLVLSQVSTKFDDEKEVNMFNTKLVESINSCGKLYLTHGVVGGTYIIRFAIGASLTHYRHVDVAWKVIQDHANALLNQGYV

SEQ ID NO: 62 - protein, 507 aa, Solanum tuberosum
MGTMKINPEHEFDGQFSINTSSSRLLDPEEFRRQGHMMVDFLADYFQNIEKYPVRSQVEPGYLKKLLPDSAPYKPEPIAKILEDVERDIFPGLTHWQSPNFFAYFPCTSSTAGILGEMLSAGLNVVGFSLIASPAATELESIVMDWLGKMISLPKTYLFSGGHGGGGVIQGTTCEAMLCTIVAAREQMLEKVGREKVDKLVVYASDQTHFSFEKAVKISGIKLENFRVIPTTKDTEFALDPKSLSRTIEQDIKSGFIPLFMCATIGTTSTTVVDPLKLLCEITKDYGIWVHVDAAYAGGACICPEFQHFLDGIENANSFSFNAHKWLFSNLDCCCLWVKDPSALTNALSTRPECLRNKATDTKQVVDYKDWQLSLSRRFRALKLWLVLRSYGIDNLRNFIRSHVKMAKHFEQLVSMDERFEIVAPRNFSMVCFRVSPLALGNKQVNKFNMELLESINSCGNIHMTHALVGGVYMIRFAIAAPLTEYKHIDMAWEVICNHANAMLDVN

SEQ ID NO: 63 - protein, 476 aa, Solanum lycopersicum
MGTLNINHELDDQIFNTINPLDPEEFRRQGHKIVNFLADYYQNIEQYPVCSQVNPGYLQNIVPNSAPNNPESLDKILKDVQNDIIPGLTHWQSPNFFAYFPSSGSTVGFVGEMLSVGFNVVGFNWISSPAATELESIVMDWFGKMLNLPNCFLFASGGGGVLQGTTCEAILCTIVAARDQMLRKISRENFGKLVVYASGQTHFSLKKSAHIAGIDPGNFRVIPTIKAKEYTLCPKSLRLAILNDLKEGNVPLFLCATIGTTSTTSVDPLRLLCDISKEFGIWVHVDAAYVGSACICPEFQVFLDGVENANSFSLNDPSALTNALSTNLEFLRNKATELNQVIDYKDWQIALSRRFRALKLWLVLRSYGVTNLRNLIRSHVNMTKHFEGLIAMDKRFEIFVPRKFAMVCFRISPLVLSQVSIKFDDEKEVNMFNTKLLESINSCSKLYLTHGIVGGTYIIRFAIGASLTHYRHVDIA

SEQ ID NO: 64 - protein, 440 aa, Daucus carota subsp. sativus
MCKPKSSPASHINWQSPNFFAYFPSSGSTAGFLGEMLSTGFNVVGFHWMASPAATELENVVTDWFGKMLQLPKSFLFSGGGGGVLQGTTCEAMLCTLVAARDKNLRQHGMENIGKLVVYCSDQTHSAMQKAAKIAGIDPKNFRTVETSRASNFQLCPRRLESAILTDIQNGLIPLYLCATVGTTSSTAVDPLPALTEVAKKYDLWVHVDAAYAGSACICPELRQYLNGVENADSFSLNAHKWFLTTLDCCCLWVKNPSALIKSLSTYPEFLRNNASETNKVVDYKDWQIMLSRRFRALKLWFVLRSYGVGQLREFIRGHVDMAKYFEGLVGKDKRFEVVVPRLFSMVCIRVRPSAMTGKSCGNDVNELNRKLLESLNESGRIYVSHTVLDGIYIIRFAIGATLTDINHVSAAWKVVQDHATALLDDTNFLAKKVADIILS

SEQ ID NO: 65 - protein, 577 aa, Oropetium thomaeum
MAILNHADDASPANDDNPATAPAMAPATNPRPLDADEFRRQGRLVVDFIADYYARVEEYPVRPSVTPGFLSRKLPETAPEQPEPGHGDAFASALRDVRDLILPGITHWQSPNHFAHFAATASNVGALGEALAAGLNINPFTWAASSAATELEVVVTDWLGKALHLPQELLFSGGGGGTLLGTSCEAMLCTVVAARDRKLGEIGEHRIGDLVVYCSDQTHFSFRKAARVAGIRRANCREIPTSLESDFALSPSALLAAVRADEAAGLVPLYLCVTVGTTPTAAVDPVRELCAAVAGRGVWVHVDAAYAGAARVCPELLRHAGAIVDGVDSFSTNPHKWLLANMDCCALWVQQPDALVAALGTDHDVILKDPAAAAAGDVVVDYKDWQVALSRRFRALKLWLLLRCHGVEGLRAHVRDGLRMAEAFEAMVRADARFEVPVRRQLSLVCFRLRPTAVIREKQQQQRGRRRDHDDDTAAANELNRRLLEAVNATGRTYMSCAVVGGVYMLRCAIGNSLTEDRHVEEAWNVVQEQASAILDAAMVVRADECTVCTAAHCVQMGMVDDILAASFPTGNEVTIR

SEQ ID NO: 66 - protein, 565 aa, Oryza sativa
MAILNHSDAAFPVAATTPLLGRRPLDAGEFRRQGRQVVDFIADYYAGINDYPVRPAVAPGFLAGKLPATAPSTPEPDALTAGLRDVRELMLPGLTHWQSPRHFAHFSATASNVGALGEALAAGLNVNPFTWEASPAATELEVVVTDWLGKALHLPERLLFAGGGGGTLLGTSCEAMLCTIVAARDEKLAEIGEERIGDLVVYCSDQTHFSFQKAARIAGIRRGNCREIPTCRESGFVLTATALQAAVAADEAAGRVPLFLCATVGTTPTAAVDPLRELCAAVEGRGVWVHVDAAYAGAACVCPEFRHAIAGAEAVDSFSTNPHKWLLANMDCCALWVARPAALVAALGTDDDVILKDAAAAARPARGDHHHHAAVDYKDWQVALSRRFRALKLWLVLRCHGVDGLRAVVRSHVRMAAALERMVRADARFEVPVPRQFALVCFRLRGGGAAAQLVGGDELTASNELNRRLLEAVNATGRAYMSSAVVGGMYVLRCAVGNSLTEEHHVREAWSVVQGQAAAVLATAGAAADTARTKDHAAGDDHGADQPHAMTTTTTMGCRSGPWEL

SEQ ID NO: 67 - protein, 509 aa, Brachypodium stacei
MAPASSTRQVITDHKTQKENSSCTVINHLLDADEFRRQGHKVIDFIADYYSGIADYPVHPSVTPGFLLNQLPADPPEDPDTFASALQDVRDLILPGMTHWQSPRHLAHFPASSSVTGALGEALAAGINAVPFMWSASPAATELEMVAVDWLGKALHLPKTLLFSGAGGGTLLGTSYRKLAETGAGRIGDLVVYGSDQTHFALRKAARIAGIRHGRCRELRTCIADMFALSPAALSAAMDADAGAGLVPLFLCATVGTTQTKAVDPIGALCAEAAPHGVWVHVDAAYGGSALVCPELARDAIDGVEAVDSFSMNAHKWLLVNTDCCALWVKRPALLVSALGTQDEDEVILRDAAAQGHDVVDYKDWAVTLTRRFRALKLWLVLRCYGVEGLREHIRGHVRMAALFEGMVNADPRFEVVTERRFALVCFRLRPDQLPDEGNKKKTMAAANELNRRLLQEVNAAALGPYMSAANVGGIYVLRCAVGSTLTEKRHVRQAWEVVQEKATSILRA

SEQ ID NO: 68 - protein, 475 aa, Amaranthus hypochondriacus
SLHDETLQGIKYVTQYYKNVEKYPVVSKVKWGYLRQILPENAPSLPESIDQILEDVDTKIVPGLTHWQSPNFFAYFPATASNAAMLGDIVCSGLNVVGFSWISSPAATELEAIVMDWMAKLLMLPPTFLFSGGGGGVIHGSTCEAIVCTQAAARDVALNIHGEEKITKLVVYASDQTHISFQKAAKLIGIPPRNFRVLPTSSATDFALSPTTLRASIEVDLSQGLVPFYICATIGATPSGAVDPIDGLGQIARDYGAWLHVDAAFAGNACICPEYRHYLDGVELADSISMNPHKWLLTNMECSCLWLKNPKLMVDSLSTKPEILNNKATQSGDVIDYKDWQIALSRRFRALKLWIVIRRYGSTYLMNHVRSDIELAKYFESLIKQDERFELVVPRKFSLVCFRMKLVGREDVETLTNQKLLEDVNSSGKAYMTHAVIGGKFVIRCAIGGTLTEKRHIDSLWKLIIEKVPLTTCEL

SEQ ID NO: 69 - protein, 553 aa, Brachypodium distachyon
MSSNSCPAAAAATFTTPPGAHPLPLDADAFRRQGRQVADFIADYYDRIEDYPVRPNVSPGFLAAQLPDAAPSWPEEPDALASALRDVRDLILPGLTHWQSPRHFAHFAATASNAGALGEFLAAGLNVNPFTWAASPAAAELEVVVTDWLGQALGLPEKLLFRGGSGGGGTLLGTSCEAMLCTIVAARDQKLLKIGEDRIGDLVVYCSDQTHFSFKKAARVAGIRRGNCRVIPTRFEDGFALSPAALAAAVRDDVARGKVPLFLCATVGTTATGAVDPVRELCAAVGAGHGSGVWVHVDAAYAGGACVCPEFRHVAAGAEEADSFSTNPHKWLLANMDCCALWIRRPGLLVAALGAGEDEDAILNKAPPAARGMQADLMVDYKDWQVPLSRRFRALKLWLVLRCHGVEGLRGVVRGHVRMAAAFEAMVRADPRFEVPVPPAFALVCFRLRPLAAHPGSSSGIDEVNGRLLEAVNGTGRAYMSGAVVGGAYVLRCAVGNSLTEDRHVREAWSVVQEQADAILAPSDDEDRCCTDQIQTEMELQRRPLGAAADVFA

SEQ ID NO: 70 - protein, 543 aa, Brachypodium distachyon
MAPASSKLHAITDDKTQQQNSSCPAASNGAIEPSNAKCAASSNHLLDADEFRRQGHKVIDFIADYYAGIADYPVHPSVTPGFLLNQLPADPPSRPEDHPDGAFGPALQDVRDVILPGMTHWQSPRHFAHFPASSSVAGVLGEALAAGINAVPFTWAASPAAAELEMVAVDWLGKALHLPESLLFSGAGGGTLLGTSCEAILCALVAARDRKLADIGTDRIGDLVVYGSDQTHFALRKAARIAGIRHDRCRELQTCLADMFALSPAALSAAMDADAGAGLVPLFLCATVGTTQTTAVDQVGALCAAAAPHGVWVHVDAAYAGSALVCPELARDAIDGIEVVDSFSMNAHKWLLANTDCCALWVKQPKLLVVSLGTQNEELILRDAAAEGHDVVDYKDWAITLTRRFRALKLWLVFRCYGVEGLREHIRAHVRMAALFEGLVKDDPRFEVVTERRFALVCFRLRAPDQLMDEGNEKKKTTAAANELNRRLLREVNGVALGPYMSAAVVGGIYILRCAVGSTLTEERHVRQAWEVVQERATSILRG

SEQ ID NO: 71 - protein, 588 aa, Sorghum bicolor
MGVAVTAEVVHARSCKGTPPVGAAASVMVWDGAGQGYSCQPVGTTTANGGTTPAAPVAIAMPSLPHPLLDADEFRRQGRLVVDFIADYYARIDEYPVRPAVAPGFLARQLPETAPARPEPDALAAALRDVRDLILPGVTHWQSPRHFAHFAATASNVGALGEALAAGLNINPFTWAASPAATELEVVVTDWLGKALHLPESLLFSGGGGGTLLGTSCEAMLCTIVAARDRKLAEVGEERMGDLVVYCSDQTHFSFQKAARIAGIRRGNCREIPTSMEAGFTLSPKALAAAVRADEAAGRVPLFLCATVGTTPTAAVDPVRELCAAVAGRGVWVHVDAAYAGAASVCPELRHAVAGVERVDSFSTNPHKWLLANMDCCALWVRRPAALTAALGTDHDVILKDPSAQAAQEGGAVVDYKDWQVALSRRFRALKLWLVLRCHGVEGLRGLVRAHVRMAAAFEAMVRTDARFEVPVPRQFALVCFRLRAAAVLVVGEKRARDGDDEVVTAGNELNRRLLEAVNATGRVYMSSAVVGGTYILRCAIGNSLTEERHVREAWSVVQEQATAILAAARRPTARTNRRTVRRAHAAL

SEQ ID NO: 72 - protein, 502 aa, Kalanchoe laxiflora
MGSLQSPHDPNAFNPMDVAELSIESRLVMDFITQYYQTLETRPVQPRVKPGFLTGQLPEKPPFHAESMEEILSDVSEKIVPGLTHWQSPNFHAYFPASSSNAGLLGEMLCSGLSVIGFTWNSSPAATELENVVVDWLADMLNLPPSFRFSGGGGGVLQSNTCEAVLCTLAAARDKVLERIGDDKINKLVVYCSDQTHFTLHKGAKLIGIRRANIKSISTRRENGFGLCPNDLRNAIKSDLEAGLVPFYLCGTIGTTALGAVDPIKELGKVAREFDLWFHIDAAYGGSACICPEFRHYLDGVELVDSISMNAHKWLLSNLDCCFLWLQNPKCLIQCLAAEGEFLKGSGEMVDYKDWQISLSRRFRAIKMWMVFRRYGVSNLMEHIRSDVSMAARFEEMVAADDRFEIVFPRKFALVCFKLNTKGSVQHGEVDGEDGLDGDSVLTRELMGRVNSSGKAYLSGVEMGRIFFIRCVIGSSLTEERHVDNLWNLIQEKTQSIMPRRA

SEQ ID NO: 73 - protein, 509 aa, Kalanchoe laxiflora
MGSLSSPRDLTKPFNPLDPTELAVESSLVTDFIAEYYRTVEQRPVQPHVTPGFLTSQLPSAAPFASESVESILQDVYDKILPGLVQWQSPNFHAYYPATCSNAGLLGEMLCSGLNVVGFTWSASPAAAELEQVVVDWMGKMMGLPQSFLFSGGGGGVLQGSTCEAVVCTLAAARDRALERVGDDMFNKLVVYCSDQTHFTLKKGSKLVGIRPANVKAIKTTKNNEYGLCPTDLRNLVASDVKAGFIPIYLCGTIGTTAFGAVDPIRELGKVAREFNMWFHVDAAYAGSAFICPEFRHYMDGVELADSFSTNPHKWLLSNMDCCVLWLKFPKRVIKSLAAEGVFLEGGSETMVDYKDWQIALSRRFRAIKLWMVIKRYGLKNLISHIRSDVSMAKRFEELLLSDRRFEVVFPRKFSLVCFKLDVMKNVPEVVDEDDGELSHDSKLTRELMASVNVTGKAFLTGVRLGRIFFIRCAIGSTLTEDRHIQDLWKLIQEKAHKICANHDLKFRV

SEQ ID NO: 74 - protein, 607 aa, Panicum hallii
MAILNHGDTTTANGSSPADAAAVAPAMPSLVQPPLDADEFRRQGRLVVDFIADYYTRIDEHPVRPAVAPGFLARQLPDTAPARPEPGDDALAAALRDVRDLILPGVTHWQSPRHFAHFAATASNVGALGEALTAGLNINPFTWAASPAATELEVVVTDWLGKALHLPESLLFSGGGGATLLGTSCEAMLCTLVAARDRKLAEIGEERIGDLVVYCSDQTHFSFQKAARIAGIRRGNYREIPTSRESGFTLSPKVLRAAVRADEAAGRVPLFLCATVGTTPTAAVDPLRELCATVAGHGVWVHVDAAYAGAACVCPEFRHAIAGAEAVDSFSTNPHKWLLANMDCCALWVRRPEALTAALGTDHDVILKDPSSERDCGRGVVDYKDWQVALSRRFRALKLWLVLRCHGVEGLRGFVRAHVRMAAAFEDMVRADARFEVPVPRQFALVCFRLRSAAAGEKRARDGDDAEPNELNRRLLEAVNATGRAYMSSAVVGGIYVLRCAIGNSLTEERHVREAWCVVQEQATVVLAAAACTEERAVHSARCADAPAAVPPVQNEGYGEPTSIAAKIFGTSIARCSIKSEASTYHSWSTLWRTLMFKLLTWIISRL

SEQ ID NO: 75 - protein, 510 aa, Prunus persica
MTSALDPVEFRRQGHMMVDFIADYYQNIDKYPVLSQVDPGYLRKRLPESAPDNPEPIETILQDVQEHIVPGLTHWQSPSFFAYFASNVSIAGFLGEMLSTGFNVVGFNWVSSPAATELESIVMDWLGNLLSLPKSFLFSGNGGGVIHGSTCEAIVCTMAASRDQMLSRIGGDNIGKLVVYGSDQTHSALQKASQIVGINPKNFRAIEATRSTTFALSPESLKLAISSDIEAGLVPLFLCATVGTTATTAVDPLGPLCDVAKHHGMWVHVDAAYAGSACICPEFRHFIDGIEGVDSFSFNAHKWFFTGLDCCCLWVKNPGALISSLSANPEFLRNKPTDSKQVVDYKDWQIALSRRFRAMKLWLVLRSYGVVNLRNFLRSHVKMAKLFEGLVAMDQRFEIVVPRNFSMVPPTTPTSNSFHQNGIEINVEKCTNEVNCKLLEAINASGRVFMTHAMVGGMYVIRCAVGVTQTEEKHIAMAWKVVQEHADVILKNNGDDGDANLKLPLLDKIA

SEQ ID NO: 76 - protein, 510 aa, Prunus persica
MGSLNFDHPQENNSAHMSGPLDLVELRRQGHMIIDFITDYYQNIEKHPVLSQVQPGYLKQRLPESAPYNPEPIETILRDVQDHIVPGLTHWQSPNHFAYFPATISTAGFLGEMLTTCFNVVGFNWMASPAATELETIVMDWLGDMLKLPNSFLFSGTGGGVLHGSTHESVVCTMAAARDQILSRIGEENIGKLVVYGSDQTHSVIQKVSQIVGIPSKNFRAIETTISSSFTLSPETLRLTVCSDMEAGLVPFYLCATVGTTATTAVDPLGPLCDVAKDYGMWVHVDAAYAGSACICPEFRQYIDGIEGANSFSFNAQKWFFTALDCCCLWVKNPSALTKSMSTDLEVLRNKASESKRVVDFKDWQIALTRRFRAIKLWLVLRSYGVANLRNFLRSHVKMAKRFEGLVRTDERFEVVVPRIFALVCFRISPSAISKANPTPSDEKCVNEVNCKLLEAINGSGWVYMTHAVVGGMYVLRCAIGASLTKEKHVAMAWKVVQEHVDAILPLTMY

SEQ ID NO: 77 - protein, 533 aa, Prunus persica
MMGSVEFEHPQENNSAHMTTSPLDPEEFRRQGHMVIDFIADYYKTIEKYPVLSQVQPGYLKKRLPESAPYDPEPIETILQDVQDHLVPGLTHWLSPNHFGYFPAAISTAAFLGEMLTTGFNVVGFNWMASPAATELENIVMDWLGDMLKLPKSFLFSGNGGGVLQGTTCEAIVCTMAAARDQMLRQIGRENIGKLVVYGSDQTHSALQKASQIVGIHPKNFRAIETTTSTSFALSPEVLKSTICSDIEAGLVPLFLCATVGTTAITAVDPLGPLCEVAKEHDMWVHVDAAYAGSAFICPEFQYFIDGVEGADSFSLNAHKWFFTTLDCCCLWVKNPSALVSSLSTNPEFLRNKATDSKQVVDYKDWQIALSRRFKAIKLWLVLRSYGVGNLRNFLRSHVKMAKIFEGLVGMDKRFEIVAPRHFSLVCFRVSPSAISKANPSLSDHDNGKLKAHNYELLNGVKCVVNEVNSKLLEAINGSGLVYMSHAVVGGMYVLRCAIGASLTEEKHVAMAWKVVQEHADAILGTKIIVDQT

SEQ ID NO: 78 - protein, 502 aa, Medicago truncatula
MNTSSSNPPQSDPQKTMNPLDLEEFKRQGYMMIDFLTDYYKNIENYPVLSKVEPGYLAKILPSSAPFQPESIESILEDVQQHIIPGITHWMSPNYYAYFPSSGSIAGFIGEMLSTGFNVVGFNWLSSPAATELETIVMNWLGKLLNLPKSFIFSSNIKGGGEIKKLSQIGKDNIGKLVVYCSDQTHSALQKATQIVGIHSENFRVIKTKGSNLFALSPDSLLSTILLDVDNGLIPYFLCATIGTTSTNAVDPIKLLCNVTKEYDIWVHVDAAYAGSVCICPEFRHCIDGIEELNSFSFNAHKWFLTNLACCCLWVKDHNALTTSLSTNPEFLRNKKSDSKEVIDYKDWQIPLSRKFNALKLWIVLRSYGVENLKNFLRNHVEMAKIFEGLVRKDERFEIVVPSKFSLVCFRISPFAISIANDSEGYYVGKMMNDAYLVNEMNHKLLDLINSSGKAYMSHGEVEGSFVIRCAIGATLTEEHHVTMTWKLVQQIASFLLGTPLN

SEQ ID NO: 79 - protein, 577 aa, Zea mays
MAILNRADTSHTTTASNGSATPAAPVAIAMPSLPHPPLDADEFRRQGRLVVDFIADYYARIDGYPVRPAVAPGFLIRQLPEAAPARPEPDALAAALRDVRDLILPGVTHWQSPRHFAHFAATASNVGALGEALAAGLNVNPFTWAASPAATELEVVVTDWLGKALHLPESLLFSGGGGGTLLGTSCEAMLCTIVAARDRKLAEVGEERIGDLVVYCSDQTHFSFQKAARIAGIRRGNCREIPTSRESGFTLSPKALAAAVRADEAAGRVPLFLCATVGTTPTAAVDPLRELCAAVAGHDVWVHVDAAYAGAACVCPEFSHVVAGVEAAESFSTNPHKWLLANMDCCALWVRRPAALTAALGTDHDVILKDPAAAQAQAQQQQCSDGGVVDYKDWQVALSRRFRALKLWLVLRCHGVEGLRGLVRAHVRMAAAFEAMVRGDARFEVHVPRQFALVCFRLRAVAVAVAGEKRAGDYDGVAAGNELNRRLLEAVNATGRVYMSSAVVGGAYILRCAIGNSLTEERHVREAWSVVQEQATAILSAATATARTNGLTVRRARCDAEADVSDVPTPQQPLPLG

SEQ ID NO: 80 - protein, 519 aa, Glycine max
MEMKNTMNRNPQSDAPIIKPLDPEEFKRQGYMMVDFLADYIRNVSHYPVLSKVEPGYLKQRLPTSAPCGPEPIESILKDVQDHIIPGLTHWQSPNFYGYFPSSGSIAGFMGEMLSAGLNVVGFNWVSSPSATELESIVMDWLGQVLNLPKSFLFCGDHGGGVVLGTTCEAILCTLVAAREKKLSQVGKENIGKLVVYGSDQTHSALQKAAQIAGIHPANFRVIKTKRSNSFALSPDSLLSTILLDVERGLIPCFLCATVGTTAIATIDPIGPLCNVAKDYGIWVHVDAAYAGSACICPEFRHCIDGVEEVNSFSLNAHKWFLTNLTCCCLWVKDHIALTKSLTVNPQFLRNKASESKRVIDYKDWQIPLSRKFNALKLWLVLRSYGVENIRNFLRNHVQMAKTFEGLVRLDKRFEIVVPPKFSLVCFRIAPSAIIANGLSKGVEACYNGKLVNDEYMVNEVNRKLLDSVNSSGDAFMTHGEVEGAFMIRCAIGGTLTEEHHVIMAWKLVQEHANSLLGL

SEQ ID NO: 81 - protein, 531 aa, Panicum virgatum
MAILNHGDTTAASGTSPAAAAVNVAPPMHSLVQPVLDADEFRRQGRLVVDFIADYYTRIDEYPVRPAVAPGFLARQLPEAAPARPEPGGDALAAALRDVRDLILPGVTHWQSPRHFAHFATTGSNVGALGEALAAGLNINPFTWAASPAATELEVVVTDWLGKALHLPERLLFSGGGGGTLLGTSCEAMLCTLVAARDRKLAEIGEERMGDLVVYCSDQTHFSFRKAARIAGIRRGNCREIPTSRESGFALQPRTLLAAVRADEAAGRVPMFLCATVGTTPTAAVDPLRELCAAVAGRGVWVHVDAAYAGAACVCPEFRGATAGAEAVDSFSTNPHKWLLANMDCCALWVRRPEALTAALGTDHDVILKDPSSERGGGVVDYKDWQVALSRRFRALKLWLVLRCHGVEGLRGLVRADARFEVPVPRQFALVCFRLRAAAAAAVGEKRGRDRDNDAEPNELNRRLLEAVNATGRAYMSSAVVGGIYVLRCAIGNSLTEERHVREAWRVVQEQATAVLAAAACTEERAVRSAR

SEQ ID NO: 82 - protein, 489 aa, Theobroma cacao
MSSASRKTFLPLEPTSFTNESKAVIDFIADYYKNIEEYPVQSGVEPGYLSAKLPDSAPYCPESLEDILKDVNDCIIPGLTHWQSPNFFAYFQANASTAGFLGEMLCSGFNVVGFNWISSPAATELESIVLDWMGKLLKLPSSFLFSGTGGGVLHGSTCEAAVCTLAAARDKALKELGGWENITKLMVYASDQTHFTFQKAAKLVGIPPSNFRFIETSLSTGFSMSSDQVRLAIEHDIKSGLVPLFLCATIGTTACGAIDPIAELGQVAREYKLWLHIDAAYAGSACICPELRHFLDGVELANSVSMNPHKWFLTNMDCCCLWITEPRLLVDSLSTDPEILRNKASEFKAVLDYKDWQVALSRRFRALKLWIVIRRHGLANLVYHIRSDISMAERFEAFVAKDDRFDIVVPRKFALVCFRLKPKQELEGLELNSRLLEAINSSGRAFMTHAVVGGIYVIRCAIGTTMTEERHVDALWKLIQEKAQGLLME

SEQ ID NO: 83 - protein, 625 aa, Fragaria vesca
MGSLDFHHVPEKTNSDPPMANPMDPEEFRRQGHIMIDFIADYYKNIEKYPVLSQVQPGYLKKLLPESAPYNPEPIETILQDVQDHIVPGITHWQSPSYFAYFPSSGSIAGFLGEMLSTGFNVVGFNWMSSPAATELERTTCEAIVCTMAAARDQMLSRIGKDNIGKLVVYGSDQTHSALKKASQIVGIHPNNFRAIKTTKSTEFALSPELLRSTICSDIDKGLVPLFLCATMGTTATTSVDPLRGLCDVAKDYDLWVHVDAAYAGSICICPEFRHFIEGVDGANSFSFNAHKWFFTTLDCCCLWVKNPTALINSLSTNPEFLRNKASDSKQVVDYKDWQVALSRRFRALKLWLVLRSYGVANLRSFLRSHVKMAEVFEKLVRENKWFEVVVPRNFAMVCFRISPSAIRKAPTDDDGIDVVINEVNSKLLEAMNTSGSVYMTHAVVGGMYVLRCAIGATMTEEKHVLMAWKCGSALERKDVAANETLSFNFQRRFDRRARQRRGHVGFRLAITMLDLKTSERDGARRWSIGAYANQITTISQANSSVAWTMEFHSCFIFFCGSIKLDTQVPNDDFVLSARWPPSFPVSGWSTINFHETIKIYVGSLDSLDSWTMEFHSCFTFFCGS

SEQ ID NO: 84 - protein, 486 aa, Gossypium raimondii
MVSASRKTFLPLDPVTFSNESKAVIDFIADYYENVEKYPVQSTVEPGYLSAMLPESAPYCPEPLQDILEDVSNCIIPGLTHWQSPNFFAYFHANASTAGFFGEMLCSGFNVVGFNWISSPAATELESIVLDWMGKMLKLPSSFLFSGTGGGVLHGSSCEAAVCVLAAARDKALKELGGWENITKLVVYASDQAHFTFQKAAKLVGIPPSNFRLIETSFSTGFSLSPENLRFVIEDNIRSGLVPLFLCATIGTTPSGAVDPIAELGKVAMEFKLWLHIDAAYAGSGCICPELRHYLDGVELANSISMNPHKWFLTNMDCCCLWIKEPKLLVDSLSTDPEILRNNASKSKAVVDCKDWQIALSRRFRALKLWVVIRRHGLANLMCHIRSDIAMAKRFEALVGEDERFEIVVPRKFALVCFRLKPKVEEEDLNCKLVEAINSSGRAFMSHAVLSGIYVIRCAIGTTLTQQHHVDALWKLIQDKAQSLLM

SEQ ID NO: 85 - protein, 495 aa, Populus trichocarpa
MGSLSTNTFSPLDPNGFTNDSKMVIDFIADYYKNIENNPVQSQVKPGYLLTQLPDTAPYCEESLEDVLKDVTDSIIPGLTHWQSPNFFAYFQANASTAGFVGEMLCTGLNVVGFNWIASPAATELESIVMDWMGKMLKLPSTFLFSGNGGGVLHGSTCEAIVCTLVAARDETLRMIGAENITKLVVYASDQTHSTLLKGVKLVGIPSSNFRCLSTSFSSEFSLSPQALEDAIENDIKAGLVPLFLCATVGTTACGAVDPVMDLGEIARKYNLWFHIDAAYAGSACICPEFRHYLDGVELADSLSMNPHKWLLTNMDCCCLWVKQPRLLIESLSSDAEFLRNNASESSDVVDYKDWQIALSRRFRALKLWIVIRRHGLANLMCHIRSDVNLAKRFESLVAKDSRFEVVVRRRFSLVCFRLKHNDECQGLELNRKLLAAVNESGRAFMTHAVVGGLFIIRCAIGSTLTEERHVDDLWKLIQEKAADLLSKKQVLLDN

SEQ ID NO: 86 - protein, 565 aa, Malus domestica
MSLLAFYSNSGERSKRVHLSASTYGNSTPNSYISLPYALFSSATQLINIHSNSSNFQMGSLISQENNSPNVPTNPLDPEEFRRQGHLVIDFIADYYKSIEKHPVLSQVQPGYLKKRLPDTAPYNPEPLETILQDVQDHIVPGITHWQSPNYFAYFPSSGSVAGFLGEMLSSGFNVVGFNWMSSPAATELESTVRDWFGNMLKLPKSFLFSGNGGDVIQGTTCEALVCAMVAARDQKLSKFGRHNIGKLVVYGSDQTHSALQKASQIVGIHPENFRSIETTRSTSFALSPESLKVIIYSDIEAGLVPLFLCATVGTTAIATVDPLGPLCGVAGDYGMWVHVDAAYAGSACICPSFDISLMASRVQIHSVSTRTNGSSPLSTVVAFGLRIPTRWNKATELKQVVDYKDWQIALSRRFRSMKLWLVLRSYGVANLRNFLRSHVKMAKIFEGLVAMDKRFEIVAPRNFSLVCFRVSPSSISNKASSDQNGKTDYCCDANGDENSVIINEVNRKLLESINVSGHVYMTHGVVGGLYMLRFAVGATLTEEHHIALAWKVVQEHADQILTKY

SEQ ID NO: 87 - protein, 523 aa, Citrus clementina
MRAGEASIIKMGSFGLSANNITHGSSFSADLEPKSFSDESKAVIDFIADYYKNIEKYPVQSKVEPGYLSARLPDTAPHSPESLDDILKDVTDCILPGLTHWQSPNFFGYFQANASTAGFLGEMLCSGFNVVGFNWLASPVATELESIVMDWMGKMLKLPSSFLFSGTGGGVLHGSTCESLVCTLAAARDKALEKLGGGFDNITKLAVYASDQTHFALQKSAKLIGIPPANFRPLRTSFSTEFSLSPDTVRAAIEDDIKSGHVPLYLCATVGTTGAGAVDPIEELGKIANEYKLWLHIDAAYAGSACICPEYRHYLNGVELADSISLNPHKWFLTNMDCCCLWVKHPSFLVDSLSTESDIMRNRSPASNTSTNAAPVIDYKDWQIALSRRFKALKLWTVIRKHGYSGLMYHIRSDVSMAKRFAAMVAKDERFEIVVPRKFALVCFRLKPKRESEGSELNRELVDALNGSGRAFLTQAMLGGVYVIRCSIGTTLTQDRHVDDLWKLIQEKADRLLSLQEPEHASR

SEQ ID NO: 88 - protein, 506 aa, Citrus clementina
MGSLNSDHELKTNSASFNNPMDSEEFRRQGHMIIDFIADYYRDVEKYPVLSQVEPGYLQKRLPESAPYNPEPIETILQDVQQHIVPGITHWQSPYYFAYFPSSGSIAGFLGEMLSSGFNVVGFNWMSSPAATELENIVMDWLGEMLKLPKSFLFSGTGGGVIQGTTCEAILCTLAAARDQILNEIGRENISRLVVYGSDQTHSALQKAAQIAGIDPKNFRAIKTTKSSSFTLTPESLQAAIDLDIQSGLIPLFLCATVGTTAITTVDPLGPLCDIAKRYSIWIHVDAAYAGSACICPEFRHFIDGIESADSFSLNAHKWFFTTLDCCCMWVKNPNALIKALSTNPEFLRNKASDSKQVVDYKDWQISLSRRFRALKLWLVLRSFGVANLRNFLRSHVGMAQLFQELVGGDNRFEIVAPRNFAVVCFRVLPSASGLGNGKANEGANELNRKLLESINASGQLYVSHGMVAGIYFIRFAVGATLTEDRHVIAAWKVVQEKLDGILATS

SEQ ID NO: 89 - protein, 467 aa, Vitis vinifera
MGSLSFNTFSPLDPQSFSEESKMVVDFIADYYKNVEKYPVQSQVDPGYLMHHCPDTAPYCPEPLETILKDVSDGIIPGLTHWQSPNFFGYFQANASTAGFLGEMLCTGLNVVGFNWIASPAATELESIAIICSLAAARDKVLKKLGHHKITKLVVYGSDQTHSTLQKASKLVGIPASNFRSLPTSFSNYFALCPDDVRTAMEEDIGAGLVPLFLCATVGTTSSGAVDPLEALGHVAKDFKVHHLNGVELAHSISMNPHKWLLTNMDCCCLWIKEPKLFVDSLSTAPEFLRNNASESKKVIDYKDWQIALSRRFRAIKVWAVVPRRFALVCFRLRPREEGESTELNSRLLMAVNGSGAAFMTHAVVGGIYIIRCAIGSTLTETRHVDSLWKLIQEKAQLVLQEPGLALEEDYIDPCIGVSATSLHAVVRWYCNYSSEINAHLVFIAFFVVVCKENRENYVLGVNGPPN

SEQ ID NO: 90 - protein, 506 aa, Petunia hybrida
MDTIKINPEFDGQFCKTTSLLDPEEFRRNGHMMVDFLADYFHNIEKYPVRSQVEPGYLERLLPDSAPIQPEPIEKILKDVRSDIFPGLTHWQSPNFFAYFPCSSSTAGILGEMLSAGLNVVGFSWIASPAATELESIVMDWLGKLINLPKTYLFSGGGGGVMQGTTCEVMLCTIVAARDKMLEKFGRENIDKLVVYASDQTHFSFQKAVKISGIKPENFRAIPTTKATEFSLNPESLRRAIQEDKKAGLIPLFLCTSIGTTSTTAVDPLKPLCEIAEEYGIWVHVDAAYAGSACICPEFQHFLDGVEHANSFSFNAHKWLFTTLDCCCLWLKDPSSLTKALSTNPEVLRNDATDSEQVVDYKDWQITLSRRFRSLKLWLVLKSYGVANLRNFIRSHIEMAKHFEELVAMDERFEIMAPRNFSLVCFRVSLLALEKKFNFVDETQVNEFNAKLLESIISSGNVYMTHTVVEGVYMIRFAVGAPLTDYPHIDMAWNVVRNHATMMLNA

SEQ ID NO: 91 - protein, 494 aa, Carica papaya
MSSLSRDLNASPLEPENFRVESKRVIDFIADYYKNIETYPVQSRVKPGYLAGRLPSSAPFSPESLETILQDIAENISPGLTHWQSPNFFGYFQANASTAGFHGEMLCSGLNVVGFNWISSPAATELESLVMDWMGNMLKLPSSFLFSGSGGGVLHGSTCEAVVCTLAAARDKTLNQLGGNYQNITKFVVYASDQTHFTLQKAAKLIGIPPSNFRSLTTSFPSGFSLSPEKLQSAIKDDIKSGYVPLYVCATVGTTAAGAVDPILELGKVAQEYNLWFHIDAAYAGSACICTEFRHYLNGVELADSISTNPHKWLLTNMECSCLWVKSPSSLVDSLSTKSEIMRNAATDSNQVIDYKDWQIALSRRFRALKLWIVIRRHGLSGLTSHIHKDIKMAELFESLVAKDKRFEIVVPRKFALVCFRFKPEKENQDLSELNSKLLNAVNSSGCAFMTHAVLEGVYTIRCAIGTTLTEEHHVVNLWKLIQEKAQSLIINEY

SEQ ID NO: 92 - protein, 452 aa, Sphagnum fallax
MSSKVAPWSRLSKPLDVEEFRTHAHRMVDFIADYHHNIQSFPVHSQLKPGYLRPLLPDTAPTEPEVVEDVFADVWNKILPGITHWQSPKFFGYYPFNVSTAGILGEILSGGVNVTGFSWITSPVVTELEIIVLDWLGKLLHLPEEFLSSGKGGGVIQGTSSEAVVCTSQHMSEAEALTKLVVYTSDQAQSCVLRACQIAGIATANFRPLPTDASSHFSLSPAVLIKAAATDVAAGLFPFFLCGKVGTTSSSAVDPLLELGDIAKRYGMWYHIDAAYAGSACICPEFRHYLNGVEKADSYNMNPHDWMLTNFDCSTLWVKNSELLVAALSNKPVYLQNEATDNNLVDCSHIRNHISIAKHFESLVRADFRFEMIVPTNFSLVCFRLRTPAGSKDNSRTLNSKLVEALNRKGDILVTHTELSGRYTLRFAVGGTHMELHHVQAAWNLRLQRQVF

SEQ ID NO: 93 - protein, 486 aa, Eucalyptus grandis
MNPLDPGEFRRQGHMVVDFLAKYYENIEKYPVLSQVEPGYLSKRLPSSAPQDEEPMEAILDDVHQHIFPGLTHWQSPNFFAYYQTNTSTAAILGEMLCAGFNVAGFNWVSSPAATELESLVMDWLGKMLDLPRPFLPFGNGGGVIEGNTSEAIICTLTAARDRVLRKLGHNSIAKLVVYGSDQTNCSFQKAARVVGIDPRNFRALKMTRSTLFGLSPDSLEKAIRLDINAGLIPLYLCATVGTTSCAAVDPLEPLCKVASKFSMWIHVDAAYAGASCICPEYRKFINGVEFADSFSFNAHKWLLTPLDCCCLWVKDPNALVKSLSTDPEYLKNEATESKQVIDYADWQLSLSRRFRALKLWLVLRSHGVQNLRSHIKNHCRLAKLFEELVEEDPQFEVVFPRNFALVCFRIHPSGVAGMLNAQLLHAINASGRVFMSHTTVGGVYVLRFAVGATLVTEKHVIMAWKVVQEHANSLLSMPASEQHSA

SEQ ID NO: 94 - DNA, 5336 bp, synthetic construct
  misc_feature 1..5336: note = pHis8-4 vector
  source 1..5336: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaacaccaccaccaccaccaccaccacggtggtgaaaacttgtacttccaggcccatggcggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 95 - DNA, 10003 bp, synthetic construct
  misc_feature 1..10003: note = pEAQ-HT vector
  source 1..10003: mol_type = other DNA
cctgtggttggcatgcacatacaaatggacgaacggataaaccttttcacgcccttttaaatatccgattattctaataaacgctcttttctcttaggtttacccgccaatatatcctgtcaaacactgatagtttgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgttaattaagaattcgagctccaccgcggaaacctcctcggattccattgcccagctatctgtcactttattgagaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaattcgcgaccggtatgcatcaccatcaccatcatcccgggcatcaccatcaccatcactagctcgaggcctttaactctggtttcattaaattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgccagcttggcgtaatcatggtcatagctgttgcgattaagaattcgagctcggtacccccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttcgactctagaggatccccttaaatcgatatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtctcgaactcttcagcatctctgtgagatggcaattcggtctaagcaagaactgctacagcttgccccaatcgaagtggaaagtaatgtatcaagaggatgccctgaaggtactgagaccttcgaaaaagaaagcgagtaaggggagctcgaattcgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatggcgcgtggccggcctacagtatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgcggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggactgtaggccggccctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacagagtagacaggctgtctcgcggccgaggggcgcagcccctgggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcggtcttgccttgctcgtcggtgatgtacactagtcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaactatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcaggtaacctcgcgcatacagccgggcagtgacgtcatcgtctgcgcggaaatggacgggcccccggcgccagatctggggaac

SEQ ID NO: 96 - DNA, 5291 bp, synthetic construct
  misc_feature 1..5291: note = pJKW 1410 vector
  source 1..5291: mol_type = other DNA
ccctgaattcgcatctagactgatgagacgtggtagagccacaaacagccggtacaagcaacgatctccaggaccatctgaatcatgcgcggatgacacgaactcacgacggcgatcacagacattaacccacagtacagacactgcgacaacgtggcaattcgtcgcaatacaacgtgagaccgaaagtgaaacgtgatttcatgcgtcattttgaacattttgtaaatcttatttaataatgtgtgcggcaattcacatttaatttatgaatgttttcttaacatcgcggcaactcaagaaacggcaggttcggatcttagctactagagaaagaggagaaatactagatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgaccaggcatcaaataaaacgaaaggctcagtcgaaagactgggcctttcgttttatctgttgtttgtcggtgaacgctctctactagagtcacactggctcaccttcgggtgggcctttctgcgtttataggtctcagctggaaatctgctcgtcagtggtgctcacactgacgaatcatgtacagatcataccgatgactgcctggcgactcacaactaagcaagacagccggaaccagcgccggcgaacaccactgcatatatggcatatcacaacagtccacgtctcaagcagttacagagatgttacgaaccactagtgcactgcagtacaaacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaggactgcgggattgctctcggtcaagcttttaaagaggccctaggggccgtgcgtggagtaaaaaggtttggatcaggatttgcgcctttggatgaggcactttccagagcggtggttgatctttcgaacaggccgtacgcagttgtcgaacttggtttgcaaagggagaaagtaggtgatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgctgcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatcatagagtaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcatcagagcgcttttggttttcaaaagcgctctgaagttcctatactttctagctagagaataggaacttcccgagcggccgcgtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtgcggccgc

SEQ ID NO: 97 - DNA, 52 bp, synthetic construct
  misc_feature 1..52: note = pHis8-4 Forward primer for Rr4HPAAS
  source 1..52: mol_type = other DNA
gaaaacttgtacttccaggcccatggcatgggcagcttgccttctcctaatg

SEQ ID NO: 98 - DNA, 52 bp, synthetic construct
  misc_feature 1..52: note = pHis8-4 Reverse primer for Rr4HPAAS
  source 1..52: mol_type = other DNA
ctcgaattcggatccgccatggctaagacacgatgctttgagctgtttcttg

SEQ ID NO: 99 - DNA, 53 bp, synthetic construct
  misc_feature 1..53: note = pEAQ-HT Forward primer for Rr4HPAAS
  source 1..53: mol_type = other DNA
gtatattctgcccaaattcgcgaccggtatgggcagcttgccttctcctaatg

SEQ ID NO: 100 - DNA, 65 bp, synthetic construct
  misc_feature 1..65: note = pEAQ-HT Reverse primer for Rr4HPAAS
  source 1..65: mol_type = other DNA
gaaaatttaatgaaaccagagttaaaggcctcgagctaagacacgatgctttgagctgtttcttg

SEQ ID NO: 101 - DNA, 55 bp, synthetic construct
  misc_feature 1..55: note = p423TEF Forward primer for Rr4HPAAS
  source 1..55: mol_type = other DNA
gcatagcaatctaatctaagttttctagaactagtatgggcagcttgccttctcc

SEQ ID NO: 102 - DNA, 51 bp, synthetic construct
  misc_feature 1..51: note = p423TEF Reverse primer for Rr4HPAAS
  source 1..51: mol_type = other DNA
cagcccgggggatccactagtctaagacacgatgctttgagctgtttcttg

SEQ ID NO: 103 - DNA, 48 bp, synthetic construct
  misc_feature 1..48: note = pHis8-4 Forward primer for RrAAS
  source 1..48: mol_type = other DNA
gaaaacttgtacttccaggcccatggcatggaggaggagttgaagccg

SEQ ID NO: 104 - DNA, 56 bp, synthetic construct
  misc_feature 1..56: note = pHis8-4 Reverse primer for RrAAS
  source 1..56: mol_type = other DNA
ctcgaattcggatccgccatggtcatgcatttatatgcttttgtagcagtgaagtg

SEQ ID NO: 105 - DNA, 48 bp, synthetic construct
  misc_feature 1..48: note = pHis8-4 Forward primer for RrPAR1
  source 1..48: mol_type = other DNA
gaaaacttgtacttccaggcccatggcatgagtttaagcggagcgggg

SEQ ID NO: 106 - DNA, 47 bp, synthetic construct
  misc_feature 1..47: note = pHis8-4 Reverse primer for RrPAR1
  source 1..47: mol_type = other DNA
ctcgaattcggatccgccatggtcagagtttggcgaaacccttttcc

SEQ ID NO: 107 - DNA, 56 bp, synthetic construct
  misc_feature 1..56: note = p425TEF Forward primer for RrPAR1
  source 1..56: mol_type = other DNA
gcatagcaatctaatctaagttttctagaactagtatgagtttaagcggagcgggg

SEQ ID NO: 108 - DNA, 46 bp, synthetic construct
  misc_feature 1..46: note = p425TEF Reverse primer for RrPAR1
  source 1..46: mol_type = other DNA
cagcccgggggatccactagttcagagtttggcgaaacccttttcc

SEQ ID NO: 109 - DNA, 55 bp, synthetic construct
  misc_feature 1..55: note = pHis8-4 Forward primer for RrPAR2
  source 1..55: mol_type = other DNA
gaaaacttgtacttccaggcccatggcatgggtttatctgaagagaagaagttag

SEQ ID NO: 110 - DNA, 54 bp, synthetic construct
  misc_feature 1..54: note = pHis8-4 Reverse primer for RrPAR2
  source 1..54: mol_type = other DNA
ctcgaattcggatccgccatggtcatttgtctttcaaactttcgacagtgtctc

SEQ ID NO: 111 - DNA, 60 bp, synthetic construct
  misc_feature 1..60: note = p426TEF Forward primer for RrUGT1
  source 1..60: mol_type = other DNA
caatctaatctaagttttctagaactagtatggtgacgaaaaaaactcacattcttatcc

SEQ ID NO: 112 - DNA, 48 bp, synthetic construct
  misc_feature 1..48: note = p426TEF Reverse primer for RrUGT1
  source 1..48: mol_type = other DNA
cagcccgggggatccactagttcaggtaagaccagacacaaacttgac

SEQ ID NO: 113 - DNA, 51 bp, synthetic construct
  misc_feature 1..51: note = p426TEF Forward primer for RrUGT2
  source 1..51: mol_type = other DNA
caatctaatctaagttttctagaactagtatgggttctgattcacggcctc

SEQ ID NO: 114 - DNA, 54 bp, synthetic construct
  misc_feature 1..54: note = p426TEF Reverse primer for RrUGT2
  source 1..54: mol_type = other DNA
cagcccgggggatccactagtctaggacaaagtctctcttctcaacttcaattc

SEQ ID NO: 115 - DNA, 49 bp, synthetic construct
  misc_feature 1..49: note = pHis8-4 Forward primer for RrUGT2
  source 1..49: mol_type = other DNA
gaaaacttgtacttccaggcccatggcatgggttctgattcacggcctc

SEQ ID NO: 116 - DNA, 55 bp, synthetic construct
  misc_feature 1..55: note = pHis8-4 Reverse primer for RrUGT2
  source 1..55: mol_type = other DNA
ctcgaattcggatccgccatggctaggacaaagtctctcttctcaacttcaattc

SEQ ID NO: 117 - DNA, 50 bp, synthetic construct
  misc_feature 1..50: note = pEAQ-HT Forward primer for RrUGT2
  source 1..50: mol_type = other DNA
gtatattctgcccaaattcgcgaccggtatgggttctgattcacggcctc

SEQ ID NO: 118 - DNA, 63 bp, synthetic construct
  misc_feature 1..63: note = pEAQ-HT Reverse primer for RrUGT2
  source 1..63: mol_type = other DNA
gaaaatttaatgaaaccagagttaaaggcctcgagctaggacaaagtctctcttctcaacttc

SEQ ID NO: 119 - DNA, 51 bp, synthetic construct
  misc_feature 1..51: note = p426TEF Forward primer for RrUGT3
  source 1..51: mol_type = other DNA
caatctaatctaagttttctagaactagtatgtcaggcacaccacacatcg

SEQ ID NO: 120 - DNA, 44 bp, synthetic construct
  misc_feature 1..44: note = p426TEF Reverse primer for RrUGT3
  source 1..44: mol_type = other DNA
cagcccgggggatccactagttcaatgcttcatcgaactccgcc

SEQ ID NO: 121 - DNA, 49 bp, synthetic construct
  misc_feature 1..49: note = pHis8-4 Forward primer for RrUGT3
  source 1..49: mol_type = other DNA
gaaaacttgtacttccaggcccatggcatgtcaggcacaccacacatcg

SEQ ID NO: 122 - DNA, 45 bp, synthetic construct
  misc_feature 1..45: note = pHis8-4 Reverse primer for RrUGT3
  source 1..45: mol_type = other DNA
ctcgaattcggatccgccatggtcaatgcttcatcgaactccgcc

SEQ ID NO: 123 - DNA, 50 bp, synthetic construct
  misc_feature 1..50: note = pEAQ-HT Forward primer for RrUGT3
  source 1..50: mol_type = other DNA
gtatattctgcccaaattcgcgaccggtatgtcaggcacaccacacatcg

SEQ ID NO: 124 - DNA, 58 bp, synthetic construct
  misc_feature 1..58: note = pEAQ-HT Reverse primer for RrUGT3
  source 1..58: mol_type = other DNA
gaaaatttaatgaaaccagagttaaaggcctcgagtcaatgcttcatcgaactccgcc

SEQ ID NO: 125 - DNA, 55 bp, synthetic construct
  misc_feature 1..55: note = p426TEF Forward primer for RrUGT4
  source 1..55: mol_type = other DNA
caatctaatctaagttttctagaactagtatgggttcacaagcctctccaaaacc

SEQ ID NO: 126 - DNA, 56 bp, synthetic construct
  misc_feature 1..56: note = p426TEF Reverse primer for RrUGT4
  source 1..56: mol_type = other DNA
cagcccgggggatccactagttcattccttgaactggagaatatctttcacaagcc

SEQ ID NO: 127 - DNA, 51 bp, synthetic construct
  misc_feature 1..51: note = p426TEF Forward primer for RrUGT5
  source 1..51: mol_type = other DNA
caatctaatctaagttttctagaactagtatggaaccgagacctcacgcag

SEQ ID NO: 128 - DNA, 58 bp, synthetic construct
  misc_feature 1..58: note = p426TEF Reverse primer for RrUGT5
  source 1..58: mol_type = other DNA
cagcccgggggatccactagtttaattagtgtcaccaagatgagttttctttagtaag

SEQ ID NO: 129 - DNA, 60 bp, synthetic construct
  misc_feature 1..60: note = p426TEF Forward primer for RrUGT6
  source 1..60: mol_type = other DNA
caatctaatctaagttttctagaactagtatggaatctgtacaaggtgttcaagaaaagc

SEQ ID NO: 130 - DNA, 48 bp, synthetic construct
  misc_feature 1..48: note = p426TEF Reverse primer for RrUGT6
  source 1..48: mol_type = other DNA
cagcccgggggatccactagttcagtttgaattcctcgacaggagcac

SEQ ID NO: 131 - DNA, 55 bp, synthetic construct
  misc_feature 1..55: note = p426TEF Forward primer for RrUGT7
  source 1..55: mol_type = other DNA
caatctaatctaagttttctagaactagtatggctgaaaacactcatgctcatgc

SEQ ID NO: 132 - DNA, 52 bp, synthetic construct
  misc_feature 1..52: note = p426TEF Reverse primer for RrUGT7
  source 1..52: mol_type = other DNA
cagcccgggggatccactagttcatttcttgaagatttgtaggtcgtggatg

SEQ ID NO: 133 - DNA, 58 bp, synthetic construct
  misc_feature 1..58: note = p426TEF Forward primer for RrUGT8
  source 1..58: mol_type = other DNA
caatctaatctaagttttctagaactagtatggcttcctcctctttagcttgtgattc

SEQ ID NO: 134 - DNA, 61 bp, synthetic construct
  misc_feature 1..61: note = p426TEF Reverse primer for RrUGT8
  source 1..61: mol_type = other DNA
cagcccgggggatccactagtttatttaactgtttcttgtttttgcaggacagaatgaatg

SEQ ID NO: 135 - DNA, 51 bp, synthetic construct
  misc_feature 1..51: note = p426TEF Forward primer for RrUGT9
  source 1..51: mol_type = other DNA
caatctaatctaagttttctagaactagtatggggtctgagccactagtcc

SEQ ID NO: 136 - DNA, 51 bp, synthetic construct
  misc_feature 1..51: note = p426TEF Reverse primer for RrUGT9
  source 1..51: mol_type = other DNA
cagcccgggggatccactagtttatgctgaaattgcatccttagcaactgg

SEQ ID NO: 137 - DNA, 47 bp, synthetic construct
  misc_feature 1..47: note = p426TEF Forward primer for RrUGT10
  source 1..47: mol_type = other DNA
caatctaatctaagttttctagaactagtatgacgaggcgccaccac

SEQ ID NO: 138 - DNA, 47 bp, synthetic construct
  misc_feature 1..47: note = p426TEF Reverse primer for RrUGT10
  source 1..47: mol_type = other DNA
cagcccgggggatccactagttcatccaaggccattgacaaaacgac

SEQ ID NO: 139 - DNA, 56 bp, synthetic construct
  misc_feature 1..56: note = p426TEF Forward primer for RrUGT11
  source 1..56: mol_type = other DNA
caatctaatctaagttttctagaactagtatggcaggcgagattctaatacttccg

SEQ ID NO: 140 - DNA, 49 bp, synthetic construct
  misc_feature 1..49: note = p426TEF Reverse primer for RrUGT11
  source 1..49: mol_type = other DNA
cagcccgggggatccactagttcacttgtgggagataatgaagtccctg

SEQ ID NO: 141 - DNA, 46 bp, synthetic construct
  misc_feature 1..46: note = p426TEF Forward primer for RrUGT12
  source 1..46: mol_type = other DNA
caatctaatctaagttttctagaactagtatggaggaggcggccag

SEQ ID NO: 142 - DNA, 49 bp, synthetic construct
  misc_feature 1..49: note = p426TEF Reverse primer for RrUGT12
  source 1..49: mol_type = other DNA
cagcccgggggatccactagtttaacacagagtccaaatgtccagcaac

SEQ ID NO: 143 - DNA, 58 bp, synthetic construct
  misc_feature 1..58: note = p426TEF Forward primer for RrUGT13
  source 1..58: mol_type = other DNA
caatctaatctaagttttctagaactagtatgctacctctcttacatgttacactaac

SEQ ID NO: 144 - DNA, 50 bp, synthetic construct
  misc_feature 1..50: note = p426TEF Reverse primer for RrUGT13
  source 1..50: mol_type = other DNA
cagcccgggggatccactagtttacaagccaatgttggtcctgagatcac

SEQ ID NO: 145 - DNA, 46 bp, synthetic construct
  misc_feature 1..46: note = p426TEF Forward primer for RrUGT14
  source 1..46: mol_type = other DNA
caatctaatctaagttttctagaactagtatggacaccaccgccgc

SEQ ID NO: 146 - DNA, 49 bp, synthetic construct
  misc_feature 1..49: note = p426TEF Reverse primer for RrUGT14
  source 1..49: mol_type = other DNA
cagcccgggggatccactagtttatccccttccaagttgagtcaacgac

SEQ ID NO: 147 - DNA, 53 bp, synthetic construct
  misc_feature 1..53: note = p426TEF Forward primer for RrUGT15
  source 1..53: mol_type = other DNA
caatctaatctaagttttctagaactagtatggctgatgctgctcaacatgtc

SEQ ID NO: 148 - DNA, 58 bp, synthetic construct
  misc_feature 1..58: note = p426TEF Reverse primer for RrUGT15
  source 1..58: mol_type = other DNA
cagcccgggggatccactagtttattgaactttgtgaaattgaagatgactcaaaagg

SEQ ID NO: 149 - DNA, 53 bp, synthetic construct
  misc_feature 1..53: note = p426TEF Forward primer for RrUGT16
  source 1..53: mol_type = other DNA
caatctaatctaagttttctagaactagtatggcagaggaaaacagaaccagc

SEQ ID NO: 150 - DNA, 57 bp, synthetic construct
  misc_feature 1..57: note = p426TEF Reverse primer for RrUGT16
  source 1..57: mol_type = other DNA
cagcccgggggatccactagttcatacagctgaagatattttggatatgaattggtc

SEQ ID NO: 151 - DNA, 49 bp, synthetic construct
  misc_feature 1..49: note = p426TEF Forward primer for RrUGT17
  source 1..49: mol_type = other DNA
caatctaatctaagttttctagaactagtatgggctcacttccttccac

SEQ ID NO: 152 - DNA, 49 bp, synthetic construct
  misc_feature 1..49: note = p426TEF Reverse primer for RrUGT17
  source 1..49: mol_type = other DNA
cagcccgggggatccactagttcagacgctaaactggaccactttttcc

SEQ ID NO: 153 - DNA, 54 bp, synthetic construct
  misc_feature 1..54: note = p426TEF Forward primer for RrUGT18
  source 1..54: mol_type = other DNA
caatctaatctaagttttctagaactagtatgggctcccgaggaaagccacatg

SEQ ID NO: 154 - DNA, 46 bp, synthetic construct
  misc_feature 1..46: note = p426TEF Reverse primer for RrUGT18
  source 1..46: mol_type = other DNA
cagcccgggggatccactagttcattttggggaattagacagcagg

SEQ ID NO: 155 - DNA, 54 bp, synthetic construct
  misc_feature 1..54: note = p426TEF Forward primer for RrUGT19
  source 1..54: mol_type = other DNA
caatctaatctaagttttctagaactagtatgacgtcatcaacacctcctcctc

SEQ ID NO: 156 - DNA, 53 bp, synthetic construct
  misc_feature 1..53: note = p426TEF Reverse primer for RrUGT19
  source 1..53: mol_type = other DNA
cagcccgggggatccactagtctaaaaaaatgctttaacatagctagcgtccg

SEQ ID NO: 157 - DNA, 50 bp, synthetic construct
  misc_feature 1..50: note = p426TEF Forward primer for RrUGT20
  source 1..50: mol_type = other DNA
caatctaatctaagttttctagaactagtatgggttcactcgacgtcgtc

SEQ ID NO: 158 - DNA, 53 bp, synthetic construct
  misc_feature 1..53: note = p426TEF Reverse primer for RrUGT20
  source 1..53: mol_type = other DNA
cagcccgggggatccactagttcatttcataatagcttcatcaatcaactcgg

SEQ ID NO: 159 - DNA, 56 bp, synthetic construct
  misc_feature 1..56: note = p426TEF Forward primer for RrUGT21
  source 1..56: mol_type = other DNA
caatctaatctaagttttctagaactagtatgaagtccaacactcatctattcctc

SEQ ID NO: 160 - DNA, 44 bp, synthetic construct
  misc_feature 1..44: note = p426TEF Reverse primer for RrUGT21
  source 1..44: mol_type = other DNA
cagcccgggggatccactagttcatacaaccggctccagttgac

SEQ ID NO: 161 - DNA, 57 bp, synthetic construct
  misc_feature 1..57: note = p426TEF Forward primer for RrUGT22
  source 1..57: mol_type = other DNA
caatctaatctaagttttctagaactagtatgaaaactcctcaaaatccacacgtag

SEQ ID NO: 162 - DNA, 55 bp, synthetic construct
  misc_feature 1..55: note = p426TEF Reverse primer for RrUGT22
  source 1..55: mol_type = other DNA
cagcccgggggatccactagttcaatcctgataaatctttgaactcatcttgctc

SEQ ID NO: 163 - DNA, 54 bp, synthetic construct
  misc_feature 1..54: note = p426TEF Forward primer for RrUGT23
  source 1..54: mol_type = other DNA
caatctaatctaagttttctagaactagtatggaaaggcagagtgatcaccaag

SEQ ID NO: 164 - DNA, 54 bp, synthetic construct
  misc_feature 1..54: note = p426TEF Reverse primer for RrUGT23
  source 1..54: mol_type = other DNA
cagcccgggggatccactagttcatttggtggatatcacatctctaacaaactg

SEQ ID NO: 165 - DNA, 45 bp, synthetic construct
  misc_feature 1..45: note = p426TEF Forward primer for RrUGT24
  source 1..45: mol_type = other DNA
caatctaatctaagttttctagaactagtatgagcaacgccgccg

SEQ ID NO: 166 - DNA, 55 bp, synthetic construct
  misc_feature 1..55: note = p426TEF Reverse primer for RrUGT24
  source 1..55: mol_type = other DNA
cagcccgggggatccactagtttagtttatgacttcattcacttgctccaacaac

SEQ ID NO: 167 - DNA, 48 bp, synthetic construct
  misc_feature 1..48: note = p426TEF Forward primer for RrUGT25
  source 1..48: mol_type = other DNA
caatctaatctaagttttctagaactagtatggcgcgccaccactttg

SEQ ID NO: 168 - DNA, 57 bp, synthetic construct
  misc_feature 1..57: note = p426TEF Reverse primer for RrUGT25
  source 1..57: mol_type = other DNA
cagcccgggggatccactagtttagcaggtaacaaggttattaaccaaatccttgag

SEQ ID NO: 169 - DNA, 57 bp, synthetic construct
  misc_feature 1..57: note = p426TEF Forward primer for RrUGT26
  source 1..57: mol_type = other DNA
caatctaatctaagttttctagaactagtatgtcatcagattccggccacattatcc

SEQ ID NO: 170 - DNA, 58 bp, synthetic construct
  misc_feature 1..58: note = p426TEF Reverse primer for RrUGT26
  source 1..58: mol_type = other DNA
cagcccgggggatccactagtctatattatttttcttaatgccatgacttgtcggacc

SEQ ID NO: 171 - DNA, 55 bp, synthetic construct
  misc_feature 1..55: note = p426TEF Forward primer for RrUGT27
  source 1..55: mol_type = other DNA
caatctaatctaagttttctagaactagtatgagttcagtcaatgctcaaaagcc

SEQ ID NO: 172 - DNA, 53 bp, synthetic construct
  misc_feature 1..53: note = p426TEF Reverse primer for RrUGT27
  source 1..53: mol_type = other DNA
cagcccgggggatccactagttcaaaagtgcattagtagtccttccacaaatc

SEQ ID NO: 173 - DNA, 53 bp, synthetic construct
  misc_feature 1..53: note = p426TEF Forward primer for RrUGT28
  source 1..53: mol_type = other DNA
caatctaatctaagttttctagaactagtatggactcggttgatctgaacaag

SEQ ID NO: 174 - DNA, 47 bp, synthetic construct
  misc_feature 1..47: note = p426TEF Reverse primer for RrUGT28
  source 1..47: mol_type = other DNA
cagcccgggggatccactagtctagttggcacttggcaacacaatcg

SEQ ID NO: 175 - DNA, 57 bp, synthetic construct
  misc_feature 1..57: note = p426TEF Forward primer for RrUGT29
  source 1..57: mol_type = other DNA
caatctaatctaagttttctagaactagtatgggatctctaggaaagaagattcaac

SEQ ID NO: 176 - DNA, 52 bp, synthetic construct
  misc_feature 1..52: note = p426TEF Reverse primer for RrUGT29
  source 1..52: mol_type = other DNA
cagcccgggggatccactagtttaggttgtaactacaattttttttttggac

SEQ ID NO: 177 - DNA, 55 bp, synthetic construct
  misc_feature 1..55: note = pHis8-4 Forward primer for RrUGT29
  source 1..55: mol_type = other DNA
gaaaacttgtacttccaggcccatggcatgggatctctaggaaagaagattcaac

SEQ ID NO: 178 - DNA, 53 bp, synthetic construct
  misc_feature 1..53: note = pHis8-4 Reverse primer for RrUGT29
  source 1..53: mol_type = other DNA
ctcgaattcggatccgccatggttaggttgtaactacaattttttttttggac

SEQ ID NO: 179 - DNA, 56 bp, synthetic construct
  misc_feature 1..56: note = pEAQ-HT Forward primer for RrUGT29
  source 1..56: mol_type = other DNA
gtatattctgcccaaattcgcgaccggtatgggatctctaggaaagaagattcaac

SEQ ID NO: 180 - DNA, 66 bp, synthetic construct
  misc_feature 1..66: note = pEAQ-HT Reverse primer for RrUGT29
  source 1..66: mol_type = other DNA
gaaaatttaatgaaaccagagttaaaggcctcgagttaggttgtaactacaattttttttttggac

SEQ ID NO: 181 - DNA, 54 bp, synthetic construct; identical to SEQ ID NO: 153
  misc_feature 1..54: note = p426TEF Forward primer for RrUGT30
  source 1..54: mol_type = other DNA

SEQ ID NO: 182 - DNA, 46 bp, synthetic construct; identical to SEQ ID NO: 154
  misc_feature 1..46: note = p426TEF Reverse primer for RrUGT30
  source 1..46: mol_type = other DNA

SEQ ID NO: 183 - DNA, 59 bp, synthetic construct
  misc_feature 1..59: note = p426TEF Forward primer for RrUGT31
  source 1..59: mol_type = other DNA
caatctaatctaagttttctagaactagtatggaatctgtacaaggtgttcaagaaaag

SEQ ID NO: 184 - DNA, 48 bp, synthetic construct; identical to SEQ ID NO: 130
  misc_feature 1..48: note = p426TEF Reverse primer for RrUGT31
  source 1..48: mol_type = other DNA

SEQ ID NO: 185 - DNA, 58 bp, synthetic construct
  misc_feature 1..58: note = p426TEF Forward primer for RrUGT32
  source 1..58: mol_type = other DNA
caatctaatctaagttttctagaactagtatggactcggttgatctgaacaagaaacc

SEQ ID NO: 186 - DNA, 62 bp, synthetic construct
  misc_feature 1..62: note = p426TEF Reverse primer for RrUGT32
  source 1..62: mol_type = other DNA
cagcccgggggatccactagtctacaatttttttttggacagaagtacgtcatttataagtc

SEQ ID NO: 187 - DNA, 56 bp, synthetic construct
  misc_feature 1..56: note = p426TEF Forward primer for RrUGT33
  source 1..56: mol_type = other DNA
caatctaatctaagttttctagaactagtatgagcttaattgaaaaaccactcacg

SEQ ID NO: 188 - DNA, 54 bp, synthetic construct
  misc_feature 1..54: note = p426TEF Reverse primer for RrUGT33
  source 1..54: mol_type = other DNA
cagcccgggggatccactagtctaacggatatgttttgtttttgagagcaggac

SEQ ID NO: 189 - DNA, 54 bp, synthetic construct
  misc_feature 1..54: note = pHis8-4 Forward primer for RrUGT33
  source 1..54: mol_type = other DNA
gaaaacttgtacttccaggcccatggcatgagcttaattgaaaaaccactcacg

SEQ ID NO: 190 - DNA, 55 bp, synthetic construct
  misc_feature 1..55: note = pHis8-4 Reverse primer for RrUGT33
  source 1..55: mol_type = other DNA
ctcgaattcggatccgccatggctaacggatatgttttgtttttgagagcaggac

SEQ ID NO: 191 - DNA, 55 bp, synthetic construct
  misc_feature 1..55: note = pEAQ-HT Forward primer for RrUGT33
  source 1..55: mol_type = other DNA
gtatattctgcccaaattcgcgaccggtatgagcttaattgaaaaaccactcacg

SEQ ID NO: 192 - DNA, 68 bp, synthetic construct
  misc_feature 1..68: note = pEAQ-HT Reverse primer for RrUGT33
  source 1..68: mol_type = other DNA
gaaaatttaatgaaaccagagttaaaggcctcgagctaacggatatgttttgtttttgagagcaggac

SEQ ID NO: 193 - DNA, 58 bp, synthetic construct
  misc_feature 1..58: note = p426TEF Forward primer for RrUGT34
  source 1..58: mol_type = other DNA
gcatagcaatctaatctaagttttctagaactagttggaccctgacgacagcgttttg

SEQ ID NO: 194 - DNA, 60 bp, synthetic construct
  misc_feature 1..60: note = p426TEF Reverse primer for RrUGT34
  source 1..60: mol_type = other DNA
cagcccgggggatccactagtttagtttttgttctcgtacaaataatgcacaaactcatc

SEQ ID NO: 195 - DNA, 46 bp, synthetic construct
  misc_feature 1..46: note = pHis8-4 Forward primer for Pc4HPAAS
  source 1..46: mol_type = other DNA
gaaaacttgtacttccaggcccatggcatgggctccatcgataatc

SEQ ID NO: 196 - DNA, 48 bp, synthetic construct
  misc_feature 1..48: note = pHis8-4 Reverse primer for Pc4HPAAS
  source 1..48: mol_type = other DNA
ctcgaattcggatccgccatggttaggataaaatattcacgatcttct

SEQ ID NO: 197 - DNA, 47 bp, synthetic construct
  misc_feature 1..47: note = pEAQ-HT Forward primer for Pc4HPAAS
  source 1..47: mol_type = other DNA
gtatattctgcccaaattcgcgaccggtatgggctccatcgataatc

SEQ ID NO: 198 - DNA, 60 bp, synthetic construct
  misc_feature 1..60: note = pEAQ-HT Reverse primer for Pc4HPAAS
  source 1..60: mol_type = other DNA
gaaaatttaatgaaaccagagttaaaggcctcgagttaggataaaatattcacgatcttc

SEQ ID NO: 199 - DNA, 55 bp, synthetic construct
  misc_feature 1..55: note = pHis8-4 Forward primer for PsTyDC
  source 1..55: mol_type = other DNA
gaaaacttgtacttccaggcccatggcatgggaagccttccgactaataaccttg

SEQ ID NO: 200 - DNA, 49 bp, synthetic construct
  misc_feature 1..49: note = pHis8-4 Reverse primer for PsTyDC
  source 1..49: mol_type = other DNA
ctcgaattcggatccgccatggctaggcaccaagtatggcatctgtatg

SEQ ID NO: 201 - DNA, 56 bp, synthetic construct
  misc_feature 1..56: note = pEAQ-HT Forward primer for PsTyDC
  source 1..56: mol_type = other DNA
gtatattctgcccaaattcgcgaccggtatgggaagccttccgactaataaccttg

SEQ ID NO: 202 - DNA, 62 bp, synthetic construct
  misc_feature 1..62: note = pEAQ-HT Reverse primer for PsTyDC
  source 1..62: mol_type = other DNA
gaaaatttaatgaaaccagagttaaaggcctcgagctaggcaccaagtatggcatctgtatg

SEQ ID NO: 203 - DNA, 48 bp, synthetic construct
  misc_feature 1..48: note = p426TEF Forward primer for AAS55083
  source 1..48: mol_type = other DNA
caatctaatctaagttttctagaactagtatggcaggcagtgggactg

SEQ ID NO: 204 - DNA, 54 bp, synthetic construct
  misc_feature 1..54: note = p426TEF Reverse primer for AAS55083
  source 1..54: mol_type = other DNA
cagcccgggggatccactagttcagtgtttaactgaggatctccactttttagc

SEQ ID NO: 205 - DNA, 59 bp, synthetic construct
  misc_feature 1..59: note = p426TEF Forward primer for EU567325
  source 1..59: mol_type = other DNA
gcatagcaatctaatctaagttttctagaactagtatgggttctgaaactcggcctttg

SEQ ID NO: 206 - DNA, 54 bp, synthetic construct
  misc_feature 1..54: note = p426TEF Reverse primer for EU567325
  source 1..54: mol_type = other DNA
cagcccgggggatccactagtctagactttctttaacttgagttcctgaagcag

SEQ ID NO: 207 - protein, 21 aa, Catharanthus roseus
VDFKNWQIATGRKFRSLKLWL

SEQ ID NO: 208 - protein, 21 aa, Capsicum annuum
VDYKDWQIGTGRKFKSLRLWL

SEQ ID NO: 209 - protein, 21 aa, Ophiorrhiza pumila
VDFKDWQIGTGRRFKALRLWL

SEQ ID NO: 210 - protein, 21 aa, Camptotheca acuminata
VDYKDWQVGTGRRFKALRLWF

SEQ ID NO: 211 - protein, 21 aa, Camptotheca acuminata
VDFKDWQVGTGRRFKALRLWF

SEQ ID NO: 212 - protein, 21 aa, Oryza sativa
TDLKDMQVGVGRRFRGLKLWM

SEQ ID NO: 213 - protein, 21 aa, Petunia hybrid
VDYKDWQITLSRRFRSLKLWL

SEQ ID NO: 214 - protein, 21 aa, Petroselinum crispum
VDYKDWQIMLSRRFRALKLWF

SEQ ID NO: 215 - protein, 21 aa, Papaver somniferum
IDYKDWQIALSRRFRSMKLWL

SEQ ID NO: 216 - protein, 21 aa, Papaver somniferum
VDYKDWQIALSRRFRSLKLWM

SEQ ID NO: 217 - protein, 21 aa, Thalictrum flavum
VDYKDWQIALSRRFRAMKLWL

SEQ ID NO: 218 - protein, 21 aa, Rosa hybrid
VDYKDWQIALSRRFRALKLWL

SEQ ID NO: 219 - protein, 21 aa, Rhodiola crenulata
VDYKDWQISLSRRFRAIKMWM

SEQ ID NO: 220 - protein, 20 aa, Papaver somniferum
LVKALSTSAEYLKNKATESK

SEQ ID NO: 221 - protein, 21 aa, Brachypodium distachyon
LIAALGTEQEYILKDSASEGH

SEQ ID NO: 222 - protein, 21 aa, Oryza sativa
LVAALGTEQEYILRDAAAEGH

SEQ ID NO: 223 - protein, 20 aa, Thalictrum flavum
LIKALSTNPEYLRNKATESH

SEQ ID NO: 224 - protein, 20 aa, Papaver somniferum
LVKALSTNPEYLRNKATESR

SEQ ID NO: 225 - protein, 20 aa, Camptotheca acuminata
LVKALSTDPEYLKNQPSESK

SEQ ID NO: 226 - protein, 20 aa, Camptotheca acuminata
LVKALSTDPEYLKNKPSESN

SEQ ID NO: 227 - protein, 20 aa, Ophiorrhiza pumila
MVKALSTNPEYLRNKRSEFD

SEQ ID NO: 228 - protein, 20 aa, Oryza sativa
LTGSLETNPEYLKNHASDSG

SEQ ID NO: 229 - protein, 20 aa, Capsicum annuum
LIQSLSTNPEYLKNKASQGN

SEQ ID NO: 230 - protein, 20 aa, Arabidopsis thaliana
LIDALKTNPEYLEFKVSKKD

SEQ ID NO: 231 - protein, 20 aa, Capsicum annuum
LIQSLSTNPEYLKNKASQGN

SEQ ID NO: 232 - protein, 20 aa, Catharanthus roseus
LLRALTTNPEYLKNKQSDLD

SEQ ID NO: 233 - protein, 20 aa, Rhodiola rosea
LTKALSTNPEYLRNQQSELN

SEQ ID NO: 234 - protein, 20 aa, Rosa hybrid
LASSLSTNPEFLRNKASDSK

SEQ ID NO: 235 - protein, 20 aa, Petroselinum crispum
LIKSLSTYPEFLKNNASETN

SEQ ID NO: 236 - protein, 20 aa, Arabidopsis thaliana
LTLALSTNPEFLKNKASQAN

SEQ ID NO: 237 - protein, 20 aa, Olea europaea
LIQSLSTNPEFLKNKASEGN

SEQ ID NO: 238 - protein, 16 aa, Rhodiola crenulata
LIESLAAEANFLKGNS

SEQ ID NO: 239 - protein, 20 aa, Rhodiola rosea
LIQSLSTYPEFLKNKASQSN

SEQ ID NO: 240 - protein, 16 aa, Rhodiola rosea
LIESLAAEANFLKGGS

SEQ ID NO: 241 - protein, 17 aa, Rhodiola rosea
VLYCSDQTHFTIHKGAK

SEQ ID NO: 242 - protein, 17 aa, Arabidopsis thaliana
VVYSSDQTHSALQKACQ

SEQ ID NO: 243 - protein, 17 aa, Arabidopsis thaliana
VVYGSDQTHSSFRKACL

SEQ ID NO: 244 - protein, 17 aa, Capsicum annuum
VVYCSDQTHSSLQKACQ

SEQ ID NO: 245 - protein, 17 aa, Oryza sativa
VVYASDQTHSALQKACQ

SEQ ID NO: 246 - protein, 17 aa, Oryza sativa
AVYAADQTHSTFFKACR

SEQ ID NO: 247 - protein, 17 aa, Camptotheca acuminata
VVYGSDQTHSTYAKACK

SEQ ID NO: 248 - protein, 17 aa, Camptotheca acuminata
VVYGSDQTHSTYAKACN

SEQ ID NO: 249 - protein, 17 aa, Capsicum annuum
VVYGSDQTHSMYAKACK

SEQ ID NO: 250 - protein, 17 aa, Ophiorrhiza pumila
VVYGSDQTHSFFQKTCK

SEQ ID NO: 251 - protein, 17 aa, Catharanthus roseus
VCYGSDQTHTMFPKTCK

SEQ ID NO: 252 - protein, 17 aa, Petroselinum crispum
VVYCSDQTHSALQKAAK

SEQ ID NO: 253 - protein, 17 aa, Rosa hybrid
VVYGSDQTHSTLQKATQ

SEQ ID NO: 254 - protein, 17 aa, Petunia hybrida
VVYASDQTHFSFQKAVK

SEQ ID NO: 255 - protein, 17 aa, Thalictrum flavum
VVYGSDQTHCALQKAAQ

SEQ ID NO: 256 - protein, 17 aa, Brachypodium distachyon
VVYCSDQTHFAFRKAAR

SEQ ID NO: 257 - protein, 17 aa, Papaver somniferum
VVYASNQTHCALQKAAQ

SEQ ID NO: 258 - protein, 490 aa, Rhodiola rosea; identical to SEQ ID NO: 2

SEQ ID NO: 259 - protein, 490 aa, Arabidopsis thaliana; identical to SEQ ID NO: 21

SEQ ID NO: 260 - protein, 547 aa, Arabidopsis thaliana
MFKPQHMYDREFGTGNGYSNGNGYTNGNGHTNGNGNYNGNGHVNGNGKANGAKVVKMKPMDSELLREQGHIMVDFIADYYKNLQDSPQDFPVLSQVQPGYLRDMLPDSAPERPESLKELLDDVSKKIMPGITHWQSPSYFAYYASSTSVAGFLGEMLNAGLSVVGFTWLTSPAATELEIIVLDWLAKLLQLPDHFLSTGNGGGVIQGTGCEAVLVVVLAARDRILKKVGKTLLPQLVVYGSDQTHSSFRKACLIGGIHEENIRLLKTDSSTNYGMPPESLEEAISHDLAKGFIPFFICATVGTTSSAAVDPLVPLGNIAKKYGIWLHVDAAYAGNACICPEYRKFIDGIENADSFNMNAHKWLFANQTCSPLWVKDRYSLIDALKTNPEYLEFKVKVSKKDTVVNYKDWQISLSRRFRSLKLWMVLRLYGSENLRNFIRDHVNLAKHFEDYVAQDPSFEVVTTRYFSLVCFRLAPVDGDEDQCNERNRELLAAVNSTGKIFISHTALSGKFVLRFAVGAPLTEEKHVTEAWQIIQKHASKFTRNDHY

SEQ ID NO: 261 - protein, 487 aa, Capsicum annuum
MEGELKPMDAEQLREYGHKMVDFIADYYKNIETLPVLSQVEPGYLRKLLPETAPAHSETLQNVLEDVQTKILPGVTHWQSPDYFAYFPSNSSVAGFLGEMLSAGINMVGFSWITSPAATELEMIVLDWLAKALKLPDEFLSTGQGGGVIQGTASEAVLVVLLAARDKVLRRVGKDAISKLVVYCSDQTHSSLQKACQIGGIHPENFRVLKTDPSRDYALSPDTLSEAVSHDMATDLIPFFFCATIGTTSSTAVDPLLDLGKIAQSNSIWFHVDAAYAGSACICPEYRGYINGVEEAHSFNMNAHKWFLTNFDCSALWVKDRSALIQSLSTNPEYLKNKASQGNLVVDYKDWQVPLGRRFRSLKLWMVLRLYGLEKLQAYIRNHIQLAKLFEKLVAQDQRFEIVTPRKFSLVCFRLLPPPSNEDYANKLNHNLLDSVNSTGKLFISHTLLSDKYILRFAVGAPLTEERHIVGAWKVLQDEAATLLSKC

SEQ ID NO: 262 - protein, 497 aa, Oryza sativa
MEGVGGGGGGEEWLRPMDAEQLRECGHRMVDFVADYYKSIEAFPVLSQVQPGYLKEVLPDSAPRQPDTLDSLFDDIQQKIIPGVTHWQSPNYFAYYPSNSSTAGFLGEMLSAAFNIVGFSWITSPAATELEVIVLDWFAKMLQLPSQFLSTALGGGVIQGTASEAVLVALLAARDRALKKHGKHSLEKLVVYASDQTHSALQKACQIAGIFSENVRVVIADCNKNYAVAPEAVSEALSIDLSSGLIPFFICATVGTTSSSAVDPLPELGQIAKSNDMWFHIDAAYAGSACICPEYRHHLNGVEEADSFNMNAHKWFLTNFDCSLLWVKDRSFLIQSLSTNPEFLKNKASQANSVVDFKDWQIPLGRRFRSLKLWMVLRLYGVDNLQSYIRKHIHLAEHFEQLLLSDSRFEVVTPRTFSLVCFRLVPPTSDHENGRKLNYDMMDGVNSSGKIFLSHTVLSGKFVLRFAVGAPLTEERHVDAAWKLLRDEATKVLGKMV

SEQ ID NO: 263 - protein, 514 aa, Oryza sativa
MGSLDTNPTAFSAFPAGEGETFQPLNADDVRSYLHKAVDFISDYYKSVESMPVLPNVKPGYLQDELRASPPTYSAPFDVTMKELRSSVVPGMTHWASPNFFAFFPSTNSAAAIAGDLIASAMNTVGFTWQASPAATEMEVLALDWLAQMLNLPTSFMNRTGEGRGTGGGVILGTTSEAMLVTLVAARDAALRRSGSDGVAGLHRLAVYAADQTHSTFFKACRLAGFDPANIRSIPTGAETDYGLDPARLLEAMQADADAGLVPTYVCATVGTTSSNAVDPVGAVADVAARFAAWVHVDAAYAGSACICPEFRHHLDGVERVDSISMSPHKWLMTCLDCTCLYVRDTHRLTGSLETNPEYLKNHASDSGEVTDLKDMQVGVGRRFRGLKLWMVMRTYGVAKLQEHIRSDVAMAKVFEDLVRGDDRFEVVVPRNFALVCFRIRAGAGAAAATEEDADEANRELMERLNKTGKAYVAHTVVGGRFVLRFAVGSSLQEEHHVRSAWELIKKTTTEMMN

SEQ ID NO: 264 - protein, 502 aa, Camptotheca acuminata
MGSLDSNYDTESPASVGQFNPLDPEEFRKQAHCIVDFIADYYKNIESYPVLSQVDPGYRHSRLGKNAPYRSEPFESILKDVQKDIIPGMTHWMSPNFFAHFPATVSSAAFVGEMLCTCFNSVGFNWLASPAATELEMVVIDWLANMLKLPKSFMFSGTGGGVLQGTTSEAILCTLIAASPMHFEIVGVKTSTSFVVYGSDQTHSTYAKACKLAGILPCNIRSIPTTADSNFSVSPLLLRRAIEADKAAGMVPLYICATVGTTSTTAIDPLSSLADVANDYGVWFHVDAAYAGSACICPEFRHYLDGIERADSLSLSPHKWLLSYLDCCCLWVKSPSLLVKALSTDPEYLKNQPSESKSVVDYKDWQVGTGRRFKALRLWFVMRSYGVANLQSHIRTDVQMAKMFEGFVKSDPRFEILVPRVFSLVCFRLNPISGSDPTGTEALNRKLLDWVNSTGRVYMTHTKVGGIYMLRFAVGATLTEKRHVSSAWKLIKEGADVLLKED

SEQ ID NO: 265 - protein, 498 aa, Camptotheca acuminata
MGSIDSNYDTESAGQCRPLEPEEFRKQAHQMVDFIADYYKNIESYPVLSQVEPGYLQSRLPETAPYRPEPFESILKDVHKDIIPGVTHWLSPNFFAYFPATVSSAAFVGEMLCTCFNAVGFNWLASPAELELEMVVMDWLASMLKLPNSFTFLGTGGGVIQGTTSEAILCTLIAARDRALESIGVDSIHKLVVYGSDQTHSTYAKACNLAGILPCNIRSIRTEAVANFSLSPDSLHREIEADVAAGMVPLYLCATVGTTSTTAIDSLSPLADVANDYGLWFHVDAAYAGSACICPEFRHYLDGIERADSLSLSPHKWLLSYLDCCCLWVKRPSVLVKALSTDPEYLKNKPSESNSVVDFKDWQVGTGRRFKALRLWFVMRSYGVANLQSHIRSDIQMAKMFEEFVNSDPRFEIVVPRVFSLVCFRLNPFSKSDPCNTELLNRKLLEWVNSTGQVYITHTKVGGVYMLRFAVGATLTEEHHVSAAWKLIREGADALLCS

SEQ ID NO: 266 - protein, 503 aa, Capsicum annuum
MGSLDSNNSTQTQSNVTKFNPLDPEEFRTQAHQMVDFIADYYKNIESYPVLSQVEPGYLRNHLPENAPYLPESLDTIMKDVEKHIIPGMTHWLSPNFFAFFPATVSSAAFLGEMLCNCFNSVGFNWLASPAMTELEMIIMDWLANMLKLPECFMFSGTGGGVIQGTTSEAILCTLIAARDRKLENIGVDNIGKLVVYGSDQTHSMYAKACKAAGIFPCNIRAISTCVENDFSLSPAVLRGIVEVDVAAGLVPLFLCATVGTTSTTAIDPISELGELANEFDIWLHVDAAYGGSACICPEFRQYLDGIERANSFSLSPHKWLLSYLDCCCMWVKEPSVLVKALSTNPEYLRNKRSEHGSVVDYKDWQIGTGRKFKSLRLWLIMRSYGVANLQSHIRSDVRMAKMFEGLVRSDPYFEVIVPRRFSLVCFRFNPDKEYEPAYTELLNKRLLDNVNSTGRVYMTHTVAGGIYMLRFAVGATFTEDRHLICAWKLIKDCADALLRNCQ

SEQ ID NO: 267 - protein, 506 aa, Ophiorrhiza pumila
MGSISENCDDSISLAAPFRPLEPEEFRKQAHVMVDFIADYYKNIENYPVLSQVEPGYLKNRLPETAPHLPESFETILKDIKKDIVPGMTNWLSPNFFAYFPATVSSAAFVGEMLCTGFNSVGFNWLASPASTELEMVVIDWLANMLKLPKSFMFHGTGGGVIQGTTSEAILCTLIAARDGALEKIGMENVGKLVVYGSDQTHSFFQKTCKVAGIFPCNIKLIPTTREDNFSMSPIALREQIEADVADGLVPIFLCTTVGTTSTAAIDPVSEVAKVANDFNIWVHVDAAYAGSACICPEFRQYLDGIELVDSFSLSPHKWLLCFLDCCCLWLKKPHLMVKALSTNPEYLRNKRSEFDGVVDFKDWQIGTGRRFKALRLWLVMRSYGVENLKRHILSDVQMAKMFEGLVKSDPRFEIIVPRAFALVCFRLNPGKGYDDEIDKEILNKELLDLINSTGRAYMTHTKAGGIYMLRFAVGTTLTEEHHVYAAWELIKECTDASLTKTNIIE

SEQ ID NO: 268 - protein, 500 aa, Catharanthus roseus
MGSIDSTNVAMSNSPVGEFKPLEAEEFRKQAHRMVDFIADYYKNVETYPVLSEVEPGYLRKRIPETAPYLPEPLDDIMKDIQKDIIPGMTNWMSPNFYAFFPATVSSAAFLGEMLSTALNSVGFTWVSSPAATELEMIVMDWLAQILKLPKSFMFSGTGGGVIQNTTSESILCTIIAARERALEKLGPDSIGKLVCYGSDQTHTMFPKTCKLAGIYPNNIRLIPTTVETDFGISPQVLRKMVEDDVAAGYVPLFLCATLGTTSTTATDPVDSLSEIANEFGIWIHVDAAYAGSACICPEFRHYLDGIERVDSLSLSPHKWLLAYLDCTCLWVKQPHLLLRALTTNPEYLKNKQSDLDKVVDFKNWQIATGRKFRSLKLWLILRSYGVVNLQSHIRSDVAMGKMFEEWVRSDSRFEIVVPRNFSLVCFRLKPDVSSLHVEEVNKKLLDMLNSTGRVYMTHTIVGGIYMLRLAVGSSLTEEHHVRRVWDLIQKLTDDLLKEA

SEQ ID NO: 269 - protein, 514 aa, Petroselinum crispum
MGSIDNLTEKLASQFPMNTLEPEEFRRQGHMMIDFLADYYRKVENYPVRSQVSPGYLREILPESAPYNPESLETILQDVQTKIIPGITHWQSPNFFAYFPSSGSTAGFLGEMLSTGFNVVGFNWMVSPAATELENVVTDWFGKMLQLPKSFLFSGGGGGVLQGTTCEAILCTLVAARDKNLRQHGMDNIGKLVVYCSDQTHSALQKAAKIAGIDPKNFRAIETTKSSNFQLCPKRLESAILHDLQNGLIPLYLCATVGTTSSTTVDPLPALTEVAKKYDLWVHVDAAYAGSACICPEFRQYLDGVENADSFSLNAHKWFLTTLDCCCLWVRNPSALIKSLSTYPEFLKNNASETNKVVDYKDWQIMLSRRFRALKLWFVLRSYGVGQLREFIRGHVGMAKYFEGLVNMDKRFEVVAPRLFSMVCFRIKPSAMIGKNDEDEVNEINRKLLESVNDSGRIYVSHTVLGGIYVIRFAIGGTLTDINHVSAAWKVLQDHAGALLDDTFTSNKLVEVLS

SEQ ID NO: 270 - protein, 508 aa, Rosa hybrid
MGSFPFHRDLQEIASSQLTKALDPEEFRKQGHMVINFIADYYQNIEKYPVLSRVEPGYLKKCLPVSAPYDPEPISTILRDVQNHIVPGLTHWQSPNFFAYFSSTASTAGFLGEILTTGFNVVGFNWVSSPAATELENIVMDWLGDMLQLPKSFHFSGNGGGVLHGSTCEAIVCTMVAARDQMLRRIGSENLGKLVVYGSDQTHSTLQKATQIVGINTENFRAIKTTKSTGFALSPEMLRLTISSDLEKGLVPLFLCATIGTTATTAIDPLEALCHVAKEYGVWVHVDAAYAGSACICPEFRHFINGVEGANSFSFNPHKWLFTGMDCCCLWVKNPSVLASSLSTNPEFLRNKASDSKQVVDYKDWQIALSRRFRALKLWLVLRSYGVANLRNFIRIHVKMAKTFEGLVRMDKRFEILVPRNFSLVCFRISPSALISSNEDDEIGMVNEVNCKLLEAINASGKAYMTHAVVGGLYVLRCAVGATLTEEKHIVEAWNVVQDHAQAILSTY

SEQ ID NO: 271 - protein, 506 aa, Petunia hybrida; identical to SEQ ID NO: 90

SEQ ID NO: 272 - protein, 518 aa, Thalictrum flavum
MGSLHVEDLDNISKCTVENPLDPEEFRRQGHMMIDFLADYYRDIEKYPVRSQVEPGYLRKEIPDSAPYNPESIETILEDVHKQIIPGITHWQSPNYFAYFPSSGSVAGFLGEMLSTGFNVVGFNWMSSPAATELESIVMDWLGKMLKLPKSFLFSGNGGGVLQGTTCEAILCTLTAARDRMLNKIGRENICKLVVYGSDQTHCALQKAAQIAGIHPNNFRAVPTTKANDYGLSASALRSTILEDIEAGLVPLFLCATVGTTSSTAVDPIGPLCKVASDYSIWVHVDAAYAGSACICPEFRHFIDGVENADSFSLNAHKWFFTTLDCCCLWVKEPSALIKALSTNPEYLRNKATESHQVVDYKDWQIALSRRFRAMKLWLVLRSYGVANLRNFLRSHVKMAKNFEGFIALDKRFEIVVPRTFAMVCFRLLPPRSPLIIKTNGYQNGNGVYHKDESRANELNRRLLESINASGSAYMTHSMVGGVYMIRFAVGASLTEERHVILAWKVVQEHADAVLATF

SEQ ID NO: 273 - protein, 533 aa, Brachypodium distachyon
MAPTSMCFDAINGAAAAQNGTAPVLATKPAAQALQCPNALNADDFRRQGHQVIDFIAEYYGGMADYPVHPSVTPGFLRNLLPASAPSRAEPDAFSSALKDIRDHILPGMTHWQSPRHFAHFPASSSTVGALGEALTAGINVVPFTWAASPAATELEMVVVDWLGKALHLPETLLFAGGGGGTLLGTSCEAILCALVAARDRKLAEIGGRRIGDLVVYCSDQTHFAFRKAARIAGILREHIREIQTCHANMFALSATALEAAMQADVEAGLVPLFVCATVGTTQTTAVDPIGELCTVTAPHGVWVHVDAAYAGSALVCPEFRHVINGVESVDSFSMNAHKWLLTNNDCCAMWVKKPSELIAALGTEQEYILKDSASEGHDIVDYKDWTMTLTRRFRALKMWLVLRCYGIDGLREHIRSHVRMAEAFENLVRADERFEVVTDRQFALVCFRLRSPEKYGGEKTANELNRSLLEEVNAVTLGPYMSSANVGGMYMLRCAVGSTLTEDCHVTDGWKVVQDRATSILRKMEIIYSVLG

SEQ ID NO: 274 - protein, 512 aa, Papaver somniferum
MGSLPTNNLESISLCSQNPLDPDEFRRQGHMIIDFLADYYKNVENYPVRSQVEPGYLKKRLPESAPYNPESIETILEDVTNDIIPGLTHWQSPNYFAYFPSSGSIAGFLGEMLSTGFNVVGFNWMSSPAATELESIVMNWLGQMLTLPKSFLFSSDGSSGGGGVLQGTTCEAILCTLTAARDKMLNKIGRENINKLVVYASNQTHCALQKAAQIAGINPKNVRAIKTSKATNFGLSPNSLQSAILADIESGLVPLFLCATVGTTSSTAVDPIGPLCAVAKLYGIWVHIDAAYAGSACICPEFRHFIDGVEDADSFSLNAHKWFFTTLDCCCLWVKDSDSLVKALSTSAEYLKNKATESKQVIDYKDWQIALSRRFRSMKLWLVLRSYGVANLRTFLRSHVKMAKHFQGLMGMDNRFEIVVPRTFAMVCFRLKPAAIFKQKIVDNDYIEDQTNEVNAKLLESVNASGKIYMTHAVVGGVYMIRFAVGATLTEERHVTGAWKVVQEHTDAILGA

SEQ ID NO: 275 - protein, 20 aa, Rhodiola rosea
ALIESLAAEANFLKGGSEMV

SEQ ID NO: 276 - protein, 24 aa, Arabidopsis thaliana
SLTLALSTNPEFLKNKASQANLVV

SEQ ID NO: 277 - protein, 26 aa, Arabidopsis thaliana
SLIDALKTNPEYLEFKVKVSKKDTVV

SEQ ID NO: 278 - protein, 24 aa, Capsicum annuum
ALIQSLSTNPEYLKNKASQGNLVV

SEQ ID NO: 279 - protein, 24 aa, Oryza sativa
FLIQSLSTNPEFLKNKASQANSVV

SEQ ID NO: 280 - protein, 24 aa, Oryza sativa
RLTGSLETNPEYLKNHASDSGEVT

SEQ ID NO: 281 - protein, 24 aa, Camptotheca acuminata
LLVKALSTDPEYLKNQPSESKSVV

SEQ ID NO: 282 - protein, 24 aa, Camptotheca acuminata
VLVKALSTDPEYLKNKPSESNSVV

SEQ ID NO: 283 - protein, 24 aa, Capsicum annuum
VLVKALSTNPEYLRNKRSEHGSVV

SEQ ID NO: 284 - protein, 24 aa, Ophiorrhiza pumila
LMVKALSTNPEYLRNKRSEFDGVV

SEQ ID NO: 285 - protein, 24 aa, Catharanthus roseus
LLLRALTTNPEYLKNKQSDLDKVV

SEQ ID NO: 286 - protein, 24 aa, Petroselinum crispum
ALIKSLSTYPEFLKNNASETNKVV

SEQ ID NO: 287 - protein, 24 aa, Rosa hybrid
VLASSLSTNPEFLRNKASDSKQVV

SEQ ID NO: 288 - protein, 24 aa, Petunia hybrida
SLTKALSTNPEVLRNDATDSEQVV

SEQ ID NO: 289 - protein, 24 aa, Thalictrum flavum
ALIKALSTNPEYLRNKATESHQVV

SEQ ID NO: 290 - protein, 25 aa, Brachypodium distachyon
ELIAALGTEQEYILKDSASEGHDIV

SEQ ID NO: 291 - protein, 24 aa, Papaver somniferum
SLVKALSTSAEYLKNKATESKQVI